(19) United States
(12) Patent Application Publication
(10) Pub. No.: US 2025/0295494 A1
(43) Pub. Date: Sep. 25, 2025

(54) ANNULOPLASTY IMPLANTS AND SYSTEMS FOR USE THEREWITH

(71) Applicant: Edwards Lifesciences Innovation (Israel) Ltd., Caesarea (IL)

(72) Inventors: Ido Halabi, Givatayim (IL); Rohan Yogee Katbamna, Irvine, CA (US); Eva Adriana de la Cruz Romito, Irvine, CA (US); Eyal Shaolian, Newport Beach, CA (US); Alidod F. Ghazvini, Costa Mesa, CA (US); Brian Patrick Murphy, Irvine, CA (US); Charles Henry Bloodworth, IV, Irvine, CA (US); Ajay Kumar Dass, Costa Mesa, CA (US); Matthew Aaron Sussman, Laguna Hills, CA (US); Yuval Kasher, Kfar Shmuel (IL); Sambhav Jain, Furlong, PA (US); Kaamran Ahsan Syed, Irvine, CA (US); Meena Francis, Trabuco Canyon, CA (US); Itschak Biran, Irvine, CA (US); Murrad Mirza Kazalbash, Tustin, CA (US); Mauricio Guerrero, Hawthorne, CA (US); Asher L. Metchik, Rolling Hills Estates, CA (US); Tiana Tran, Westminster, CA (US); Timothy Allen Dalton, Garden Grove, CA (US); Zhicheng Fang, Irvine, CA (US); Julia Akiko Roche, Costa Mesa, CA (US); Ehud Aviv, Costa Mesa, CA (US); Willa Ni, Costa Mesa, CA (US); Rupesh Gajanan Nawalakhe, Trabuco Canyon, CA (US); Jeffrey Michael Koslosky, Coto De Caza, CA (US); Haim Brauon, Beit Dagan (IL); Jacob Timothy Morrison, Ladera Ranch, CA (US); Danny Barrientos Baldo, JR., San Diego, CA (US); Roman Plut, Rishon LeZion (IL); Aviv Galon, Tel Aviv (IL); Yaron Herman, Givat Ada (IL); Sirous Darabian, Rancho Santa Margarita, CA (US)

(21) Appl. No.: 19/231,308

(22) Filed: Jun. 6, 2025

Related U.S. Application Data
(63) Continuation of application No. PCT/IB2023/062298, filed on Dec. 6, 2023.
(60) Provisional application No. 63/386,597, filed on Dec. 8, 2022.
(60) Provisional application No. 63/580,700, filed on Sep. 5, 2023.
(60) Provisional application No. 63/598,509, filed on Nov. 13, 2023.

(51) Int. Cl. A61F 2/24

(52) U.S. Cl. CPC A61F 2/2466; A61F 2/2445; A61F 2220/0016

(57) ABSTRACT

A catheter device includes a flexible tube and an extracorporeal unit. The tube has a distal opening that is configured to be transluminally advanced into a subject, and a proximal end. The extracorporeal unit is coupled to the proximal end, and includes (i) a body, and (ii) a series of cartridges, distributed along a proximal-distal axis of the body, with a distalmost cartridge being closest to the proximal end of the tube. A series of anchors includes a leading anchor and other anchors, each anchor being (i) housed by a corresponding cartridge, with the leading anchor housed by the distalmost cartridge, and (ii) coupled to a tether such that the tether extends along the body, parallel with the proximal-distal axis. Other embodiments are also described.

### CROSS-REFERENCES TO RELATED APPLICATIONS

The present application is a Continuation of International Patent Application PCT/IB2023/062298 to Halabi et al., filed Dec. 6, 2023, and titled “Annuloplasty implants and systems for use therewith,” which published as WO 2024/121770; and which claims priority to:
Provisional U.S. Patent Application 63/386,597 to Halabi et al., filed Dec. 8, 2022, and titled “Annuloplasty implants and systems for use therewith”;
Provisional U.S. Patent Application 63/580,700 to Halabi et al., filed Sep. 5, 2023, and titled “Annuloplasty implants and systems for use therewith”; and
Provisional U.S. Patent Application 63/598,509 to Halabi et al., filed Nov. 13, 2023, and titled “Annuloplasty implants and systems for use therewith”.

Each of the above references is incorporated herein by reference in its entirety for all purposes.

### BACKGROUND

There are various potential ways of repairing a heart valve and/or other anatomy. Annuloplasty is a repair procedure that involves remodeling/reshaping tissue of an annulus. This can be done by pulling tissue about the annulus to a new shape. Anchors can be used to facilitate medical procedures including annuloplasty, other remodeling of tissues, securing implants, etc. In some instances, it may be desirable to use an anchor for a procedure in which there is no line-of-sight to the target.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Some of the systems, apparatuses, devices, methods, techniques, etc. described herein, and implementations and applications thereof, include or are configured to be used with an implant that includes multiple anchors slidably coupled to a tether (e.g., a line, wire, ribbon, rope, cable, braid, contraction member, contraction member, suture, etc.).

In some implementations, the implant can be a tissue-adjustment implant that contracts tissue upon tensioning of the tether. In some implementations, the implant can be usable and/or configured for use at a heart of a subject (e.g., a living subject, a simulation, etc.). For example, the implant can be an annuloplasty implant.

In some implementations, the implant can be configured and/or used to close another opening (e.g., an opening to an appendage, an opening to a left atrial appendage, an opening to a passageway, etc.) or reshape another region of tissue (e.g., ventricular remodeling, atrial remodeling, muscle remodeling, etc.)

In some implementations, a delivery system is provided for advancement and anchoring of the anchors (e.g., an implant that includes the anchors threaded on the tether). In some implementations, the delivery system can comprise a catheter device and/or an anchor driver.

In some implementations, the catheter device can include a tube, and an extracorporeal unit, e.g., at a proximal end of the tube. The anchors can be mounted, in a series, on the extracorporeal unit. In some implementations, the tether can be threaded through the anchors in this arrangement.

In some implementations, a series of cartridges (or anchor holders) can be mounted on the extracorporeal unit, and can hold the anchors in the series. In some implementations, one or more (e.g., one, some, or all) cartridges can be configured to facilitate bringing its corresponding anchor to a proximal opening of the tube for advancement, by the driver, through the tube to the site at which the anchor is to be anchored. In some implementations, the extracorporeal unit and/or the cartridges can be configured to facilitate verification of engagement between the driver and the anchor prior to advancement.

The term cartridge as used herein is interchangeable with the term anchor holder, the cartridges/anchor holders herein can be configured in a variety of ways (e.g., from a simple receptacle or hole for holding an anchor to much more involved or elaborate configurations and mechanisms).

In some implementations, anchors are configured (e.g., shaped) to be slidable along a tether (e.g., a line, wire, contraction member, etc.) both (i) while aligned (i.e., parallel or coaxial) with the tether, and (ii) while oriented orthogonal to the tether. This helps facilitate, inter alia, (i) advancement of the anchor along the tether while aligned with the tether during transcatheter delivery, and (ii) subsequent sliding of the tether with respect to the anchor after implantation, e.g., while the tether is orthogonal to the anchor.

In some implementations, each anchor can include (i) a tissue-engaging element, (ii) and a head at a proximal end of the tissue-engaging element.

In some implementations, each anchor of the series of anchors is configured to be the same or similar. In some implementations, some anchors are of a first type or first configuration, while one or more other anchors are of a second type or second configuration. In some implementations, each anchor is different.

In some implementations, the tissue-engaging element can be a screw-in tissue-engaging element, e.g., can be helical, screw-like, threaded, etc.

In some implementations, the tissue-engaging element can comprise one or more hooks, barbs, darts, staples, clips, protrusions, arms, expandable portions, threaded portions, rivets, pledgets, combinations of two or more of these, etc.

In some implementations, the head can comprise or define an interface via which the anchor driver can engage and apply an anchoring force (e.g., torque) to the anchor.

In some implementations, each anchor can comprise an eyelet, or another connector that defines an aperture therethrough, coupled to the head of the anchor. In some implementations, the anchor can be slidably coupled to the tether by the eyelet being threaded onto the tether. In some implementations, the eyelet can be disposed laterally from the axis of the anchor. The eyelet can be configured in a variety of different ways.

In some implementations, the eyelet is flexible in a manner that facilitates smooth sliding along the tether both (i) when the anchor is parallel with the tether and (ii) when the anchor is in an orthogonal orientation with respect to the tether.

In some implementations, the eyelet comprises and/or is formed from a textile (e.g., a polyfilament structure), such as a yarn.

In some implementations, the eyelet comprises and/or is formed from a polymer (e.g., a polymer suture, a polymer yarn, a polymer filament, etc.).

In some implementations, the eyelet is pivotable over the anchor head. In some implementations, the eyelet comprises and/or is formed from a textile that is shaped such that the eyelet is pivotable over the anchor head.

In some implementations, the eyelet is connected to two places on the collar in a manner that defines a hinge axis on which the two places lie, and the eyelet is pivotable about the hinge axis.

In some implementations, the eyelet comprises and/or is formed from a textile and/or a polymer, wherein the textile and/or polymer is also configured and/or formed to define a collar that circumscribes part of the head of the anchor and/or a stock or neck region of the head (or stock or neck region proximate the head), coupling (directly or indirectly) the eyelet to the head and/or a stock or neck region of the head (or stock or neck region proximate the head) (e.g., coupling the eyelet directly to the head, coupling the eyelet directly to a stock or neck region of and/or proximate the head, coupling the eyelet to a bushing on or associated with the head and/or a stock or neck region of and/or proximate the head).

In some implementations, the collar can be configured to facilitate the eyelet revolving around the head of the anchor, e.g., by the collar rotating about an axis of the anchor. In some implementations, this can be augmented by a bushing on which the collar can be mounted.

In some implementations, the collar that circumscribes part of the head of the anchor and/or a stock or neck region of the head (or stock or neck region proximate the head) is formed by one or more loops (e.g., 1 loop, 2 loops, 3 loops, etc.) of the textile and/or polymer that loop around the part of the head of the anchor and/or a stock or neck region of the head (or stock or neck region proximate the head). This configuration can be used whether or not looped on a bushing, e.g., a bushing around the head, stock, neck, etc.

In some implementations, the eyelet of each anchor is saddle shaped.

In some implementations, each anchor comprises a spacer, extending away from the anchor head along the tether, e.g., distally, toward the preceding anchor in the series. In some implementations, the spacer can inhibit approximation of the anchor and the preceding anchor.

In some implementations, the spacer can be coupled to the head and/or the eyelet of the anchor. In some implementations, the spacer can be integrally formed with the head and/or the eyelet of the anchor.

In some implementations, the spacer can be revolvable around the head and/or around axis of the anchor. In some implementations, the spacer can be rotatable with respect to the axis of the anchor.

In some implementations, a specialized leading anchor is provided, which is fixed to the tether but that nonetheless facilitates deflection of the tether with respect to the axis of the leading anchor. For example, the leading anchor (e.g., its head) can define a socket configured to receive a stopper (e.g., a bead) that is fixed to an end of the tether.

In some implementations, the tube of the catheter device has, at its distal end, one or more resilient ribs, nubs, or nodules that engage the tissue-engaging element of the anchor in a manner that controls advancement of the anchor distally out of the tube. For example, the ribs can allow the anchor to be advanced distally only while the tissue-engaging element is being rotated, e.g., can inhibit non-rotational axial advancement. However, the ribs can be less inhibitive of non-rotational axial retraction.

In some implementations, tensioning of the tether slides the tether through the eyelet of each anchor, drawing the anchors toward each other and thereby contracting the tissue to which the anchors are anchored, e.g., the annulus of a heart valve. In order to lock the tension into the tether, a lock (which may, in some implementations, be considered a stopper) is advanced along the tether and locked to the tether, e.g., at the most recently-anchored anchor.

In some implementations, the lock is configured such that its actuation both locks the lock to the tether and cuts (e.g., trims) the tether so that excess tether can be withdrawn.

In some implementations, the lock can be introduced onto the tether without access to either end of the tether.

In some implementations, a tensioner is provided that can engage an intermediate region of the tether (e.g., without access to either end of the tether), and apply tension to the tether from there. In some implementations, the tensioner is mounted or mountable on the extracorporeal unit of the catheter tool, which provides access to the intermediate portion of the tether.

In some implementations, the tensioner can be used to assess a mid-procedure state and/or behavior of the implant, and/or can be used to apply the tension that will be locked-in to the implant, e.g., toward the end of the procedure.

In accordance with some implementations, a system and/or an apparatus includes an implant that includes a tether and/or an anchor. In some implementations, the anchor can include an anchor head, a tissue-engaging element, and/or a textile.

In some implementations, the anchor head can include a stock. The stock can be configured in a variety of ways, e.g., as a core, a rod, a tube, a neck, a winch, a peg, etc.

In some implementations, the tissue-engaging element is coupled to the stock, extends distally away from the anchor head to define an anchor axis of the anchor, and is configured to be driven along the anchor axis into tissue of a subject (e.g., a living subject, a simulation, etc.).

In some implementations, the textile can be shaped to define a collar and/or an eyelet.

In some implementations, the textile can comprise one or more polymers.

In some implementations, the collar can circumscribe the stock. In some implementations, the collar can be looped around the stock. In some implementations, the collar can circumscribe and/or loop around the stock one or more times (e.g., 1 time, 2 times, 3 times, etc.).

In some implementations, the tether can be threaded through the eyelet.

In some implementations, the eyelet is revolvable about the anchor axis by the collar rotating about the anchor axis.

In some implementations, the textile is or comprises a fabric.

In some implementations, the fabric is a substantially flat fabric sheet.

In some implementations, the collar and the eyelet are formed by cutting the fabric sheet.

In some implementations, the textile is a fabric that is woven in a manner that integrally defines the collar and the eyelet.

In some implementations, the eyelet has an eyelet-aperture through the fabric, the collar has a collar-aperture through the fabric, and/or the fabric is woven in a manner that provides the eyelet-aperture and the collar-aperture.

In some implementations, the fabric has general warp strands and reinforcement warp strands, the reinforcement warp strands being stronger and fewer than the general warp strands, and the fabric being woven such that the reinforcement warp strands skirt the eyelet-aperture and the collar-aperture.

In some implementations, the fabric has general weft strands and reinforcement weft strands, the reinforcement weft strands being stronger and fewer than the general weft strands, and the fabric being woven such that the reinforcement weft strands skirt the eyelet-aperture and the collar-aperture.

In some implementations, the textile is further shaped to define a spacer through which the tether is threaded, the spacer inhibiting approximation between the anchor and an adjacent anchor of the series.

In some implementations, the textile includes a textile tube that is (i) wrapped around the anchor head in a manner that defines the collar, (ii) formed into a loop in a manner that defines the eyelet, and (iii) threaded coaxially onto the tether in a manner that defines the spacer.

In some implementations, the implant is sterilized.

In some implementations, the textile includes filaments of a synthetic polymer.

In some implementations, the textile includes filaments of a natural fiber.

In some implementations, the anchor head includes an interface that is coupled to the tissue-engaging element via the stock, the tissue-engaging element being configured to be driven along the anchor axis into the tissue by an anchoring force applied to the interface.

In some implementations, the collar and the eyelet are formed integrally during formation of the textile.

In some implementations, the eyelet is slidable along the tether.

In some implementations, the anchor is a second anchor, the implant further including a leading anchor coupled to the tether.

In some implementations, the textile is a yarn.

In some implementations, the collar and the eyelet are defined by respective loops of the textile and/or yarn. In some implementations, one or more of the collar and/or the eyelet are defined by multiple loops of the textile and/or yarn.

In some implementations, the collar and the eyelet are formed integrally during formation of the yarn.

In some implementations, the collar and the eyelet are formed by knotting the yarn.

In some implementations, the collar is formed by securing the yarn into a loop. In some implementations, the collar is formed by securing the yarn into multiple loops.

In some implementations, the eyelet is formed by securing the yarn into a loop. In some implementations, the eyelet is formed by securing the yarn into multiple loops.

In some implementations, the anchor further includes a bushing that is disposed concentrically between the eyelet and the stock.

In some implementations, the bushing is rotatable about the stock.

In some implementations, the bushing is annular.

In some implementations, the textile defines (e.g., the polyfilament structure includes) a knot, and the bushing defines a recess that is shaped to receive the knot.

In some implementations, the recess is defined by a cropped part of the bushing, the cropped part having a reduced radius from the anchor axis.

In some implementations, the recess faces laterally away from the anchor axis.

In some implementations, the recess is a cubby defined by a bulge of the bushing, the bulge bulging laterally.

In some implementations, the recess faces medially toward the anchor axis.

In some implementations, the bushing defines a radially-facing groove, the eyelet residing in the groove.

In some implementations, the bushing is shaped such that part of the groove is covered in a manner that secures the eyelet in the groove.

In accordance with some implementations, a system useable and/or for use with a tissue of a subject (e.g., a living subject, a simulation, etc.) can include a catheter device, a tether, and/or a series of anchors.

In some implementations, the catheter device can include a flexible tube that has a distal opening that is configured to be transluminally advanced toward the tissue, and/or a proximal end that defines a proximal opening. Alternatively or additionally, the catheter device can include an extracorporeal unit, coupled to the proximal end of the tube.

In some implementations, the catheter device can include a body, and/or a series of cartridges (or anchor holders). In some implementations, the series of cartridges/anchor holders are distributed along the body in a manner that defines a proximal-distal axis. In some implementations, a distalmost cartridge of the series of cartridges can be the anchor disposed closest to the proximal opening. In some implementations, the series of cartridges/anchor holders are distributed along the body in a manner that defines a proximal-distal axis with one row or column of cartridges/anchor holders along the axis, while other arrangements have two or more parallel rows or columns of cartridges/anchor holders. In some implementations, the series of cartridges/anchor holders are distributed along the body in a manner that does not define a proximal-distal axis (e.g., in a curved manner, in an angled matter, in a zig zag manner, etc.)

In some implementations, each anchor of the series of anchors can be housed by a corresponding cartridge/anchor holder of the series of cartridges/anchor holders.

In some implementations, the series of anchors includes a leading anchor and other anchors. In some implementations, the leading anchor can be housed by the distalmost cartridge.

In some implementations, the series of anchors can be coupled to the tether such that the tether extends along the body, parallel with the proximal-distal axis.

In some implementations, the cartridges of the series of cartridges are imbricated.

In some implementations, the anchors of the series of anchors are imbricated.

In some implementations, the system is sterilized.

In some implementations, the catheter device is sterilized.

In some implementations, the flexible tube is flared toward the distal opening.

In some implementations, at least some of the anchors of the series each include an anchor head and/or a tissue-engaging element that extends distally away from the anchor head to define an anchor axis of the anchor. In some implementations, the tissue-engaging element can be configured to be driven along the anchor axis into tissue of a subject (e.g., a living subject, a simulation, etc.).

In some implementations, at least some of the anchors of the series each include a textile and/or polymer, shaped to define an eyelet threaded onto the tether in a manner that slidably couples the anchor to the tether. The textile and/or polymer can be the same as or similar to other textiles and/or polymers described anywhere herein. The eyelet can be the same as or similar to other eyelets described anywhere herein.

In some implementations the anchor includes an anchor head; and/or a helical tissue-engaging element, extending away from the anchor head to define an anchor axis of the anchor, and configured to be screwed along the anchor axis into tissue of a subject (e.g., a living subject, a simulation, etc.).

In some implementations, the tube can have a distal portion that includes the distal opening. The tube can define, along a tube axis of the tube, a channel through which the anchor is slidable toward the distal opening.

In some implementations, the tube can alternatively or additionally define, at the distal portion, a grip zone at which the tube has a grip surface that inhibits sliding of the anchor through the grip zone by gripping a lateral surface of the helical tissue-engaging element. In some implementations, the system further includes an anchor driver configured to slide the anchor distally through the channel to the grip zone, and/or to drive the anchor through the grip zone by screwing the tissue-engaging element over the grip surface.

In some implementations, the grip surface is configured such that, as the driver screws the tissue-engaging element over the grip surface, the tissue-engaging element temporarily compresses parts of the grip surface with which the tissue-engaging element is in contact.

In some implementations, the grip surface comprises and/or is formed from a polymer.

In some implementations, the tube is lined with the polymer.

In some implementations, the tube comprises and/or is formed from the polymer.

In some implementations, the polymer is a thermoplastic elastomer.

In some implementations, the polymer is a block copolymer.

In some implementations, the block copolymer is polyether block amide.

In some implementations, the grip surface is provided by at least one resilient nub that protrudes medially into the channel.

In some implementations, the grip surface is provided by at least one resilient rib that protrudes medially into the channel.

In some implementations, the rib extends medially into the channel in a manner that defines, adjacent the rib, a niche in the grip zone. In some implementations, the system is configured such that, while the anchor driver screws the tissue-engaging element over the grip surface, the rib excludes the tissue-engaging element from the niche, and/or the tether extends through the grip zone sheltered within the niche, laterally from the tissue-engaging element.

In some implementations, the anchor further includes an eyelet, mounted on the head so as to be revolvable about the anchor axis. The eyelet can be the same as or similar to other eyelets described anywhere herein.

In some implementations, proximal from the rib, the tube can further define an abutment that protrudes medially into the channel in a manner that, as the anchor driver screws the tissue-engaging element over the grip surface, inhibits revolution of the eyelet about the anchor axis.

In some implementations, a unitary structure defines both the rib and the abutment.

In some implementations, the rib protrudes further medially into the channel than the abutment.

In some implementations, the abutment is longer, along the channel, than the rib.

In some implementations, the rib has a proximal face that is shaped to define a shoulder.

In some implementations, the rib has a distal face that is tapered.

In some implementations, the rib is a first of multiple ribs defined by the distal portion in the grip zone.

In some implementations, the multiple ribs are exactly two ribs.

In some implementations, the multiple ribs are exactly three ribs.

In some implementations, the multiple ribs are exactly four ribs.

In some implementations, the multiple ribs are exactly five ribs.

In some implementations, the multiple ribs are exactly six ribs.

In some implementations, the multiple ribs are distributed circumferentially around the tube axis.

In some implementations, the multiple ribs are distributed along the tube axis.

In some implementations, the rib extends around at least part of the tube axis.

In some implementations, the rib is toroidal, extending circumferentially around the entire tube axis.

35. The system according claim a9a, wherein the rib extends alongside the tube axis.

In some implementations, the rib is parallel with the tube axis.

In some implementations, the system further includes multiple tubular spacers threaded on the tether, each of the spacers connected via a corresponding connector to a corresponding anchor of the series.

In some implementations, each of the spacers is arranged on the tether such that, upon advancement of the corresponding anchor distally along the tether toward the proximal opening, the spacer trails the anchor.

In some implementations, a first of the spacers is connected to the leading anchor, and is less axially compressible than at least another of the spacers.

In some implementations, each of the anchors includes an anchor head and/or a tissue-engaging element that extends distally away from the anchor head to define an anchor axis of the anchor. The tissue-engaging element can be configured to be driven along the anchor axis into tissue of a subject (e.g., a living subject, a simulation, etc.).

In some implementations, at least some of the anchors of the series each include a textile and/or polymer, shaped to define an eyelet threaded onto the tether in a manner that slidably couples the anchor to the tether. The textile and/or polymer can be the same as or similar to other textiles and/or polymers described anywhere herein. The eyelet can be the same as or similar to other eyelets described anywhere herein.

In some implementations, the system further includes multiple tubular spacers threaded on the tether, each of the spacers connected via a corresponding connector to a corresponding anchor of the series.

In some implementations, each of the connectors is defined by the textile and/or polymer of the corresponding anchor.

In some implementations, each of the spacers is arranged on the tether such that, upon advancement of the corresponding anchor distally along the tether toward the proximal opening, the spacer trails the anchor.

In some implementations, the anchor head includes an interface that is coupled to the tissue-engaging element, the tissue-engaging element being configured to be driven along the anchor axis into the tissue by an anchoring force applied to the interface.

In some implementations, the textile and/or polymer is configured or shaped such that the eyelet is pivotable over the anchor head.

In some implementations, the textile is or comprises a fabric.

In some implementations, the textile is or comprises a polymer.

In some implementations, the textile includes filaments of a synthetic polymer.

In some implementations, the textile includes filaments of a natural fiber.

In some implementations, the textile is a yarn.

In some implementations, the eyelet is formed by securing the textile and/or yarn into a loop. In some implementations, the eyelet is formed by securing the textile and/or yarn into multiple loops.

In some implementations, the textile and/or polymer is further shaped to define a collar that couples the eyelet to the anchor head. In some implementations, the textile and/or polymer is configured as one or more loops (e.g., 1 loop, 2 loops, 3 loops, etc.) forming the collar.

In some implementations, the textile is a yarn.

In some implementations, the collar and the eyelet are defined by respective loops of the yarn.

In some implementations, the collar and the eyelet are formed integrally during formation of the yarn.

In some implementations, the collar and the eyelet are formed by knotting the yarn.

In some implementations, the collar is formed by securing the yarn into a loop.

In some implementations, the eyelet is formed by securing the yarn into a loop.

In some implementations, the collar and the eyelet are formed integrally during formation of the textile.

In some implementations, the eyelet is revolvable about the anchor axis by the collar rotating about the anchor axis.

In some implementations, the eyelet is connected to two places on the collar in a manner that defines a hinge axis on which the two places lie, and the eyelet is pivotable about the hinge axis.

In some implementations, the catheter device further includes a de-slacker, coupled to the tether, and configured to eliminate slack in the tether.

In some implementations, the de-slacker is disposed at a proximal part of the extracorporeal unit.

In some implementations, the extracorporeal unit defines an aperture, the tether extending from the de-slacker, through the aperture and along the body.

In some implementations, the aperture faces along the series of cartridges to the proximal opening.

In some implementations, the aperture is aligned with the proximal opening.

In some implementations, the system further includes an anchor driver. In some implementations, the anchor driver can include a flexible shaft, and a drive head at a distal end of the shaft. In some implementations, the anchor driver is configured to, for each of the anchors sequentially, beginning with the leading anchor: (i) engage the drive head with the anchor, (ii) remove the anchor from the corresponding cartridge, and/or (iii) while the anchor remains coupled to the tether, advance the anchor into the proximal opening and through the tube toward the tissue, and anchor the anchor to the tissue.

In some implementations, the extracorporeal unit is configured such that, for each of the anchors, removal, by the anchor driver, of the anchor from the corresponding cartridge moves the anchor away from the proximal-distal axis.

In some implementations, the extracorporeal unit is configured such that, for each of the anchors, removal, by the anchor driver, of the anchor from the corresponding cartridge pulls part of the tether away from the proximal-distal axis.

In some implementations, the tether extends along the body in a manner that defines a tether axis that is parallel with the proximal-distal axis, and the extracorporeal unit is configured such that, for each of the anchors, removal, by the anchor driver, of the anchor from the corresponding cartridge pulls part of the tether away from the tether axis.

In some implementations, the tether extends along the body such that, along the body, the tether is straight, and the extracorporeal unit is configured such that, for each of the anchors, removal, by the anchor driver, of the anchor from the corresponding cartridge reshapes part of the tether away from being straight.

In some implementations, the tether extends along the body such that, along the body, the tether is straight, and the extracorporeal unit is configured such that, for each of the other anchors, removal, by the anchor driver, of the other anchor from the corresponding cartridge forms part of the tether into a V-shape.

In some implementations, the extracorporeal unit is shaped to define a rest in which the shaft is restable while the anchor driver anchors the anchor to the tissue.

In some implementations, the rest is positioned proximally from the series of cartridges.

In some implementations, the rest is shaped and positioned such that, while the anchor driver anchors the anchor to the tissue and the shaft is resting in the rest, at least a portion of the shaft extends along the extracorporeal unit alongside the tether.

In some implementations, the rest is shaped and positioned such that, while the anchor driver anchors the anchor to the tissue and the shaft is resting in the rest, at least a portion of the shaft extends along the extracorporeal unit alongside the proximal-distal axis.

In some implementations, one or more (e.g., one, some, or all) of the cartridges is shaped to define a window, the drive head being advanceable through the window to engage, inside the cartridge, the anchor housed by the cartridge.

In some implementations, the window has a beveled rim that facilitates translational alignment of the drive head with the anchor.

In some implementations, the window is shaped to allow the drive head to reach the anchor housed by the cartridge only when the drive head is rotationally aligned with the anchor.

In some implementations, for each of the cartridges, the cartridge includes a chassis and a tray. In some implementations, the cartridge can have a closed state in which the cartridge securely houses the corresponding anchor, with the corresponding anchor seated in the tray. In some implementations, the anchor driver is configured to remove the corresponding anchor from the cartridge by applying a pulling force to the anchor such that the cartridge transitions into an open state by the tray sliding with respect to the chassis in a manner that exposes the corresponding anchor from the cartridge.

In some implementations (i) the system includes multiple spacers threaded on the tether alternatingly with the anchors of the series such that each of the spacers is disposed adjacent to a corresponding cartridge of the series, and/or (ii) for each of the cartridges (a) the tray is shaped to define a catch that, in the closed state of the cartridge, obstructs the corresponding spacer from sliding distally away from the cartridge, and/or (b) transitioning of the cartridge into the open state displaces the catch so that the catch ceases to obstruct the corresponding spacer from sliding distally away from the corresponding cartridge.

In some implementations, the window is defined at least partly by the chassis and at least partly by the tray.

In some implementations, the cartridge defines a threshold force, and is configured to transition into the open state only upon the pulling force exceeding the threshold force.

In some implementations, the cartridge is configured to resist returning from the open state into the closed state.

In some implementations, the tether has (i) a distal end at the leading anchor, and (ii) a proximal end secured within the extracorporeal unit, and releasable from within the extracorporeal unit so as to be threadable proximally into an aperture of the lock, through the lock and the head, and into the shaft of the adjustment tool.

In some implementations, the adjustment tool includes an uptake assembly that includes: (i) at a working end of the uptake assembly, a gripper disposed proximally from the lock such that, in a receiving state of the uptake assembly, threading of the proximal end of the tether proximally into the aperture of the lock, through the lock and the head, and into the shaft of the adjustment tool, causes the working end of the uptake assembly to receive the proximal end of the tether; and/or (ii) a knob, mounted on a body of the adjustment-tool extracorporeal unit, and/or operably coupled to a proximal part of the gripper such that operation of the knob transitions the uptake assembly into a grip state in which the gripper grips the tether.

In some implementations, the mounting of the knob on the adjustment-tool extracorporeal unit can be such that transitioning of the uptake assembly into the grip state releases the knob from the adjustment-tool extracorporeal unit. In some implementations, once released from the adjustment-tool extracorporeal unit, the knob can be removed from the adjustment-tool extracorporeal unit in a manner that withdraws the working end of the uptake assembly, along with the proximal end of the tether, proximally through and out from the shaft and the adjustment-tool extracorporeal unit such that the tether becomes positioned through the lock, the head, the shaft, and the adjustment-tool extracorporeal unit.

In some implementations, the lock is biased to lock, the adjustment tool includes an obstructor tube extending distally through the shaft and into the head such that a distal part of the obstructor tube is disposed within the lock in a manner that constrains the lock unlocked. In some implementations, while the knob of the uptake assembly remains mounted on the adjustment-tool extracorporeal unit, the working end of the uptake assembly can be disposed within the obstructor tube, such that removal of the knob from the adjustment-tool extracorporeal unit withdraws the working end of the uptake assembly, along with the proximal end of the tether, proximally through and out from the obstructor tube such that the tether becomes positioned through the lock, the head, the obstructor tube within the shaft, and the adjustment-tool extracorporeal unit.

In some implementations, the lock is biased to lock. In some implementations, the adjustment tool includes a guillotine (or cutting tool) within the tool head and proximal from the lock, and/or an obstructor extending distally through the shaft and the guillotine such that a distal part of the obstructor is disposed within the lock in a manner that constrains the lock unlocked.

In some implementations, the adjustment-tool extracorporeal unit includes a lock-and-cut subassembly that includes a locking block, coupled to the obstructor, and/or a lock-and-cut controller.

In some implementations, withdrawal of the working end of the uptake assembly along with the proximal end of the tether, proximally through and out from the shaft and the adjustment-tool extracorporeal unit, leaves the tether positioned through the lock and the guillotine (or cutting tool) such that (a) subsequent locking of the lock locks the lock to the tether, and (b) subsequent actuation of the guillotine (or cutting tool) cuts the tether proximally from the lock.

In some implementations, the lock-and-cut controller is operatively coupled to the locking block such that operation of the lock-and-cut controller draws the locking block proximally such that the obstructor becomes withdrawn from the lock and the lock responsively locks to the tether.

In some implementations, the lock includes a latch that, via engagement with the tool head, retains the lock within the tool head. In some implementations, the obstructor and the lock can be configured such that (i) while the distal part of the obstructor is disposed within the lock in a manner that constrains the lock unlocked, the distal part of the obstructor also obstructs the latch from disengaging from the tool head, and/or (ii) upon withdrawal of the obstructor from the lock, the lock becomes deployable from the tool head.

In some implementations the shaft of the adjustment tool is a primary shaft, and/or the adjustment tool further includes a cutter shaft that extends from the adjustment-tool extracorporeal unit through the primary shaft to the guillotine (or cutting tool).

In some implementations, I lock-and-cut subassembly can further include an adapter, coupled to the cutter shaft, and shaped and positioned with respect to the locking block such that (i) operation of the lock-and-cut controller to by a first amount draws the locking block proximally such that the obstructor becomes withdrawn from the lock and the lock responsively locks to the tether while the guillotine remains unactuated, and/or (ii) further operation of the lock-and-cut controller, beyond the first amount, engages the locking block with the adapter such that, via the locking block, the adapter, and the cutting shaft, the further operation of the lock-and-cutter controller actuates the guillotine (or cutting tool).

In some implementations, the cutter shaft is coupled to the guillotine (or cutting tool) via a swivel connector.

In some implementations, the adjustment-tool extracorporeal unit includes a tensioning subassembly that includes a tensioning block; a clamp, attached to the tensioning block; and/or a tensioning controller. In some implementations, while the knob of the uptake assembly remains mounted on the adjustment-tool extracorporeal unit, the gripper can extend from the knob, distally through the clamp and the shaft to the working end.

In some implementations, withdrawal of the working end of the uptake assembly, along with the proximal end of the tether, proximally through and out from the shaft and the adjustment-tool extracorporeal unit can withdraw the gripper from the clamp, leaving the tether positioned through the clamp such that subsequent operation of the clamp locks the tether to the tensioning block.

In some implementations, the tensioning controller can be operatively coupled to the tensioning block such that, while the tether remains locked to the tensioning block, operation of the tensioning controller applies tension to the tether by drawing the tensioning block and the tether proximally.

In some implementations, the adjustment-tool extracorporeal unit includes a distance indicator by which a position of the adjustment block with respect to the body of the adjustment-tool extracorporeal unit indicates a distance by which operation of the tensioning controller has drawn the tensioning block proximally.

In some implementations, the tensioning subassembly further includes a spring; a stock, driven by the tensioning controller such that operation of the tensioning controller causes the stock to push, via the spring, the adjustment block proximally; and/or a tension indicator by which a position of the adjustment block with respect to the stock indicates a magnitude of the tension that operation of the tensioning controller has applied to the tether.

In some implementations, the adjustment tool includes an uptake assembly that includes a sleeve extending distally through the shaft and terminating proximally from the lock; a gripper extending distally through the sleeve and having a widened distal portion disposed distally outside of the sleeve, the sleeve and the gripper being shaped and positioned such that threading of the proximal end of the tether proximally into the distal-facing aperture of the lock, through the lock and the head, and into the shaft of the adjustment tool advances the proximal end of the tether proximally around the widened distal portion of the gripper and into the sleeve; and/or a knob.

In some implementations, the knob can be mounted on the adjustment-tool extracorporeal unit, and/or operably coupled to a proximal part of the sleeve and to a proximal part of the gripper such that operation of the knob grips the tether within the sleeve by transitioning the uptake assembly into a grip state by drawing the widened distal portion of the gripper proximally into the sleeve.

In some implementations, the mounting of the knob on the adjustment-tool extracorporeal unit can be such that transitioning of the uptake assembly into the grip state releases the knob from the adjustment-tool extracorporeal unit.

In some implementations, once released from the adjustment-tool extracorporeal unit, the knob can be removable from the adjustment-tool extracorporeal unit in a manner that pulls the sleeve and the gripper, along with the proximal end of the tether, proximally through the shaft and the adjustment-tool extracorporeal unit, and out of the adjustment tool such that the tether extends through the lock, the head, the shaft, and the adjustment-tool extracorporeal unit.

In some implementations, the adjustment tool includes an obstructor tube disposed within the lock. The lock can include (i) a housing, shaped to define a distal-facing aperture via which the tether is insertable through the lock and into the obstructor tube, and/or (ii) a spring-loaded clamp, disposed within the housing, and biased to clamp onto the tether within the lock, the presence of the obstructor tube within the lock obstructing the clamp from clamping onto the tether within the lock.

In some implementations, the lock further includes a tubular leader that extends, from the distal-facing aperture, away from the body, the tether being insertable through the aperture via the leader.

In some implementations, the leader includes a helical coil.

In some implementations, the leader includes a protuberant and smooth rim.

In some implementations, the leader has a flared distal end.

In some implementations, the leader includes a sleeve.

In some implementations, the leader is rigid.

In some implementations, the leader is flexible.

In some implementations, the leader can comprise and/or be formed from a metal.

In some implementations, the leader can comprise and/or be formed from a polymer.

In some implementations, each anchor of the series includes a head, slidably coupled to the tether, and/or a tissue-engaging element, extending away from the head to define an anchor axis of the anchor. In some implementations, each anchor is housed by its corresponding cartridge such that the anchor axis lies obliquely with respect to the proximal-distal axis.

In some implementations, for each anchor of the series of anchors, the anchor is oriented with the head proximal from the tissue-engaging element.

In some implementations, for each anchor of the series of anchors, the anchor is oriented with the tissue-engaging element closer than the head to the proximal opening.

In some implementations, the anchor axes of the series of anchors collectively define a common anchor plane on which the anchor axes lie.

In some implementations, the proximal-distal axis is parallel with the common anchor plane.

In some implementations, the proximal-distal axis lies on the common anchor plane.

In some implementations, the tether extends, along the extracorporeal body, parallel with the common anchor plane.

In some implementations, one or more (e.g., one, some, or all) of the cartridges has a closed state in which the cartridge securely houses the corresponding anchor. In some implementations, one or more (e.g., one, some, or all) of the cartridges can define a respective cartridge vector that is oblique with respect to the proximal-distal axis. In some implementations, one or more (e.g., one, some, or all) of the cartridges is, by at least part of the cartridge being slid along the cartridge vector, transitionable into an open state in which the corresponding anchor is removable from the cartridge.

In some implementations, the cartridge defines a threshold force, and is configured to transition into the open state only upon the pulling force exceeding the threshold force.

In some implementations, the cartridge is configured to resist returning from the open state into the closed state.

In some implementations, the cartridge vector is oblique with respect to the proximal-distal axis.

In some implementations, the cartridge vectors of the series of cartridges collectively define a common cartridge plane on which the cartridge vectors lie.

In some implementations, the proximal-distal axis is parallel with the common cartridge plane.

In some implementations, the proximal-distal axis lies on the common cartridge plane.

In some implementations, the tether extends along the body, parallel with the common cartridge plane.

In accordance with some implementations, a system and/or an apparatus including an implant that includes a tether, a first anchor, and/or a second anchor.

In some implementations, the first anchor can be coupled to the tether and/or configured to anchor the tether to tissue of a subject (e.g., a living subject, a simulation, etc.).

In some implementations, the second anchor can be coupled to the tether. In some implementations, the second anchor can include an anchor head, a tissue-engaging element, and/or a spacer.

In some implementations, the anchor head can include an interface.

In some implementations, the tissue-engaging element extends distally away from the anchor head to define an anchor axis of the anchor, the tissue-engaging element being configured to be driven along the anchor axis into tissue of a subject (e.g., a living subject, a simulation, etc.) by an anchoring force applied to the interface.

In some implementations, the spacer can extend away from the anchor head along the tether toward the first anchor in a manner that inhibits approximation of the second anchor and the first anchor.

In some implementations, the implant is sterilized.

In some implementations, the spacer is axially compressible.

In some implementations, the spacer is longer than the tissue-engaging element.

In some implementations, the second anchor is fixedly coupled to the tether.

In some implementations, the second anchor is slidably coupled to the tether.

In some implementations, the implant is configured such that, once the first anchor has anchored the tether to the tissue, the second anchor is advanceable along the tether toward the first anchor while the spacer faces toward the first anchor.

In some implementations, the implant is configured such that, once the tissue-engaging element has been driven into the tissue, the first anchor is advanceable along the tether toward the second anchor while the spacer faces toward the first anchor.

In some implementations, the tether has a distal end, and the spacer extends away from the anchor head distally along the tether.

In some implementations, the tether has a distal end, and the spacer extends away from the anchor head proximally along the tether.

In some implementations, the interface is disposed on the anchor axis.

In some implementations, the tissue-engaging element is helical, defines the anchor axis by extending in a helix around and along the anchor axis, and is configured to be screwed into the tissue of the subject.

In some implementations, the spacer resists axial compression.

In some implementations, the spacer is mounted to be revolvable around the anchor axis.

In some implementations, the anchor includes a collar that circumscribes the anchor axis. In some implementations, the spacer is coupled to the collar.

In some implementations, the spacer is mounted to be revolvable around the anchor axis by rotation of the collar about the anchor axis.

In some implementations, the anchor head includes a stock that fixedly couples the interface to the tissue-engaging element. In some implementations, the collar circumscribes, and is rotatable about, the stock. The stock can be configured in a variety of ways, e.g., as a core, a rod, a tube, a neck, a winch, a peg, etc.

In some implementations, the second anchor is slidable along the tether while in a delivery state in which the spacer extends away from the anchor head alongside the tissue-engaging element.

In some implementations, at a distal end of the tissue-engaging element, the tissue-engaging element has a sharp point, and/or in the delivery state, the spacer extends beyond the sharp point.

In some implementations, the tissue-engaging element can comprise one or more hooks, barbs, darts, staples, clips, protrusions, arms, expandable portions, threaded portions, rivets, pledgets, helixes, screws, screw-like portions, combinations of two or more of these, etc.

In some implementations, the spacer is pivotable, from the delivery state, to become substantially orthogonal to the tissue-engaging element.

In some implementations, the spacer is flexible in deflection.

In some implementations, the spacer is resilient.

In some implementations, the spacer is tubular.

In some implementations, the spacer is defined by a helical wire shaped as a coil.

In some implementations, for at least some of the spacers, the spacer includes a coil that is substantially helical.

In some implementations, for at least some of the spacers, the coil can comprise and/or be formed from a wire that is bent to define the coil such that a transverse cross-section through a turn of the coil is substantially elliptical.

In some implementations, for at least some of the spacers, the coil can comprise and/or be formed from a tube that is cut to define the coil such that a transverse cross-section through a turn of the coil is substantially quadrilateral.

In some implementations, for at least some of the spacers, the spacer has a first end, a second end, and a middle therebetween, the first end and the second end being narrower than the middle.

In some implementations, for at least some of the spacers, the spacer is tapered from its middle towards its ends.

In some implementations, the taper is sufficiently shallow to prevent adjacent turns of the coil from axially overlapping upon axial compression of the spacer.

In some implementations, the coil can comprise and/or be formed from a metal.

In some implementations, the coil can comprise and/or be formed from a polymer.

In some implementations, the anchor further includes a snood disposed around the anchor head in a manner that preserves accessibility to the interface.

In some implementations, the snood is absorbent.

In some implementations, the snood includes a sponge.

In some implementations, the snood includes a multilaminar material.

In some implementations, the snood includes cellulose sheets.

In some implementations, the snood is impregnated with a substance and is configured to progressively release the substance within the subject.

In some implementations, the substance includes a medicament.

In some implementations, the substance includes a radiopaque dye.

In some implementations, the system/apparatus further includes a delivery tool that includes an anchor driver and a percutaneously-advanceable tube, anchor driver configured to engage the interface, to advance the second anchor through the tube, and to drive the anchor into the tissue by applying the anchoring force to the interface.

In some implementations, the anchoring force includes torque, and the anchor driver is configured to drive the anchor into the tissue by applying the torque to the interface.

In some implementations, the tube defines an internal channel that has a keyhole-shaped orthogonal cross-section that defines a major channel-region and a minor channel-region. In some implementations, the major channel-region has a larger cross-sectional area than does the minor channel-region. In some implementations, the anchor driver is configured to advance the second anchor through the internal channel with the anchor head sliding snugly through the major channel-region, and the spacer sliding snugly through the minor channel-region.

In some implementations, the spacer is configured to restrain the tether within the minor channel-region as the second anchor is advanced by the anchor driver through the internal channel.

In accordance with some implementations, a system and/or an apparatus (e.g., for use with tissue, for use with tissue of a subject, for repair, etc.) includes an implant, an anchor driver, and/or an adjustment tool.

In some implementations, the implant can include a radiopaque tether, biased toward assuming a regular wavy shape.

In some implementations, the implant can include multiple anchors, each including an anchor head and/or a tissue-engaging element extending distally from the anchor head. The head and/or tissue-engaging element can be configured to be the same as or similar to other heads and/or tissue-engaging elements herein respectively.

In some implementations, the anchor driver can be configured to anchor the multiple anchors to the tissue (i) by, for each of the multiple anchors, via engagement with the anchor head, driving the tissue-engaging element into the tissue, and/or (ii) such that the anchor head of each of the multiple anchors is threaded onto the tether, with the tether assuming its regular wavy shape.

In some implementations, the adjustment tool can be configured to apply tension to the tether in a manner that straightens the tether and draws the multiple anchors toward each other, and/or lock the tension in the tether.

In some implementations, the implant is sterilized.

In some implementations, the anchor driver is sterilized.

In some implementations, the adjustment tool is sterilized.

In some implementations, the tether includes a drawn filled tube that has a radiopaque core.

In some implementations, the tether includes a cable that includes a radiopaque strand and a shape memory strand.

In some implementations, the tether includes a shape memory alloy, and is shape-set to the regular wavy shape.

In some implementations, the regular wavy shape is sinusoidal, and the tether is biased toward assuming the sinusoidal shape.

In some implementations, the regular wavy shape is a zigzag, and the tether is biased toward assuming the zigzag shape.

In accordance with some implementations, a system and/or an apparatus includes an implant, the implant including an anchor, a tether, and/or a stopper.

In some implementations, the anchor can include an anchor head, and/or a tissue-engaging element extending distally from the anchor head. The head and/or tissue-engaging element can be configured to be the same as or similar to other heads and/or tissue-engaging elements herein respectively.

In some implementations, the anchor head can include a socket.

In some implementations, the stopper can be attached to an end of the tether, and secured within the socket in a manner that couples the anchor to the end of the tether.

In some implementations, the implant is sterilized.

In some implementations, the stopper is bulbous.

In some implementations, the stopper is substantially spherical.

In some implementations, the anchor is a leading anchor, and the implant further includes one or more successive anchors.

In some implementations, the stopper is rotatable within the socket.

In some implementations, the stopper is snap-fitted into the socket.

In some implementations, the stopper is a bead.

In some implementations, the stopper is attached to the end of the tether by crimping.

In some implementations, the stopper is attached to the end of the tether by welding.

In some implementations, the stopper is attached to the end of the tether by brazing.

In some implementations, the anchor is a fixed anchor, and the implant further includes one or more sliding anchors, each of the sliding anchors being slidably coupled to the tether.

In some implementations, each of the one or more sliding anchors includes an eyelet, and is slidably coupled to the tether by the eyelet being threaded onto the tether. The eyelet(s) can be the same as or similar to other eyelets described anywhere herein.

In some implementations, the anchor head includes a casing that defines the socket. In some implementations, the casing further defines a window into the socket. In some implementations, the tether extends, from the stopper, through the window to exit the socket.

In some implementations, across from the window, the socket has an open side.

In some implementations, the casing includes a cantilever that obstructs the stopper from exiting the socket via the open side.

In some implementations, the window extends at least a fifth of the way around the stopper.

In some implementations, the window curves in an arc around the socket.

In some implementations, the window is sized, and the socket is configured, to allow the tether to pivot with respect to the anchor head via rotation of the stopper within the socket.

In some implementations, the window is sized, and the socket is configured, to allow pivoting of the tether with respect to the anchor head to rotate the stopper within the socket.

In some implementations, the tissue-engaging element extends distally from the anchor head to define an anchor axis along which the tissue-engaging element is advanceable into tissue of a subject (e.g., a living subject, a simulation, etc.).

In some implementations, the window is shaped to allow the tether to pivot between: (i) an axial state in which the tether extends through the window in a trajectory that is parallel with the anchor axis, and/or (ii) a lateral state in which the tether extends through the window in a trajectory that is orthogonal with the anchor axis.

In some implementations, the end of the tether does not protrude from the stopper.

In some implementations, the end of the tether is flush with an external surface of the stopper.

In some implementations, the end of the tether is disposed within the stopper.

In accordance with some implementations, a system useable and/or for use with a heart of a subject (e.g., a living subject, a simulation, etc.), the system includes an implant and a delivery tool.

In some implementations, the implant can include an anchor that has a head, and a helical tissue-engaging element extending distally away from the head to define an anchor axis of the anchor. In some implementations, the head can define an interface of the anchor.

In some implementations, the delivery tool can include a catheter device and/or an anchor driver.

In some implementations, the catheter device can include an extracorporeal portion at a proximal part of the catheter device, and/or a flexible tube extending distally from the extracorporeal portion.

In some implementations, the flexible tube can have a distal portion that is configured to be transluminally advanced to the heart. In some implementations, the flexible tube can have a distal portion that has a distal opening. In some implementations, the flexible tube can have a distal portion that defines, along a tube axis of the tube, a channel through which the anchor is slidable toward the distal opening.

In some implementations, the flexible tube can have a distal portion that defines, proximal from the distal opening, a grip zone at which the distal portion has resilient rib that extends medially into the channel in a manner that inhibits sliding of the anchor through the grip zone by gripping the helical tissue-engaging element.

In some implementations, the anchor driver can be configured, via engagement with the interface, to slide the anchor distally through the channel toward the grip zone, and/or to drive the anchor through the grip zone by screwing the tissue-engaging element over the rib.

In some implementations, the implant is sterilized.

In some implementations, the catheter device is sterilized.

In some implementations, the anchor driver is sterilized.

In some implementations, the distal opening has a rim, and the tube is shaped such that the rim is undulating.

In some implementations, the distal portion is flared toward the distal opening.

In some implementations, the rib is configured such that, as the driver screws the tissue-engaging element over the rib, the tissue-engaging element compresses parts of the rib with which the tissue-engaging element is in contact.

In some implementations, the rib has a proximal face that is shaped to define a shoulder.

In some implementations, the rib has a distal face that is tapered.

In some implementations, the anchor further includes an eyelet, mounted on the head so as to be revolvable about the anchor axis. The eyelet can be the same as or similar to other eyelets described anywhere herein.

In some implementations, the implant can further include a tether, threaded through the eyelet such that the eyelet is slidable along the tether.

In some implementations, the rib extends medially into the channel in a manner that defines, adjacent the rib, a niche in the grip zone. In some implementations, the system is configured such that, while the anchor driver screws the tissue-engaging element over the rib, the tissue-engaging element is excluded from the niche, and/or the tether extends through the grip zone within the niche, laterally from the tissue-engaging element.

In some implementations, proximal from the rib, the tube further defines an abutment that protrudes medially into the channel in a manner that, as the anchor driver screws the tissue-engaging element over the rib, inhibits revolution of the eyelet about the anchor axis.

In some implementations, a unitary structure defines both the rib and the abutment.

In some implementations, the rib protrudes further medially into the channel than the abutment.

In some implementations, the abutment is longer, along the channel, than the rib.

In some implementations, the rib comprises and/or is formed from a polymer.

In some implementations, the tube is lined with the polymer.

In some implementations, the tube comprises and/or is formed from the polymer.

In some implementations, the polymer is a thermoplastic elastomer.

In some implementations, the polymer is a block copolymer.

In some implementations, the block copolymer is polyether block amide.

In some implementations, the rib is a first of multiple ribs defined by the distal portion in the grip zone.

In some implementations, the multiple ribs are exactly two ribs.

In some implementations, the multiple ribs are exactly three ribs.

In some implementations, the multiple ribs are exactly four ribs.

In some implementations, the multiple ribs are exactly five ribs.

In some implementations, the multiple ribs are exactly six ribs.

In some implementations, the multiple ribs are distributed circumferentially around the tube axis.

In some implementations, the multiple ribs are distributed along the tube axis.

In some implementations, the rib extends around at least part of the tube axis.

In some implementations, the rib is toroidal, extending circumferentially around the entire tube axis.

In some implementations, the rib extends alongside the tube axis.

In some implementations, the rib is parallel with the tube axis.

In accordance with some implementations, a system useable and/or for use with a heart of a subject (e.g., a living subject, a simulation, etc.) includes an implant and a delivery tool.

In some implementations, the implant can include an anchor that has a head, and a helical tissue-engaging element extending distally away from the head to define an anchor axis of the anchor. In some implementations, the head can define an interface of the anchor.

In some implementations, the delivery tool can include a catheter device and/or an anchor driver.

In some implementations, the catheter device can include an extracorporeal portion at a proximal part of the catheter device, and/or a flexible tube extending distally from the extracorporeal portion.

In some implementations, the flexible tube can have a distal portion that: (i) is configured to be transluminally advanced to the heart, and (ii) has a distal opening. In some implementations, the flexible tube can have a distal portion that defines, along a tube axis of the tube, a channel through which the anchor is slidable toward the distal opening.

In some implementations, the flexible tube can have a membrane, disposed over the distal opening, and having one or more slits that divide the membrane into multiple flaps.

In some implementations, the anchor driver can be configured, via engagement with the interface, to slide the anchor distally through the channel and distally through the membrane via the one or more slits, the membrane being configured such that the flaps transiently separate responsively to passage of the anchor through the membrane.

In some implementations, the implant is sterilized.

In some implementations, the catheter device is sterilized.

In some implementations, the anchor driver is sterilized.

In some implementations, the membrane has multiple slits.

In some implementations, the multiple slits divide the membrane into four flaps.

In some implementations, the multiple slits converge to define a convergence point.

In some implementations, the membrane has a hole at the convergence point.

In some implementations, the anchor driver is configured to slide the anchor distally through the channel such that the tissue-engaging element aligns with the hole.

In some implementations, the membrane defines a notch, disposed eccentrically.

In some implementations, the notch extends laterally from the convergence point.

In some implementations, the notch is defined in a single one of the flaps.

In some implementations, the notch is defined partly in one of the flaps and partly in another of the flaps.

In some implementations, the head lies on the anchor axis. In some implementations, the anchor includes an eyelet, mounted laterally from the anchor axis. The eyelet can be the same as or similar to other eyelets described anywhere herein.

In some implementations, the anchor driver is configured to slide the anchor distally through the channel such that the eyelet aligns with the notch.

In accordance with some implementations, a system and/or apparatus can be configured for use with a tether secured to a tissue of a subject (e.g., a living subject, a simulation, etc.). In some implementations, the system/apparatus includes a tool and/or a lock.

In some implementations, the lock can define a passage through the lock, the passage configured to receive the tether therethrough. In some implementations, the lock can have an unlocked state in which the lock is transluminally slidable along the tether to the tissue by the tether sliding through the passage.

In some implementations, the lock can include a clamp face and a blade, and can further include an interface, engageable by the tool in a manner that configures the tool to actuate the lock by applying an actuating force to the interface.

In some implementations, the lock can be configured such that, while the tether is disposed through the passage, actuation of the lock (i) locks the tether to the lock by clamping the clamp face to the tether, and (ii) cuts the tether with the blade.

In some implementations, the tool is sterilized.

In some implementations, the lock is sterilized.

In some implementations, the actuating force is torque, and the tool is configured to actuate the lock by applying torque to the interface.

In some implementations, the lock includes an opposing face, actuation of the lock locking the tether to the lock by advancing the clamp face toward the opposing face.

In some implementations, the lock is configured such that, following the clamping of the tether between the clamp face and the opposing face, further actuation of the lock causes the clamp face to push the opposing face to move along with the clamp face.

In some implementations, actuation of the lock clamps the clamp face to the tether via axial movement of the clamp face, and/or cuts the tether via axial movement of the blade.

In some implementations, actuation of the lock clamps the clamp face to the tether via planar movement of the clamp face, and/or cuts the tether via planar movement of the blade.

In some implementations, the lock includes a mechanical linkage that includes a first bar and a second bar, the first bar providing the clamp face and the second bar providing the blade.

In some implementations, the mechanical linkage is a planar linkage.

In some implementations, the first bar is hingedly connected to the second bar.

In some implementations, the mechanical linkage is configured such that actuation of the lock clamps the tether between the clamp face and the second bar.

In some implementations, the lock includes a casing, and the mechanical linkage is configured such that actuation of the lock clamps the tether between the clamp face and the casing.

In some implementations, the blade faces away from the first bar.

In some implementations, the interface is coupled to a threaded rod that cooperates with the mechanical linkage as a linear actuator, such that rotation of the interface rotates the threaded rod and pivots the first bar with respect to the second bar.

In some implementations, the actuation of the lock clamps the clamp face to the tether prior to cutting the tether with the blade by the lock being configured such that: a first amount of the actuation clamps the clamp face to the tether, and/or further actuation of the lock, beyond the first amount of the actuation, is required for the blade to cut the tether.

In some implementations, the lock is configured such that the actuation of the lock clamps the clamp face to the tether prior to cutting the tether with the blade, by a distance-of-movement of the blade required to cut the tether being greater than a distance-of-movement of the clamp face required to clamp the clamp face to the tether.

In some implementations, the lock is configured such that the actuation of the lock clamps the clamp face to the tether prior to cutting the tether with the blade, by the lock including a mechanism that, responsively to actuation of the lock, moves the blade at a different rate to the clamp face.

In some implementations, the lock includes an opposing face, actuation of the lock locking the tether to the lock by clamping the tether between the clamp face and the opposing face.

In some implementations: the lock includes a spring, and/or the further actuation of the lock strains the spring, the straining of the spring functionalizing the blade.

In some implementations: the opposing face is supported by a compressible member.

In some implementations, the further actuation of the lock drives the clamp face to compress the compressible member in a manner that: maintains the tether clamped between the clamp face and the opposing face, and/or enables movement of the blade to cut the tether.

In some implementations, the lock defines, extending from a first end of the passage to a second end of the passage, lateral access via which the tether is introducible sideways into the passage.

In some implementations, the lock includes a casing that defines at the first end of the passage, an entrance into the passage. In some implementations, the lock includes a casing that defines at the second end of the passage, an exit from the passage.

In some implementations, the lock includes a casing that defines, connecting the entrance to the exit, a lateral slit that provides the lateral access.

In some implementations, actuation of the lock cuts the tether by revolving the blade around an axis.

In some implementations, actuation of the lock clamps the clamp face to the tether by moving the clamp face along the axis.

In accordance with some implementations, a system and/or an apparatus can be configured for use with a tether secured to a tissue (e.g., of a living subject, of a simulation, etc.). In some implementations, the apparatus includes a malleable lock and/or a tool.

In some implementations, the malleable lock can be shaped to define a passage therethrough.

In some implementations, the tool can include a shaft, a collet, and a grasper.

In some implementations, the collet can be housed within the shaft, and/or the lock can be held within the collet.

In some implementations, the grasper can be extendable distally through the passage and out of the shaft, and/or can be configured to grasp a bight of the tether and draw the bight proximally through the lock and into the shaft, thereby retaining the tether as a loop within the shaft.

In some implementations, the tool can be configured to, while the tether remains retained as the loop within the shaft: (i) be intracorporeally advanced distally along the tether such that progressive regions of the tether are fed around the grasper, (ii) subsequently lock the lock to the tether by actuating the collet to crimp the lock, (iii) subsequently release the lock from the collet and the tether from the grasper, and/or (iv) be subsequently withdrawn from the subject.

In some implementations, the tool is sterilized.

In some implementations, the lock is sterilized.

In some implementations, the tool is configured to actuate the collet via rotation of the shaft.

In some implementations, the grasper is a hook.

In some implementations, the grasper is a snare.

In accordance with some implementations, a system useable and/or for use with a subject includes a catheter device, a tether, a series of anchors, an anchor driver, and/or a tensioner.

The catheter device can include a tube that has: (i) a distal opening that is configured to be transluminally advanced into the subject, and (ii) a proximal end that defines a proximal opening.

In some implementations, the catheter device can include an extracorporeal unit that is coupled to the proximal end of the tube.

In some implementations, the tether can have a leading end and a second end, the second end being coupled to the extracorporeal unit.

In some implementations, the series of anchors can include a leading anchor, coupled to the leading end of the tether, and multiple successive anchors, slidably coupled to the tether.

In some implementations, the anchor driver can be configured to: (i) advance the leading anchor with the leading end of the tether through the tube and anchor the leading anchor to tissue of the subject, and (ii) subsequently, for each of the successive anchors sequentially, advance the successive anchor along the tether and through the tube.

In some implementations, the tensioner can be configured to: (i) engage an intermediate region of the tether, the intermediate region of the tether being at the extracorporeal unit and between the leading end and the second end, and/or (ii) apply tension to the tether by pulling on the intermediate region of the tether.

In some implementations, the catheter device is sterilized.

In some implementations, the tether is sterilized.

In some implementations, the anchors of the series are sterilized.

In some implementations, the anchor driver is sterilized.

In some implementations, the tensioner is sterilized.

In some implementations, the tensioner includes a gripper, configured to grip the tether in a manner that defines an isolated region of the tether between the grip and the second end, and that isolates the isolated region from the tension applied by the tensioner.

In some implementations, the tensioner includes a sheave, and is configured to engage the intermediate region of the tether by engaging the sheave with the tether.

In some implementations, the tensioner is a component of the extracorporeal unit.

In some implementations, the tensioner includes a linear actuator.

In some implementations, the tensioner includes a knob and complementary screw threads, and is actuatable via rotation of the knob.

In some implementations, the tensioner is configured to apply the tension to the tether by pulling the intermediate region of the tether laterally.

In some implementations, the tensioner includes a force gauge that indicates a magnitude of the tension.

In some implementations, the extracorporeal unit includes a winch, the second end of the tether operatively coupled to the winch.

In some implementations, the winch is spring-loaded in a manner that reduces slack in the tether.

In some implementations, the de-slacker includes a deactivation switch that is user-operable to deactivate the de-slacker in a manner that allows slack to be introduced to the tether and not taken up by the winch.

In some implementations, the anchors are mounted on the extracorporeal unit.

In some implementations, each of the anchors is stored in a respective cartridge that is mounted on the extracorporeal unit.

In some implementations, the system further includes multiple spacers threaded on the tether, alternatingly with the anchors of the series.

In some implementations, the system further comprises at least one free spacer, separate from the tether, and manually threadable onto the tether between anchors without access to an end of the tether.

In some implementations, each of the spacers is tubular, and is threaded on the tether by the tether extending through a lumen defined by the spacer.

In some implementations, the tube is substantially axially incompressible.

In some implementations, the tube is a flexible sleeve that is substantially axially compressible.

In some implementations, the tube is a fabric tube.

In some implementations, the tube has a sidewall that is inflatable in a manner that adjusts a compressibility of the spacer.

In some implementations, each of the spacers is a ribbon, and is threaded on the tether by the tether weaving along the ribbon.

In some implementations, each of the spacers is inflatable in a manner that adjusts a compressibility of the spacer.

In some implementations, each of the spacers has a body and one or more laterally-positioned eyelets through which the tether is threaded such that the body is mounted laterally from the tether.

In some implementations, the system further includes multiple connectors, each connecting a corresponding one of the spacers to a corresponding anchor of the series.

In some implementations, the system further includes a disconnector, housed within and removable from a compartment in the extracorporeal unit, and configured to cut one or more of the connectors.

In some implementations, each of the connectors provides a frangible connection between the corresponding spacer and the corresponding anchor.

In some implementations, the frangible connection is configured to be broken by pulling the connector away from the corresponding spacer.

In some implementations, each of the spacers includes a helical coil, and the frangible connection is provided by the connector being tucked between adjacent turns of the helical coil of the corresponding spacer.

In some implementations, each of the spacers includes a helical coil, and the frangible connection is configured to be broken by rotating the spacer to unwind the helical coil from the corresponding connector.

In some implementations, each of the spacers is arranged on the tether such that, upon advancement of the corresponding anchor distally along the tether toward the proximal opening, the spacer trails the anchor.

In some implementations, a first of the spacers is connected to the leading anchor, and is less axially compressible than at least another of the spacers.

In some implementations, each of the anchors includes (i) an anchor head; (ii) a tissue-engaging element extending distally away from the anchor head to define an anchor axis of the anchor and/or configured to be driven along the anchor axis into tissue of a subject (e.g., a living subject, a simulation, etc.); and/or (iii) a textile and/or polymer, shaped to define an eyelet, the anchor being coupled to the tether by the eyelet being coupled to the tether. The textile and/or polymer can be the same as or similar to other textiles and/or polymers described anywhere herein. The eyelet can be the same as or similar to other eyelets described anywhere herein.

In some implementations, for at least some of the anchors, the textile and/or polymer is a unitary piece of textile and/or polymer that is further shaped to define a spacer that extends away from the anchor head along the tether.

In some implementations, the unitary piece of textile is a strip along which the tether is woven.

In some implementations, the unitary piece of textile further defines a collar that rotatably couples the unitary piece of textile to the anchor such that the eyelet and the spacer are revolvable around the anchor axis.

In some implementations, the unitary piece of textile further defines a narrowed neck between the collar and the spacer that facilitates pivoting of the spacer.

In some implementations, the spacer extends away from the anchor head along the tether toward a subsequent anchor of the series.

In some implementations, the tissue-engaging element is porous.

In some implementations, the tissue-engaging element is formed to be helical and porous via additive manufacturing.

In some implementations, the tissue-engaging element can comprise and/or be formed from titanium.

In some implementations, the tissue-engaging element can comprise and/or be formed from a structural material and has a coating with which the structural material is coated, the coating being (i) of a material other than the structural material, and (ii) etched in a manner that provides the tissue-engaging element with a textured surface.

In some implementations, the coating material is a gold coating.

In some implementations, the coating is laser etched.

In some implementations, the coating is ion-beam etched.

In some implementations, the tensioner is configured: (i) to be actuated to apply the tension, and/or (ii) to subsequently maintain the tension.

In some implementations, the tensioner includes a latch, and is configured to maintain the tension by the latch being latched after the tensioner is actuated.

In some implementations, the tensioner includes a ratchet, the ratchet maintaining the tension.

In some implementations, the tensioner is reversibly mountable on the extracorporeal unit.

In some implementations, the extracorporeal unit defines an access site at which, once the anchor driver advances the leading anchor with the leading end of the tether through the tube and anchors the leading anchor, the intermediate region of the tether extends through the access site.

In some implementations, the tensioner is reversibly mountable at the access site of the extracorporeal unit.

In accordance with some implementations, a system and/or an apparatus includes an anchor that includes an anchor head, a tissue-engaging element, and an eyelet. These can be the same as or similar to other heads, tissue-engaging elements, and/or eyelets elsewhere herein.

In some implementations, the anchor head can include a stock. The stock can be configured in a variety of ways, e.g., as a core, a rod, a tube, a winch, a peg, a neck, etc.

In some implementations, the tissue-engaging element can be coupled to the stock, can extend distally away from the anchor head to define an anchor axis of the anchor, and/or can be configured to be driven along the anchor axis into tissue of a subject (e.g., a living subject, a simulation, etc.).

In some implementations, the eyelet can be mounted eccentrically from the stock, and can be saddle-shaped.

In some implementations, the anchor is sterilized.

In some implementations, the system/apparatus further includes a tether threaded through the eyelet.

In some implementations, the eyelet is revolvable about the anchor axis.

In some implementations, the anchor further includes a collar that circumscribes the stock, the eyelet being revolvable about the anchor axis by the collar rotating about the anchor axis.

In accordance with some implementations, a system and/or an apparatus includes an anchor useable and/or for use with tissue of a heart of a subject (e.g., a living subject, a simulation, etc.), the anchor including an anchor head and a tissue-engaging element. These can be the same as or similar to other anchors, heads, and/or tissue-engaging elements elsewhere herein.

In some implementations, the anchor head can define an interface, and can be formed substantially from a polymer.

In some implementations, the tissue-engaging element can extend distally away from the anchor head to define an anchor axis of the anchor, can be configured to be driven along the anchor axis into tissue of a subject (e.g., a living subject, a simulation, etc.) by an anchoring force applied to the interface, and/or can be formed substantially from the polymer.

In some implementations, the anchor is sterilized.

In some implementations, the anchor head includes a metal pin that serves as part of the interface.

In some implementations, the polymer is a polyaryletherketone.

In some implementations, the polymer is polyether ether ketone.

In some implementations, in at least part of the anchor, a radiopaque substance is mixed with the polymer.

In some implementations, the radiopaque substance is barium sulfate.

In some implementations, the anchor head includes a stock that fixedly couples the interface to the tissue-engaging element, and the anchor further includes a collar and/or an eyelet. The stock can be configured in a variety of ways, e.g., as a core, a rod, a tube, a neck, a winch, a peg, etc.

In some implementations, the collar and/or the eyelet can be formed substantially from the polymer.

In some implementations, the collar can be rotatably coupled to the anchor head by circumscribing the stock.

In some implementations, the eyelet can be mounted eccentrically by being coupled to the collar and can be revolvable about the anchor axis by rotation of the collar around the stock.

In some implementations, the collar and the eyelet are formed as a monolithic piece of the polymer.

In some implementations, the tissue-engaging element includes a central shaft and an external self-tapping screw thread extending helically around and along the central shaft.

In some implementations, the central shaft defines a distal point and has a tapered region that tapers distally toward the distal point.

In some implementations, the distal point lies on the anchor axis.

In some implementations, the central shaft tapers more steeply at the distal point, compared with at the tapered region.

In some implementations, the screw thread protrudes laterally from the central shaft by a distance, and the central shaft has a diameter that is 2-4 times greater than the distance.

In some implementations, the diameter of the central shaft is approximately 3 times greater than the distance.

In accordance with some implementations, a system includes an implant that includes a tether and/or an anchor. The anchor can include an anchor head, a tissue-engaging element, and/or a textile (and/or polmer).

In some implementations, the anchor head includes a stock, and/or an interface. The tissue-engaging element can be coupled to the interface via the stock, can extend distally away from the anchor head to define an anchor axis of the anchor, and/or can be configured to be driven along the anchor axis into tissue of a subject (e.g., a living subject, a simulation, etc.) by an anchoring force applied to the interface.

In some implementations, the textile (and/or polymer) is shaped to define an eyelet through which the tether is threaded. The eyelet can be pivotable over the interface. The textile (and/or polymer) can be the same as or similar to other textiles (and/or polymers) described anywhere herein. The eyelet can be the same as or similar to other eyelets described anywhere herein.

In some implementations, the textile is a fabric.

In some implementations, the textile is a yarn.

In some implementations, the textile is elongate, has two ends and a bight therebetween, and is shaped to define a loop at each end, the loops being threaded onto the stock such that the bight defines the eyelet.

In some implementations, the anchor further includes a collar that circumscribes the stock, the eyelet being connected to the collar such that the eyelet is revolvable about the anchor axis by the collar rotating about the anchor axis.

In some implementations, the textile is elongate, having two ends and a bight therebetween, the ends being connected to the collar such that the bight defines the eyelet.

In some implementations, the eyelet extends from two places on the collar, and/or is pivotable over the interface by pivoting about a hinge axis on which the two places lie.

In some implementations, the collar is defined by the textile.

In some implementations, the collar is rigid.

In some implementations, the collar is shaped to define at least one bore through which the textile passes.

In some implementations, the collar is shaped to define at least one tab to which the textile is tied.

In some implementations, the collar is flexible.

In some implementations, the collar is defined by a flexible tube. In some implementations, the flexible tube has a tube-lumen along the tube, the tube-lumen having two ends, an end-opening at each end of the tube, and/or a transverse channel. The stock can extend transversely through the tube via the transverse channel. In some implementations, the textile defines a closed loop onto which the tube is threaded by the textile extending through the lumen and out of both end-openings.

In accordance with some implementations, a system useable and/or for use with tissue of a heart of a subject (e.g., a living subject, a simulation, etc.) includes an implant, which implant can include a tether and/or multiple anchors. In some implementations, the tether can have a series of beads fixedly distributed along it.

In some implementations, each of the anchors can include a tissue-engaging element, and/or a head coupled to the tissue-engaging element. In some implementations, the tissue-engaging element can define an anchor axis of the anchor.

In some implementations, the head has a geometry that (i) facilitates sliding of the head over and along the tether while the anchor axis is parallel with the tether by allowing the beads to pass through the head, and/or (ii) inhibits sliding of the head over and along the tether while the anchor axis is transverse to the tether by obstructing the beads from passing through the head.

In some implementations, the system further includes an anchor driver, configured to, implant the implant at the tissue such that the tether is nonparallel with the tissue-engaging element of each of the anchors by, for each of the anchors sequentially, via engagement with the head, (i) transluminally sliding the head over and along the tether to the heart while the anchor axis is parallel with the tether, and (ii) driving the tissue-engaging element into the tissue.

In some implementations, the system further includes an adjustment tool, configured to apply tension to the tether after the implant has been implanted at the tissue, the implant being configured such that tensioning of the tether after the implant has been implanted at the tissue causes at least one of the beads to become obstructed by the head of at least one of the anchors.

In some implementations, each of the beads is oval.

In some implementations, each of the beads is a prolate spheroid.

In some implementations, each of the beads is radiopaque.

In some implementations, each of the beads is echogenic.

In some implementations, the implant is sterilized.

In some implementations, the anchor driver is sterilized.

In some implementations, the adjustment tool is sterilized.

In accordance with some implementations, a system useable and/or for use with a tissue of a subject (e.g., a living subject, a simulation, etc.) includes a catheter device, a tether, and/or a series of anchors. In some implementations, the catheter device can include a tube and/or an extracorporeal unit. In some implementations, the tube can have a distal opening that is configured to be transluminally advanced into the subject, and/or a proximal end that defines a proximal opening.

In some implementations, the extracorporeal unit can be coupled to the proximal end of the tube.

In some implementations, the extracorporeal unit includes a body and/or a series of cartridges (or anchor holders). In some implementations, the series of cartridges are distributed along the body in a manner that defines a proximal-distal axis. In some implementations, the series of cartridges/anchor holders are distributed along the body in a manner that defines a proximal-distal axis with one row or column of cartridges/anchor holders along the axis, while other arrangements have two or more parallel rows or columns of cartridges/anchor holders. In some implementations, the series of cartridges/anchor holders are distributed along the body in a manner that align with a proximal-distal axis (e.g., in a curved manner, in an angled matter, in a zig zag manner, etc.)

The cartridges/anchor holders herein can be configured in a variety of ways (e.g., from a simple receptacle or hole for holding an anchor to more involved or elaborate configurations and mechanisms).

In some implementations, one or more (e.g., one, some, or all) of the cartridges can define a respective cartridge vector that is oblique with respect to the proximal-distal axis, have a closed state, and/or be transitionable into an open state by at least part of the cartridge being slid along its cartridge vector.

In some implementations, the tether can extend along the body.

In some implementations, each anchor of the series of anchors is coupled to the tether, is housed by a corresponding cartridge of the series of cartridges, and/or is removable from the corresponding cartridge upon transitioning of the corresponding cartridge into its open state.

In some implementations, the cartridge vectors of the series of cartridges collectively define a common cartridge plane on which the cartridge vectors lie.

In some implementations, the proximal-distal axis is parallel with the common cartridge plane.

In some implementations, the proximal-distal axis lies on the common cartridge plane.

In some implementations, the tether extends along the body, parallel with the common cartridge plane.

In accordance with some implementations, a system useable and/or for use with a tissue of a subject (e.g., a living subject, a simulation, etc.) includes a catheter device, a tether, and/or a series of anchors.

In some implementations, the catheter device can include a tube and/or an extracorporeal unit. In some implementations, the tube can have a distal opening that is configured to be transluminally advanced into the subject, and/or a proximal end that defines a proximal opening. In some implementations, the extracorporeal unit can be coupled to the proximal end of the tube.

In some implementations, the extracorporeal unit includes a body and/or a series of cartridges (or anchor holders). In some implementations, the series of cartridges can be distributed along the body in a manner that defines a proximal-distal axis.

In some implementations, the tether can extend along the body.

In some implementations, each anchor of the series of anchors is coupled to the tether, is housed by a corresponding cartridge/anchor holder of the series of cartridges/anchor holders.

In some implementations, each anchor is releasable from the corresponding cartridge by pulling of the anchor such that at least part of the corresponding cartridge slides along a respective cartridge vector that is oblique with respect to the proximal-distal axis.

In accordance with some implementations, a system useable and/or for use with a tissue of a subject (e.g., a living subject, a simulation, etc.) includes a catheter device, a tether, and/or a series of anchors. In some implementations, the catheter device can include a tube and/or an extracorporeal unit. In some implementations, the tube can have a distal opening that is configured to be transluminally advanced into the subject, and/or a proximal end that defines a proximal opening.

In some implementations, the extracorporeal unit can be coupled to the proximal end of the tube.

In some implementations, the tether can extend along the extracorporeal unit.

In some implementations, the series of anchors can be distributed along the body in a manner that defines a proximal-distal axis.

In some implementations, each anchor of the series of anchors includes a head, coupled to the tether; and/or a tissue-engaging element, extending away from the head to define an anchor axis of the anchor.

In some implementations, each anchor of the series of anchors is mounted on the extracorporeal unit such that the anchor axis lies obliquely with respect to the proximal-distal axis.

In some implementations, the tether extends along the extracorporeal unit alongside the proximal-distal axis.

In some implementations, the extracorporeal unit includes a series of cartridges (or anchor holders), distributed along the proximal-distal axis, and/or each of the anchors is mounted on the extracorporeal unit by being housed by a corresponding cartridge of the series of cartridges.

In some implementations, for each anchor of the series of anchors, the anchor is oriented with the head proximal from the tissue-engaging element.

In some implementations, for each anchor of the series of anchors, the anchor is oriented with the tissue-engaging element closer than the head to the proximal opening.

In some implementations, the anchor axes of the series of anchors collectively define a common anchor plane.

In some implementations, the proximal-distal axis is parallel with the common anchor plane.

In some implementations, the proximal-distal axis lies on the common anchor plane.

In some implementations, the tether extends, along the extracorporeal body, parallel with the common anchor plane.

In some implementations, an anchor usable and/or for use at a tissue of a heart of a subject (e.g., a living subject, a simulation, etc.) includes a head and/or a tissue-engaging element.

In some implementations, the tissue-engaging element may extend away from the head to define an anchor axis of the anchor along which the tissue-engaging element is configured to be driven into the tissue, and/or may be formed, by additive manufacturing, to be porous.

In some implementations, the tissue-engaging element is helical and configured to be screwed along the anchor axis into the tissue.

In some implementations, the tissue-engaging element is a dart.

In some implementations, the tissue-engaging element is a staple.

In some implementations, the tissue-engaging element can comprise and/or be formed from titanium.

In some implementations, the tissue-engaging element is formed by powder bed fusion.

In some implementations, the tissue-engaging element can comprise and/or be formed by electron beam melting.

In some implementations, the tissue-engaging element can comprise and/or be formed by powder-fed directed-energy deposition.

In some implementations, the tissue-engaging element can be formed from a structural material, and/or can have a coating with which the structural material is coated, the coating being (i) of a material other than the structural material, and/or (ii) etched in a manner that provides the tissue-engaging element with a textured surface.

In some implementations, the tissue-engaging element is helical and configured to be screwed along the anchor axis into the tissue.

In some implementations, the tissue-engaging element is a dart.

In some implementations, the tissue-engaging element is a staple.

In some implementations, the structural material is coated with the coating by electroplating.

In some implementations, the structural material is steel.

In some implementations, the coating is gold.

In some implementations, the tissue-engaging element is etched by laser etching.

In some implementations, the tissue-engaging element is etched by ion-beam etching.

In some implementations, the tissue-engaging element is etched sufficiently shallowly that the coating is not completely penetrated by the etching.

In some implementations, a method includes (i) absorbing a substance into a snood disposed around a head of an anchor, the head including an interface, the anchor including a tissue-engaging element that is coupled to the interface, and the snood being disposed around the anchor head in a manner that preserves accessibility to the interface; and/or (ii) performing a procedure on a subject. In some implementations, the procedure can include (i) to a heart of the subject, transluminally advancing the anchor with the snood carrying the absorbed substance; and/or (ii) driving the tissue-engaging element into tissue of the heart by applying an anchoring force to the interface.

In some implementations, the substance includes a medicament, and absorbing the substance into the snood includes absorbing the medicament into the snood.

In some implementations, the substance includes a radiopaque dye, and absorbing the substance into the snood includes absorbing the radiopaque dye into the snood.

In some implementations, the procedure is performed in an operating theater, and the step of absorbing is performed in the operating theater.

In some implementations, the step of absorbing is performed no more than two hours prior to transluminally advancing the anchor.

In some implementations, advancing the anchor includes advancing the anchor using a driver that is engaged with the interface; driving the tissue-engaging element includes driving the tissue-engaging element by applying the anchoring force to the interface using the driver; and/or absorbing the substance includes absorbing the substance while the driver is engaged with the interface.

In some implementations, absorbing the substance includes dipping the anchor into the substance using the driver while the driver is engaged with the interface.

In accordance with some implementations, this disclosure relates to a method, including manufacturing textile components usable for and/or for use with implantable anchors. In some implementations, the method includes: (i) weaving a textile into an elongate form that includes a first tubular structure and a second tubular structure that are connected to, and parallel with, each other; and/or (ii) slicing the elongate form into transverse slices.

In some implementations, each slice can define a respective textile component that includes a first ring that is derived from the first tubular structure and that is configured to serve as a collar of the anchor; and/or a second ring that is derived from the second tubular structure, that is connected to the first ring, and that is configured to serve as an eyelet of the anchor.

In some implementations, weaving the textile into the elongate form includes weaving the textile into the elongate form such that the first tubular structure has a larger inner diameter than the second tubular structure.

In some implementations, weaving the textile into the elongate form includes weaving the textile into the elongate form such that the first tubular structure and the second tubular structure extend in parallel along a warp axis of the textile.

In some implementations, the method further includes, for each of the textile components, rotatably mounting the first ring on a head of the anchor such that the first ring couples the second ring to the head in a manner in which the second ring is revolvable around the head.

In some implementations, a method includes manufacturing textile components for implantable anchors by weaving a first elongate form that includes a first tubular structure; weaving a second elongate form that includes a second tubular structure; slicing the first elongate form into first transverse slices, each defining a first ring that is derived from the first tubular structure; slicing the second elongate form into second transverse slices, each defining a second ring that is derived from the second tubular structure; and/or for each of the textile components, forming the textile component by interlinking one of the first rings with one of the second rings such that the first ring is configured to serve as a collar of the anchor, and the second ring is configured to serve as an eyelet of the anchor.

In some implementations, weaving the second elongate form includes weaving the second elongate form such that the second tubular structure has a smaller inner diameter than the first tubular structure.

In some implementations, weaving the first elongate form includes weaving the textile into the first elongate form such that the first tubular structure extends along a warp axis of the textile.

In some implementations, weaving the second elongate form includes weaving the textile into the second elongate form such that the second tubular structure extends along a warp axis of the textile.

In some implementations, the method further includes, for each of the textile components, rotatably mounting the first ring on a head of the anchor such that the first ring couples the second ring to the head in a manner in which the second ring is revolvable around the head.

In some implementations, a method includes weaving a textile into a strip having a first slit and a second slit defined therethrough; and/or rotatably mounting the strip on a head of an implantable anchor by placing the head through the first slit such that the first slit serves as a collar-aperture, and the second slit serves as an eyelet-aperture that is revolvable around the head.

In some implementations, weaving the textile includes weaving the textile such that the first slit is longer than the second slit.

In some implementations, weaving the textile includes weaving the textile such that the first slit and the second slit are collinear with each other.

In some implementations, weaving the textile includes weaving the textile such that the first slit and the second slit are parallel with a warp axis of the textile.

In some implementations, a system useable and/or for use with a tissue of a subject (e.g., a living subject, a simulation, etc.) includes a catheter device and/or a series of anchors. In some implementations, the catheter device can include a flexible tube and/or an extracorporeal unit. In some implementations, the flexible tube can have a distal opening that is configured to be transluminally advanced toward the tissue, and/or a proximal end that defines a proximal opening.

In some implementations, the extracorporeal unit can be coupled to the proximal end of the tube, and can include a body, and/or a series of cartridges (or anchor holders), mounted on the body in an imbricated manner. In some implementations, each anchor of the series can be housed by a corresponding cartridge/anchor holder of the series of cartridges/anchor holders.

In some implementations, the system further includes a tether threaded through each of the anchors of the series.

In some implementations, a system useable and/or for use with a tissue of a subject (e.g., a living subject, a simulation, etc.) includes a catheter device and/or a series of anchors. The catheter device can include a flexible tube and/or an extracorporeal unit. The flexible tube can have a distal opening that is configured to be transluminally advanced toward the tissue. In some implementations, the extracorporeal unit can be coupled to a proximal end of the tube.

In some implementations, the series of anchors can be mounted on a body or stock in an imbricated manner.

In some implementations, the system further includes a tether threaded through each of the anchors of the series.

In some implementations, an implant includes a tether and/or a series of anchors. Each of the anchors can include an anchor head; a tissue-engaging element; and/or a textile through which the tether is threaded in a manner that slidably couples the anchor to the tether.

In some implementations, a system for treating a subject includes a support assembly; a first catheter; an implant catheter; an implant; and/or an adjustment tool. In some implementations, the support assembly can include a track. In some implementations, the first catheter can include a first-catheter flexible tube, and/or a first-catheter extracorporeal unit, coupled to a proximal part of the first-catheter flexible tube, and/or slidably mountable on the track such that the first-catheter flexible tube extends distally away from the track and into the subject.

In some implementations, the implant catheter can include an implant-catheter flexible tube, and/or an implant-catheter extracorporeal unit, coupled to a proximal part of the implant-catheter flexible tube. In some implementations, the implant-catheter extracorporeal unit can be slidably mountable on the track proximally from the first-catheter extracorporeal unit such that (i) the implant-catheter flexible tube extends distally away from the track and through the first-catheter flexible tube, and/or (ii) a distance along the track between the implant-catheter extracorporeal unit and the first-catheter extracorporeal unit is adjustable.

In some implementations, the implant can be mounted on the implant catheter, and/or can be transluminally implantable in the subject using the implant catheter.

In some implementations, the adjustment tool can include a flexible shaft, and/or an adjustment-tool extracorporeal unit, coupled to a proximal part of the flexible shaft.

In some implementations, the adjustment tool can be configured to be switched with the implant catheter subsequently to implantation of the implant such that (i) the adjustment-tool extracorporeal unit becomes slidably mounted on the track proximally from the first-catheter extracorporeal unit (ii) the flexible shaft becomes disposed through the first-catheter flexible tube, extending distally away from the track and toward the implant, and/or (iii) a distance along the track between the adjustment-tool extracorporeal unit and the first-catheter extracorporeal unit is adjustable.

In some implementations, the system further includes a second catheter that includes a second-catheter flexible tube and/or a second-catheter extracorporeal unit, coupled to a proximal part of the second-catheter flexible tube. In some implementations, he second-catheter extracorporeal unit can be slidably mountable on the track proximally from the first-catheter extracorporeal unit such that the second-catheter flexible tube extends distally away from the track and through the first-catheter flexible tube, and/or a distance along the track between the second-catheter extracorporeal unit and the first-catheter extracorporeal unit is adjustable.

In some implementations, the implant-catheter extracorporeal unit can be slidably mountable on the track proximally from the first-catheter extracorporeal unit and the second-catheter extracorporeal unit such that (i) the implant-catheter flexible tube extends distally away from the track and, within the second-catheter flexible tube, through the first-catheter flexible tube, and/or (ii) a distance along the track between the implant-catheter extracorporeal unit and the second-catheter extracorporeal unit is adjustable.

In some implementations, the adjustment-tool can be configured to be switched with the implant catheter and the second catheter subsequently to implantation of the implant such that (i) the adjustment-tool extracorporeal unit becomes slidably mounted on the track proximally from the first-catheter extracorporeal unit, and/or (ii) the flexible shaft becomes disposed through the first-catheter flexible tube, absent the second-catheter flexible tube, extending distally away from the track and toward the implant.

In some implementations, this disclosure is directed to a system comprising a catheter device, an extracorporeal unit, a tether, and a series of anchors.

In some implementations, the catheter device can comprise a flexible tube, the flexible tube comprising a distal opening positioned at a distal end of the flexible tube and a proximal opening positioned at a proximal end of the flexible tube.

In some implementations, the extracorporeal unit, can be coupled to the proximal end of the flexible tube. In some implementations, the extracorporeal unit can comprise a body, and a series of cartridges or anchor holders.

In some implementations, the series of cartridges/anchor holders is distributed along a proximal-distal axis of the body in a manner that defines a proximal-distal axis. In some implementations, a distalmost cartridge or anchor holder of the series of cartridges/anchor holders is closest to the proximal opening.

In some implementations, the series of cartridges/anchor holders are distributed along the body in a manner that defines a proximal-distal axis with one row or column of cartridges/anchor holders along the axis, while other arrangements have two or more parallel rows or columns of cartridges/anchor holders. In some implementations, the series of cartridges/anchor holders are distributed along the body in a manner that does not align with a proximal-distal axis (e.g., in a curved manner, in an angled matter, in a zig zag manner, etc.)

In some implementations, an anchor of the series of anchors is housed in a cartridge or anchor holder of the series of cartridges/anchor holders and is coupled to the tether such that the tether extends along the body, parallel with the proximal-distal axis.

In some implementations, the anchor comprises an anchor head, a tissue-engaging element extending distally away from the anchor head to define an anchor axis of the anchor, the tissue-engaging element configured to be driven along the anchor axis into the tissue. In some implementations the anchor further comprises a textile, shaped to define an eyelet threaded onto the tether in a manner that slidably couples the anchor to the tether.

In some implementations, the series of anchors comprises a leading anchor and one or more subsequent anchors, such that the leading anchor is housed in the distalmost cartridge or anchor holder and is fixed to the tether. In some implementations, the one or more subsequent anchors are configured to be slidably coupled to tether.

In some implementations, the anchor comprises: a head, slidably coupled to the tether, and a tissue-engaging element, extending away from the head to define an anchor axis of the anchor. In some implementations, the anchor is housed in the cartridge or anchor holder such that the anchor axis lies obliquely with respect to the proximal-distal axis. In some implementations, the anchor is housed in the cartridge or anchor holder such that the anchor axis is perpendicular with respect to the proximal-distal axis.

In some implementations, the cartridge or anchor holder has a closed state in which the cartridge/anchor holder securely houses the anchor. In such implementations, the cartridge/anchor can define a respective cartridge/anchor holder vector that is oblique with respect to the proximal-distal axis. In some implementations, the cartridge/anchor holder is transitionable into an open state in which the corresponding anchor is removable from the cartridge/anchor holder in response to at least part of the cartridge sliding along the cartridge/holder vector.

In some implementations, the cartridge/anchor holder is associated with a threshold force, and is further configured to transition into the open state responsive to the anchor being pulled with a force that exceeds the threshold force.

In some implementations, the tether comprises (i) a distal end coupled to a leading anchor, and (ii) a proximal end releasably secured within the extracorporeal unit.

In some implementations, the extracorporeal unit comprises a de-slacker that comprises a winch that is spring-loaded in a manner that takes up slack in the tether.

In some implementations, the de-slacker comprises a deactivation switch configured to deactivate the de-slacker in a manner that allows slack to be introduced to the tether and not taken up by the winch.

In some implementations, the system further comprises multiple spacers threaded on the tether, alternatingly with anchors of the series of anchors.

In some implementations, a spacer of the multiple spacers is tubular, and is threaded on the tether by the tether extending through a lumen defined by the spacer.

In some implementations, the spacer is arranged on the tether such that, upon advancement of the anchor distally along the tether toward the proximal opening, the spacer trails the anchor.

In some implementations, a first spacer of the multiple spacers is connected to a leading anchor of the series of anchors, and the first spacer of the multiple spacers is less axially compressible than at least another spacer of the multiple spacers.

In some implementations, the anchor of the series of anchors comprises: an anchor head; and a helical tissue-engaging element, extending away from the anchor head to define an anchor axis of the anchor, and configured to be screwed along the anchor axis into the tissue.

In some implementations, the tube can alternatively or additionally define, at the distal portion, a grip zone at which the tube has a grip surface that inhibits sliding of the anchor through the grip zone by gripping a lateral surface of the helical tissue-engaging element. In some implementations, the system further includes an anchor driver configured to slide the anchor distally through the channel to the grip zone, and/or to drive the anchor through the grip zone by screwing the tissue-engaging element over the grip surface.

In some implementations, the system further includes an anchor driver configured to slide the anchor distally through the channel to the grip zone, and/or to drive the anchor through the grip zone by screwing the tissue-engaging element over the grip surface.

In some implementations, the grip surface is configured such that, as the driver screws the helical tissue-engaging element over the grip surface, the helical tissue-engaging element temporarily compresses parts of the grip surface with which the helical tissue-engaging element is in contact.

In some implementations, the grip surface comprises at least one resilient nub that protrudes medially into the channel. Alternatively or additionally, in some implementations, the grip surface comprises at least one resilient rib that protrudes medially into the channel.

In some implementations, the anchor further includes an eyelet, mounted on the head so as to be revolvable about the anchor axis. Proximal from the rib, the tube can further define an abutment that protrudes medially into the channel in a manner that, as the anchor driver screws the tissue-engaging element over the grip surface, inhibits revolution of the eyelet about the anchor axis.

In some implementations, the anchor includes an anchor head, a tissue-engaging element, and a textile (and/or a polymer). In such implementations, the tissue-engaging element can extend distally away from the anchor head to define an anchor axis of the anchor, and can be configured to be driven along the anchor axis into the tissue. In some implementations, the textile (and/or polymer), can be shaped to include an eyelet, the anchor being coupled to the tether via the eyelet. The textile (and/or polymer) can be the same as or similar to other textiles (and/or polymers) described anywhere herein. The eyelet can be the same as or similar to other eyelets described anywhere herein.

In some implementations, the anchor head includes an interface that is coupled to the tissue-engaging element, the tissue-engaging element being configured to be driven along the anchor axis into the tissue by an anchoring force applied to the interface.

In some implementations, the catheter device further includes a de-slacker, coupled to the tether, and configured to eliminate slack in the tether.

In some implementations, the system further includes an anchor driver comprising a flexible shaft, and a drive head at a distal end of the shaft. In some implementations, the anchor driver is configured to engage the drive head with the anchor, remove the anchor from the corresponding cartridge, and while the anchor remains coupled to the tether, advance the anchor into the proximal opening and through the flexible tube toward the tissue, and anchor the anchor to the tissue.

In some implementations, the anchor driver is configured to remove the anchor from the cartridge/anchor holder by applying a pulling force to the anchor such that the cartridge/anchor holder transitions into an open state.

In some implementations, the system further includes an elongate adjustment tool and a lock. In some such implementations, the adjustment tool can be configured to advance the lock distally along the tether into the subject and toward the tissue, apply tension to the tether, lock the tension in the tether by locking the lock to the tether, cut the tether proximally from the lock, and/or leave the lock in the subject locked to the tether.

In some implementations, the lock is configured to be placed onto and advanced along the tether by the adjustment tool without access to an end of the tether.

In some implementations, the lock includes a frame, a first set of hooked fingers, and/or a second set of hooked fingers. In some such implementations, the first set of hooked fingers extend from a first side of the frame toward a second side of the frame, the second side being opposite the first side. In some such implementations, the second set of hooked fingers extend from the second side toward the first side, the fingers of the second set arranged along the frame alternatingly with the fingers of the first set.

In some implementations, the lock (i) has an unlocked state in which the frame is constrained to be narrowed and in which the tether is placeable and/or slidable between the fingers of the first and second sets of fingers, and/or (ii) is lockable to the tether by unconstraining the frame to widen such that the first and second sides of the frame responsively move away from each other, pulling with them the first and second sets of fingers, respectively.

In some implementations, the first and second sides of the frame, moving away from each other, pull with them the first and second sets of fingers such that the tether becomes clamped between the fingers of the first set and the fingers of the second set.

In some implementations, the first and second sides of the frame, moving away from each other, pull with them the first and second sets of fingers such that the tether becomes forced into a tortuous path.

In some implementations, the adjustment tool is configured to advance the lock distally along the tether into the heart of the subject and toward the tissue while maintaining the lock in the unlocked state by constraining the frame to be narrowed.

In some implementations, the extracorporeal unit comprises a catheter-device extracorporeal unit. In some implementations, the adjustment tool comprises an adjustment-tool extracorporeal unit, a shaft extending distally from the adjustment-tool extracorporeal unit, and a tool head at a distal end of the shaft. In some such implementations, the adjustment tool is configured to advance the lock distally along the tether into the subject and toward the tissue while the lock is housed within the tool head.

In some implementations, the tether has (i) a distal end at a leading anchor of the series of anchors, and (ii) a proximal end secured within the extracorporeal unit, and is releasable from within the extracorporeal unit so as to be threadable proximally into an aperture of the lock, through the lock and into the shaft of the adjustment tool.

In some implementations, the adjustment tool includes an uptake assembly and a knob. In some such implementations the adjustment tool includes, at a working end of the uptake assembly, a gripper disposed proximally from the lock such that, in a receiving state of the uptake assembly, threading of the proximal end of the tether proximally into the aperture of the lock, through the lock and the tool head, and into the shaft of the adjustment tool, causes the working end of the uptake assembly to receive the proximal end of the tether.

In some implementations, the knob can be mounted on a body of the adjustment-tool extracorporeal unit. In some implementations the knob can be operably coupled to a proximal part of the gripper such that operation of the knob transitions the uptake assembly into a grip state in which the gripper grips the tether. In some implementations, the knob is mounted on the adjustment-tool extracorporeal unit such that transitioning the uptake assembly into the grip state releases the knob from the adjustment-tool extracorporeal unit.

In some implementations, once released from the adjustment-tool extracorporeal unit, the knob can be removable from the adjustment-tool extracorporeal unit in a manner that withdraws the working end of the uptake assembly, along with the proximal end of the tether, proximally through and out from the shaft and the adjustment-tool extracorporeal unit such that the tether becomes positioned through the lock, the tool head, the shaft, and the adjustment-tool extracorporeal unit.

In some implementations, the lock is biased to lock, the adjustment tool includes an obstructor tube extending distally through the shaft and into the head such that a distal part of the obstructor tube is disposed within the lock in a manner that constrains the lock unlocked. While the knob of the uptake assembly remains mounted on the adjustment-tool extracorporeal unit, the working end of the uptake assembly can be disposed within the obstructor tube, such that removal of the knob from the adjustment-tool extracorporeal unit withdraws the working end of the uptake assembly, along with the proximal end of the tether, proximally through and out from the obstructor tube such that the tether becomes positioned through one or more of the lock, the tool head, the obstructor tube within the shaft, and the adjustment-tool extracorporeal unit.

In some implementations, (i) the lock is biased to lock, and/or (ii) the adjustment tool includes a guillotine/cutting tool within the tool head and proximal from the lock, and/or an obstructor extending distally through the shaft and the guillotine/cutting tool such that a distal part of the obstructor is disposed within the lock in a manner that constrains the lock unlocked.

In some implementations, the adjustment-tool extracorporeal unit includes a lock-and-cut subassembly that includes a locking block, coupled to the obstructor, and/or a lock-and-cut controller.

In some implementations, withdrawal of the working end of the uptake assembly along with the proximal end of the tether, proximally through and out from the shaft and the adjustment-tool extracorporeal unit, leaves the tether positioned through the lock and the guillotine such that (a) subsequent locking of the lock locks the lock to the tether, and (b) subsequent actuation of the guillotine cuts the tether proximally from the lock.

In some implementations, the lock-and-cut controller is operatively coupled to the locking block such that operation of the lock-and-cut controller draws the locking block proximally such that the obstructor becomes withdrawn from the lock and the lock responsively locks to the tether.

In some implementations, the adjustment-tool extracorporeal unit includes a tensioning subassembly that includes a tensioning block; a clamp, attached to the tensioning block; and/or a tensioning controller. In some implementations, while the knob of the uptake assembly remains mounted on the adjustment-tool extracorporeal unit, the gripper can extend from the knob, distally through the clamp and the shaft to the working end.

In some implementations, withdrawal of the working end of the uptake assembly, along with the proximal end of the tether, proximally through and out from the shaft and the adjustment-tool extracorporeal unit can withdraw the gripper from the clamp, leaving the tether positioned through the clamp such that subsequent operation of the clamp locks the tether to the tensioning block.

In some implementations, the tensioning controller is operatively coupled to the tensioning block such that, while the tether remains locked to the tensioning block, operation of the tensioning controller applies tension to the tether by drawing the tensioning block and the tether proximally.

In some implementations, the adjustment tool includes an uptake assembly that includes a sleeve extending distally through the shaft and terminating proximally from the lock; a gripper extending distally through the sleeve and having a widened distal portion disposed distally outside of the sleeve, the sleeve and the gripper being shaped and positioned such that threading of the proximal end of the tether proximally into the shaft of the adjustment tool advances the proximal end of the tether proximally around the widened distal portion of the gripper and into the sleeve; and/or a knob.

In some implementations, the knob can be mounted on the adjustment-tool extracorporeal unit, and/or operably coupled to a proximal part of the sleeve and to a proximal part of the gripper such that operation of the knob grips the tether within the sleeve by transitioning the uptake assembly into a grip state by drawing the widened distal portion of the gripper proximally into the sleeve.

In some implementations, the mounting of the knob on the adjustment-tool extracorporeal unit can be such that transitioning of the uptake assembly into the grip state releases the knob from the adjustment-tool extracorporeal unit.

In some implementations, once released from the adjustment-tool extracorporeal unit, the knob can be removable from the adjustment-tool extracorporeal unit in a manner that pulls the sleeve and the gripper, along with the proximal end of the tether, proximally through the shaft and the adjustment-tool extracorporeal unit, and out of the adjustment tool such that the tether extends through the lock, the head, the shaft, and the adjustment-tool extracorporeal unit.

In some implementations, a system, useable and/or for use with a tissue of a subject, includes a catheter device, a tether, and/or a series of anchors. The catheter device can include a flexible tube and/or an extracorporeal unit. The flexible tube can have a distal opening that is configured to be transluminally advanced toward the tissue, and/or a proximal end that defines a proximal opening. The extracorporeal unit can be coupled to the proximal end of the tube, and/or can include a body, and/or a series of cartridges.

In some implementations, the series of anchors can be coupled to the tether. In some implementations, each anchor of the series of anchors can include an anchor head, and a tissue-engaging element that extends distally away from the anchor head to define an anchor axis of the anchor, and/or can be housed by a corresponding cartridge of the series of cartridges.

In some implementations, the series of anchors includes a first subset of the anchors and a second subset of the anchors.

In some such implementations, the first subset contains a first 2-6 of the anchors of the series. In some implementations, for each of the anchors of the first subset, the tissue-engaging element has a first width.

In some implementations, the second subset can contain more (e.g., a greater number of) anchors than does the first subset.

In some implementations, for each of the anchors of the second subset, the tissue-engaging element has a second width that is smaller than the first width.

In some implementations, for each of the anchors of the series, the tissue-engaging element is a helical tissue-engaging element that extends helically away from the anchor head and that is configured to be screwed along the anchor axis into the tissue.

In some implementations, the first 2-6 of the anchors of the series is a first 4 of the anchors of the series, and the second subset contains the first 4 anchors of the series.

In some implementations, the second subset contains 4-18 of the anchors of the series.

In some implementations, the second subset contains a remainder of the anchors of the series.

In some implementations, the system further includes an anchor driver that includes a flexible shaft, and/or a drive head at a distal end of the shaft. In some implementations, the anchor driver can be configured to, for each of the anchors of the series sequentially, beginning with the anchors of the first subset: (i) engage the drive head with the anchor head, (ii) remove the anchor from the corresponding cartridge, and/or (iii) while the anchor remains coupled to the tether, advance the anchor into the proximal opening and through the flexible tube toward the tissue, and drive the tissue-engaging element into the tissue.

In some implementations, a system and/or apparatus, useable and/or for use with a tissue of a subject, includes an implant and/or an anchor driver. The implant can include a tether and/or a series of anchors. The series of anchors can be coupled to the tether. Each anchor of the series of anchors can include (i) an anchor head, and/or (ii) a tissue-engaging element that extends distally away from the anchor head to define an anchor axis of the anchor.

In some implementations, the series of anchors includes a first subset of the anchors of the series, which contains a first 2-6 of the anchors of the series. The tissue-engaging element of each anchor of the first subset may have a first width.

In some implementations, the series of anchors further includes a second subset of the anchors of the series. The second set can contain more anchors than does the first subset. The tissue-engaging element of each anchor of the second subset may have a second width that is smaller than the first width.

The anchor driver can include a flexible shaft and/or a drive head at a distal end of the shaft. The anchor driver can be configured to, for each of the anchors of the series sequentially, beginning with the anchors of the first subset, (i) engage the drive head with the anchor head, and/or (ii) while the anchor remains coupled to the tether, advance the anchor transluminally toward the tissue, and drive the tissue-engaging element into the tissue.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

Any methods of using the systems, assemblies, apparatuses, devices, etc. herein can be performed on a living subject (e.g., human, or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

The present invention will be more fully understood from the following detailed description of implementations thereof, taken together with the drawings, in which:

### BRIEF DESCRIPTION OF THE DRAWINGS

FIGS. 1 and 2A-D are schematic illustrations of a delivery tool for implanting an implant in a subject, including aesthetic features, in accordance with some implementations;

FIGS. 3A-B are schematic illustrations of an anchor and a corresponding spacer, including aesthetic features, in accordance with some implementations;

FIGS. 4A-D are schematic illustrations of a leading anchor, including aesthetic features, in accordance with some implementations;

FIGS. 5A-C are schematic illustrations of an anchor, including aesthetic features, in accordance with some implementations;

FIGS. 6A-B, 7, 8A-B, 9, 10, and 11A-K are schematic illustrations of eyelets, in accordance with some implementations;

FIGS. 12 and 13A-F are schematic illustrations of a distal portion of a flexible tube (including aesthetic features) and use thereof, in accordance with some implementations;

FIGS. 14A-E and 15A-C are schematic illustrations of variants of the flexible tube, including aesthetic features, in accordance with some implementations;

FIG. 16 is a schematic illustration of a system including a membrane, including aesthetic features, in accordance with some implementations;

FIGS. 17A-B are schematic illustrations of an anchor, including aesthetic features, in accordance with some implementations;

FIGS. 18A-B are schematic illustrations showing implantation of an implant, including aesthetic features, in accordance with some implementations;

FIGS. 19A-G are schematic illustrations of system having a tool and a lock, including aesthetic features, and being used to apply and lock in tension to a tether of an implant, in accordance with some implementations;

FIGS. 20A-C are schematic illustrations of a system having a tool and a lock, including aesthetic features, and being used to apply and lock in tension to a tether of an implant, in accordance with some implementations;

FIGS. 21, 22A-E, 23, 24A-D, 25A-C, 26, and 27A-C are schematic illustrations of various locks or lockers, including aesthetic features, in accordance with some implementations;

FIGS. 28, 29A-B, and 30 are schematic illustrations of tensioners, including aesthetic features, in accordance with some implementations;

FIGS. 31 and 32A-B are schematic illustrations of an implant being implanted, in accordance with some implementations;

FIGS. 33A-B, 34A-D, 35A-C, 36A-C, 37A-G, 38A-C, 39A-I, 40A-C, and 41A-L which are schematic illustrations of a system, including aesthetic features thereof, and techniques for use therewith, in accordance with some implementations;

FIGS. 42A-B are schematic illustrations of part of an extracorporeal unit, including aesthetic features thereof, and techniques for use therewith, in accordance with some implementations;

FIGS. 43, and 44A-B are schematic illustrations of techniques for changing the presence of a spacer between the final anchor and the lock of an implant, in accordance with some implementations;

FIGS. 45A-D are schematic illustrations of various locks that comprise a leader, including aesthetic features thereof, in accordance with some implementations;

FIG. 46 is a schematic illustration of an anchor-spacer assembly comprising an anchor and a spacer, including aesthetic features thereof, in accordance with some implementations;

FIGS. 47A-H are schematic illustrations of various spacers and anchor-spacer assemblies, including aesthetic features thereof, in accordance with some implementations;

FIGS. 48, 49, and 50 are schematic illustrations of textile collars and eyelets, such as textile components that include both the collar and the eyelet, including aesthetic features thereof, in accordance with some implementations;

FIGS. 51A-C, 52A-C, 53, and 54 are schematic illustrations of various spacers, including aesthetic features thereof, in accordance with some implementations;

FIG. 55 is a schematic illustration of an anchor that comprises a tissue-engaging element, including aesthetic features thereof, in accordance with some implementations;

FIG. 56 is a flow chart showing at least some steps of a technique for manufacturing a tissue-engaging element, in accordance with some implementations;

FIG. 57 is a schematic illustration of a catheter device, including aesthetic features thereof, whose extracorporeal unit comprises an integrated tensioner, in accordance with some implementations; and

FIGS. 58-59 are schematic illustrations of an implant, including aesthetic features thereof, in accordance with some implementations.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure can be practiced without specific details being presented herein. Furthermore, well-known features can be omitted or simplified in order not to obscure the disclosure.

Throughout the specification, identical names are used to denote different implementations of an element. Unless stated otherwise, implementations and applications of the devices, systems, and techniques described herein can include any variant in which an element is substituted with another identically-named element. Furthermore, throughout the figures, the presence or absence of different suffixes for the same reference numerals are used to denote different variants of the same elements. Unless stated otherwise, implementations and applications of the devices, systems, and techniques described herein can include any variant in which an element is substituted with another element having the same reference numeral, whether denoted with or without a suffix.

In order to avoid undue clutter from having too many reference numbers and lead lines on a particular drawing, some elements are introduced via one or more drawings and not explicitly identified in every subsequent drawing that contains that element.

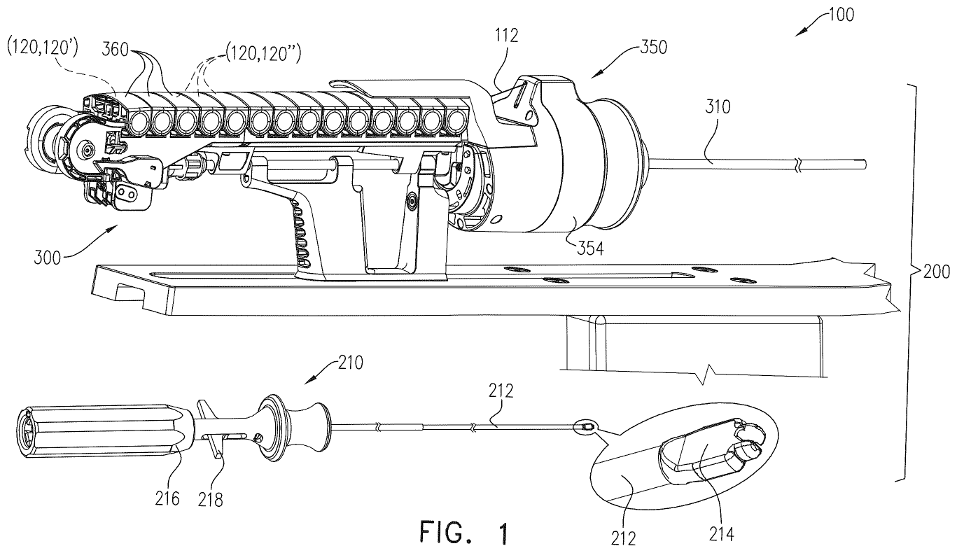

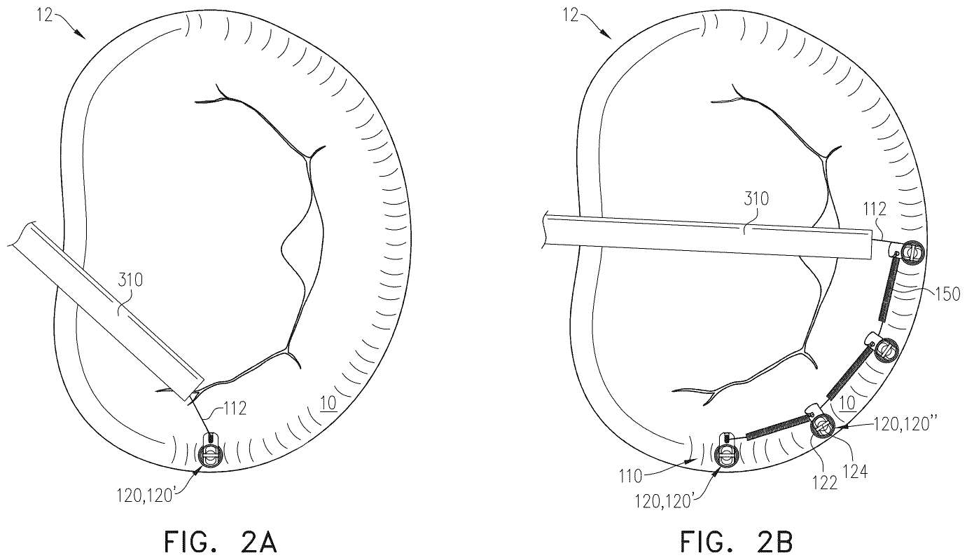

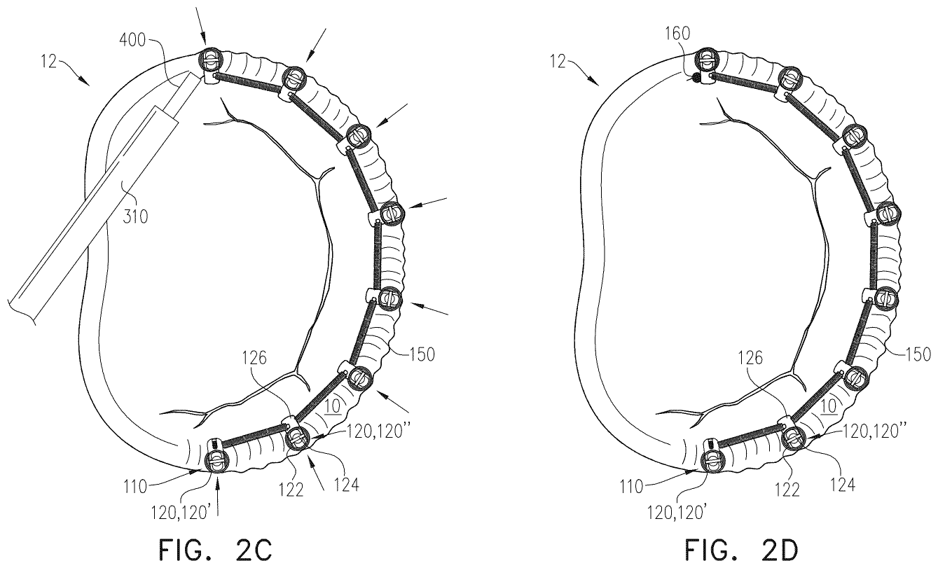

Reference is made to FIGS. 1, and 2A-D, which are schematic illustrations of a delivery tool 200 for implanting an implant 110 in a subject, in accordance with some implementations. Implant 110 and delivery tool 200 can be components of a system 100. Nevertheless, because implant 110 is largely obscured in FIG. 1 due to being loaded within delivery tool 200, refence numeral 110 has been omitted from FIG. 1. It is to be noted that tether 112 (described hereinbelow) of implant 110 is visible and therefore labeled in FIG. 1.

In the description of system 100, the implant of the system is described and shown as implant 110, which is described in more detail hereinbelow. However, it is to be understood that system 100 can comprise other implants, mutatis mutandis, e.g., delivery tool 200 can be used to implant other implants, mutatis mutandis.

For example, system 100 can comprise other implants that comprise, or are anchored with, multiple anchors such as, but not limited to, implants and/or anchors described herein, and/or implants and/or anchors described in WO 2021/084407 to Kasher et al. and/or WO 2022/172149 to Shafigh et al., each of which is incorporated herein by reference (e.g., implants that comprise multiple anchors slidably coupled to, e.g., threaded onto-a tether). Alternatively or additionally, delivery tool 200 and/or components thereof can be used, mutatis mutandis, to facilitate implantation of an implant (e.g., an annuloplasty structure) described in International Patent Application Publication WO 2014/064694 to Sheps et al., and/or International Patent Application Publication WO 2016/174669 to Iflah et al., each of which is incorporated herein by reference. Furthermore, and more generally, system 100 and/or techniques described for use therewith can be used in combination with one or more of the systems and/or techniques described in the references referenced in this paragraph.

In some implementations, the systems, apparatuses, devices, implants, etc. herein can be configured and/or used for annuloplasty, e.g., the implant can be an annuloplasty implant. In some implementations, the systems, apparatuses, devices, implants, etc. herein can be configured and/or used to close an opening (e.g., an opening to an appendage, an opening to a passageway, etc.) and/or to reshape another region of tissue (e.g., for ventricular remodeling, atrial remodeling, muscle remodeling, etc.)

FIG. 1 shows an overview of delivery tool 200, which comprises an anchor driver 210, and a catheter device 300. Catheter device 300 comprises a flexible tube 310 (e.g., a catheter), configured to be advanced into a subject, and an extracorporeal unit 350 (e.g., an extracorporeal control unit), coupled to tube 310, and configured to remain outside the body of the subject. In some implementations, extracorporeal unit 350 defines, or is coupled to, a handle of device 300. In some implementations, extracorporeal unit 350 shares one or more features with one or more of the extracorporeal units described in International Patent Application Publication WO 2022/064401 to Halabi et al., and/or International Patent Application Publication WO 2022/172149 to Shafigh et al., each of which is incorporated herein by reference. Furthermore, catheter device 300 can be used, mutatis mutandis, to facilitate implantation of any of the implants described in US Patent Application Publication 2021/0145584 to Kasher et al., and/or WO 2022/172149 to Shafigh et al., each of which is incorporated herein by reference.

FIGS. 2A-D show delivery tool 200 being used to implant implant 110 at a heart valve 12 of a subject (e.g., a living subject, a simulation, etc.). In the illustrated example, valve 12 is a mitral valve, but it is to be understood that the valve can be another atrioventricular valve (e.g., a tricuspid valve, as shown in later figures) or another valve, e.g., a pulmonary valve, aortic valve, and/or other valve. Furthermore, although in the illustrated example implant 110 is implanted at an upstream surface of valve 12 (e.g., along the annulus of the valve), the scope of the disclosure includes other implantation sites, such as a downstream surface of the valve (e.g., along the subannular groove). Still further, implant 110 can be implanted at a site other than a heart valve, such as within an atrium or within a ventricle of the heart, e.g., to contract the atrium or ventricle.

As described hereinabove, implant 110 comprises multiple anchors 120 and a tether 112 on which the anchors are threaded. As described in more detail hereinbelow, during implantation only a distal portion of tether 112 remains implanted in the subject, while a proximal portion of the tether is removed from the subject, e.g., with catheter device 300. Nonetheless, for the sake of simplicity, tether 112 is described herein as a component of implant 110.

Tether 112 can take various forms, e.g., monofilament, polyfilament, a line, a wire, a ribbon, a rope, a cable, a braid, a suture, etc. Tether 112 can comprise a metal (e.g., nitinol or surgical steel), a synthetic polymer (e.g., nylon, polyester, polypropylene, polybutester), and/or natural fiber (e.g., silk). Tether 112 can be considered to be a contraction member.

Anchors 120 are distributed in a series along tether 112, e.g., threaded onto the tether. Moreover, and as shown, catheter device 300 can be provided with tether 112 and anchors 120 loaded therein, with the anchors threaded onto the tether. When thus provided, the series of anchors can be at extracorporeal unit 350, e.g., mounted on/in the extracorporeal unit.

In some implementations, each anchor 120 can be disposed in a respective cartridge or anchor holder 360 to facilitate handling of the anchor, such as engagement of anchor driver 210 with the anchor and/or positioning of the engaged anchor appropriately for advancement into tube 310. This is illustrated in FIG. 1 by parenthesizing the reference numerals of the anchors within cartridges 360. In some implementations, once a given anchor has been anchored, its cartridge is discarded, e.g., by releasing it from extracorporeal unit 350. Examples of such cartridge-based mounting of anchors are described in WO 2022/064401 to Halabi et al., and International Patent Application Publication WO 2022/172149 to Shafigh et al., each of which is incorporated herein by reference.

The term cartridge as used herein is interchangeable with the term anchor holder, the cartridges/anchor holders herein can be configured in a variety of ways (e.g., from a simple receptacle or hole for holding an anchor to more involved or elaborate configurations and mechanisms).

Delivery tool 200 can be used to implant implant 110 by anchor driver 210 being used, for each of the anchors consecutively, to engage the anchor (e.g., at extracorporeal unit 350), to advance the anchor distally through tube 310 and into the subject, and to anchor the anchor to internal tissue of the subject, e.g., to tissue 10 of the annulus of valve 12. For example, and as shown, implant 110 can be an annuloplasty implant, implanted by distributing anchors 120 around at least a portion of an annulus of valve 12.

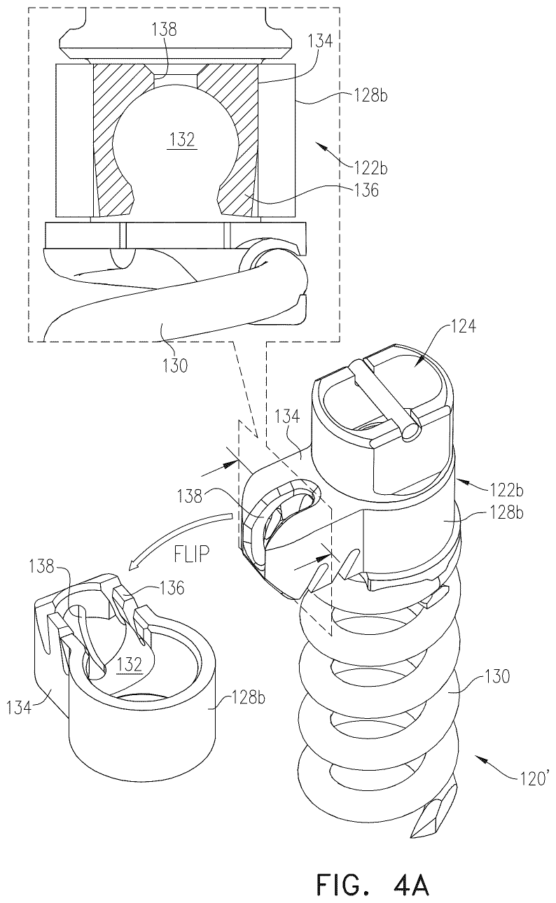

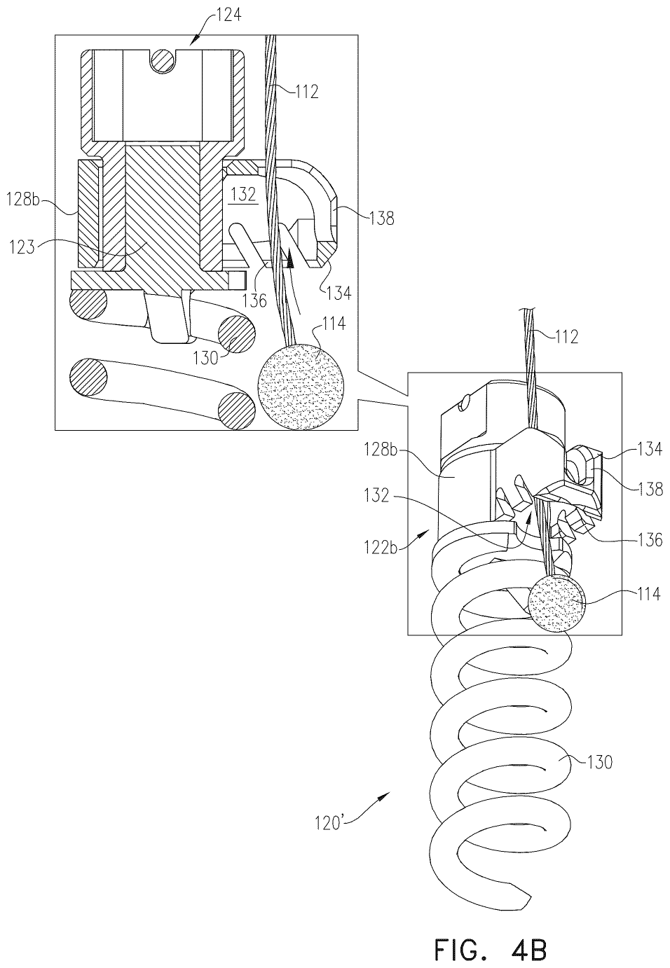

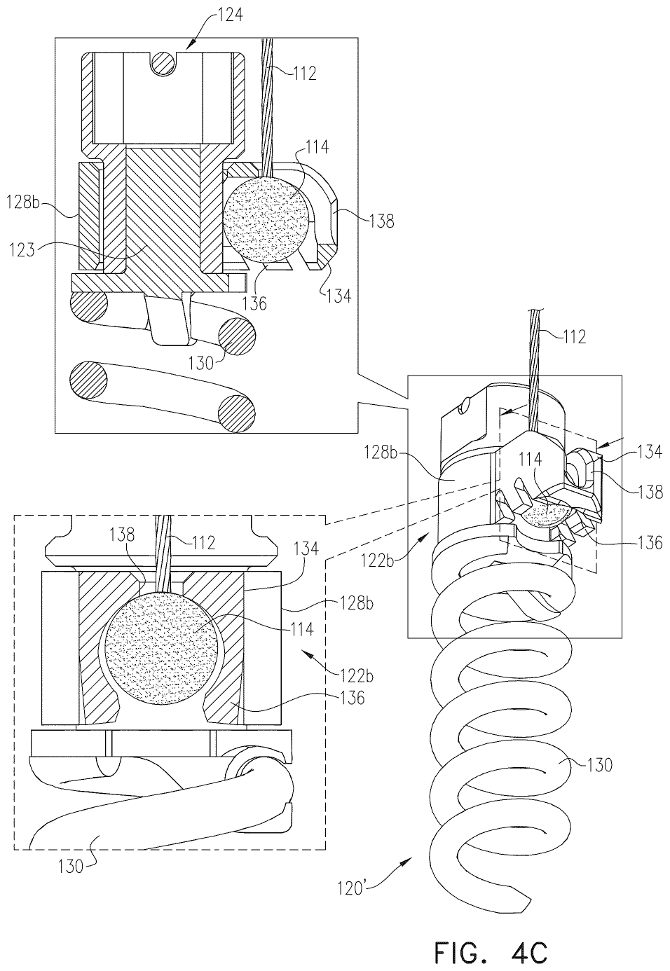

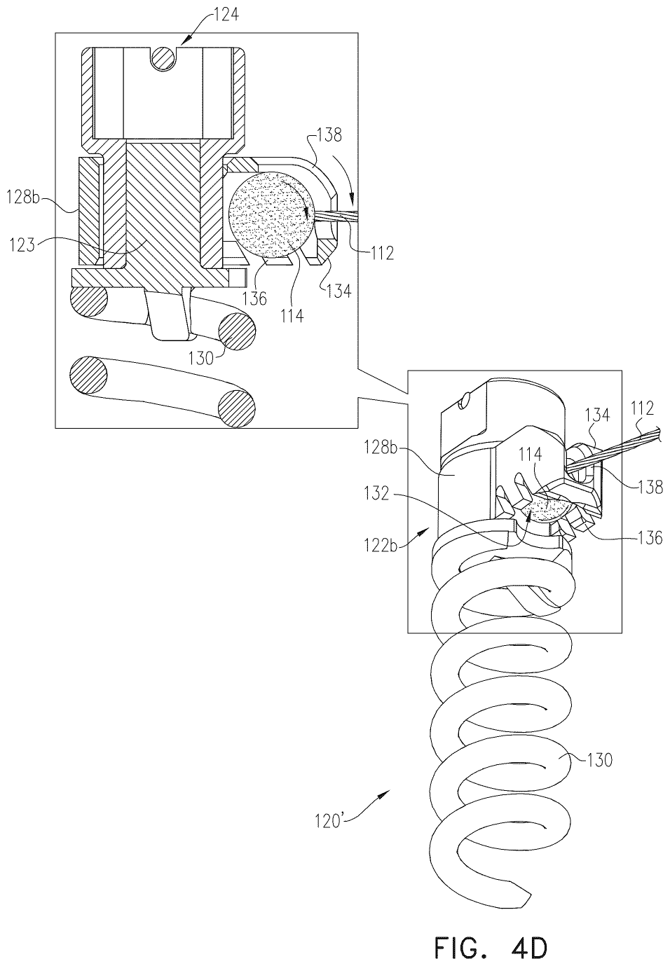

In some implementations, a distal end of tether 112 can be advanced distally into the subject along with the first anchor (herein the “leading anchor”), whereas successive anchors can be advanced by sliding them distally along the tether toward the leading anchor. Suffix ‘is used for the leading anchor, and suffix “is used for the successive anchors. Therefore, reference numeral 120 is used when referring to the anchors generically, reference numeral 120’ is used when referring specifically to the leading anchor, and reference numeral 120” is used when referring specifically to the successive anchors. In some implementations, leading anchor 120′ is identical to successive anchors 120″, whereas for other implementations the leading anchor can be specialized, e.g., can differ in one or more aspects, such as described with reference to FIGS. 4A-B.

FIG. 2A shows leading anchor 120′ having been anchored to tissue 10, e.g., to the annulus in the vicinity of a commissure of valve 12. At this point, tether 112 extends from leading anchor 120′, proximally through tube 310, to extracorporeal unit 350, where successive anchors 120″ are threaded on the tether. As noted above, this can be achieved by the distal end of tether 112 being advanced along with leading anchor 120′. For example, the distal end of tether 112 can be fixed to (e.g., may be non-slidable with respect to) leading anchor 120′.

In some implementations, the advancement and anchoring of leading anchor 120′ can be performed by the use of anchor driver 210. Anchor driver 210 (FIG. 1) can comprise an elongate and flexible shaft 212, and a drive head 214 coupled to a distal end of the shaft. While drive head 214 is engaged with anchor 120, anchor driver 210 is advanced to push the anchor through tube 310 to tissue 10. Anchor driver 210 is then used to anchor the anchor to tissue 10 by driving a tissue-engaging element of the anchor into the tissue, by applying an anchoring force to the anchor. For example, in some implementations, e.g., in which the tissue-engaging element of the anchor is helical or screw-like, anchor driver 210 applies torque to the anchor to screw the anchor into the tissue.

In some implementations, the tissue-engaging element can comprise one or more hooks, barbs, darts, staples, clips, protrusions, arms, expandable portions, threaded portions, rivets, pledgets, helixes, screws, screw-like portions, combinations of two or more of these, etc.

In some implementations, anchor driver 210 can further comprise a handle 216 and/or an actuator (e.g., a trigger) 218 that is operatively coupled to drive head 214 to control engagement of the drive head with anchor 120. 218 As shown, actuator 218 can be a component of handle 216. In some implementations, this operative coupling can be provided by a pull-rod that extends from actuator 218 to drive head 214, where a distal end of the pull-rod maintains engagement of the drive head with anchor 120 until actuator 218, and thereby also the pull-rod, are pulled proximally by the operator in order to disengage the drive head from the anchor.

In some implementations, after each anchor 120 has been anchored, anchor driver 210, once disengaged from the anchored anchor, is withdrawn proximally through tube 310 such that drive head 214 can be engaged with a subsequent anchor (e.g., at extracorporeal unit 350) in order to advance and anchor that subsequent anchor. FIG. 2B shows four anchors 120 (leading anchor 120′ and three successive anchors 120″) having been anchored to tissue 10, such that implant 110 begins to lie along part of the annulus of valve 12. Whereas tether 112 can be advanced with leading anchor 120′ (e.g., is pulled along by the leading anchor) successive anchors 120″ are advanced distally along (e.g., slid over and along) the tether toward the leading anchor.

FIG. 2C shows eight anchors 120 (leading anchor 120′ and seven successive anchors 120″) having been anchored to tissue 10, such that implant 110 lies along the posterior annulus of valve 12, approximately from commissure to commissure. It is to be understood that this number of anchors and this positioning is purely an illustrative example, and that the use of more or fewer anchors, and/or other placements are possible—at valve 12 and/or at another location. FIG. 2C also shows tether 112 being tensioned (e.g., by being pulled from outside of the subject), thereby drawing anchors 120 toward each other (e.g., shortening the length of implant 110) and contracting the annulus of valve 12, e.g., to improve coaptation of the leaflets of the valve. In some implementations, a tool or adjustment tool 400 (e.g., a tension/contraction and/or locking tool) can be used to facilitate this tensioning, e.g., by providing a reference force against the most recently-anchored anchor. In some implementations, tool 400 can be considered to be a component of system 100.

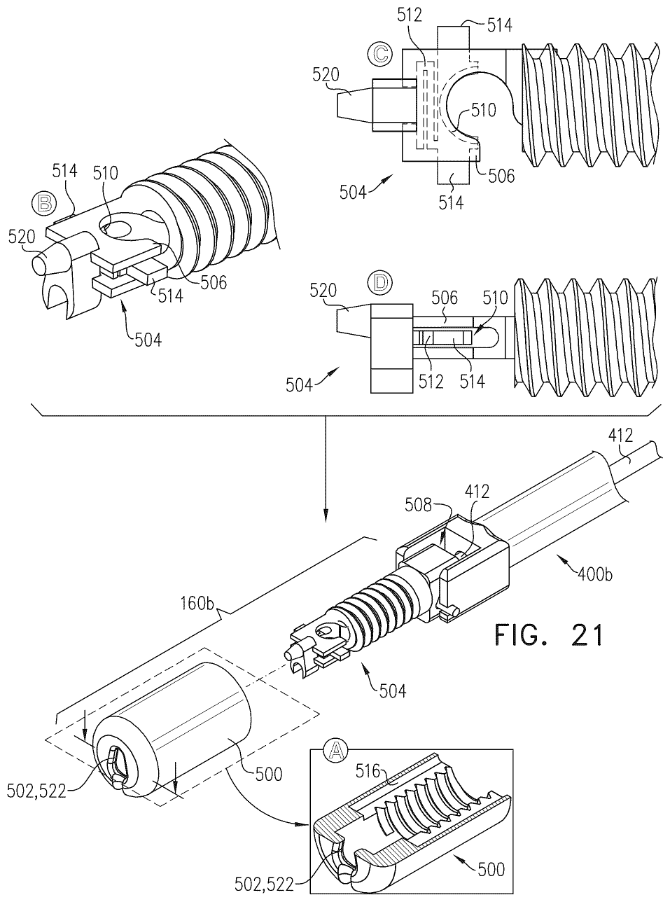

In some implementations, subsequently, the tension applied to tether 112 is locked in by locking a lock 160 (which may, in some implementations, be considered a stopper and/or be referred to as a stopper) to the tether (FIG. 2D), e.g., at the most recently-anchored anchor. At this point, tether 112 can be cut, and excess tether (e.g., tether proximal from lock 160) can be removed, e.g., as shown. In FIG. 2D lock 160 is shown as a discrete component. However, in some implementations, the final anchor 120 can include and/or can serve as a lock or stopper, i.e., can be a specialized anchor. The locking of lock 160 and/or the cutting of tether 112 can be performed directly or indirectly by tool 400. In some implementations, lock 160 can include a blade that cuts tether 112 upon actuation by tool 400, e.g., as described hereinbelow with reference to FIGS. 21-27C.

In some implementations, lock 160 and/or tool 400 is advanced, and/or tether 112 is tensioned, via tube 310, e.g., as shown. However, in some implementations, tube 310 can first be withdrawn from the subject.

In addition to its tissue-engaging element, in some implementations, each anchor 120 can have a head 122 from which the tissue-engaging element can extend distally in a manner that defines an anchor axis ax1 of the anchor. Head 122 can be rigidly attached to the tissue-engaging element. In some implementations, head 122 can comprise or define an interface 124 to which drive head 214 is reversibly engageable, anchor driver 210 applying the anchoring force (e.g., torque) via this engagement. Interface 124 of anchor 120 can be fixedly coupled to the tissue-engaging element of the anchor. For example, for implementations in which the tissue-engaging element is a screw-in (e.g., helical) tissue-engaging element, the tissue-engaging element can be screwed in by application of torque to interface 124. Interface 124 can be disposed on anchor axis ax1.

In some implementations, the tissue-engaging element can comprise one or more hooks, barbs, darts, staples, clips, protrusions, arms, expandable portions, threaded portions, rivets, pledgets, helixes, screws, screw-like portions, combinations of two or more of these, etc.

In some implementations, each anchor of a series of anchors has the same type of head and/or same type of tissue-engaging element. In some implementations, some anchors of the series of anchors have different types of heads and/or different types of tissue-engaging elements from other anchors of the series of anchors (e.g., some have a first type of head and/or tissue-engaging element, while one or more anchors have a different second type of head and/or tissue-engaging element.)

For those anchors 120 that are slidably coupled to tether 112 (e.g., successive anchors 120″) this slidable coupling can be provided by an eyelet 126 of the anchor, the tether being threaded through the eyelet. Eyelet 126 can be a component of, or coupled to, head 122 of the anchor.

In some implementations, and as shown in FIGS. 2A-D, implant 110 can comprise one or more spacers (or dividers) 150 between anchors 120. For example, and as shown, each spacer 150 can be disposed in a respective inter-anchor space between two adjacent anchors 120, e.g., can be bookended by the two adjacent anchors. Spacers 150 can be present in every inter-anchor space (e.g., as shown), or in only a subset of inter-anchor spaces. Spacers 150 can be threaded onto tether 112.

In some implementations, spacer 150 is flexible in deflection, e.g., elastically (e.g., may be resilient) or plastically. Distinct from this flexibility, spacer 150 may resist axial compression (e.g., can be axially incompressible), or can be axially compressible to some degree. In the example shown, spacer 150 is tubular, and is defined by a coil. In some implementations, spacer 150 has one or more characteristics of the spacers described in in WO 2021/084407 to Kasher et al. or WO 2022/172149 to Shafigh et al., each of which is incorporated herein by reference.

During contraction of implant 110, each spacer 150 can inhibit approximation of the anchors by which it is bookended. In some implementations (e.g., in some implementations in which spacer 150 is axially incompressible), this inhibition can take the form of defining a discrete minimum inter-anchor distance between the bookending anchors. In some implementations (e.g., in some implementations in which spacer 150 is axially compressible), this inhibition can be provided over a continuum of inter-anchor distances. In some implementations, spacer 150 can be configured to combine both of these forms of inhibition. Spacers 150 can advantageously distribute contraction and/or forces along implant 110 and/or between anchors 120.

The configuration and/or distribution of spacers 150 within implant 110 can be selected according to particular needs. For example, in some implementations, spacers 150 can be configured and/or distributed in order to achieve greater homogeneity of contraction and/or force across the entirety of the implant. In some implementations, the configuration and/or distribution can be selected in order to concentrate contraction and/or force on one or more regions of the implant and/or of the tissue.

In some implementations, spacers 150 are separate from anchors 120. For example, spacers 150 can be threaded onto tether 112 separately from the anchors 120, and/or can be coupled to the anchors only via the tether.

Reference is again made to FIGS. 1-2D. In some implementations, catheter device 300 (e.g., extracorporeal unit 350 thereof) comprises a de-slacker 354 that, during implantation of implant 110, reduces slack on tether 112 (e.g., prevents the tether from becoming slack) and/or generally manages the tether. It is hypothesized that this may advantageously reduce a likelihood of tether 112 becoming twisted or entangled, or of inadvertent engagement of the tether with an anchor. In some implementations, de-slacker 354 comprises a spring-loaded winch. Such a configuration may, compared with a human operator manually pulling on a proximal end of the tether, advantageously provide greater control over and/or consistency of the magnitude of tension applied to the tether, and may further advantageously reduce the number of human operators required.

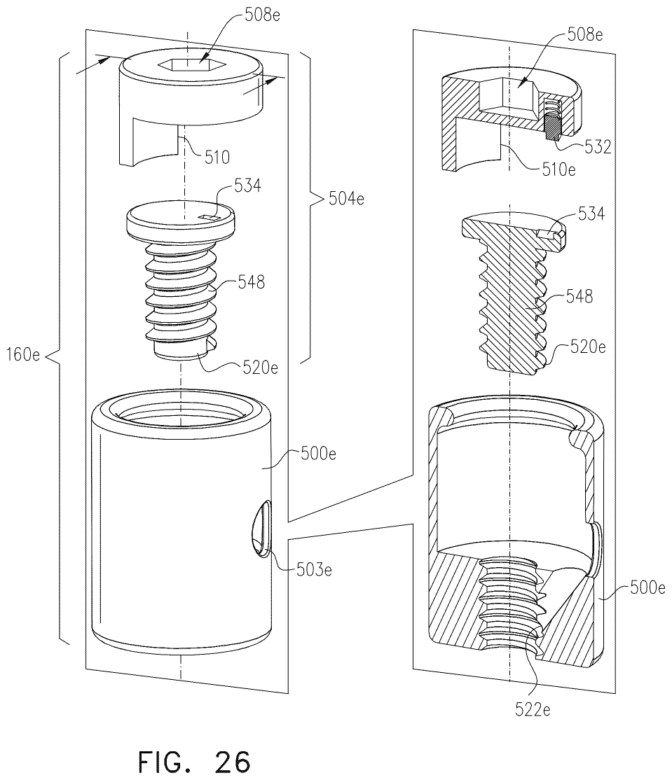

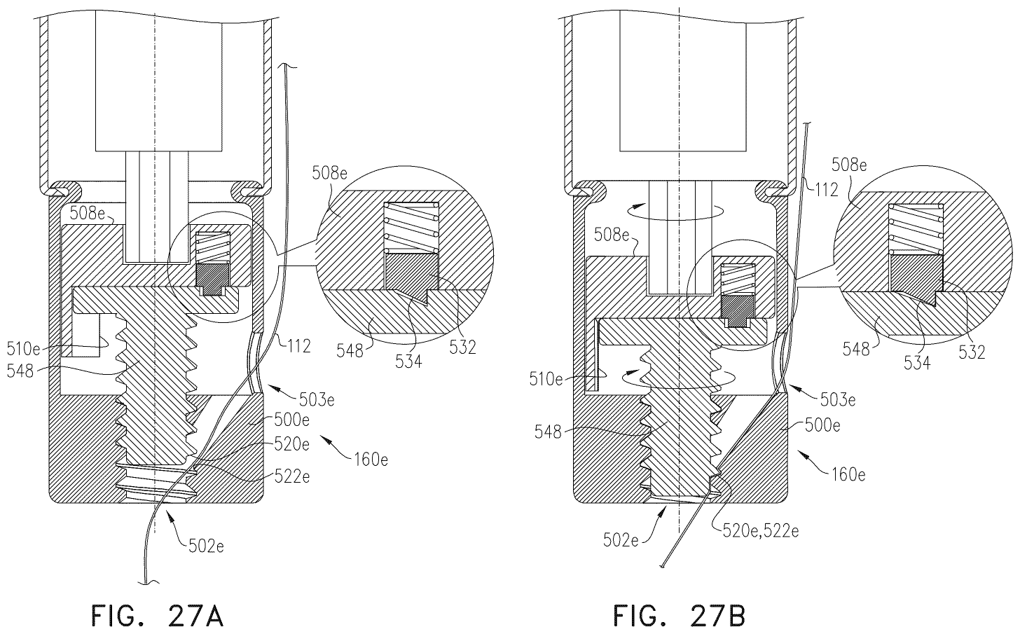

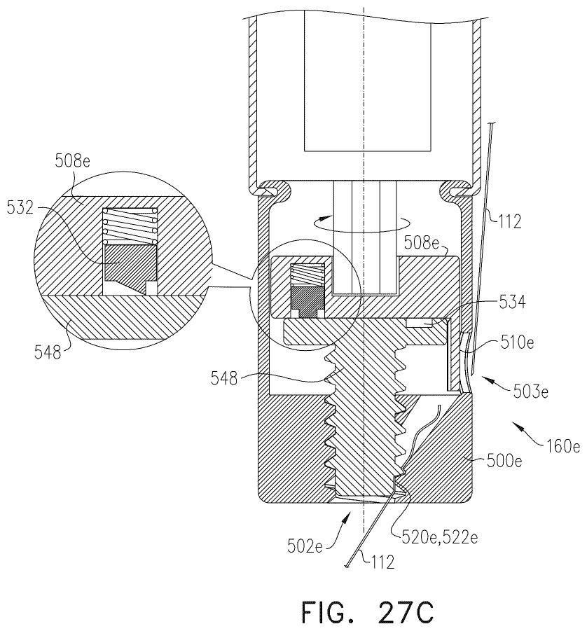

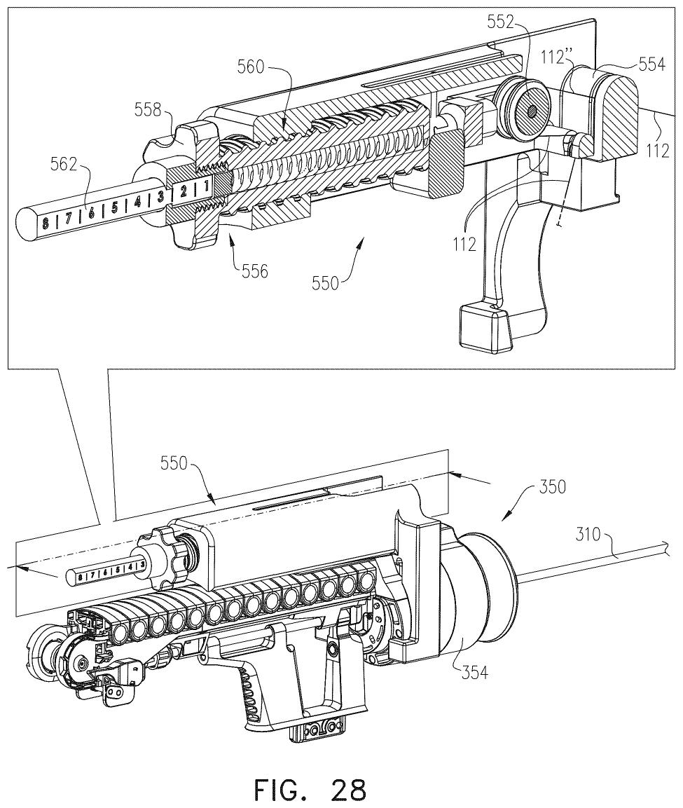

In some implementations, de-slacker 354 can share one or more features with (e.g., can be as described for) the “tensioner” described in International Patent Application (PCT) Publication WO 2022/064401 to Halabi et al., which is incorporated herein by reference. Although de-slacker 354, at least while serving this de-slacking function, may not apply sufficient tension on tether 112 to affect (e.g., contract) the tissue in which anchors 120 are anchored, in some implementations the de-slacker can nonetheless be considered to apply a small amount of tension to the tether, e.g., sufficient to reduce/eliminate slack, but insufficient to materially affect the tissue during implantation. Thus, in some implementations, de-slacker 354 can be considered to be a “tensioner”. However, for the sake of clarity, throughout the present application the term “tensioner” is reserved for tensioners that are, in fact, configured to tension tether 112 sufficiently to contract the tissue. Some such tensioners are described hereinbelow, e.g., with reference to FIGS. 28-30.

In some implementations, de-slacker 354 can be deactivated by the operator, such that the de-slacker ceases to reduce slack on tether 112. In some implementations, de-slacker 354 is configured such that deactivation also allows tether 112 to be pulled out of the de-slacker without opposition from the de-slacker.

In some implementations, and as mentioned hereinbelow, de-slacker 354 can be locked by the operator, such locking preventing tether 112 from being pulled out of the de-slacker. In some implementations, such locking also deactivates the de-slacker, e.g., such that any slack introduced into the tether is not reduced by the de-slacker. In some implementations catheter device 300 (e.g., extracorporeal unit 350 thereof) can be provided with a similar locking function separate from de-slacker 354—even in some implementations in which the catheter device does not comprise a de-slacker. Irrespective of whether the locking function is part of a de-slacker, it may be advantageous, inter alia, for implementations in which a tensioner is used, e.g., as described with reference to FIGS. 28-30.

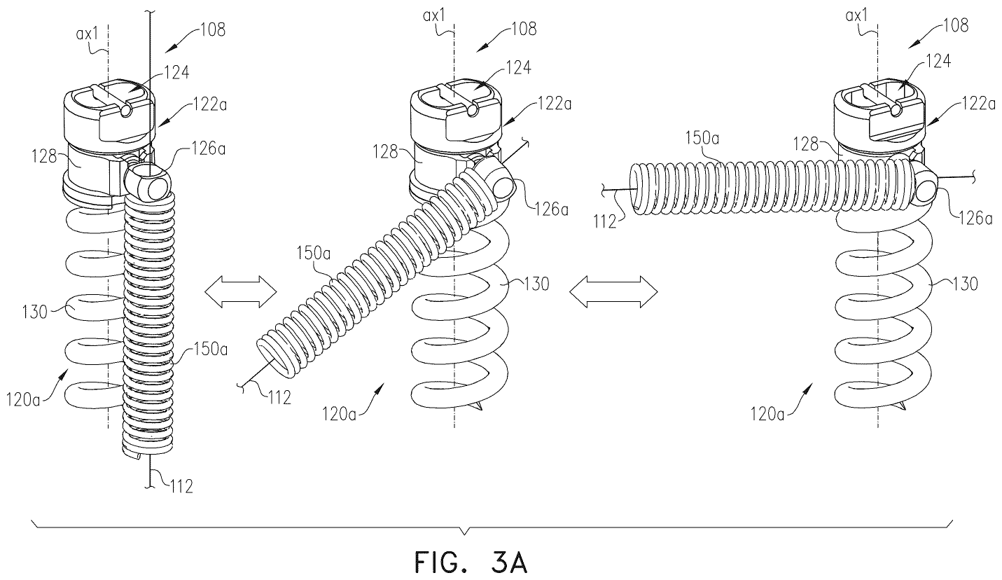

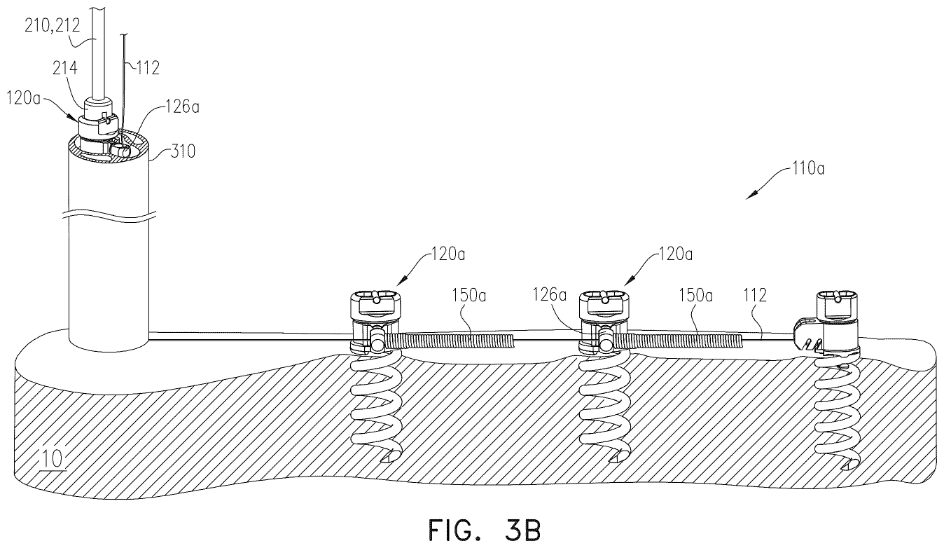

Reference is additionally made to FIGS. 3A-B, which are schematic illustrations of an anchor 120a and a corresponding spacer 150a, in accordance with some implementations. FIG. 3A shows anchor 120a and spacer 150a threaded onto tether 112, and FIG. 3B shows a step in the implantation of an implant 110a that comprises such anchors and spacers, in accordance with some implementations. In some implementations in which the implant 110 includes spacers, rather than the spacers being separate from the anchors, the spacers are coupled to the anchors, e.g., independently of tether 112. Spacer 150a is an example of this, in which the spacer is attached (e.g., welded, brazed, soldered, glued, and/or sewn) to anchor 120a, e.g., to a head 122a of the anchor, such as to an eyelet 126a thereof.

In some implementations, as in the example shown, anchor 120a has a helical tissue-engaging element 130, configured to be screwed into tissue. Tissue-engaging element 130 defines anchor axis ax1 by extending in a helix around and along the anchor axis.

In the example shown, anchors 120a are configured, and threaded onto tether 112, to be advanced along the tether with spacer 150a leading, e.g., pointing toward the preceding anchor. However, in some implementations, anchors 120a are configured, and threaded onto tether 112, to be advanced along the tether with spacer 150a trailing, e.g., pointing toward the subsequent anchor (once the subsequent anchor is also advanced).

In some implementations, spacer 150a can be considered to be a component of anchor 120a. For example, spacer 150a can be considered to be part of (e.g., an extension of) eyelet 126a. Similarly, in some implementations, eyelet 126a can be considered to define spacer 150a.

In some implementations, anchor 120a and spacer 150 can be considered to collectively define an anchor-spacer assembly 108. It is to be noted that, in this context, “assembly” denotes that the system is provided with these components connected to each other, and that they remain connected during their advancement and implantation.

Once implant 110 has been implanted, anchor axis ax1 of each anchor 120 can be substantially rotationally offset from tether 112 (e.g., substantially orthogonal to the tether). In FIG. 3B, such an offset is observable for the three anchors that are shown as having been anchored to tissue 10. However, during advancement of each anchor 120 to the heart (e.g., through tube 310), anchor axis ax1 of the anchor can be less offset from tether 112 (e.g., can be substantially parallel with the tether 112). In FIG. 3B, this is observable for the anchor that is shown within tube 310. Therefore, eyelet 126 (including variants thereof, such as eyelet 126a) can be configured to allow passage of tether 112 therethrough (and therefore sliding of anchor 120 along the tether) both while anchor axis ax1 is substantially parallel with tether 112 (e.g., during advancement through tube 310), and while the anchor axis is substantially orthogonal to the tether (e.g., upon anchoring and/or during contraction).

In some implementations, eyelet 126a provides this functionality by being rotatably mounted, with spacer 150a pivoting responsively to rotation of the eyelet to which it is coupled. FIG. 3A illustrates this rotatable mounting by showing three example rotational orientations. The left-side example shows eyelet 126a (and spacer 150a) rotationally oriented in a manner that facilitates passage of tether 112 while anchor axis ax1 is substantially parallel with the tether (e.g., a delivery state of anchor 120a), the right-side example shows a rotational orientation that facilitates passage of the tether while the anchor axis is substantially orthogonal to the tether, and the center example shows a rotational orientation partway between the other two orientations, e.g., the anchor axis is offset from, but not orthogonal to, the tether.

Alternatively or additionally, eyelet 126 and/or spacer 150 can be mounted so as to be revolvable about anchor axis ax1. This characteristic can advantageously facilitate rotation of interface 124 and tissue-engaging element 130 (in order to screw the tissue-engaging element into the tissue) while tether 112 remains relatively still, e.g., without winding the tether onto anchor 120. In some implementations, and as shown for anchor 120a, this characteristic is achieved by the eyelet and/or the spacer (e.g., by virtue of its coupling to the eyelet) being coupled to a collar 128 that is rotatable around the anchor axis. For example, anchor head 122 (e.g., anchor head 122a) can comprise a stock 123 that fixedly couples interface 124 to tissue-engaging element 130 (and that can lie on anchor axis ax1), and collar 128 can circumscribe, and be rotatable about, the stock. Stock 123, of a different anchor, is visible in FIGS. 4A-D. The stock can be configured in a variety of ways, e.g., as a core, a rod, a tube, a neck, a winch, a peg, etc.

In some implementations, the tissue-engaging element can comprise one or more hooks, barbs, darts, staples, clips, protrusions, arms, expandable portions, threaded portions, rivets, pledgets, helixes, screws, screw-like portions, combinations of two or more of these, etc.

In some implementations, and as shown, each anchor 120a is configured to be advanced (e.g., has a delivery state) with spacer 150a extending away from anchor head 122a along tether 112 and/or alongside tissue-engaging element 130. Similarly, in some implementations, and as shown, each anchor 120a is advanced with spacer 150a extending away from anchor head 122a toward the preceding anchor (e.g., distally and/or facing toward the preceding anchor).

In some implementations, each anchor 120a can be configured to be advanced (e.g., has a delivery state) with spacer 150a extending proximally away from anchor head 122a (e.g., alongside shaft 212), e.g., facing toward the next anchor to be advanced.

In some implementations, and as shown, in order to accommodate passage of anchor 120a and spacer 150, the internal channel of tube 310 has a keyhole-shaped orthogonal cross-section that defines a minor channel-region, and a major channel-region that has a larger cross-sectional area than the minor channel-region. In some implementations, anchor 120a is advanced through the channel by driver 210 with anchor head 122 and/or tissue-engaging element 130 sliding snugly through the major channel-region, and with eyelet 126a and/or spacer 150a sliding snugly through the minor channel-region. Spacer 150a can be configured to restrain tether 112 within the minor channel-region as anchor 120a is advanced through the channel. Inter alia, this may advantageously reduce a likelihood of tissue-engaging element 130 undesirably engaging tether 112.

In some implementations, spacer 150 is longer than tissue-engaging element 130 and/or can extend beyond a distal end (e.g., a sharp point) of the tissue-engaging element-see, for example, the left-side image of FIG. 3A. Inter alia, this may advantageously further reduce a likelihood of tissue-engaging element 130 undesirably engaging tether 112.

Reference is now made to FIGS. 4A-D, which are schematic illustrations of leading anchor 120′, in accordance with some implementations. As described hereinabove, in some implementations, leading anchor 120′ can be specialized. FIGS. 4A-B show an example in which specialization of leading anchor 120′ facilitates fixation of the leading anchor to tether 112, e.g., to the distal end of the tether.

In some implementations, leading anchor 120′ comprises a tissue-engaging element (e.g., tissue-engaging element 130), and an anchor head 122b that comprises a socket 132. A stopper 114 is fixedly attached to tether 112, e.g., to the distal end of the tether. This fixation can be achieved by compression (e.g., crimping), welding, brazing, soldering and/or gluing. Leading anchor 120′ is fixed to tether 112 by stopper 114 being secured within socket 132.

FIG. 4A is a perspective view of leading anchor 120′ and includes a “flip” view that shows socket 132 (e.g., an interior thereof), e.g., from an underside of the socket. FIG. 4A also includes an inset cross-section through socket 132.

In some implementations, socket 132 can be defined by a casing 134, e.g., the socket can be a recess defined by the casing. In some implementations, casing 134 can be revolvable about the anchor axis of anchor 120c and/or about a stock of anchor head 122c, e.g., by being coupled to a collar 128b that is rotatably mounted. In some implementations, and as shown, casing 134 and collar 128b can be formed from a single unitary piece of stock material. Such rotatable mounting of casing 134 via collar 128b can be as described for the rotatable mounting of eyelet 126 via collar 128, mutatis mutandis. In some implementations, casing 134 and/or collar 128b can be considered to be components of head 122b.

FIG. 4B shows stopper 114, fixed to tether 112, being introduced into socket 132 in accordance with some implementations. FIGS. 4C-D show stopper 114, fixed to tether 112, in different rotational orientations within socket 132, in accordance with some implementations.

In some implementations, at least one cantilever 136 (e.g., a component of casing 134) retains stopper 114 within socket 132. For example, and as shown in FIGS. 4B-C (e.g., the transition therebetween), stopper 114 can be introduced into socket 132 via an open side of the socket, and cantilever(s) 136 can obstruct stopper 114 from exiting the socket via the open side. In some implementations, and as shown, stopper 114 is snap-fitted into socket 132, e.g., with cantilever(s) 136 providing the snap-fit functionality by transiently moving to accommodate movement of the stopper into the socket. Thus, cantilever(s) 136 can be resilient, and biased to provide a gap that is less wide than stopper 114 (e.g., is smaller than the diameter of the stopper).

In some implementations, stopper 114 is introduced into socket 132 by pulling on tether 112. For example, casing 134 can define a window 138 into socket 132, through which tether 112 is threaded and pulled, thereby pulling stopper 114 into the socket. FIGS. 4B-C (e.g., the transition therebetween) can be considered to represent this. Window 138 is discrete from the open side of socket 132 and can be across from the open side of the socket. For example, and as shown, at least part of window 138 can be at an opposite side of socket 132 from the open side. Thus, once stopper 114 is disposed within socket 132, tether 112 extends, from the stopper, through window 138 to exit the socket.

In some implementations, stopper 114 and socket 132 are shaped and dimensioned such that the stopper is rotatable while secured within the socket. For example, stopper 114 can be bulbous (e.g., can be a bead and/or can be substantially spherical), and the stopper and socket can function as a ball-and-socket joint.

In some implementations, window 138 can be sized and/or shaped to accommodate at least some such rotation, and the accompanying pivoting of tether 112 with respect to casing 134 (e.g., and with respect to head 122b in general). For example, window 138 can extend partway around socket 132 and/or stopper 114 therewithin, e.g., can curve in an arc. For example, window 138 can be elongate and/or can extend at least a fifth of the way around socket 132 and/or stopper 114 therewithin. This can facilitate tether 112 pivoting between (i) an axial state in which the tether extends through window 138 in a trajectory that is parallel with the anchor axis (FIG. 4C), and (ii) a lateral state in which the tether extends through the window in a trajectory that is rotationally offset from (e.g., orthogonal to) the anchor axis (FIG. 4D). For example, tether 112 can be in the axial state during advancement of the anchor through tube 310, and/or can be in the lateral state following implantation.

Due to the above-described rotatability of casing 134 and/or collar 128b, the casing can responsively turn to face the first successive anchor of the implant upon tensioning of tether 112. This, in combination with the above-described rotatability of stopper 114, and the size and shape of window 138, can advantageously allow tether 112 to lie in a substantially straight line between stopper 114 and the first successive anchor of the implant, thereby reducing potentially tether-damaging bending of the tether and pressing of the tether against components of the leading anchor.

In some implementations, in order to facilitate the above-described rotatability of stopper 114 within socket 132, the end of the tether does not protrude from the stopper. For example, the end of tether 112 can be flush with an external surface of the stopper. This can be achieved, for example, by cutting and/or grinding away excess tether 112 after the stopper has been secured to the tether. In some implementations, the end of tether 112 can even be within the stopper.

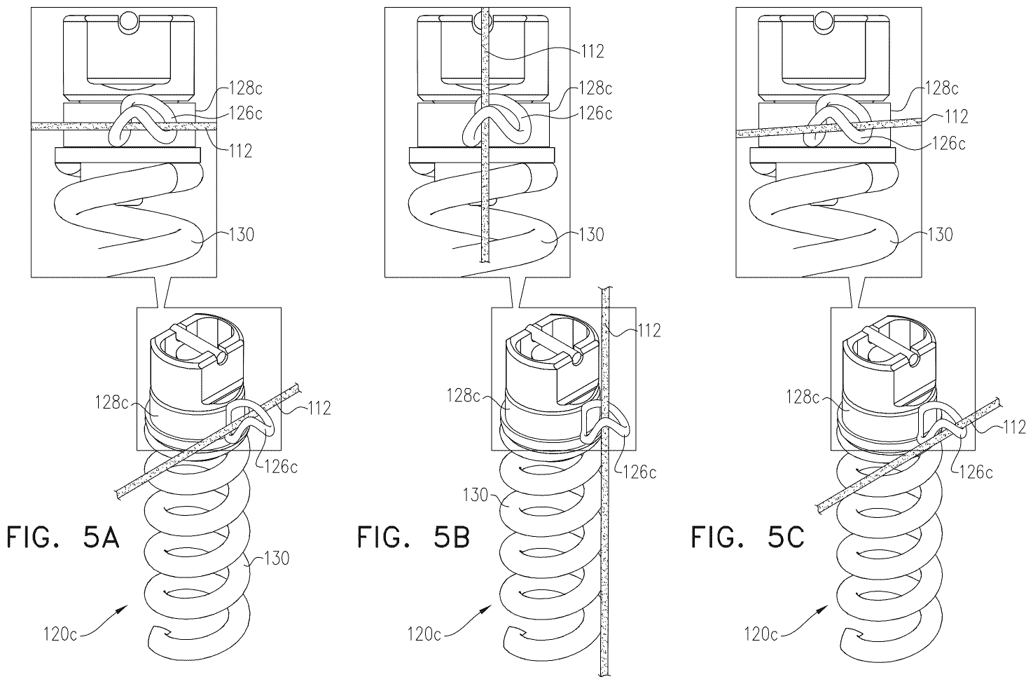

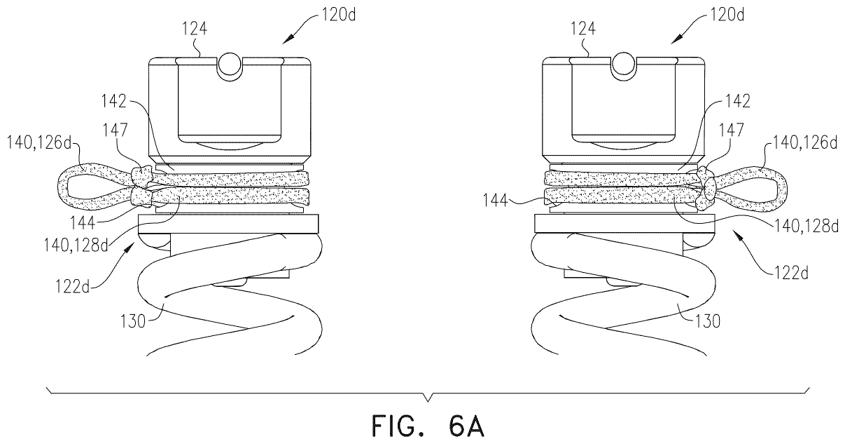

Reference is now made to FIGS. 5A-C, which are schematic illustrations of an anchor 120c, in accordance with some implementations. As described hereinabove, it may be advantageous for an anchor of implant 110 (or a similar implant) to facilitate the tether of the implant being pivotable between an axial state (e.g., for transcatheter advancement) and a lateral state (e.g., upon implantation) with respect to the anchor. It may further be advantageous for such an anchor, especially when the anchor is serving as a successive anchor of the implant, to be slidable along the tether both in the axial state and in the lateral state. Anchor 120c provides such a feature through its eyelet 126c, which is saddle shaped. Similarly to eyelet 126 described hereinabove, eyelet 126c can be mounted eccentrically, and/or can be revolvable about the anchor axis and/or stock 123 of the anchor, e.g., via rotation of a collar 128c.

FIG. 5B shows tether 112 substantially parallel with the anchor-axis of anchor 120c (e.g., as it may be during advancement of the anchor through tube 310), FIG. 5A shows the tether deflected with respect to the anchor-axis (e.g., as it may be upon implantation of the anchor), and FIG. 5C shows the tether deflected in the opposite direction with respect to the anchor-axis (e.g., in the opposite direction to that shown in FIG. 5A). In each of FIGS. 5A-C, eyelet 126c provides a straight, clear line-of-sight through the eyelet, so that the eyelet can slide smoothly along the tether irrespective of the rotational orientation of the tether with respect to the anchor axis.

Although anchor 120c can serve as a successive anchor (e.g., similarly to successive anchor 120″), and is described in this context, it is to be understood that it can also be used as a leading anchor.

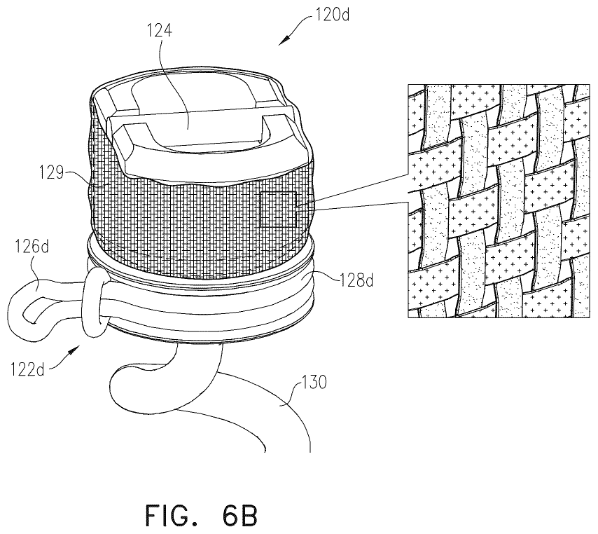

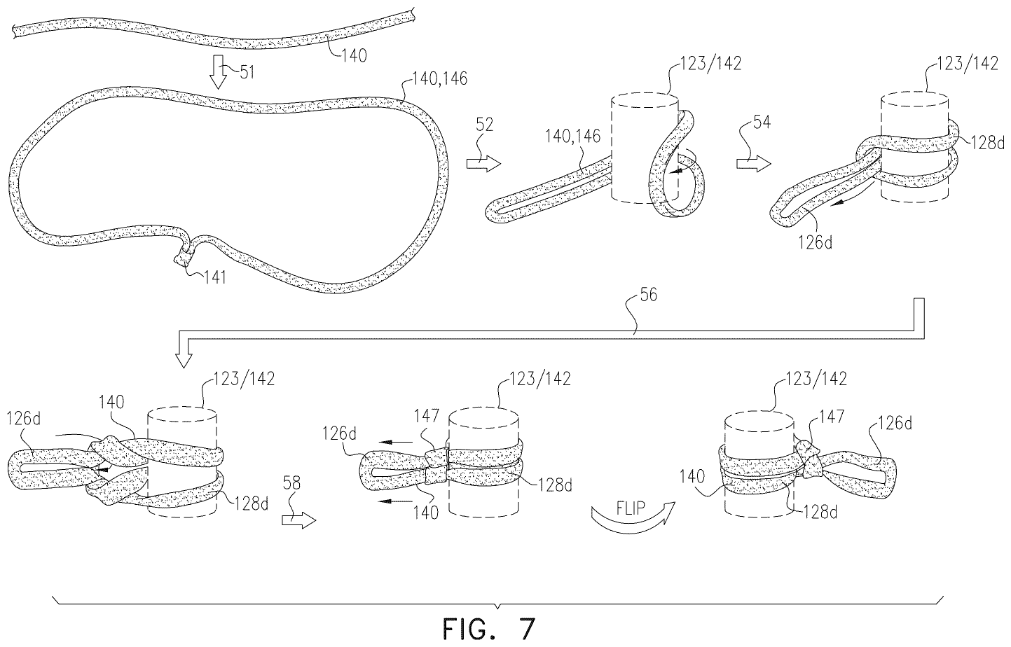

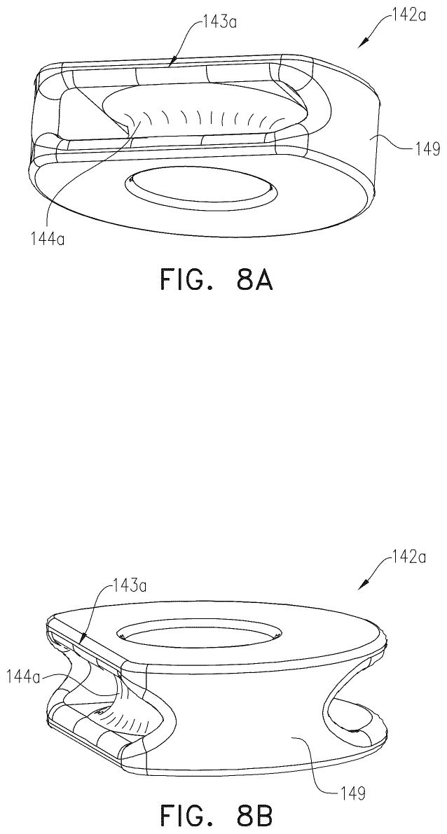

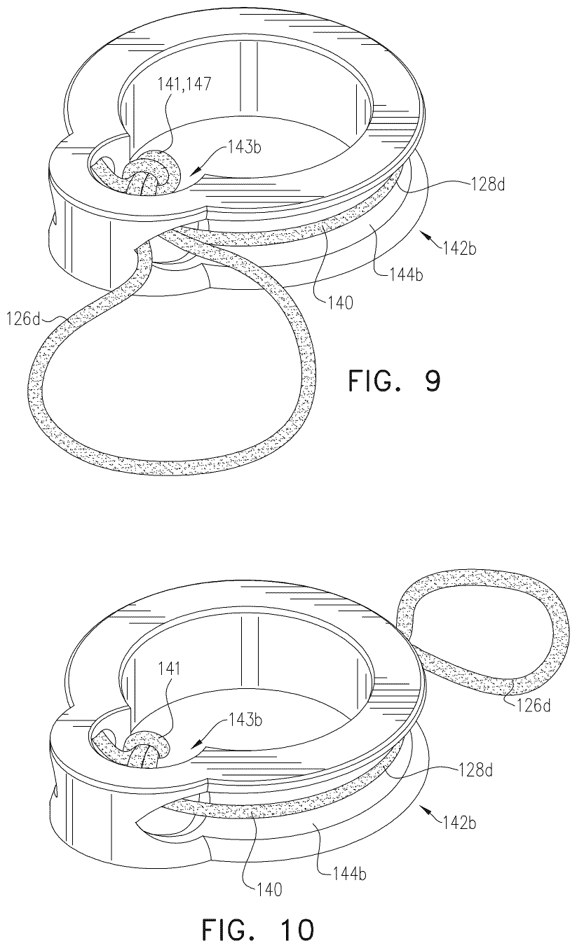

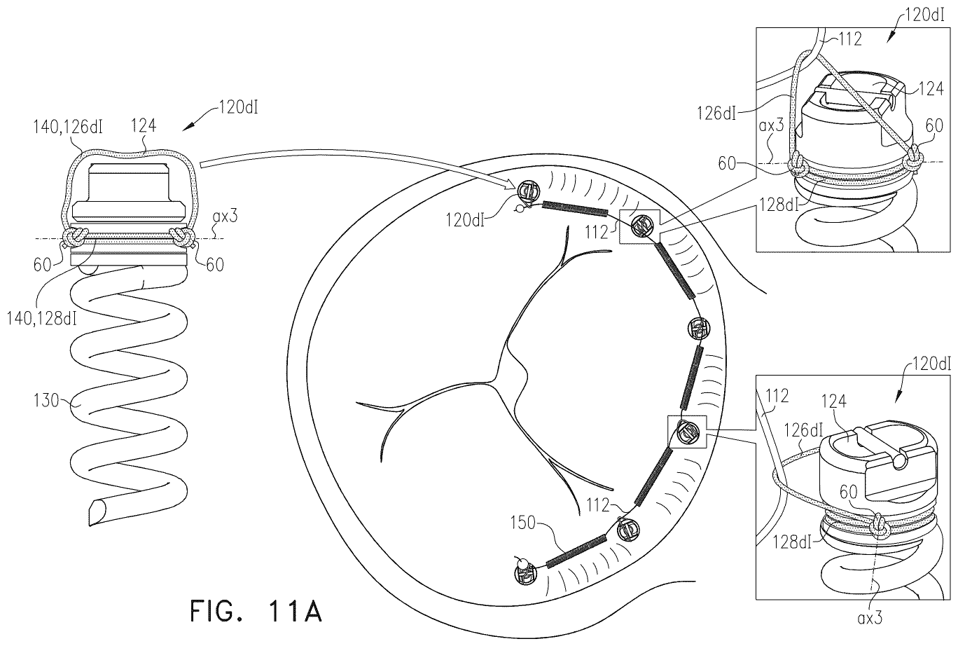

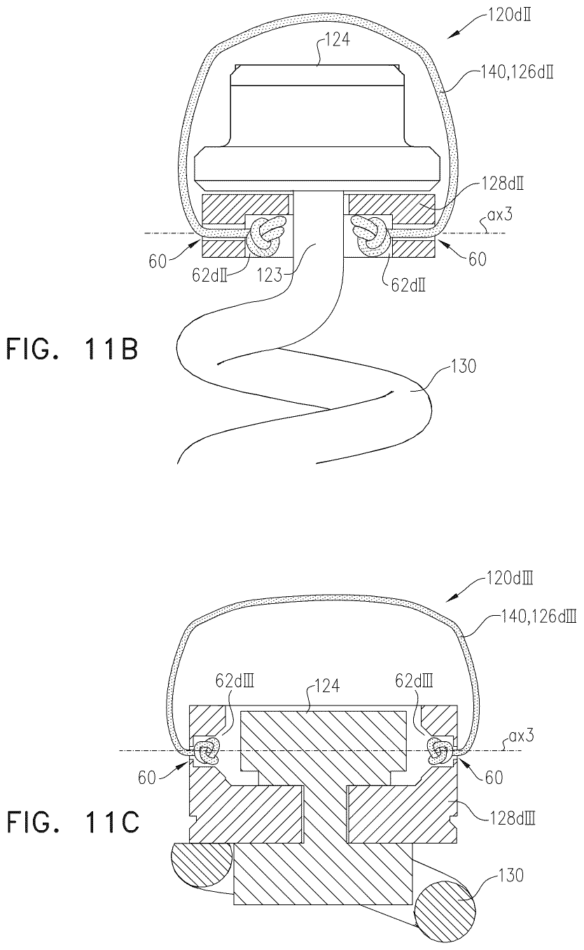

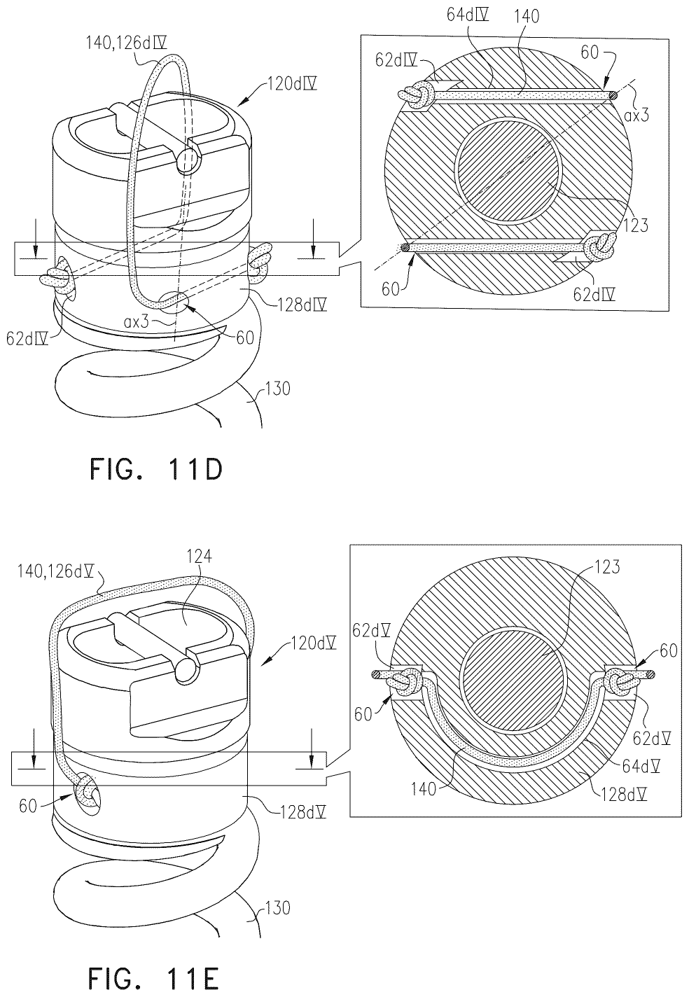

Reference is made to FIGS. 6A-B, 7, 8A-B, 9, 10, and 11A-K, which are schematic illustrations of example eyelets, e.g., eyelets that are formed from textile (e.g., from a polyfilament structure) and/or a polymer, in accordance with some implementations.

An eyelet formed from a textile can be flexible and strong, and may thereby advantageously provide (i) a high degree of freedom of deflection of the anchor axis with respect to tether 112, (ii) smooth sliding of the eyelet over and along the tether, (iii) low wear on the tether, and/or (iv) long-term durability of the eyelet.

FIG. 6A shows two opposite views of an anchor 120d, whose eyelet 126d comprises and/or is formed from a textile and/or polymer 140. As a result of being formed from textile and/or polymer 140, eyelet 126 can be highly flexible. Furthermore, eyelet 126 may be less wearing on tether 112, e.g., compared to a metallic eyelet. In some implementations, and as shown, textile and/or polymer 140 is a yarn (e.g., a suture).

In some implementations, textile 140 is a fabric (e.g., a woven fabric or a non-woven fabric). In some implementations, textile 140 can comprise filaments of a natural fiber and/or filaments of a synthetic polymer.

While the term “textile” is commonly used in this disclosure, in some implementations, a polymer can be configured in the same way described with respect to the various textiles herein, e.g., to form the eyelets and/or collars herein, even if the polymer might not be configured as a common textile. In some implementations, the polymer may not comprise any polyfilament structure, any fabric, any weave, etc.

Additionally (or alternatively), a collar 128d of anchor 120d can be formed from textile 140. FIG. 6A shows eyelet 126d and collar 128d having both been formed from textile 140, each being defined by respective loops (one or more loops each) into which the textile has been secured.

In some implementations, textile 140 is formed into eyelet 126d by knotting/tying the textile (e.g., the yarn). In some implementations, textile 140 is formed into collar 128d by knotting/tying the textile (e.g., the yarn). In some implementations, textile 140 is coupled to head 122d of the anchor by knotting/tying.

As for other collars described herein, collar 128d can be configured to be rotatable about the anchor axis of anchor 120d, e.g., about stock 123 of head 122d.

In some implementations, anchor 120d (e.g., head 122d thereof) comprises an optional bushing 142, disposed medially from collar 128d (e.g., concentrically between the eyelet and stock 123). In some implementations, bushing 142 is configured to facilitate rotation of collar 128d, e.g., by the bushing being rotatable about stock 123. Bushing can be made from a polymer such as polyether ether ketone (PEEK).

In some implementations, bushing 142 can be generally annular. In some implementations, bushing 142 can define a radially-facing (e.g., circumferential) groove 144 in which the eyelet resides, the groove stabilizing the eyelet on the bushing, e.g., preventing slippage of the eyelet off of the head of the anchor.

In some implementations, and as shown in FIG. 6B, a snood 129 is disposed around anchor head 122d (e.g., the anchor head is dressed in the snood) in a manner that preserves accessibility to interface 124. Snood 129 can be dimensioned and positioned so as not to engage or interfere with collar 128d, eyelet 126d, or tether 112.

In some implementations, snood 129 can comprise (e.g., be formed from) a textile (e.g., textile 140), a sponge, and/or a multilaminar material (e.g., layered cellulose sheets). The material from which snood 129 is formed can optionally be configured to promote tissue growth thereon.

In some implementations, snood 129 can be absorbent (e.g., defining pores or pockets), such that it can carry a substance to the site of anchoring and then progressively release the substance at the site. In some implementations, the substance is absorbed into the snood in the same facility (e.g., in the same operating theater) in which the procedure that uses the anchor will be performed. In some implementations, the substance is absorbed into the snood by the person (e.g., the physician) who will perform the procedure that uses the anchor. In some implementations, the substance is absorbed into the snood no more than two hours (e.g., no more than one hour, such as no more than ten minutes, such as no more than 2 minutes) prior to performing the procedure (e.g., prior to transluminally advancing the anchor into the subject). In some implementations, the substance is absorbed into the snood while the driver that will be used to advance and/or anchor the driver is engaged with interface 124, e.g., by using the driver to dip the anchor into the substance.

In some implementations, the substance comprises a medicament. In some implementations, the substance comprises a radiopaque dye.

It is to be understood that a snood such as snood 129 can be used with the head of any tissue anchor including, but not limited to, other tissue anchors described herein.

FIG. 7 shows at least some steps of a technique for forming collar 128d and/or eyelet 126d from textile 140, in accordance with some implementations. In this example, textile 140 is an elongate structure, e.g., a yarn, such as a suture. A length of textile 140 is formed into a closed loop 146 (step 51). This can be achieved by tying and/or by heating (e.g., melting/fusing) the textile. Loop 146 can therefore have a visible join 141, such as a knot. For simplicity, join 141 is not shown in the remaining steps of FIG. 7. In some implementations, multiple loops may be formed and/or used.

For implementations in which the anchor has bushing 142, loop 146 is then wrapped around the bushing (step 52) and passed through itself (step 54) such that two lengths of the polyfilament yarn extend in parallel around the bushing to form collar 128d. This step can be considered connecting the loop to bushing 142 using a cow hitch. For implementations in which the anchor has no bushing, loop 146 can instead be wrapped around part of the head of the anchor (e.g., another part), such as around stock 123. In FIG. 7, the cylinder drawn in broken lines is intended to represent bushing 142 and/or the part of the head (e.g., stock 123). The part of loop 146 that extends away from collar 128d forms eyelet 126d. This part of loop 146 can be passed through the loop again, e.g., to prevent unlooping (step 56). Before and/or after this second looping, loop 146 is pulled tight (step 58).

At the intersection between collar 128d and eyelet 126d (e.g., where loop 146 loops through itself once or more), textile 140 (e.g., loop 146) can define a knot (or other bulky feature) 147. FIGS. 8A-B, 9, and 10 are schematic illustrations of bushings that are shaped to define, inter alia, a recess 143 that is shaped to receive or otherwise accommodate knot 147 and/or join 141, e.g., so as to protect it or obscure it from exposure to the bloodstream. FIG. 8 shows a bushing 142a that has a recess 143a that is defined by a cropped part of the bushing that has a reduced radius (e.g., from the anchor axis), e.g., compared with other parts of the bushing. Whereas recess 143a faces laterally away from the anchor axis (when bushing 142a is mounted on the anchor), FIGS. 9 and 10 show a bushings 142b that defines a recess 143b that faces medially toward the anchor axis. As shown, recess 143b can be a cubby, defined by a bulge of bushing 142b that bulges laterally.

FIG. 9 shows an arrangement in which knot 147 is disposed in recess 143b, and eyelet 126d extends out of a window in the recess. Join 141 can also be disposed in recess 143b.

FIG. 10 shows an arrangement in which join 141, but not knot 147, is disposed in recess 143. In this arrangement, knot 147 can be disposed elsewhere in a groove 144b of bushing 142b, e.g., on an opposite side of the bushing from recess 143. In FIG. 10, knot 147 is hidden behind bushing 142b.

In some implementations, bushing 142 is shaped such that part of the groove is covered in a manner that secures collar 128d in the groove. Such a covering 149 is shown for bushing 142a. In some implementations, the lateral bulge that defines recess 143b can be considered to serve as such a covering of bushing 142b.

FIGS. 11A-K show additional anchors (e.g., variants of anchor 120) whose anchors whose eyelets are formed from a textile 140, in accordance with some implementations. In some implementations, these anchors can be considered variants of anchor 120d (and their eyelets variants of eyelet 126d). Roman numeral suffixes are used to identify each such variant.

Whereas FIGS. 6A-10 show eyelet 126d connected to, and extending from, a single place on collar 128, for several of the variants shown in FIGS. 11A-K the eyelet is connected to, and/or extends from, two places 60 on the collar, i.e., a pair of places. Places 60 can be circumferentially separated from each other. In some implementations, places 60 are on opposite sides of the collar from each other. The pair of places 60 can define and/or lie on a hinge axis ax3, e.g., such that the eyelet is pivotable about the hinge axis. In some implementations, this allows the eyelet to pivot over interface 124 of the head of the anchor. This can advantageously allow the eyelet to orient itself according to the relative position of tether 112. This, in turn, can allow smoother sliding of the tether through the eyelet, e.g., during contraction of the implant.

An example of this is anchor 120dI (FIG. 11A), in which textile 140 has been arranged (e.g., wrapped and/or tied) to define an eyelet 126dI and a collar 128dI. In this example, textile 140 is a yarn in which knots define places 60 at which eyelet 126dI extends from collar 128dI, and between which hinge axis a3 is defined.

FIGS. 11B-G shows anchors that each has a collar at least partly defined by a component other than textile 140. For the anchors shown in FIGS. 11B-F, these collars can have one or more features in common with bushing 142, described hereinabove.

In some implementations, textile 140 is elongate and has two ends and a bight therebetween, and the ends are connected to the collar such that the bight defines the eyelet. Examples of such an arrangement are shown in FIGS. 11B-D and 11F.

FIG. 11B shows an anchor 120dII that comprises a flexible eyelet 126dII defined by textile 140, and a rigid collar 128dII. FIG. 11C shows an anchor 120dIII that comprises a flexible eyelet 126dIII defined by textile 140, and a rigid collar 128dIII. Each of collars 128dII and 128dIII has two openings that define places 60 at which the corresponding eyelet extends from the collar, and between which hinge axis a3 is defined. In these variants, textile 140 can be a yarn that has a knot at each end, the knot serving as a stopper that retains the eyelet attached to the collar.

For anchor 120dII, the knots are introduced into the collar from a distal side of the collar, and can be disposed in recesses 62dII on the distal side of the collar (e.g., facing tissue-engaging element 130). Recesses 62dII can be distal to interface 124.

For anchor 120dIII, the knots are introduced into the collar from a proximal side of the collar, and can be disposed in recesses 62dIII on the proximal side of the collar (e.g., facing away from tissue-engaging element 130). Interface 124 can be disposed between recesses 62dIII (e.g., as shown), or can even be distal to the recesses.

FIG. 11D shows an anchor 120dIV that is similar to anchors 120dII and 120dIII except as described. For anchors 120dII and 120dIII, textile 140 is shown as extending outwardly (e.g., laterally) from its knotted ends, e.g., pulling on the eyelet pulls the knots in an outward direction. For anchor 120dIV, collar 128dIV and textile 140 can be configured such that the textile extends inwardly from its knotted ends, e.g., pulling on the eyelet pulls the knots in an inward direction. For anchors 120dII and 120dIII, the knots are local to places 60. For anchor 120dIV, the knots are remote from places 60, e.g., such that, between each knot and a corresponding place 60, textile 140 extends (e.g., as a chord) through collar 128dIV. For example, collar 128dIV can define recesses 62dIV, each of which is on a far side of the collar from its corresponding place 60, with a corresponding bore 64dIV cut through the collar therebetween, and textile 140 can extend through the bore. In the example shown, bores 64dIV are parallel with each other, on opposite sides of stock 123.

FIG. 11E shows an anchor 120dV that, similar to anchor 120dV, has a bore 64dV that extends through its collar 128dV. However, bore 64dV connects the recesses 62dV of the collar to each other. In this manner, textile 140 can be arranged (e.g., tied) in a closed loop that is threaded through bore 64dV. In some implementations, using knots to tie textile 140 to itself in this manner may advantageously provide strengthened attachment of the eyelet to the collar, compared to using knots as stoppers. In the example shown, a single bore 64dV circumscribes partway around stock 123. Similarly to anchors 120dII and 120dIII, recesses 62dV can be local to places 60.

FIG. 11F shows an anchor 120dVI that is similar to anchors 120dII and 120dIII, except that, in place of recesses, its collar 128dVI defines one or more tabs 66 onto which textile 140 is tied in order to secure the eyelet 126dVI (defined by the textile) to the collar. In the example, a pair of tabs 66 are disposed on opposite sides of the collar, such that securing corresponding ends of textile 140 to the tabs forms the bight of the textile into eyelet 126dVI.

FIG. 11G shows an anchor 120dVII that is similar to anchor 120dV except that, whereas collar 128dV is rigid, collar 128dVII is flexible. Such flexibility may reduce wear on textile 140. In the example shown, collar 128dVII is defined by a flexible tube that has a bore 64dVII along the tube. At each end of the tube is an end-opening that defines a respective one of places 60. In some implementations, and as shown, textile 140 defines a closed loop onto which the tube is threaded by the textile extending through bore 64dVII and out of both end-openings. Collar 128dVII can be secured by stock 123 extending transversely through the flexible tube. For example, the flexible tube can define a transverse channel (e.g., a pair of holes in the material of the tube, facing each other), and the stock can extend transversely through the tube via the transverse channel. Collar 128dVII can be made of a flexible polymer and/or fabric. For example, collar 128dVII can comprise a piece of polymeric tubing.

FIG. 11H shows a textile 140dVIII in which a collar 128dVIII and an eyelet 126dVIII are formed integrally during formation of the textile, e.g., during the weaving, knitting, or braiding of the textile. For example, as textile 140dVIII is formed progressively from one end to another, the weave/knit/braid can separate to form eyelet 126dVIII, merge to close the eyelet and form a junction 147dVIII (analogous to knot 147), diverge again to form collar 128dVIII, and merge again at the other end to close the collar. Integral formation of the collar and eyelet can be advantageous with respect to production efficiency and/or consistency. It is to be understood that the formation can alternatively be performed in the opposite direction. In some implementations, collar 128 is mounted on a bushing. For other implementations, collar 128 is mounted directly on stock 123.

In some implementations, textile 140dVIII can be formed as a substantially flat sheet (e.g., resembling a ribbon), or can be formed using tubular weaving, e.g., with eyelet 126dVIII and collar 128dVIII each being defined by two tubular components alongside each other, and junction 147dVIII being generally tubular.

FIG. 11I shows an anchor 120dIX that comprises a textile 140dIX. Similarly to some other textiles described hereinabove, textile 140dIX defines both the eyelet 126dIX of the anchor and the collar 128dIX of the anchor. Rather than the eyelet and the collar being formed by tying an elongate textile into an arrangement, eyelet 126dIX and collar 128dIX are formed (e.g., cut) from a sheet of textile 140dIX (e.g., a sheet of fabric). For example, textile 140dIX can be cut to define a disc having a transverse hole 68 and an arc slit 69 extends circumferentially partway around the transverse hole. In the example shown, slit 69 extends circumferentially most of the way around transverse hole 68. The region of the disc lateral from slit 69 serves as eyelet 126dIX, while the region of the disc medial from the slit serves as collar 128dIX, places 60 being at the ends of slit 69. In this manner, a unitary structure including both the eyelet and the collar of the anchor can be formed simply by cutting a sheet of textile. This can facilitate manufacturing of such anchors, e.g., by allowing many such unitary structures to be cut from a single sheet and by requiring little or no further manufacturing steps. Collar 128dIX can be mounted simply by threading tissue-engaging element 130 through hole 68 until stock 123 becomes disposed through the hole.

FIG. 11J shows an anchor 120dX that comprises a textile 140dX. Similarly to some other textiles described hereinabove, textile 140dX defines both the eyelet 126dX of the anchor and the collar 128dX of the anchor. Textile 140dX is elongate, with an eye 70 at each end and a bight therebetween. Textile 140dX can be a yarn whose ends are looped (e.g., tied) to form eyes 70. Alternatively, and as shown, eyes 70 can be integrally formed, e.g., during the formation of textile 140dX (e.g., as described with reference to FIG. 11H, mutatis mutandis) or by being cut from a sheet of textile (e.g., as described with reference to FIG. 11I, mutatis mutandis). The first image of FIG. 11J shows textile 140dX alone. The second and third images show the mounting of textile 140dX. Tissue-engaging element 130 is threaded through eyes 70 and the eyes are worked proximally along the tissue-engaging element (second image) until stock 123 becomes disposed through the eyes (third image). Thus, eyes 70 collectively serve as the collar 128dX of the anchor. Because eyes 70 are brought together, the bight of the textile becomes looped and serves as the eyelet 126dX of the anchor. Anchor 120dX can have similar manufacturing advantages to those described for anchor 120dIX.

FIG. 11K shows an anchor 120dXI in which, rather than the textile itself defining the eyelet of the anchor, the textile serves to connect a discrete rigid eyelet 126dXI to the collar of the anchor. Eyelet 126dXI can be a metallic or polymeric ring. In some implementations, such an arrangement can benefit both from the advantages of a flexible textile 140 and the advantages of a rigid eyelet. In the example shown, the collar is collar 128dII, but it is to be understood that this is purely an illustrative example, and that any of the other anchors described herein can be modified in this manner, mutatis mutandis.

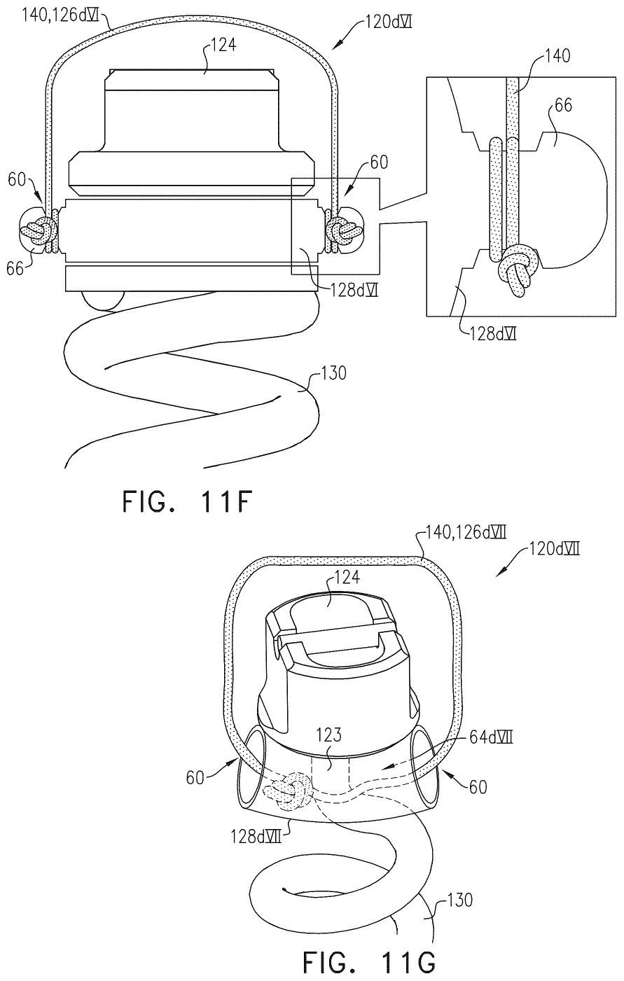

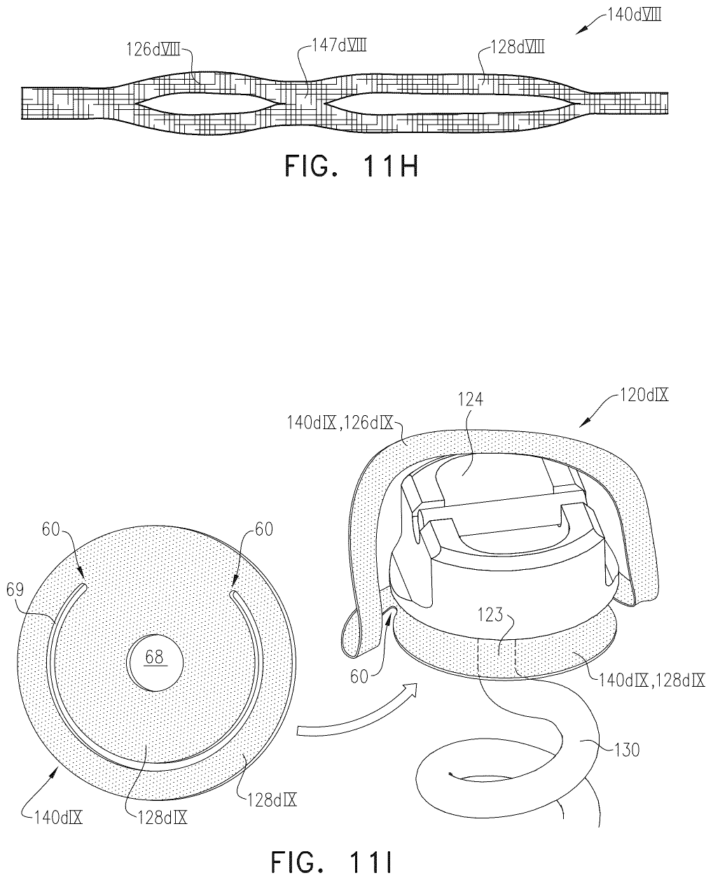

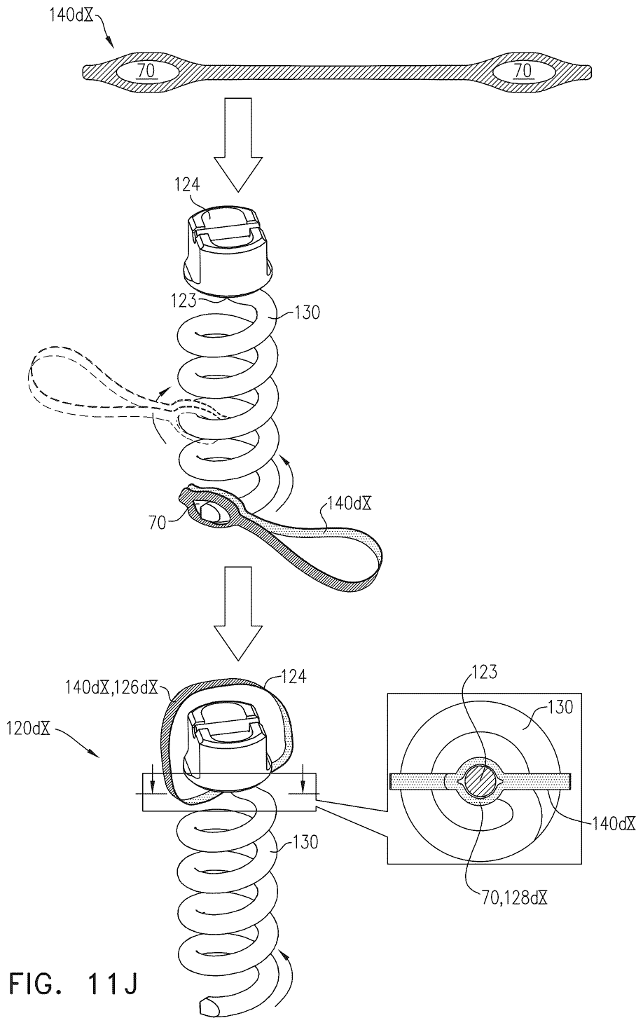

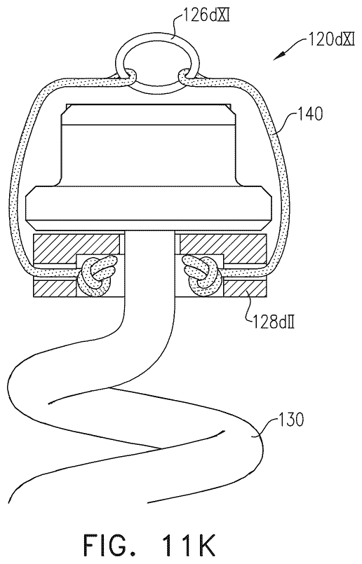

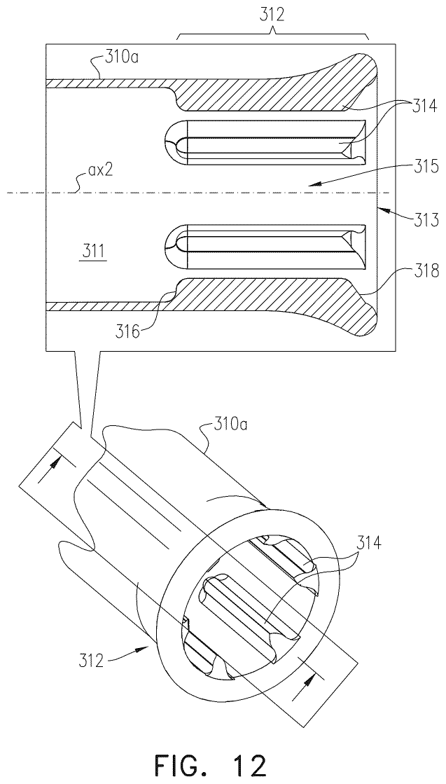

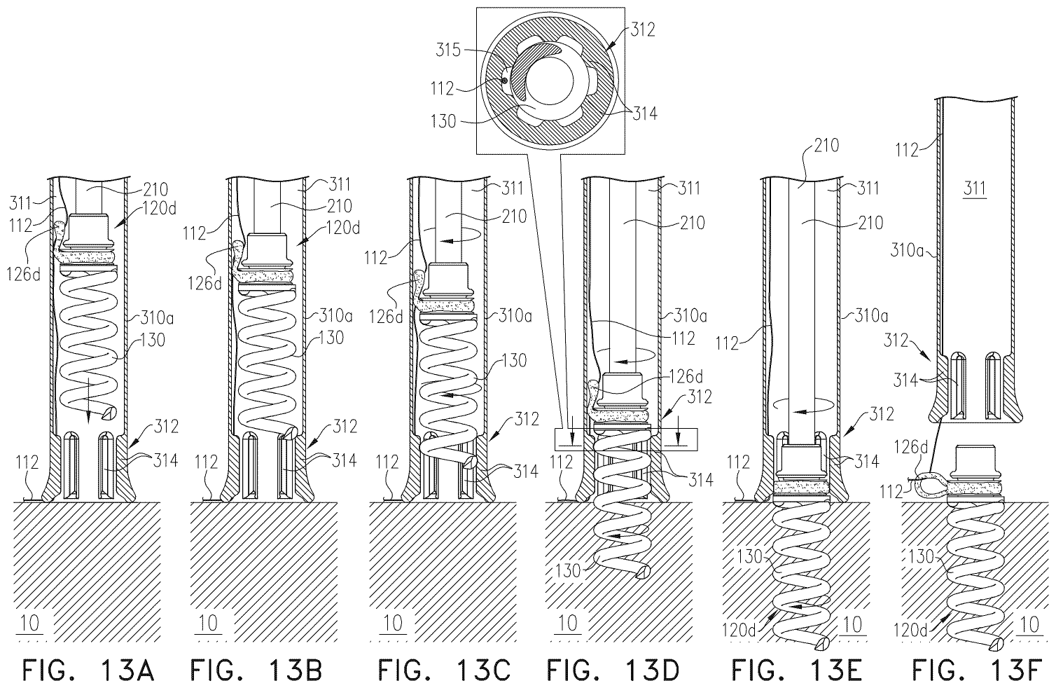

Reference is made to FIGS. 12, and 13A-F, which are schematic illustrations of a distal portion of a flexible tube 310a (e.g., a catheter), and use thereof, in accordance with some implementations. Tube 310a is a component of a catheter device, and can be considered a variant of tube 310 described hereinabove. Moreover, tube 310a can be used, mutatis mutandis, in place of tube 310 in catheter device 300, described hereinabove, and/or as the tube of any other catheter device, including those described herein, mutatis mutandis. FIG. 12 shows a close-up of the distal portion of tube 310a, and FIGS. 13A-F show an anchor 120d being delivered and anchored via the tube.

Whereas the internal channel of tube 310 can be keyhole-shaped, tube 310a has an internal channel 311 that may not be keyhole-shaped, e.g., it can be circular in cross-section. In some implementations, anchor 120d is particularly suited to delivery via tube 310a, e.g., because of the flexibility of its eyelet 126d.

Tube 310a defines a grip zone 312 at the distal end of the tube, e.g., proximal from a distal opening 313 of the tube, out of which the anchor (e.g., anchor 120d) is eventually advanced. At grip zone 312, tube 310a has at least one grip surface that inhibits sliding of the anchor through the grip zone by gripping a lateral surface of the helical tissue-engaging element of the anchor, e.g., tissue-engaging element 130. This grip surface can be provided by one or more resilient ribs (or nubs, or nodules) 314 that protrude medially into channel 311. In the example shown, grip zone 312 of tube 310a has six ribs 314. However, it is to be understood that grip zone 312, or a variant thereof, can have more ribs or fewer ribs, such as four ribs, three ribs (e.g., as shown for grip zone 312d), two ribs, or one rib. For implementations in which grip zone 312 has multiple ribs, the multiple ribs can be distributed circumferentially around a central tube axis ax2 of tube 310a. In some implementations this distribution is even (e.g., with equal circumferential spacing between each pair of adjacent ribs). In some implementations, this distribution is uneven (e.g., with some of the ribs being closer to each other, and some being further apart).

In order to implant anchor 120d, an anchor driver, such as driver 210, via engagement with the anchor (e.g., the interface of the anchor) slides the anchor distally through channel 311 toward grip zone 312 (FIG. 13A). Once anchor 120d arrives at grip zone 312, ribs 314 grip tissue-engaging element 130 (FIG. 13B). This gripping inhibits sliding of anchor 120d through grip zone 312 in the absence of rotation, e.g., inhibits solely axial sliding. In order to advance anchor 120d further distally, and therefore through grip zone 312, driver 210 rotates tissue-engaging element 130 (e.g., by applying torque to the interface of the anchor) such that tissue-engaging element 130 slides helically over ribs 314, which deform (e.g., compress) to accommodate this sliding (FIGS. 13C-D). That is, driver 210 screws the tissue-engaging element over ribs 314. The shape, strength, and/or resilience of ribs 314 can be optimized to resist axial sliding but to allow helical sliding of tissue-engaging element 130 thereover.

FIG. 13D shows tissue-engaging element 130 being driven (e.g., screwed) into tissue 10. In some implementations, and as shown, this may occur while some of the tissue-engaging element remains gripped by ribs 314. Thus, the grip zone 312 can be dimensioned and/or positioned (e.g., relative to the anchor) such that at least the distal tip of tissue-engaging element 130 can exit distal opening 313 of tube 310a while the tissue-engaging element remains within the grip zone (e.g., remains gripped by ribs 314). In this state, rotation of tissue-engaging element 130 can draw (e.g., pull) the head of the anchor through grip zone 312. As the inherent counterforce to this, tissue-engaging element thereby pulls proximally on tissue 10. In some implementations, this mutual pulling can be utilized improve anchoring reliability, e.g., as a mechanical/tactile and/or imaging-based anchoring indication. For example, if the tissue-engaging element has been driven into tissue that is unsuitably weak (e.g., leaflet tissue, or diseased tissue), the anchor may not be pulled out of the tube. Conversely, full/successful anchoring in suitable tissue may be indicated by (i) the arrival of the head of the anchor at the distal opening of the tube (or the exit of the head from the distal opening), and/or (ii) bulging of the tissue into the distal end of the tube (e.g., drawn into the distal end of the tube by the anchor). The operator may disengage from the anchor only once one or more indications of full/successful anchoring have been observed.

For implementations in which the anchor is coupled to a tether (e.g., in which the anchor and the tether are components of an implant, and/or the anchor is threaded onto the tether), while tissue-engaging element 130 is disposed in the grip zone (e.g., is gripped by ribs 314), the tether can be disposed within a niche 315 defined adjacent to a rib (e.g., between ribs). For example, and as shown, while driver 210 screws tissue-engaging element 130 over rib(s) 314, the tissue-engaging element can be excluded from niche 315 (e.g., due to its interaction with at least one rib) while tether 112 extends through the grip zone sheltered within the niche, laterally from the tissue-engaging element, e.g., as shown in the inset of FIG. 13D. This may advantageously prevent the tether from being engaged by the tissue-engaging element and/or can maintain the slidability of the tether through the grip zone even as the anchor passes through the grip zone.

FIG. 13E shows tissue-engaging element 130 having been fully anchored in tissue 10, with the head of anchor 120d disposed within grip zone 312. In some implementations, ribs 314 can be shaped and/or dimensioned to interact with (e.g., engage and/or grip) the head of the anchor.

In some implementations, and as shown, the head of the anchor passes through grip zone 312 substantially unimpeded by grip zone 312, e.g., without contacting ribs 314. As shown, eyelet 126d can pass through grip zone 312 between ribs 314. FIG. 13F shows tube 310 being retracted from anchor 120d subsequently to the anchor having been anchored.

In some implementations, ribs 314 are formed from a polymer. In some implementations, tube 310a comprises the same polymer, e.g., is formed from the polymer, and/or is lined with the polymer. Thus, ribs 314 can be formed integrally with tube 310a, which can be advantageous for manufacturing. The polymer can be a thermoplastic elastomer. The polymer can be a block copolymer, such as polyether block amide.

In some implementations, and as shown, one or more of ribs 314 has a proximal face 316 that is shaped to define a shoulder. This can facilitate the inhibition of anchor 120d through grip zone 312 in the absence of rotation, e.g., by tissue-engaging element 130 abutting the shoulder (e.g., see FIG. 13B). In some implementations, one or more of ribs 314 has a distal face 318 that is tapered. This can facilitate retraction of anchor 120d through grip zone 312 into tube 310, e.g., should it be determined that such retraction is required. For example, with respect to tube axis ax2, proximal face 316 can be disposed at a steeper angle than distal face 318. In some implementations, proximal face 316 is disposed at least 75 degrees and/or no more than 90 degrees with respect to axis ax2 (e.g., can be substantially orthogonal with respect to axis ax2). In some implementations, distal face 318 is disposed at least 20 degrees (e.g., at least 40 degrees) and/or no more than 70 degrees (e.g., no more than 60 degrees) with respect to axis ax2.

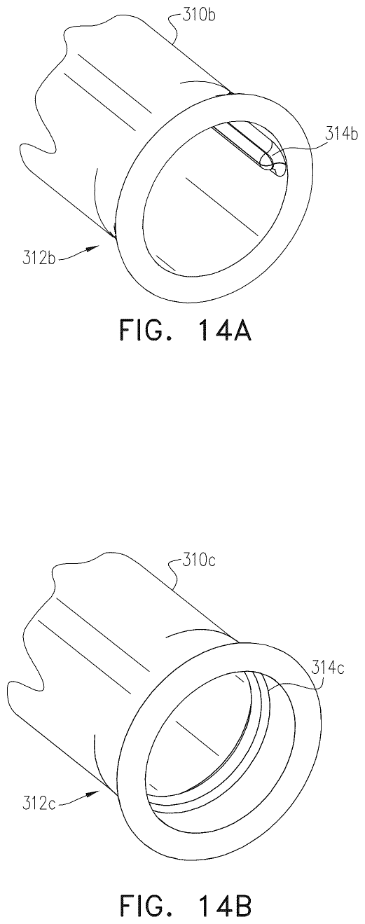

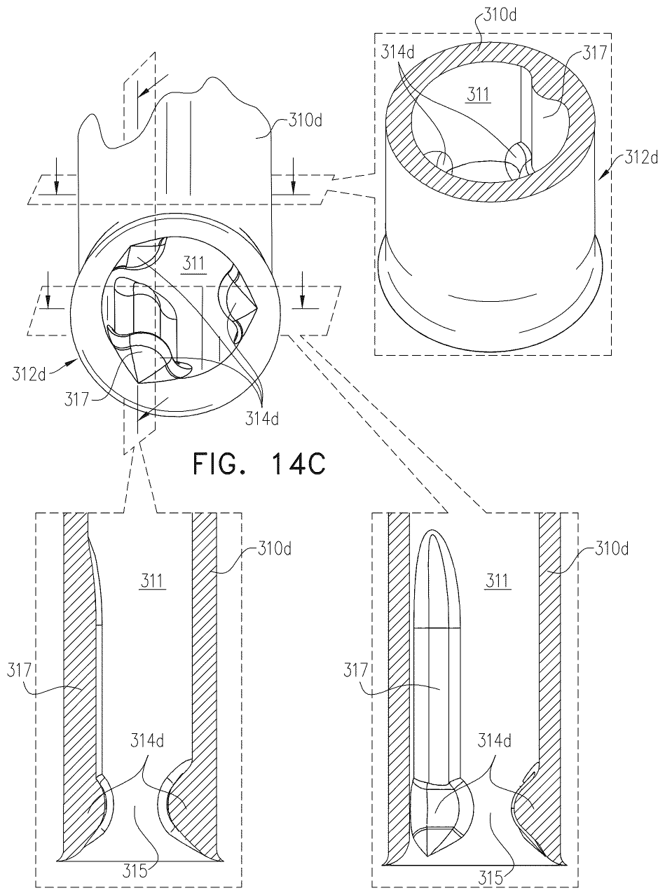

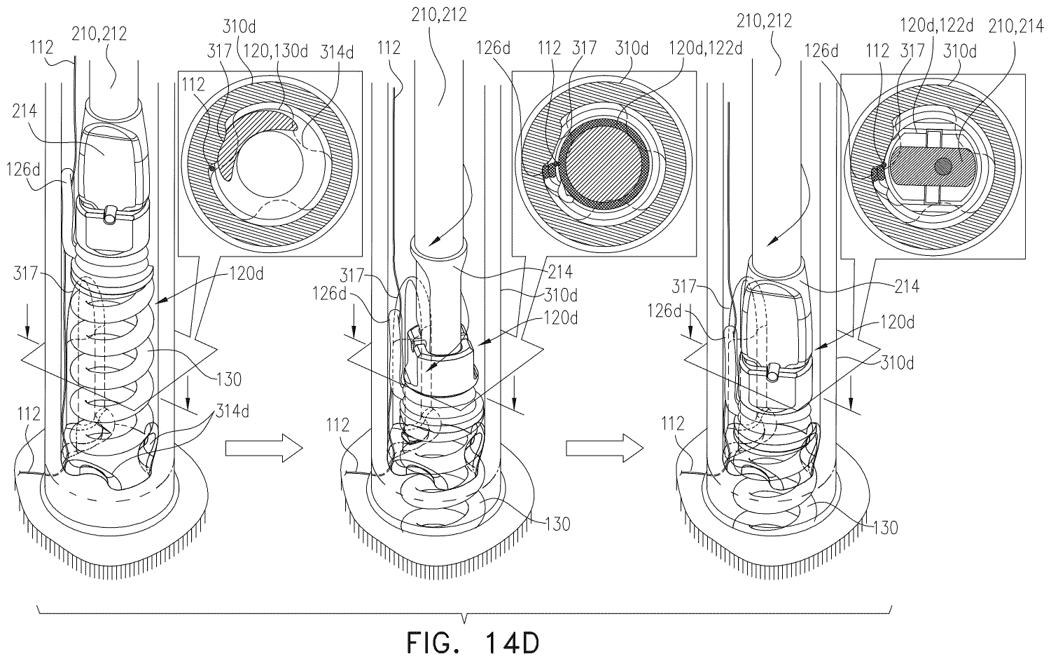

Reference is now additionally made to FIGS. 14A-E, and 15A-C, which are schematic illustrations of variants of tube 310 or tube 310a, in accordance with some implementations.

As noted hereinabove, the grip zone of a tube may have only one rib. FIG. 14A shows the distal portion of a tube 310b, which can be as described for tube 310a, except that its grip zone 312b has only one rib 314b. In some implementations, rib 314b can be identical to rib 314, described hereinabove, or similar thereto. In some implementations, rib 314b can differ from rib 314 in order to accommodate the lack of any other (e.g., opposing) ribs, e.g., rib 314 can protrude further into the channel of the tube.

FIG. 14B shows the distal portion of a tube 310c, which can be as described for tube 310b, except that, whereas ribs 314 and 314b extend alongside tube axis ax2 (e.g., parallel with the tube axis), the rib(s) 314c of grip zone 312c of tube 310c extend around at least part of the tube axis. For example, and as shown, rib(s) 314c can extend circumferentially around the entire tube axis, e.g., can be toroidal.

In the example shown, grip zone 312c of tube 310c has one rib 314c. However, it is to be understood that grip zone 312c, or a variant thereof, can have more ribs, such as two ribs, three ribs, four ribs, or more. For implementations in which grip zone 312c has multiple ribs, the multiple ribs can be distributed along tube axis ax2 of tube 310c. In some implementations this distribution is even (e.g., with equal axial spacing between each pair of adjacent ribs). In some implementations, this distribution is uneven (e.g., with some of the ribs being closer to each other, and some being further apart).

Despite being circumferential rather than axial, rib 314c can also have a proximal face that defines a shoulder and/or a distal face that is tapered, e.g., as described for rib 314, mutatis mutandis. In some implementations in which grip zone 312c has multiple ribs 314c, only a subset of the ribs (e.g., only a proximal-most rib) can have a proximal face that defines a shoulder. In some implementations in which grip zone 312c has multiple ribs 314c, only a subset of the ribs (e.g., only a distal-most rib) can have a distal face that is tapered.

Ribs 314 (e.g., ribs 314b) can be elongate, as shown, but can be longer or shorter than shown. Moreover, they can be sufficiently short that they resemble nodules, e.g., as shown for ribs 314d. Similarly, each rib 314c can comprise one or more sub-ribs (e.g., nodules) each of which circumscribes less than the entirety of the tube axis. For example, multiple sub-ribs can be distributed around the tube axis.

FIGS. 14C-D show the distal portion of a tube 310d having a grip zone 312d that comprises one or more ribs 314d, in accordance with some implementations. In addition to the ribs, grip zone 312 further comprises at least one abutment 317 which, in the illustrated example, is designated abutment 317d. Abutment 317 protrudes medially into channel 311 of tube 310d, but less so than ribs 314d, i.e., a height h2 of the abutment is smaller than a height h1 of ribs 314d. Due to this difference, abutment 317 interacts less with tissue-engaging element 130 than do ribs 314d. For example, abutment 317 may not obstruct or grip tissue-engaging element 130 (e.g., may not inhibit axial sliding of the tissue-engaging element). Abutment 317 functions to inhibit eyelet 126d from revolving around the anchor axis as the anchor is screwed into the tissue.

FIG. 14D shows an anchor 120d being screwed into the tissue (e.g., analogous to FIGS. 13C-E). Whereas interface 124 and tissue-engaging element 130 rotate as the tissue-engaging element is screwed through grip zone 312d and into tissue 10, any associated revolution of eyelet 126d is inhibited upon the eyelet abutting abutment 317. As tissue-engaging element 130 moves helically distally, eyelet 126d slides linearly alongside abutment 317.

As shown, abutment 317 can be disposed proximally from ribs 314d, e.g., such that it interacts with the eyelet of the anchor while the ribs interact with the tissue-engaging element of the anchor. In some implementations, and as shown for abutment 317d, the abutment can be longer (i.e., extend further along the axis of the tube) than the ribs, i.e., a length d4 of the abutment is greater than a length d3 of ribs 314d. This can allow the eyelet to remain disposed against the abutment as progressively proximal parts of the tissue-engaging element arrive at, and pass, the ribs.

In some implementations, and as shown for abutment 317d, each abutment can extend from a respective rib, e.g., the abutment and the rib can be defined by a unitary structure. Alternatively, abutment 317 can be a discrete structure.

Although abutment 317 (e.g., abutment 317d) is shown in combination with ribs 314d, it is to be understood that grip zones having other ribs 314 can also utilize such an abutment.

Tubes 310a, 310b, 310c, and 310d are shown as being flared toward their respective distal openings. This optional feature can similarly be applied to other tubes, such as tube 310, e.g., tubes that may not include a grip zone. Such flaring may advantageously provide the tube with an atraumatic characteristic, e.g., compared to a tube with a straight end. Alternatively or additionally, such flaring may advantageously reduce pressing of the end of the tube on tether 112, e.g., as shown in FIG. 15A for tube 310d. Alternatively or additionally, such flaring may facilitate sliding of tether 112 over the rim of the distal opening (e.g., reducing gripping of the tether by the rim of the distal opening), thereby advantageously reducing a likelihood of movement of the tube pulling deleteriously hard on tether 112 and the previously-anchored anchors. Alternatively or additionally, such flaring may advantageously facilitate retraction of an anchor back into the tube, should it be required.

In some implementations, the distal end of the tube (e.g., of the grip zone) can be more flexible than more proximal regions of the tube. Like the flaring, this may provide an atraumatic characteristic to the tube. This may also facilitate retraction of an anchor back into the tube, should it be required. FIG. 14E shows an example of this, in which the distal end of tube 310d (e.g., of grip zone 312d) plasticly deforms at 309 to accommodate re-entry of the anchor, e.g., when the tube is imperfectly aligned with the anchor.

In some implementations, in addition to and/or alternatively to flaring, a tube can be shaped such that the rim of the distal opening of the tube is undulating. FIGS. 15B and 15C shows examples of this, with a tube 310f (FIG. 15C) having more such undulations than a tube 310e (FIG. 15B). Similar to the flaring, the undulations may advantageously reduce pressing of the end of the tube on tether 112. Whether provided by flaring or by undulations, this reduced pressing may, inter alia, facilitate de-slacker 354 reducing slack (e.g., maintain a minimal tension) on the regions of the tether beyond tube 310.

Referring again to grip zone 312 and its variants. In some implementations, the grip zone (e.g., its ribs) is configured to substantially prevent distal advancement of tissue-engaging element 130 in the absence of rotation. In some implementations, the grip zone (e.g., its ribs) are configured primarily to provide tactile feedback, e.g., to resist distal advancement in the absence of rotation, but to allow such non-rotational distal advancement should sufficient axial (pushing) force be applied. In either case, the presence of grip zone 312 may advantageously reduce the likelihood of premature and/or inadvertent advancement of the anchor out of the tube through which it has been delivered.

It is to be noted that grip zone 312 may have advantages over other components or features that are intended to similarly facilitate control of advancement of an anchor out of the distal end of a delivery tube, or to maintain separation between the tissue-engaging element of the anchor and the tether onto which the anchor is threaded. For example, a spur (which may be relatively rigid) intended to facilitate control of advancement of an anchor out of the distal end of a delivery tube may require particular rotational orientations of the anchor (or parts thereof) relative to the delivery tube and/or may impede retraction of the anchor into the delivery tube, should such retraction be deemed necessary.

Similarly, a keyhole-shaped channel of the delivery tube intended to maintain separation between the tissue-engaging element of the anchor and the tether onto which the anchor is threaded may require particular rotational orientations of the delivery tube relative to the tissue and/or previously-anchored anchors, and/or may impede retraction of the anchor into the delivery tube, e.g., by requiring rotational alignment between the anchor and the keyhole-shaped lumen.

Although grip zone 312 is described and shown for a tube whose channel is substantially circular in cross-section, it is to be understood that, in some implementations, grip zone 312, or a similar grip zone, can be provided on other tubes, including those with non-circular (e.g., keyhole-shaped) channels. Similarly, although grip zone 312 has been described and shown for facilitating delivery of anchor 120d, it is to be understood that, in some implementations, grip zone 312, or a similar grip zone, can be used to facilitate delivery of other anchors such as, but not limited to, other anchors described herein.

FIGS. 15A-C also illustrate anchors 120d having been implanted as components of an implant that further comprises tether 112. One anchor 120d is shown serving as a leading anchor 120d′, and another (identical) anchor is shown serving as a successive anchor 120d″. A stopper 114d is shown fixed to the distal end of tether 112, maintaining the tether coupled to anchor 120d′, e.g., by preventing the tether from sliding out of eyelet 126d. However, other means of maintaining this coupling can be used. For example, tether 112 can be looped through eyelet 126d and back on itself, with the loop being closed by knotting, crimping, or another suitable means.

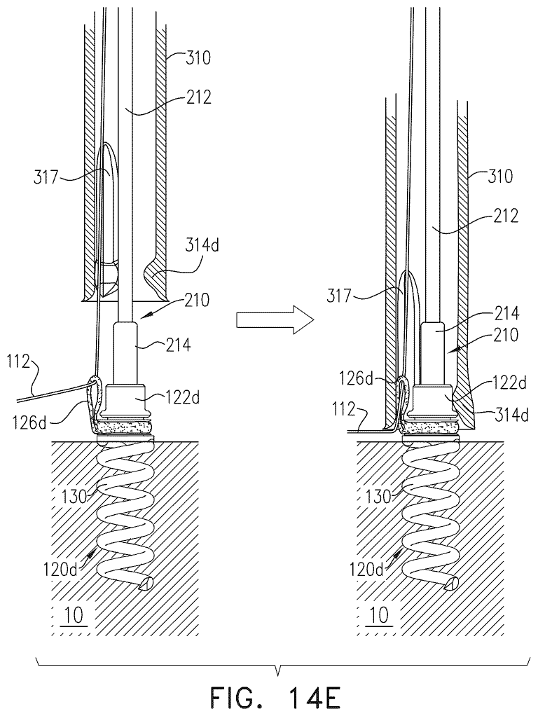

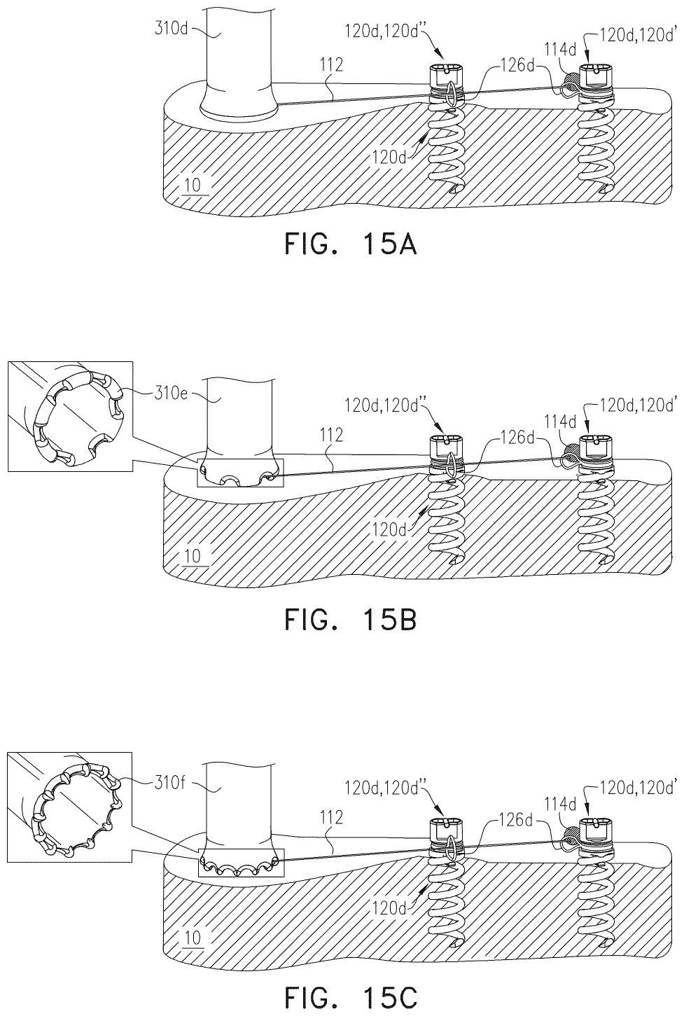

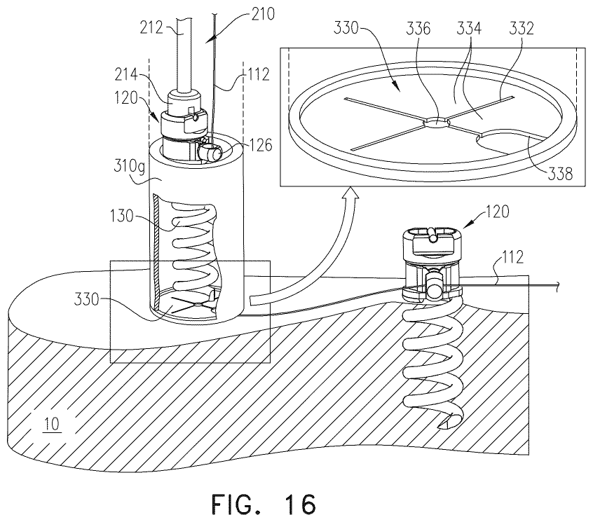

Reference is made to FIG. 16, which is a schematic illustration of a membrane 330, in accordance with some implementations. Membrane 330 is shown as being disposed over a distal opening of a flexible tube 310g, but it is to be understood that the membrane can be used with any of the tubes described herein, mutatis mutandis. Membrane 330 has one or more slits 332 (e.g., multiple slits) that divide the membrane into multiple flaps 334. In the example shown, membrane 330 has four slits 332, but variants of membrane 330 can have one, two, three, five, six, or more slits. In the example shown, membrane 330 is divided into four flaps 334, but variants of membrane 330 can have two, three, five, six, or more flaps.

In some implementations, and as shown, slits 332 converge to define a convergence point. In some implementations, membrane 330 can have a hole 336 at the convergence point.

In some implementations, to deliver and anchor anchor 120, driver 210 slides the anchor distally through the channel of tube 310g, and distally through membrane 330 via the one or more slits, with flaps 334 transiently separating responsively to passage of the anchor through the membrane. Tube 310g, membrane 330, anchor 120, and/or driver 210 can be configured (e.g., shaped and/or sized) such that tissue-engaging element 130 aligns with hole 336.

In some implementations, membrane 330 defines a notch 338, disposed eccentrically, and positioned to substantially align with eyelet 126 of the anchor. Notch 338 may or may not begin at (e.g., extend laterally from) the convergence point of slits 332 or hole 336. Notch 338 may be present irrespective of the convergence (or not) of slits 332. In some implementations, notch 338 is defined in a single one of flaps 334. In some implementations, and as shown, notch 338 can be defined partly in one of the flaps, and partly in another one of the flaps, e.g., the notch can be at least partly coincident with one or more of slits 332.

Membrane 330 can advantageously augment control of the position of tether 112. Membrane 330 can advantageously reduce a likelihood of tether 112 becoming twisted and/or tangled with anchor 120. Membrane 330 can be radiolucent or echogenic, in order to advantageously improve visualization of the implantation procedure, e.g., to visually verify that the distal end of tube 310 is disposed against tissue 10, and/or to identify the position of anchor 120 with respect to the distal opening of the tube. Membrane 330 may advantageously facilitate de-anchoring and/or retraction of an anchor 120, such as by obstructing tissue 10 from becoming pulled into tube 310, e.g., “wiping” the tissue off of tissue-engaging element 130.

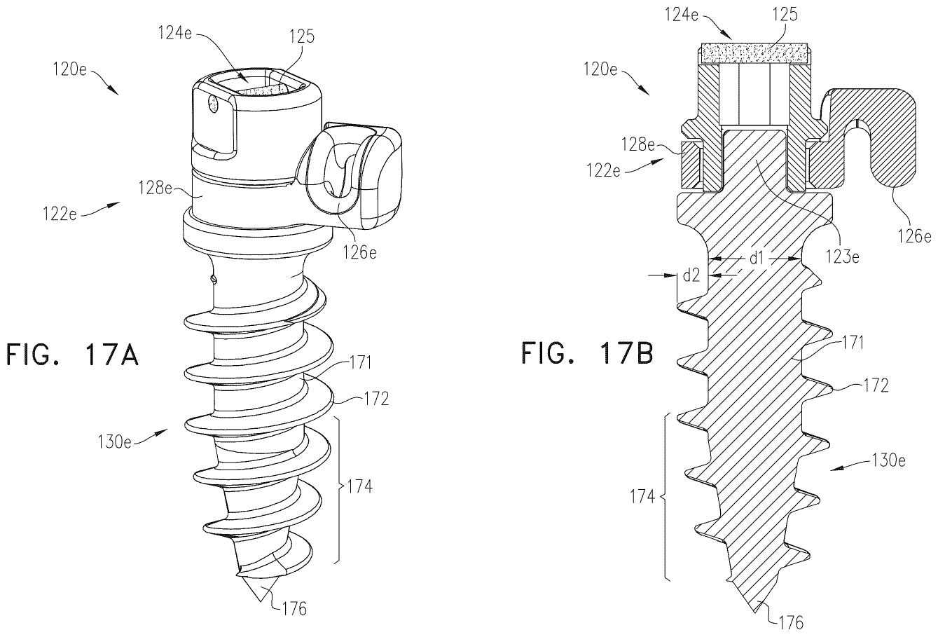

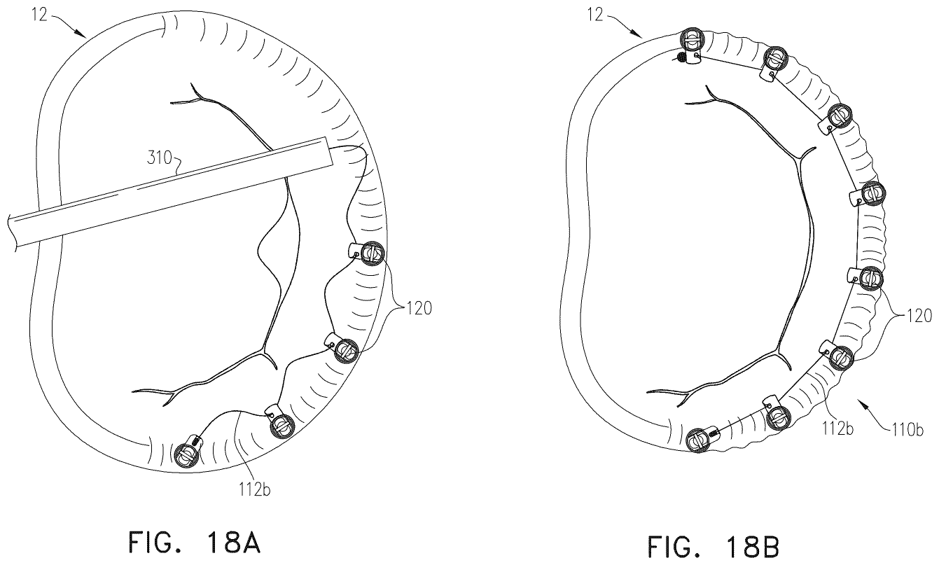

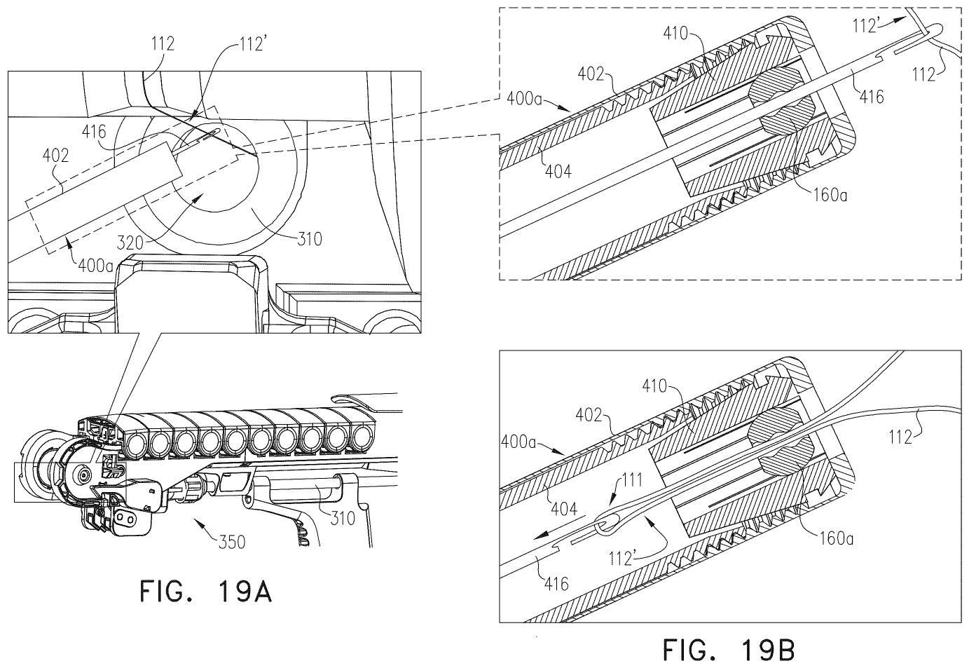

Reference is now made to FIGS. 17A-B, which are schematic illustrations of an anchor 120e, in accordance with some implementations. FIG. 17A is a perspective view, and FIG. 17B is a cross-sectional view. Anchor 120e comprises a head 122e and a tissue-engaging element 130e. Whereas the head and/or the tissue-engaging element of other anchors 120 described herein can be formed substantially from a metal such as stainless steel, head 122e and tissue-engaging element 130e are formed substantially from a polymer, e.g., a rigid polymer.

Head 122e defines an interface 124e, which can be as described for interface 124 except for being formed substantially from the polymer. In some implementations, interface 124 and/or interface 124e comprises a pin, e.g., that is orthogonal to the anchor axis and that is grasped by the anchor driver. For interface 124e, this pin can be a metal pin 125, e.g., the interface is formed substantially from the polymer except for the pin.

In some implementations, anchor 120e comprises an eyelet 126e that is revolvable about the anchor axis of the anchor, e.g., by being attached to a rotatably mounted collar 128e. In some implementations, anchor 120e can comprise a stock 123e that fixedly couples interface 124e to tissue-engaging element 130e (and that can lie on the anchor axis), and collar 128e can circumscribe, and be rotatable about, the stock. In some implementations, eyelet 126e, collar 128e, and/or stock 123e are also formed substantially from the polymer. As shown, collar 128e and eyelet 126e can be formed as a monolithic piece of the polymer.

In some implementations, rather than eyelet 126e and/or collar 128e, an eyelet and/or a collar formed from a textile are used instead. For example, eyelet 126d and/or collar 128d (described hereinabove) can be used in combination with tissue-engaging element 130e and/or head 122e, mutatis mutandis.

In some implementations, the polymer from which the components of anchor 120e are formed is a polyaryletherketone, such as polyether ether ketone (PEEK). In some implementations, one or more of the components of anchor 120e are (or the anchor as a whole is) formed via molding. In some implementations, one or more of the components of anchor 120e are (or the anchor as a whole is) formed via additive manufacturing, e.g., 3D printing.

In some implementations, in one or more of the parts (e.g., components) of anchor 120e, a radiopaque substance such as barium sulfate is mixed with the polymer so as to improve the visibility of the anchor in fluoroscopic images.

In some implementations, tissue-engaging element 130e is shaped to accommodate being formed from the polymer. For example, rather than being helical (e.g., like a corkscrew), tissue-engaging element 130e can comprise a central shaft 171 with an external self-tapping screw thread 172 extending helically around and along it, e.g., as shown. In some implementations, and as shown, central shaft 171 has a tapered region 174 that tapers toward a distal point 176 which can lie on the anchor axis. As shown, the tapering of shaft 171 can be steeper at distal point 176 than at tapered region 174.

In some implementations, screw thread 172 protrudes laterally from shaft 171 by a distance d2 that is 2-4 times (e.g., approximately 3 times) the diameter d1 of the shaft. d1 can be the diameter of the shaft at the thickest part of shaft 171. d2 can be the greatest distance by which screw thread 172 protrudes laterally from the shaft.

Reference is now made to FIGS. 18A-B, which are schematic illustrations showing implantation of an implant 110b, in accordance with some implementations. Implant 110b can be considered to be a variant of implant 110, e.g., can be as described for implant 110 except as noted. In particular, implant 110b comprises a tether 112b, which can be considered to be a variant of tether 112. Tether 112b is radiopaque, and is biased toward assuming a wavy shape, e.g., a regular wavy shape, such as sinusoid or zigzag. For example, tether 112b can comprise a shape memory alloy such as nitinol that is shape-set to the wavy shape. In some implementations, the joint characteristics of radiopacity and shape bias are provided by tether 112b having a radiopaque material alongside a shape memory material. For example, in some implementations, tether 112b can comprise a cable that includes at least one radiopaque strand and at least one shape memory strand. In some implementations, tether 112b can comprise a drawn filled tube that has a radiopaque core, e.g., with the outer sheath comprising nitinol.

Implant 110b can be implanted, mutatis mutandis, as described for implant 110, and/or using delivery tool 200. However, the waviness of tether 112b can facilitate implantation by providing, in fluoroscopic images, an indication of scale. For example, if the wavelength of the wavy shape is known, it can be used to measure distance along the tissue, e.g., like a ruler. Alternatively or additionally, an anchor can be positioned according to the number of waves between it and the preceding anchor. In the example shown, each successive anchor is positioned one full wavelength after the preceding anchor (FIG. 18A).

In some implementations, the biasing (e.g., shape-setting) of tether 112b is (i) sufficiently strong to provide reliable fluoroscopic guidance, but (ii) sufficiently weak (e.g., the tether is sufficiently flexible) that its waviness does not materially inhibit subsequent tensioning of the tether in order to draw the anchors together and contract the tissue (FIG. 18B). As shown in FIG. 18B, tether 112b can straighten (e.g., partially or completely) upon tensioning.

For implementations in which implant 110b is implanted using catheter device 300, de-slacker 354 can reduce (e.g., eliminate) slack without materially diminishing the waviness of tether 112b. This can be achieved, for example, by configuring tether 112b with sufficiently strong biasing, and/or by configuring de-slacker 354 to pull less strongly than it would for tether 112.

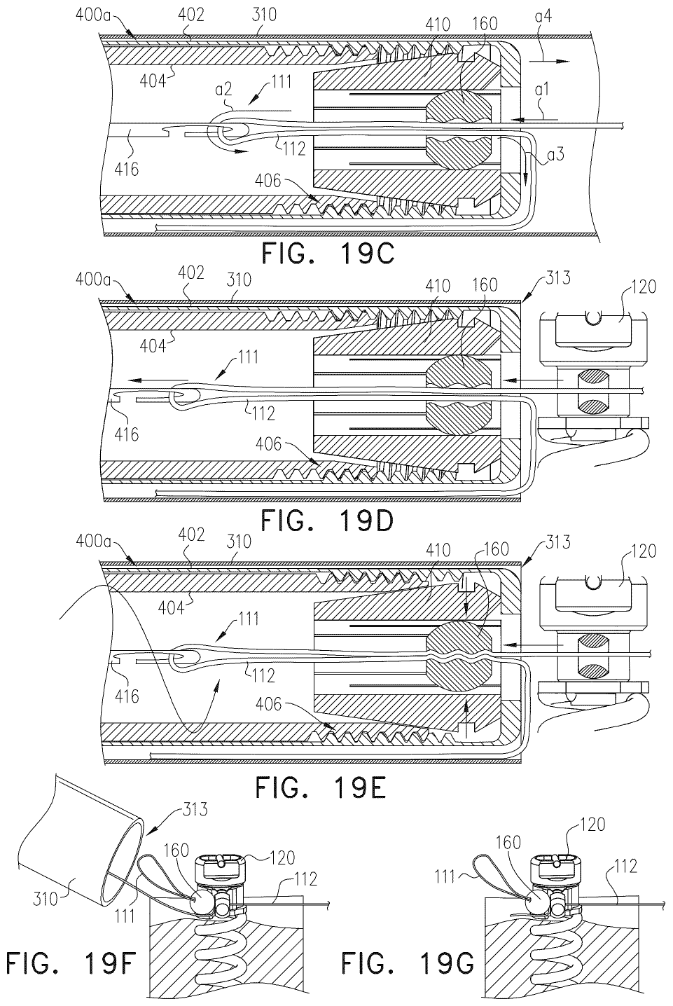

Reference is now made to FIGS. 19A-G, which are schematic illustrations of a tool 400a (e.g., a contraction and/or locking tool) and a lock 160a being used to apply and/or lock in tension to tether 112, in accordance with some implementations. Tool 400a and lock 160a can be the same as or similar to (e.g., variants of) tool 400 and lock 160.

It is to be noted that tool 400a and lock 160a advantageously can be used without access to the proximal end of tether 112, e.g., they can be used while the proximal end of the tether remains within and/or engaged by the extracorporeal unit of the catheter tool (e.g., de-slacker 354 thereof). This can confer certain advantages, such as the ability to apply a lock prior to any cutting of tether 112, and/or while catheter device 300 (e.g., tube 310 thereof) remains in place. In some implementations, this can allow the application of multiple lockers along the implant, rather than solely at the proximal end of the implant.

In some implementations, tool 400a and/or lock 160a can therefore be used in combination with, and/or to facilitate, systems and/or techniques described in Provisional U.S. Patent Application 63/370,609 to Biran et al., filed Aug. 5, 2022, and titled “Variable tissue contraction;” and/or International Patent Application PCT/IB2023/055323 to Guerrero et al., filed May 24, 2023, and titled “Variable tissue contraction,” each of which is incorporated herein by reference.

FIG. 19A shows tool 400a grasping tether 112 without access to the proximal end of the tether. Herein this is referred to as grasping a bight 112′ of the tether, i.e., not the distal end, not the proximal end, but a portion therebetween, irrespective of whether that portion is curved. In the example shown, bight 112′ is at extracorporeal unit 350, e.g., proximal from the proximal opening 320 of tube 310, via which tether 112 and anchors 120 are advanced into the tube. It is to be understood that the state shown in FIG. 19A is subsequent to at least one anchor 120, and typically multiple anchors, having been advanced and anchored to tissue.

Tool 400a comprises a shaft 402 and a collet 410. Tool 400a also comprises a grasper 416, e.g., a hook or a snare. Collet 410 is housed within shaft 402. Lock 160a is malleable and is shaped to define a passage therethrough. During use, lock 160a can be held within collet 410, e.g., as shown. Tool 400 can be provided with lock 160a already held within collet 410, and/or can be configured to engage and/or accept a lock during use.

Grasper 416 is extendable distally through lock 160a (e.g., through its passage) and out of shaft 402, so that it can grasp tether 112 (FIG. 19A). Tool 400 can be provided with grasper 416 already extended through lock 160a, or grasper 416 can be extended through the lock during use.

Grasper 416 can then draw tether 112 (e.g., bight 112′) proximally through lock 160a (e.g., through its passage) and into shaft 402, thereby forming and/or retaining the tether (e.g., the bight) into/as a loop 111 within the shaft (FIG. 19B).

While tether 112 is retained as loop 111 within shaft 402, tool 400 can be intracorporeally advanced distally along tether 112 (e.g., through tube 310) such that progressive regions of the tether are fed around grasper 416 (FIG. 19C). Thus, progressive regions of tether 112 are fed through loop 111, i.e., become part of the loop as tool 400 arrives, and then cease to be part of the loop as tool 400 progresses distally beyond. In FIG. 19C this is represented by arrows indicating feeding of tether 112 into the tool (arrow a1), around grasper 416 (arrow a2), and out of tool (arrow a3) as tool 400 is advanced distally (arrow a4). As shown, the part of the tether that is emerging from lock 160a and/or tool 400 (in the vicinity of arrow a3) curves proximally past the lock/tool, toward the extracorporeal part of the delivery tool (e.g., toward extracorporeal unit 350). Thus, during advancement of lock 160a, tether 112 is formed into an S-shape at and/or by the lock and/or delivery tool 400.

At this point, and typically once tool 400a has reached the most recently-anchored anchor, tether 112 is tensioned in order to draw anchors 120 toward each other and contract the tissue to which they are anchored. In some implementations, this can be achieved by grasper 416 being pulled proximally relative to shaft 402 and/or shaft 404 (FIG. 19D). In some implementations, this can be achieved by pulling on the proximal end of tether 112 (e.g., at extracorporeal controller), e.g., by using a tensioner such as described hereinbelow. The tensioning of tether 112 can be coincident with (e.g., facilitated by) lock 160 and/or the distal end of tool 400 abutting part of the implant of which the tether is a component, e.g., abutting the most recently-anchored anchor 120, such as abutting its eyelet 126. This abutment can provide a reference force for the tensioning. The tensioning of tether 112 can be coincident with (e.g., facilitated by) lock 160 and/or the distal end of tool 400 being disposed at or beyond distal opening 313 of tube 310, e.g., to facilitate their abutment with anchor 120.

In some implementations, tool 400a can lock lock 160a to tether 112, e.g., in order to lock in the tension applied to tether 112. This can be achieved by actuating collet 410 to crimp (e.g., crush) lock 160a (FIG. 19E). In some implementations, and as shown, collet 410 is actuated by relative movement of two other components of tool 400a. For example, and as shown, shaft 402 can be a first shaft, and tool 400a can further comprise a second shaft 404, with relative movement (e.g., axial movement) between the two shafts actuating the collet. As shown, such relative movement can include rotation, e.g., with shafts 402 and 404 having complementary threading 406 such that the relative rotation is translated into axial movement that actuates the collet. In the particular example shown, shaft 402 is an outer shaft and has an inner thread, and shaft 404 is an inner shaft and has an outer thread, with rotation of shaft 404 within shaft 402 driving shaft 404 distally in a manner than squeezes the collet.

In some implementations, once lock 160a has been locked to tether 112, tool 400a can release the lock from collet 410, and release tether 112 from grasper 416, and can then be withdrawn from the subject (FIG. 19F). At this point, and should it be desired, it is possible to advance and anchor additional anchors 120. If no further anchors are to be advanced and anchored, excess tether 112 can be trimmed (e.g., cut). This trimming is shown in FIG. 19G, e.g., subsequently to withdrawal of tool 400a. This can represent the trimming being performed using a tool other than tool 400a. However, in some implementations, the trimming is performed using tool 400a, e.g., tool 400a can include a blade.

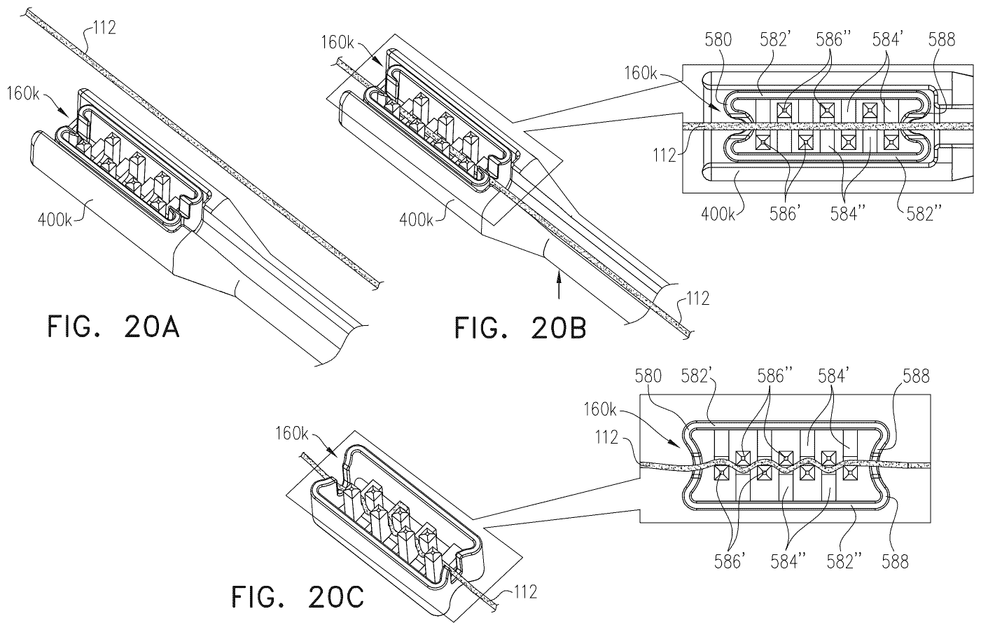

Reference is now made to FIGS. 20A-C, which are schematic illustrations of a tool 400k and a lock 160k, being used to apply and/or lock in tension to a tether of an implant, in accordance with some implementations. Like tool 400a and lock 160a, tool 400k and lock 160k can advantageously be used without access to the proximal end of tether 112, e.g., they can be used while the proximal end of the tether remains within and/or engaged by the extracorporeal unit of the catheter tool. This can confer certain advantages, such as the ability to apply a lock prior to any cutting of tether 112, and/or while catheter device 300 (e.g., tube 310 thereof) remains in place. In some implementations, this can allow the application of multiple lockers along the implant, rather than solely at the proximal end of the implant. In some implementations, tool 400k and/or lock 160k can therefore be used in combination with, and/or to facilitate, systems and/or techniques described in Provisional U.S. Patent Application 63/370,609 to Biran et al., filed Aug. 5, 2022, and titled “Variable tissue contraction;” and/or PCT Publication WO 2023/228098 to Guerrero et al., filed May 24, 2023, and titled “Variable tissue contraction,” each of which is incorporated herein by reference.

In some implementations, lock 160k comprises a casing 580 (e.g., a body or frame) that is biased to assume a widened state (FIG. 20C) but that is compressible into a narrowed state (FIGS. 20A-B) in which opposing sides 582′ and 582″ of the casing are closer to each other than in the widened state. Multiple (e.g., 2-4) extensions or fingers 584′ extend from side 582′ toward side 582″, and multiple (e.g., 2-4) extensions or fingers 584″ extend from side 582″ toward side 582′. Fingers 584′ and 584″ can be arranged alternatingly along a longitudinal axis of lock 160k.

In some implementations, one or more extensions or fingers 584′ are hooked—e.g., each finger has (e.g., terminates in) a hook 586′, and one or more fingers 584″ are hooked—e.g., each finger has (e.g., terminates in) a hook 586″.

In some implementations, in the narrowed state of casing 580 as shown in FIG. 20A-B, hooks 586′ can be spaced laterally apart from hooks 586″, thereby defining an unobstructed passage through lock 160k. That is, hooks 586′ are distributed along one side of the passage, and hooks 586″ are disposed along the other side of the passage.

In some implementations, hooks 586′ and 586″ can face in substantially the same direction, e.g., toward an open face of lock 160k. This orientation of the 586′ and 586″ can provide lateral access via which tether 112 is introducible sideways into the passage of the lock, i.e., to become disposed between hooks 586′ and hooks 586″ (FIG. 20B). In some implementations, tool 400k may also provide lateral access (e.g., may define a lateral opening along a substantial proportion of its length).

In some implementations, tool 400k is configured to constrain casing 580 in its narrowed state (FIG. 20A). For example, and as shown, at a distal end of tool 400k the tool may define a chamber that is dimensioned according to the narrowed state of casing 580. When casing 580 is in this narrowed state, lock 160k is in an unlocked state, and tool 400k may be used to place the lock laterally onto tether 112, and to advance the lock transluminally along the tether (FIG. 20B)—e.g., through tube 310 to the most recently-anchored anchor 120.

Once lock 160k has been positioned at the desired position along tether 112, and/or once a desired tension has been applied to the tether, the lock is deployed from tool 400k (e.g., ejected from the chamber of the tool), thereby unconstraining casing 580, which responsively widens toward its widened state (FIG. 20C). As casing 580 widens by sides 582′ and 582″ moving away from each other, the sides pull their respective fingers 584 with them, reducing (e.g., eliminating and/or obstructing) the lateral spacing (i.e., the passage) between hooks 586′ and hooks 586″. Reducing (e.g., eliminating and/or obstructing) the passage can inhibit sliding of tether 112 through lock 160k by forcing tether 112 into a tortuous path and/or clamping the tether between the hooks. Thus, in the widened state of casing 580, lock 160k may be considered to be in a locked state.

In some implementations, the biasing of casing 580 toward its widened state can be provided by resilient ends 588 of the casing. In some implementations, the resilient ends 588 of the casing can be strained by compression of the casing into its narrowed state. In some implementations, ends 588 can be shaped to define an entrance into, and an exit out of, the passage of the lock.

It is to be noted that lock 160k can be monolithic—e.g., can be manufactured (e.g., cut and shaped) from a single piece of stock material.

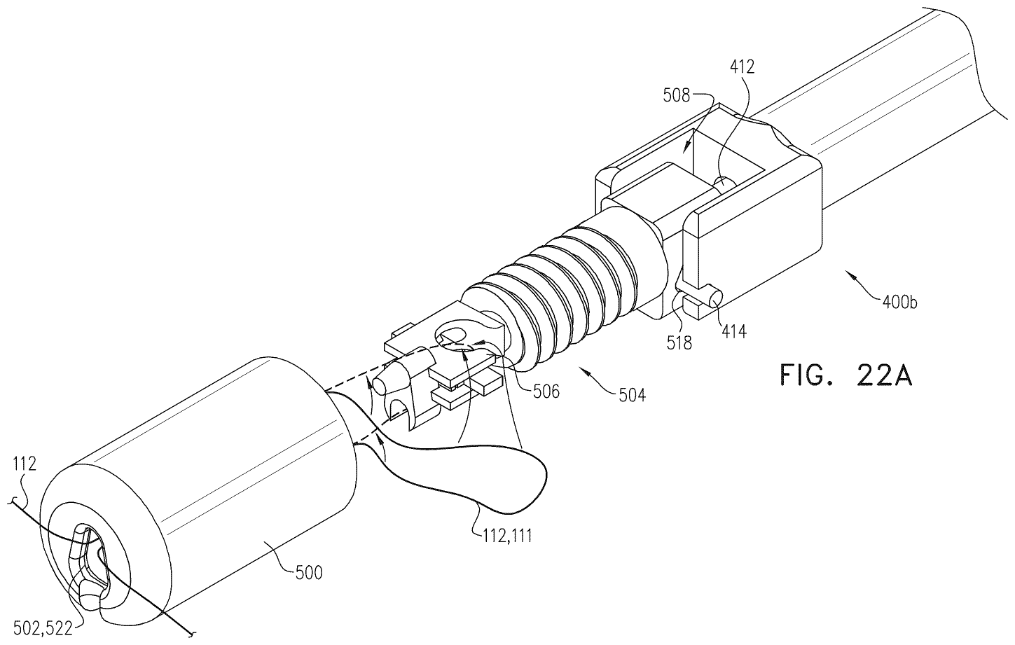

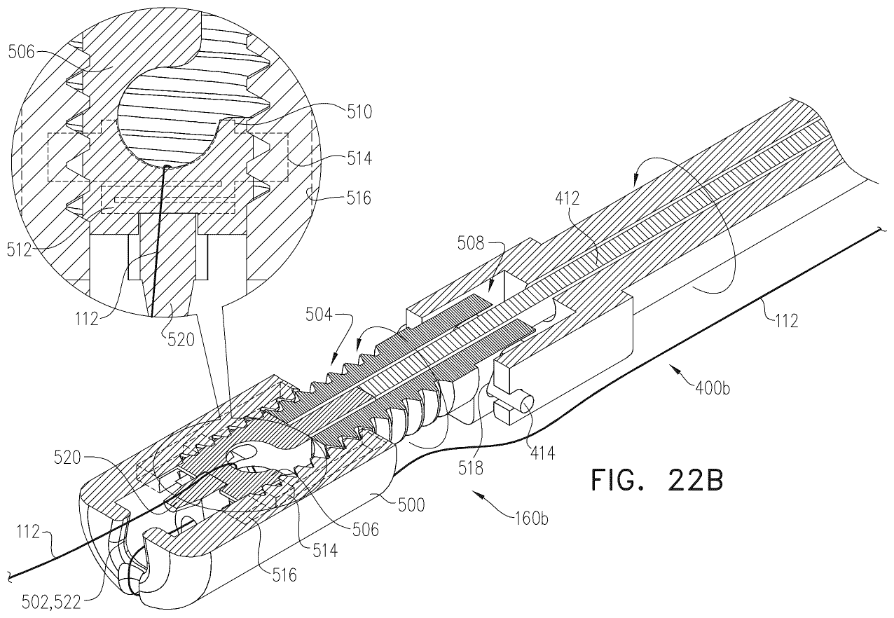

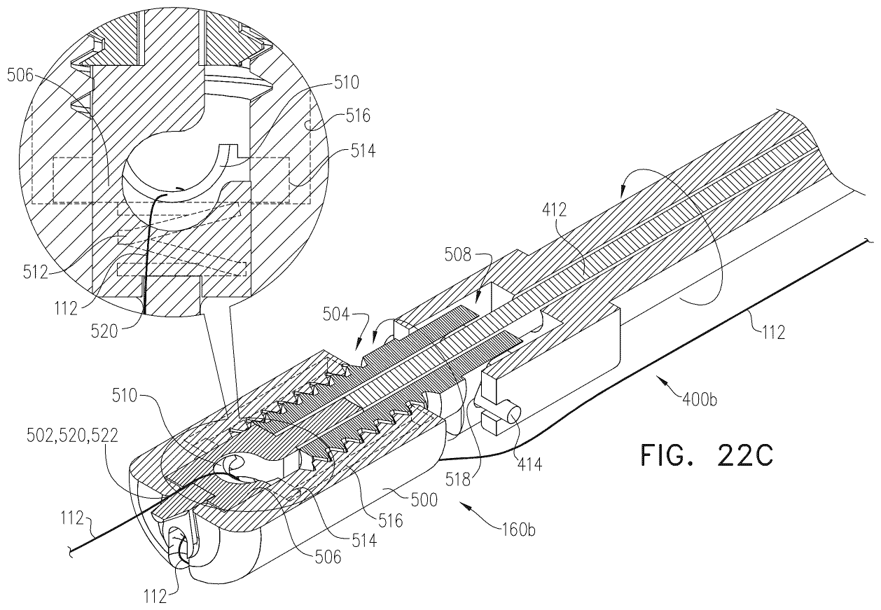

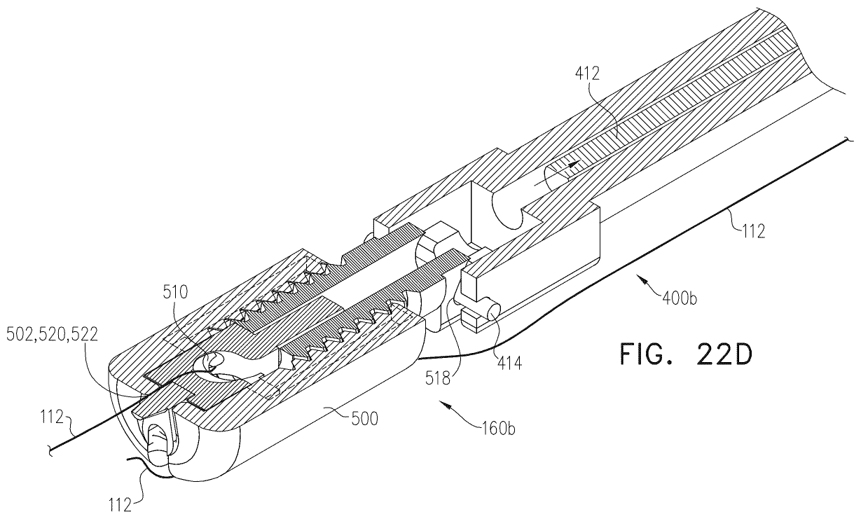

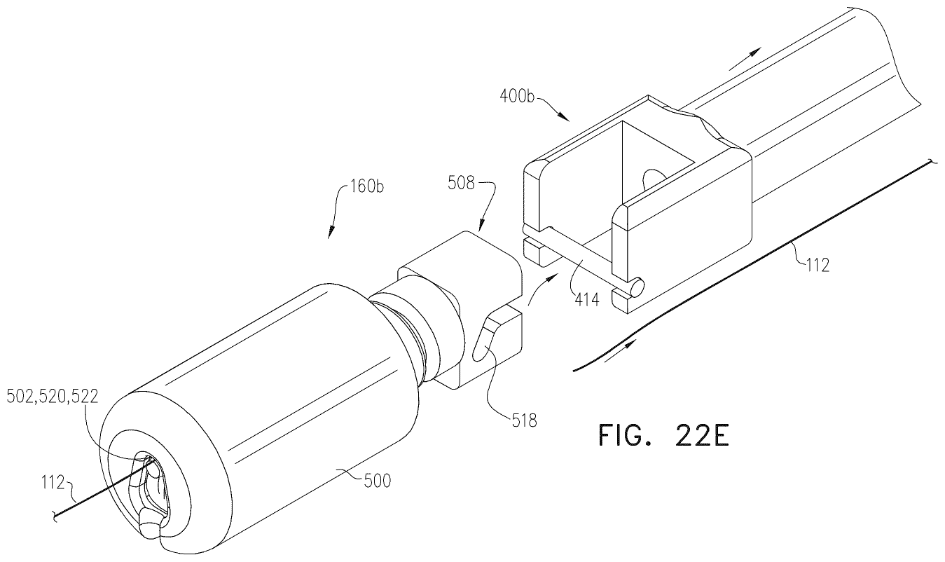

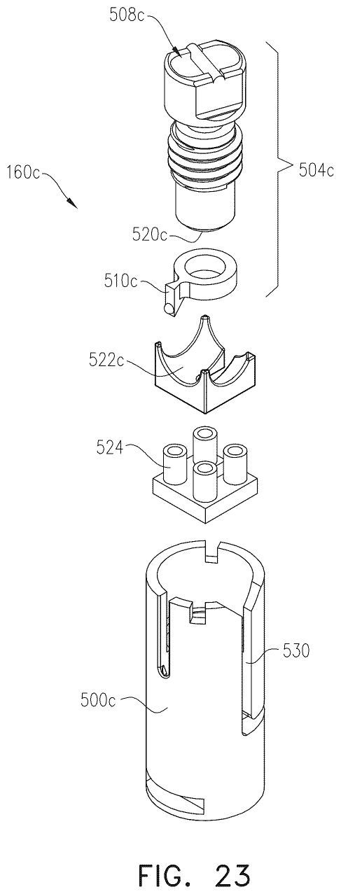

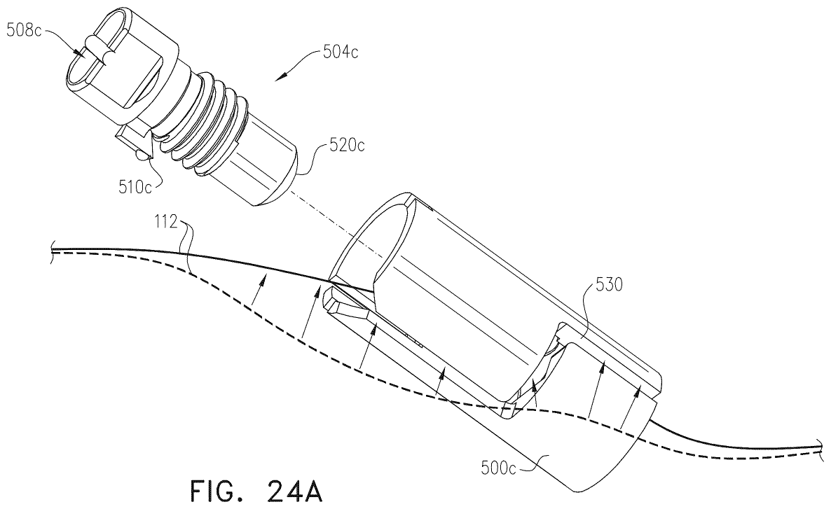

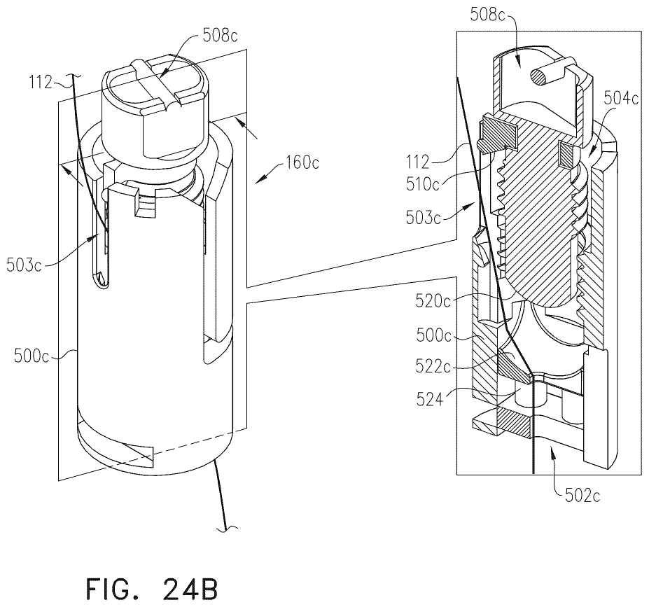

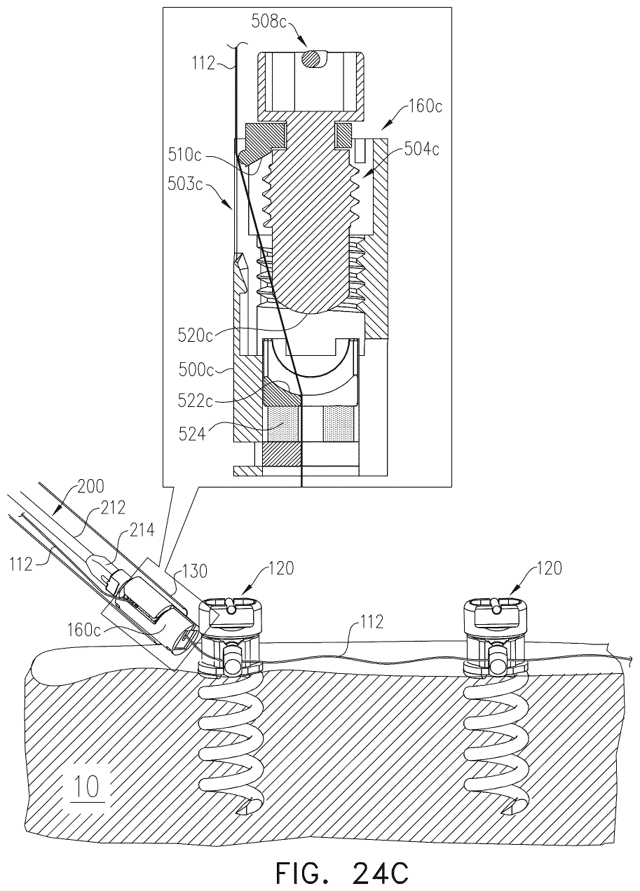

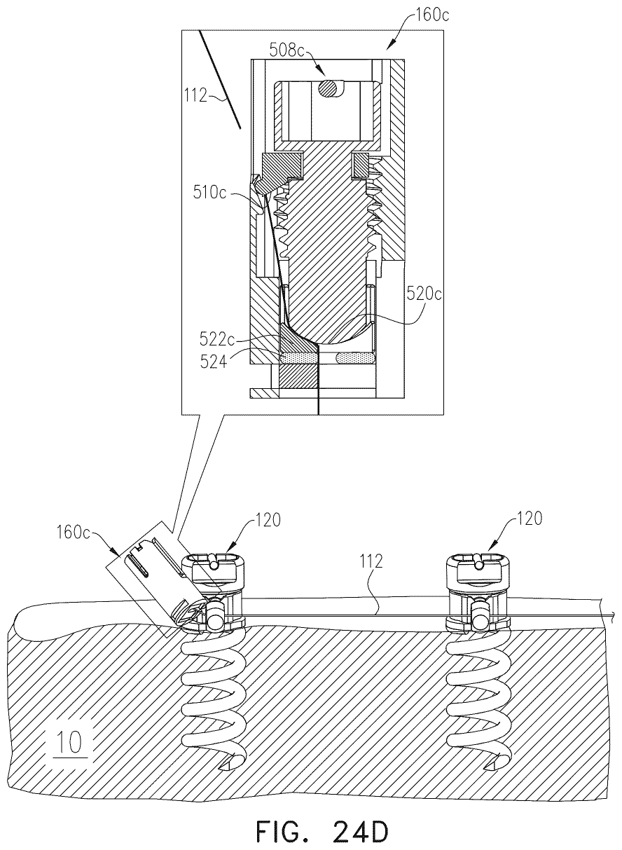

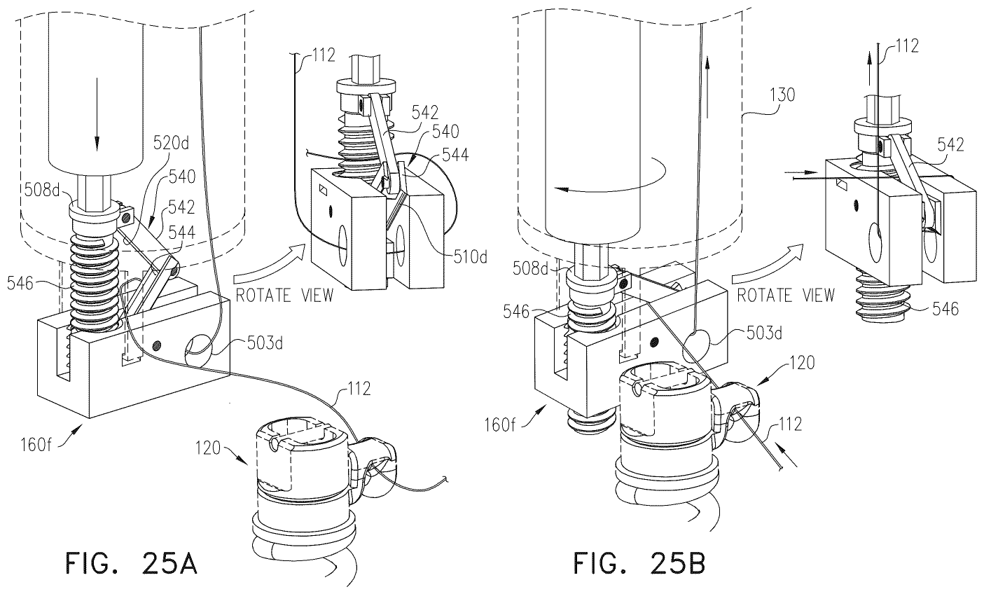

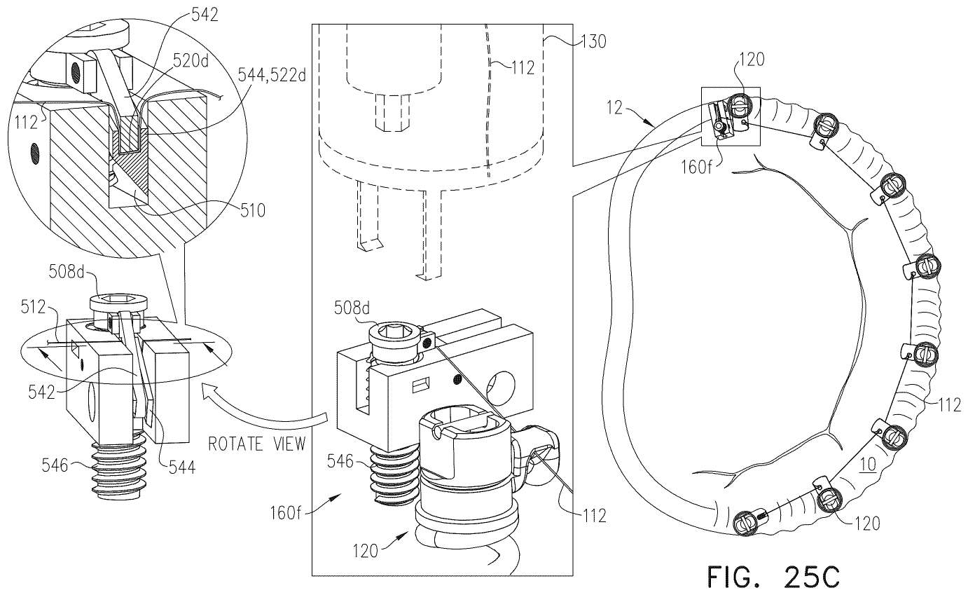

Reference is made to FIGS. 21, 22A-E, 23, 24A-D, 25A-C, 26, and 27A-C, which are schematic illustrations of locks, in accordance with some implementations. In addition to serving as a lock by locking to a tether, each of these locks includes a blade that cuts the tether in order to trim excess tether. In particular, each of these locks is configured to be used with a corresponding tool (e.g., a contraction and/or locking tool), that is configured to actuate the lock in a manner that both locks the lock to the tether and cuts the tether with the blade.

In some implementations, the lock defines a passage through the lock, the passage configured to receive the tether (e.g., tether 112) therethrough.

In some implementations, the lock has an unlocked state in which the lock is transluminally slidable along the tether to the tissue by the tether sliding through the passage.

In some implementations, the lock comprises: a clamp face, a blade, and/or an interface. In some implementations, the interface is engageable by the tool in a manner that configures the tool to actuate the lock by applying an actuating force to the interface.

In some implementations, the interface is configured such that, while the tether is disposed through the passage, actuation of the lock (i) locks the tether to the lock by clamping the clamp face to the tether, and (ii) cuts the tether with the blade.

In some implementations, and as shown, the lock is actuated via application of torque, e.g., to its interface. This torque can be translated into axial movement of the clamp face, e.g., via a screw. However, as detailed hereinbelow, the clamp face and its movement, as well as the blade and its movement, can differ between these various locks.

FIGS. 21, and 22A-E are schematic illustrations of a lock (which may, in some implementations, be considered a stopper) 160b and a tool 400b useable and/or for use therewith, in accordance with some implementations. The main image of FIG. 21 shows components of lock 160b and tool 400b. Inset A shows a cross-section through a casing 500 of lock 160b. Insets B, C, and D show, respectively, perspective, top, and side close-up views of an inner subassembly 504 of lock 160b, comprising a blade 510 and a clamp face 520. FIGS. 22A-E show at least some steps in the use of tool 400b and lock 160b, in accordance with some implementations.

In order to use lock 160b, the lock is slidably connected to tether 112. This can be achieved by positioning a bight of the tether, as loop 111, about a grasper 506 of inner subassembly 504 inside casing 500 (FIGS. 22A-B). In some implementations, this is achieved by advancing loop 111b through a window 502 of casing 500, and then hooking grasper 506 through the loop. For example, and as shown, loop 111 can be advanced entirely through casing 500, the hooking is performed outside the casing before the loop, hooked on grasper 506, is returned, with the grasper, into the casing. However, it is to be understood that this technique can be performed in another order of steps, and/or that other techniques can be used in order to arrive at the arrangement in which loop 111 is disposed about grasper 506 inside casing 500.

As shown, grasper 506 can be shaped as, or to define, a hook. It is to be noted that blade 510 is functionally obscured (e.g., sheathed) by (e.g., within) grasper 506. As shown, blade 510 can be hook-shaped, e.g., corresponding to the shape of grasper 506. Because of this arrangement, the position of loop 111 about (e.g., the hooking of the loop onto) grasper 506 can, for at least some implementation, be viewed as positioning the loop about (e.g., hooking the loop onto and/or around) blade 510. However, because of the obscuring of the blade, in this state of lock 160b tether 112 is not cut.

In some implementations, inner subassembly 504 comprises a spring 512 that maintains the functional obscuring/sheathing of blade 510. In the example shown, spring 512 is a tension spring, but it is to be understood that other spring formats can be used, mutatis mutandis. In some implementations, and as shown, blade 510 and spring 512 can be formed from a single piece of stock material, such as being cut from a single sheet of metal.

Similar to as described for lock 160a, the coupling of lock 160b to tether 112 can be performed at the extracorporeal portion of the delivery tool (e.g., at the extracorporeal unit of the catheter device) and/or without access to the proximal end of the tether. In this state, lock 160b can be advanced into proximal opening 320 and through tube 310. FIG. 22B represents such sliding, albeit without showing tube 310. FIG. 22B can be considered analogous to FIG. 19C. Note the arrangement of tether 112 in FIG. 22B is similar to that in FIG. 19C, with the part of the tether that is ahead of lock 160b (e.g., that has not yet entered the lock) being substantially straight, and the tether emerging from the lock to curve proximally past the lock and tool 400b, toward the extracorporeal part of the delivery tool (e.g., toward extracorporeal unit 350). Thus, during advancement of lock 160b, tether 112 is formed into an S-shape at and/or by the lock and/or delivery tool 400b.

It is to be noted that window 502 can provide and/or serve as an entrance and/or an exit to the passage through lock 160b through which tether 112 will slide.

Once appropriate tension has then been applied to tether 112 (e.g., as described hereinabove), lock 160b is then actuated to lock the lock to the tether and cut the tether (FIG. 22C). This can be achieved by operating tool 400b to apply an actuating force to an interface 508 of the locker. Actuation of lock 160b can involve relative axial movement between inner subassembly 504 and casing 500, e.g., movement of the inner subassembly distally with respect to the casing. In the example shown, casing 500 and inner subassembly 504 have complementary threading, and the actuating force is torque that rotates inner subassembly 504 relative to the casing, the complementary threading translating the relative rotation into axial movement.

In some implementations, the axial movement of inner subassembly 504 relative to casing 500 both (i) locks lock 160b to tether 112 by clamping clamp face 520 to the tether, and (ii) cuts the tether with blade 510. In some implementations, the locking can occur as the axial movement closes a gap between clamp face 520 and an opposing face 522 that can be provided by casing 500 (e.g., a rim of window 502). For example, and as shown, clamp face 520 can be provided by a plug or protrusion (e.g., a conical or frustoconical structure) that clamps tether 112 by protruding through window 502, e.g., in a manner that substantially plugs the opening.

In some implementations, the cutting is facilitated by the axial movement functionalizing blade 510, e.g., by exposing/unsheathing the blade from grasper 506. This functionalization can occur due to one or more tabs 514 inhibiting the blade from moving axially, e.g., the blade can be left behind as the component within which it was sheathed (e.g., grasper 506) moves axially. For implementations in which inner subassembly 504 has spring 512, this inhibition of axial movement of the blade is sufficient to overcome the retention force provided by the spring, i.e., the spring becomes strained. It is to be understood that, in some implementations, a plastically-deformable component can be used in place of spring 512.

In some implementations, tabs 514 are defined by, or coupled to, blade 510, and become obstructed after inner subassembly 504 has moved a predefined distance axially. In the example shown, casing 500 defines one or more grooves 516 within which tabs 514 are slidable until the predefined axial distance, at which point the tabs reach the end of the grooves, and blade 510 ceases to move axially despite grasper 506 (and the rest of inner subassembly 504) continuing to move axially.

In some implementations, the mere exposure/functionalization of blade 510 is sufficient to cut tether 112. In some implementations, an additional action, such as tugging on tether 112 and/or tool 400b may be required.

In some implementations, lock 160b can be configured such that its actuation locks it to tether 112 prior to cutting the tether. For example, a first amount of actuation can clamp the clamp face to the tether, and further actuation, beyond the first amount of actuation, may be required for the blade to cut the tether. Such a configuration can be provided, for example, by the shape (e.g., tapering), compressibility, and/or resilience of clamp face 520.

In some implementations, tool 400b can then be disengaged from lock 160b (e.g., from interface 508 thereof), and withdrawn. In the example shown, this is achieved by retraction of a lock-rod 412 of tool 400b (FIG. 22D), which allows a transverse lock-bar 414 of tool 400b to exit an oblique slot 518 of lock 160b, e.g., of interface 508 thereof (FIG. 22E). FIG. 22E also shows the trimmed proximal part of tether 112 being withdrawn, while lock 160b remains locked to the distal part of the tether that remains as a component of the implant.

Although lock 160b is shown as having a particular interface 508, in some implementations, the interface of a lock can be compatible with (e.g., engageable by) drive head 214 of anchor driver 210. For example, the interface of a lock can share features with (e.g., can be identical to and/or similar to) the interface of any of the anchors described herein. Thus, in some implementations, driver 210 can also serve as the tool for advancing and locking (e.g., actuating) a lock 160. Lock 160c, described hereinbelow, is shown has having such an anchor-driver-compatible interface, but it is to be understood that other locks can be adapted to have such an interface.

FIGS. 23 and 24A-D show a lock (which may, in some implementations, be considered a stopper) 160c; FIGS. 25A-C show a lock (which may, in some implementations, be considered a stopper) 160f; and FIGS. 26 and 27A-C show a lock (which may, in some implementations, be considered a stopper) 160e. Actuation of lock 160b moves its blade axially, e.g., the clamp face and the blade can move simultaneously and coaxially. In contrast, although actuation of lock 160c moves its blade axially, in the example shown the clamp face and blade of lock 160c do not move coaxially; instead, the axis along which the blade moves in parallel to that along which the clamp face moves. Lock 160f is configured such that its blade has planar movement, e.g., deflects and/or translates within a plane on which lies the axis of movement of its clamp face. Lock 160e is configured such that its blade revolves around the axis of movement of its clamp face.

FIG. 23 shows an exploded view of lock 160c, and FIGS. 24A-D show at least some steps in the use of the lock. For the sake of simplicity, the tool with which lock 160c is used is not shown. However, as noted above, anchor driver 210 can serve as this tool. Lock 160c comprises a casing 500c, and an inner subassembly 504c that comprises a blade 510c and a clamp face 520c.

In order to use lock 160c, the lock is slidably connected to tether 112, such that the tether is slidable through a passage defined through the lock (FIG. 24A). This can be achieved by introducing tether 112 sideways into the lock, e.g., lock 160c can define lateral access via which the tether is introducible sideways into the passage of the lock. For example, and as shown, lock 160c (e.g., casing 500c thereof) can define a lateral slit 530 that provides such lateral access. Similarly to some other locks described herein, this may advantageously allow lock 160c to be used without access to the proximal end of tether 112—e.g., while the proximal end of the tether remains within and/or engaged by the extracorporeal unit of the catheter tool.

In some implementations, and as shown, inner subassembly 504c is introduced into casing 500c subsequently to the introduction of tether 112 (FIG. 24B). However, in some implementations, inner subassembly 504c can be at least partly coupled to (e.g., disposed within) casing 500c prior to the introduction of tether 112. The passage through lock 160c has an entrance 502c at one end and an exit 503c at the other end. As lock 160c is advanced along tether 112 and the tether is tensioned, the tether slides into entrance 502c and out of exit 503c (FIG. 24C).

In some implementations, once sufficient tension has been applied to tether 112, lock 160c is actuated so that (i) clamp face 520c clamps to the tether, and (ii) blade 510c cuts the tether (FIG. 24D). Similarly to lock 160b, this actuation can be achieved by applying torque to interface 508c, e.g., such that complementary threading between inner subassembly 504c and casing 500c translates the torque into axial movement of both clamp face 520c and blade 510c. Although clamp face 520 can rotate while advancing, and although blade 510c can be mounted eccentrically, the blade can move in a simple axial direction, e.g., due to being mounted rotationally (e.g., via a collar, as shown).

In some implementations, lock 160c can be configured such that its actuation locks it to tether 112 prior to cutting the tether. For example, a first amount of actuation can clamp the clamp face to the tether, and further actuation, beyond the first amount of actuation, can be required for the blade to cut the tether. Such a configuration can be provided, for example, by the shape (e.g., curvature), compressibility, and/or resilience of opposing face 522c. For example, a compressible member 524 can support (or define) opposing face 522c, such that the further amount of actuation maintains tether 112 clamped between clamp face 520 and the opposing face as the clamp face pushes the opposing face to move along with the clamp face in a manner that enables blade 510c to move and cut the tether. That is, lock 160c can become locked to tether 112 upon clamp face 520c reaching opposing face 522c (state not shown), but only upon further actuation of the lock, and thereby compression of compressible member 524, does blade 510c move sufficiently to cut the tether (FIG. 24D).

In some implementations, the compressible member can comprise one or more polymers, fabrics, shape memory materials, foams, elastic portions, balloons, bladders, seals, stents, springs, combinations of two or more of these, etc.

FIGS. 25A-C show a lock 160f that comprises a mechanical linkage 540 that includes a first bar 542 and a second bar 544. In some implementations, and as shown, mechanical linkage 540 is a planar linkage. The blade 510d and/or the clamp face 520d of lock 160f is/are provided (e.g., defined) by the bars of the mechanical linkage. In the example shown, first bar 542 defines clamp face 520d, second bar 544 defines blade 510d. Bar 542 can be hingedly connected to bar 544, e.g., as is observable from the transition from FIG. 25A to FIG. 25B.

As for other locks, this actuation is performed once sufficient tension has been applied to tether 112 (FIG. 25B). Actuation of lock 160f can be achieved by applying torque to an interface 508d of the lock. Again, threading can be utilized to translate the torque into axial movement. For example, and as shown, interface 508d can be coupled to a threaded rod 546 that cooperates with mechanical linkage 540 as a linear actuator, such that rotation of the interface rotates the threaded rod and pivots bar 542 with respect to bar 544. External threading of rod 546 can be complementary to internal threading of casing 500d.

FIG. 25A shows lock 160f having been threaded onto tether 112, e.g., with the tether passing between bars 542 and 544 (and optionally rod 546), and looping back through a passage through casing 500d, and out of the casing at an exit 503d. In this arrangement, lock 160f is advanced to anchor 120 (e.g., as described for other locks, mutatis mutandis). Tether 112 is tensioned, and then lock 160f is actuated (FIGS. 25B-C). FIG. 25C illustrates that this actuation locks tether 112 to lock 160f by clamping clamp face 520d to tether 112, and cuts the tether with blade 510d.

In some implementations, and as shown, mechanical linkage 540 is configured such that actuation of lock 160f clamps tether 112 between clamp face 520d (e.g., bar 542) and bar 544. In some implementations, bar 544 thereby provides (e.g., defines) an opposing face 522d of lock 160f. In some implementations, mechanical linkage 540 is configured such that actuation of lock 160f clamps tether 112 between clamp face 520d (e.g., bar 542) and a casing 500d of the lock.

In some implementations, and as shown, blade 510d faces away from bar 542.

Lock 160f can be configured such that its actuation locks it to tether 112 prior to cutting the tether. For example, a first amount of actuation can clamp the clamp face to the tether, and further actuation, beyond the first amount of actuation, can be required for the blade to cut the tether. Such a behavior can be provided by mechanical linkage 540 being configured such that a distance-of-movement of the blade required to cut the tether is greater than a distance-of-movement of the clamp face required to clamp the clamp face to the tether. Alternatively or additionally, mechanical linkage 540 can be configured to move the blade at a different rate to the clamp face.

FIGS. 26, and 27A-C show a lock 160e in which actuation of the lock cuts tether 112 by revolving a blade 510e of the lock around an axis. In some implementations, that axis is the axis along which a clamp face 520e of the lock moves upon actuation of the lock. That is, in some implementations, actuation of lock 160e (i) clamps clamp face 520e to tether 112 by moving the clamp face along an axis, and (ii) cuts the tether with the blade by revolving the blade around that axis.

Like the preceding locks, lock 160e can be actuated by applying torque to an interface 508e of the lock. In some implementations, and as shown, interface 508e is fixed to interface 508e, such that rotation of the interface revolves the blade around the axis of rotation of the interface. Also like the preceding locks, axial movement of clamp face 520e is achieved by a screw thread translating torque into axial movement. In the case of lock 160e, the lock comprises a threaded rod 548 whose external screw thread complements an internal screw thread of a casing 500e of the lock. Operative coupling between interface 508e and rod 548 transfers torque from the interface to the rod.

In some implementations, as shown, threaded rod 548 can provide (e.g., define) clamp face 520e of lock 160e. In some implementations, threaded rod can be operatively coupled to another component that provides (e.g., defines) the clamp face. Similarly to other locks described herein, casing 500e can provide an opposing face 522e against which clamp face 520e clamps tether 112.

Rod 548 and/or blade 510e may be considered components of an inner subassembly 504e of lock 160e.

FIG. 27A shows lock 160e having been threaded onto tether 112, e.g., with the tether passing into an entrance 502e, along a passage within the lock, and out of an exit 503e. In this arrangement, lock 160e is advanced to anchor 120 (e.g., as described for other locks, mutatis mutandis). Tether 112 is tensioned, and then lock 160e is actuated (FIGS. 27B-C).

Lock 160e can be configured such that its actuation locks it to tether 112 prior to cutting the tether. For example, a first amount of actuation can clamp the clamp face to the tether (FIG. 27B), and further actuation, beyond the first amount of actuation, may be required for the blade to cut the tether (FIG. 27C). In some implementations, such a configuration can be provided by the operative coupling between interface 508e and rod 548 being via a slip mechanism. For example, lock 160e can become locked to tether 112 upon clamp face 520e reaching opposing face 522e (FIG. 27B), at which point the opposing face resists further advancement, and therefore rotation, of rod 548. Due to this resistance, upon further actuation of lock 160e (e.g., upon further torque being applied to interface 508e) the slip mechanism is overcome, resulting in interface 508e rotating, and blade 510e revolving, while rod 548 remains stationary (FIG. 27C).

In some examples, as shown, the slip mechanism of lock 160e comprises a spring-loaded detent 532 that protrudes into a notch 534, e.g., with the detent and/or the notch having a sloped edge. In this example, the overcoming of the slip mechanism results in detent 532 slipping out of notch 534 (e.g., as the spring of the detent becomes compressed), allowing interface 508e to rotate without transferring torque to rod 548. Detent 532 is shown as being attached to interface 508e and notch 534 is shown as being defined in rod 548, but it is to be understood that the inverse arrangement, and other arrangements, are possible, mutatis mutandis.

Although, during the first amount of actuation, blade 510e revolves as rod 548 advances axially, the blade may not cut tether 112 until sufficient actuation brings the blade into an appropriate axial position with respect to the passage of tether 112 through the lock.

In some implementations, the slip mechanism provides lock 160e with some functional flexibility. For example, irrespective of the rotational position of blade 510e at the moment that clamp face 520e reaches opposing face 522e and rod 548 can therefore no longer rotate, application of further torque to interface 508e can still revolve the blade through tether 112. The slip mechanism can also confer reliability on lock 160e because blade 510e can be repeatedly revolved to ensure cutting of tether 112, without affecting the locking of the lock to the tether.

It is to be noted that features of the various locks described herein can be combined and/or substituted with those of each other.

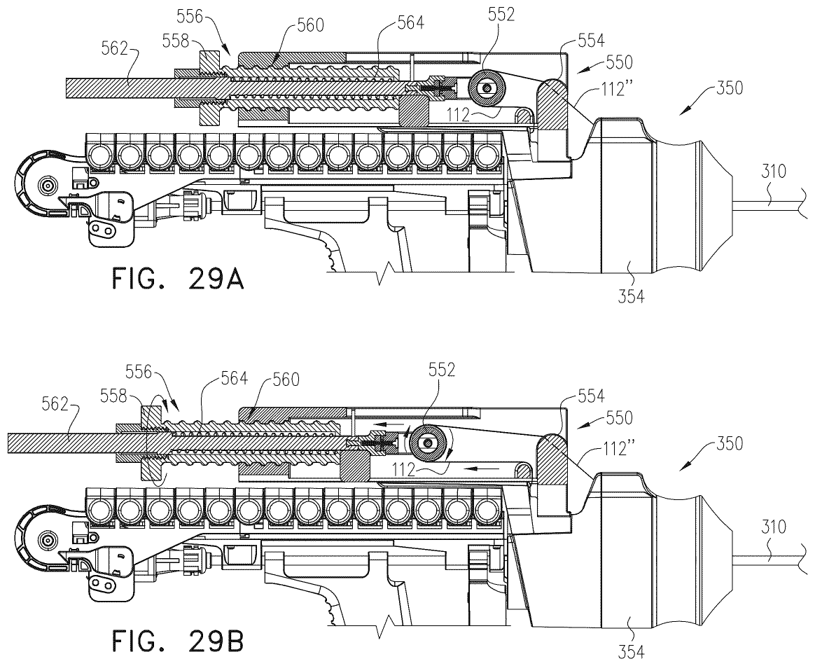

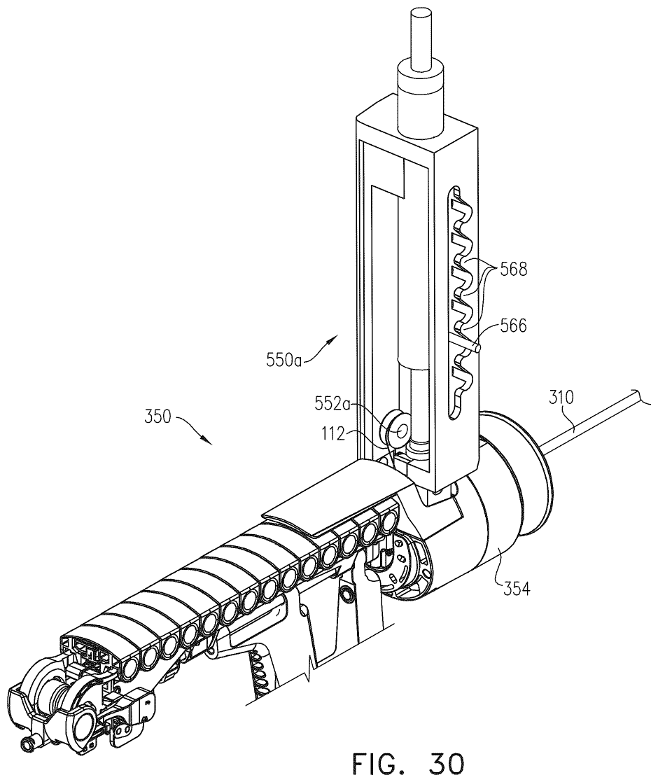

Reference is now made to FIGS. 28, 29A-B, and 30, which are schematic illustrations of tensioners, in accordance with some implementations. In some implementations, these tensioners are configured to engage an intermediate region or bight of tether 112 (e.g., without requiring access to an end of the tether) and to apply tension to the tether by pulling on the intermediate region or bight. The tensioners can be mounted or reversibly mountable on an extracorporeal unit of a catheter device such as catheter device 300. In some implementations, the tensioner can be a component of the extracorporeal unit. The tensioners can advantageously be used to assess an implant 110 (or variant thereof) during its implantation. For example, tensioning of tether 112 between the anchoring of one anchor of implant 110 and the anchoring of another, while imaging the heart valve at which the implant is being implanted, can provide useful information regarding the behavior of the implant and the valve. Such information can be used to direct subsequent parts of the procedure, e.g., deciding whether to add another anchor and, if so, identifying an optimal anchoring site for it. Such an advantage can be further augmented for implementations in which the tensioner indicates the magnitude of the applied tension, and/or includes means for controlling the magnitude of tension applied.

FIGS. 28 and 29A-B show a tensioner 550, mounted on catheter device 300, in accordance with some implementations. Via its engagement with tether 112, tensioner 550 can be operated to apply tension to the tether mid-procedure, and without access to either end of the tether. Tensioner 550 can engage with a segment of the tether that is exposed at extracorporeal unit 350. Thus, extracorporeal unit 350 can define an access site at which tether 112 is exposed. In the example shown, this exposed segment of tether 112 is immediately outside of de-slacker 354, e.g., between the de-slacker and anchors 120 (see also FIG. 1).

In some implementations, the engagement of tensioner 550 can be provided by looping the tether around a bearing of the tensioner, the bearing being movable (e.g., linearly) in order to pull on the tether. The pulling of the tether can intrinsically urge the tether to slide over the bearing, and therefore in some implementations the bearing comprises a sheave 552 or other rotating bearing that facilitates such sliding. FIG. 29B shows tensioner 550 being operated to apply tension to tether 112 by moving sheave 552, e.g., linearly-such that the sheave pulls on the tether.

In some implementations, and as shown, tensioner 550 comprises a gripper 554 that is configured to grip tether 112, e.g., at one side of sheave 552. This gripping isolates, from the tension applied by tensioner 550, a region of the tether beyond the gripper, e.g., defining an isolated region 112″ of the tether. This can be particularly advantageous for implementations in which the catheter device includes de-slacker 354, because isolating the de-slacker from tensioner 550 may prevent de-slacker from letting out tether 112 in response to the pulling by the tensioner. That is, the tether 112 that is pulled by tensioner 550 is taken from the distal part of the tether, allowing accurate measurement of the tension and/or length of tether pulled, and accurate assessment of the effect thereof. In some implementations, rather than gripper 554 isolating the de-slacker, a similar isolating effect can be achieved by locking the de-slacker. For example, and as described hereinabove, de-slacker 354 can comprise a lock, actuation of which locks the winch of the de-slacker, i.e., does not allow tether to be pulled out of the de-slacker.

The movement (e.g., linear movement) of sheave 552 can be achieved by various means. In the example shown, tensioner 550 comprises a linear actuator 556, e.g., rotation of a knob 558 being translated, by complementary screw threads 560, into linear movement. Using linear actuator 556 advantageously means that tensioner 550 automatically maintains the tension that it has applied, e.g., it requires active reversal in order to release the tension in the tether.

In some implementations, tensioner 550 can further comprise a force gauge 562 that indicates a magnitude of the tension being applied. Force gauge 562 can, for example, be a spring-based mechanical force gauge in which a spring 564 becomes strained, to a known degree, by the tension on tether 112.

FIG. 30 shows a tensioner 550a, which can be considered to be a variant of tensioner 550. Similarly to tensioner 550, tensioner 550a can have a rotating bearing such as a sheave 552a that is moved (e.g., linearly) in order to apply tension to tether 112, which is looped around the rotating bearing. Tensioner 550a can be as described for tensioner 550, except that (i) it applies the tension by pulling the engaged region of the tether laterally (e.g., away from the overall axis of the catheter device; upwards in the figure), and (ii) application of tension is performed by the operator applying a linear force, e.g., by gripping and moving a peg 566. Peg 566 can cooperate with another component of tensioner 550a, such as its housing, to serve as a latch or ratchet having one or more stable positions in which the tensioner maintains the tension applied. For example, and as shown, the housing of tensioner 550a can be shaped to define a row of teeth 568 within which peg 566 can rest.

Although tensioners 550 and 550a are described as being usable and/or for use mid-procedure, it is to be noted that, in some implementations, they, or similar devices, can be used to apply, to tether 112, the tension that is locked into the tether (e.g., by lock 160 or a variant thereof), i.e., the tension that will remain in implant 110. This locking-in can be that which is performed toward the end of the procedure, but the tensioners can be used similarly for implementations in which tension is applied and locked in mid-procedure (e.g., between anchors).

In some implementations, once all anchors 120 have been anchored, tether 112 can be tensioned, locked, and trimmed while catheter device 300 remains in place. That is, in some implementations, catheter device 300 can provide transluminal access for advancement and anchoring of anchors 120, tensioning of tether 112, advancement of lock 160 and its locking to the tether, and trimming of the tether.

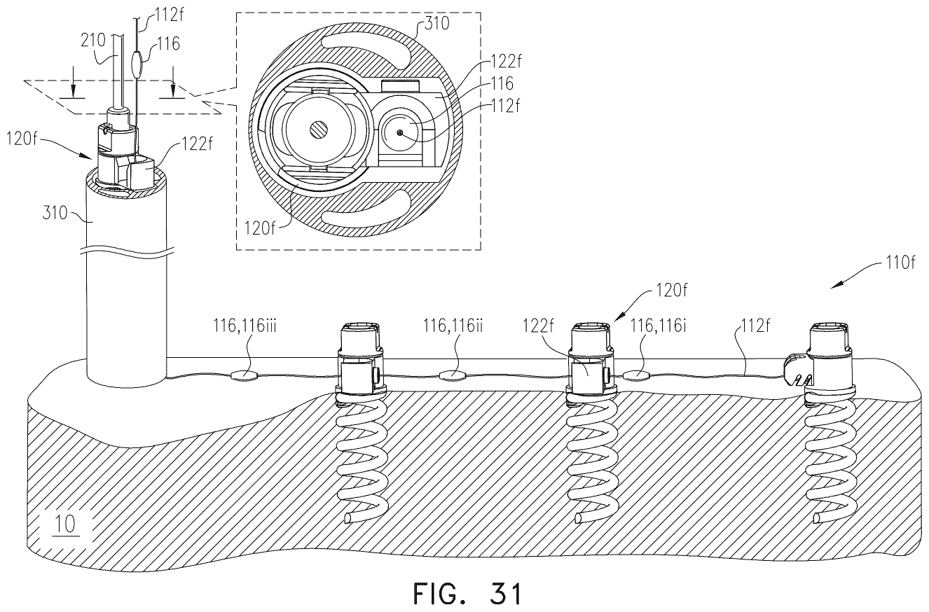

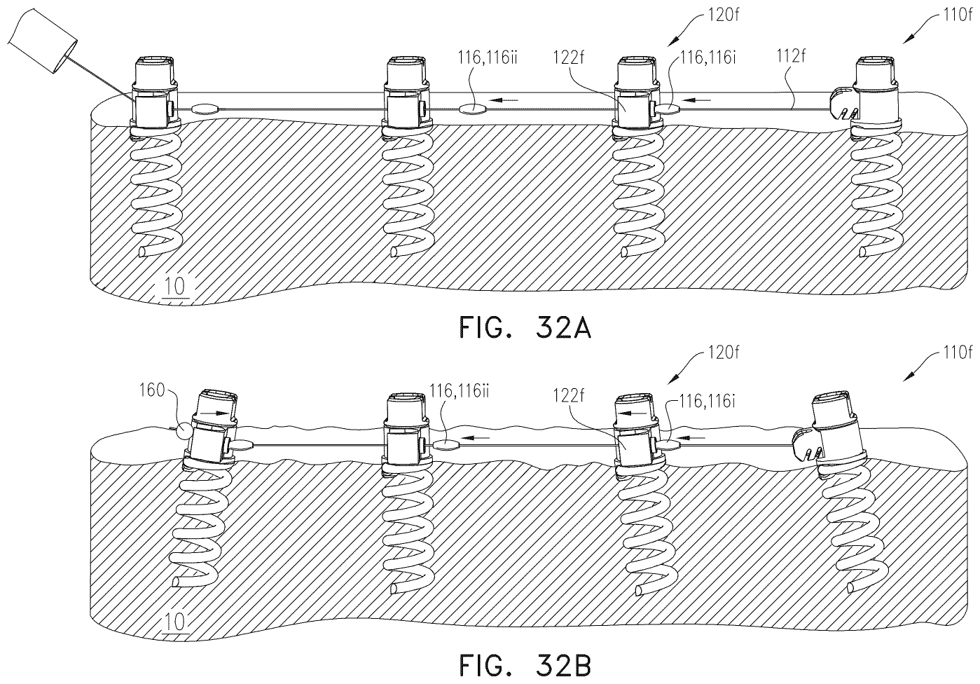

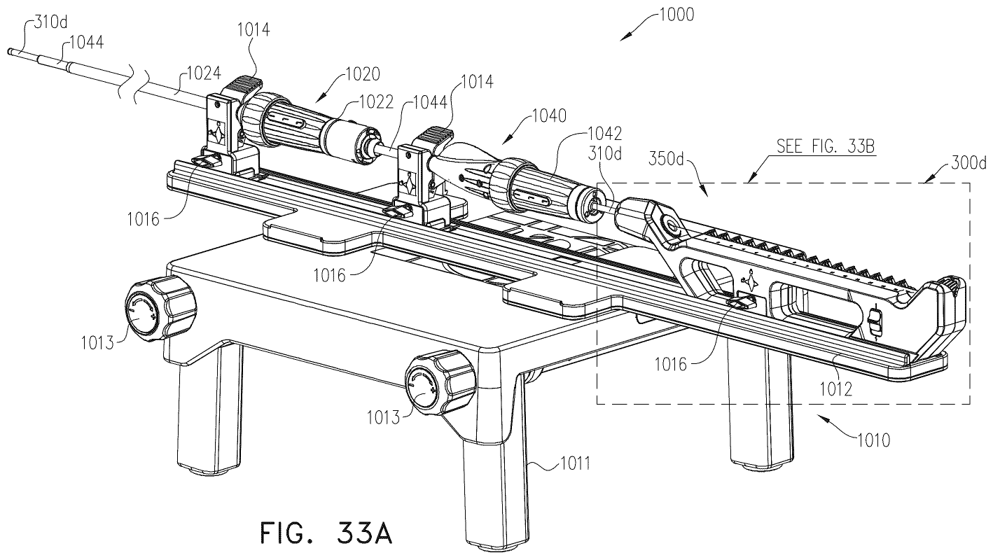

Reference is made to FIGS. 31 and 32A-B, which are schematic illustrations of an implant 110f being implanted, in accordance with some implementations. Implant 110f is a variant of implant 110 comprising a series of beads 116 distributed along, and fixed to, its tether 112f. In some implementations, beads 116 can be considered to be components of tether 112f, e.g., the tether can be manufactured (e.g., extruded, molded, spun, or woven) to include the beads. In some implementations, beads 116 can be considered to be attached to tether 112f, e.g., by threading, tying, adhering, welding, soldering, etc.

In some implementations, implant 110c comprises multiple anchors 120f. Anchor 120f is a variant of anchor 120 whose head 122f has a geometry that provides a particular interaction with beads 116. Head 122f (e.g., an eyelet thereof) is configured has a geometry that (i) facilitates sliding of the head over and along the tether while the anchor axis is parallel with the tether by allowing the beads to pass through the head, and (ii) inhibits sliding of the head over and along the tether while the anchor axis is transverse to the tether by obstructing the beads from passing through the head. This geometry can be provided, at least in part, by beads 116 being oval or prolate spheroids, e.g., as shown.

Although the eyelet of head 122f is typically revolvable about the anchor axis of anchor 120f, it may not be rotatably mounted in a manner that allows the eyelet to rotate with respect to the anchor axis. For example, the eyelet can be fixedly mounted on a collar that is rotatable about the anchor axis.

As described hereinabove, each anchor (except the first) is advanced over and along tether 112 toward tissue 10. FIG. 31 shows this, with three anchors 120f already anchored to tissue 10, and one anchor 120f being advanced, within tube 310, over and along tether 112f toward the tissue 10 while the anchor axis is parallel with the tether, and with beads 116 therefore passing through head 122f (e.g., the eyelet of the head). For the sake of this illustration, the “first” anchor is the right-most in the figure, the “second” anchor is the anchor immediately to its left, the “third” anchor is the anchor immediately to the left of the second anchor, and the “fourth” anchor (which is the final anchor in this particular illustration) is the left-most and is within tube 310. Similarly, the first three beads 116 are numbered 116i, 116ii, and 116iii to facilitate the following description.

In some implementations, beads 116 can be radiopaque and/or echogenic and can therefore serve as a measurement guide during implantation, such as with respect to spacing between anchors, e.g., as described for wavy tether 112b, mutatis mutandis.

In some implementations, beads 116 can additionally or alternatively affect the forces experienced by the anchors of the implant upon tensioning of the tether of the implant, e.g., similarly or analogously to spacers 150. For example, the position, along the tether, of an anchor 120f with respect to a bead 116, can affect whether that anchor experiences greater or lesser pulling than do other anchors of the implant. FIGS. 32A-B illustrate this. FIG. 32A shows a little tension having been applied to tether 112f, removing slack in the tether and drawing the bead 116i into contact with the head of the second anchor. This tension also draws bead 116ii toward the head of the third anchor but, because the initial spacing between bead 116ii and the third anchor was greater than that between bead 116i and the second anchor (see FIG. 31), bead 116ii does not reach the head of the third anchor.

FIG. 32B shows additional tension having been applied, and locked into, to tether 112f. As noted above, the geometry of head 122f obstructs beads 116 from passing through the head while the anchor axis is transverse to tether 112f. Thus, bead 116i abuts against the head of the second anchor and the pulling force that would otherwise be experienced by the first anchor alone is shared by the second anchor-illustrated by the first and second anchors having tilted leftward together. This may advantageously enhance anchoring of the end of tether 112f, e.g., reducing a likelihood of the first anchor becoming pulled out of the tissue by tension in the tether. In the example shown, bead 116f does not reach the third anchor, and the third anchor is therefore shown remaining upright. However, it is to be understood that many arrangements of implant 110f are possible, permitting force distribution to be tailored to particular circumstances, e.g., by varying the initial space between each anchor and the bead immediately distal to it.

Although exactly one bead 116 is shown between each anchor and the next, it is to be noted that this is merely illustrative, e.g., a more or less dense distribution of beads can be used.

Although the distribution of beads 116 is shown as homogonous along tether 112f, it is to be noted that this is merely illustrative. In some implementations a denser distribution of beads may exist along certain regions of the tether (e.g., toward one or both ends of the tether), while a less dense distribution of beads (perhaps even no beads) may exist along other regions of the tether (e.g., toward the middle of the tether).

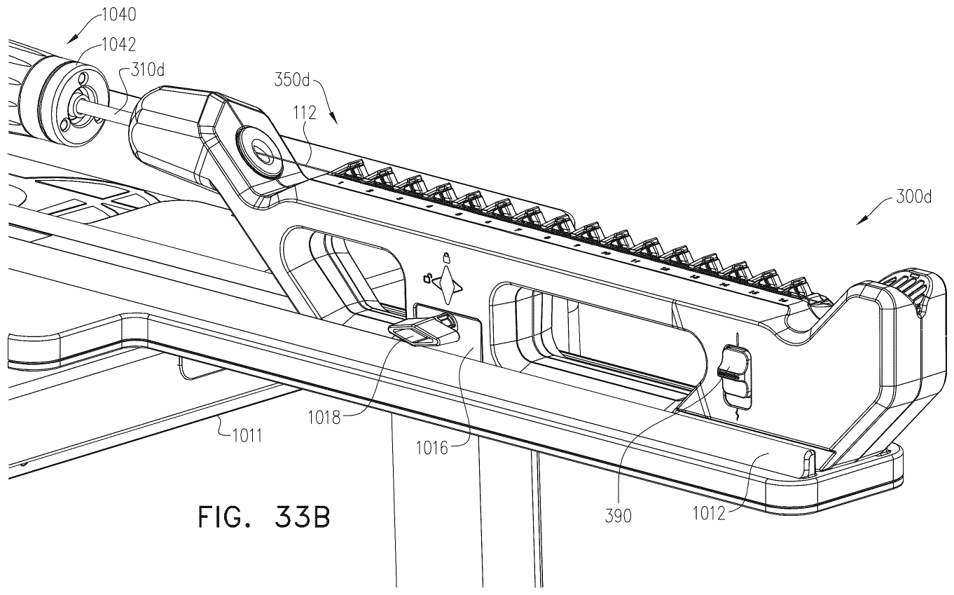

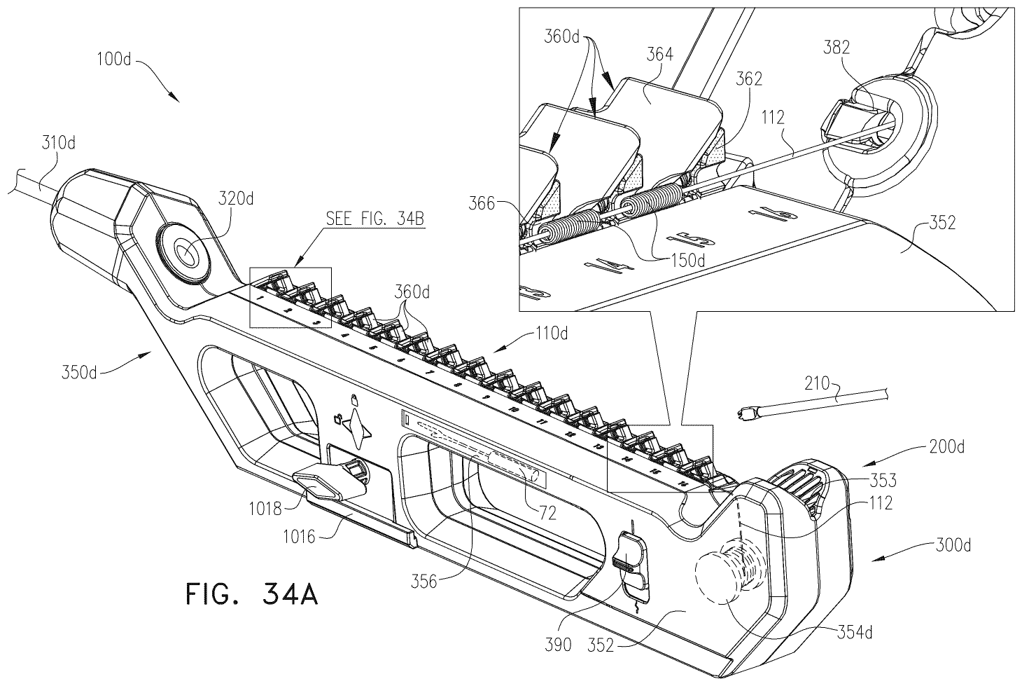

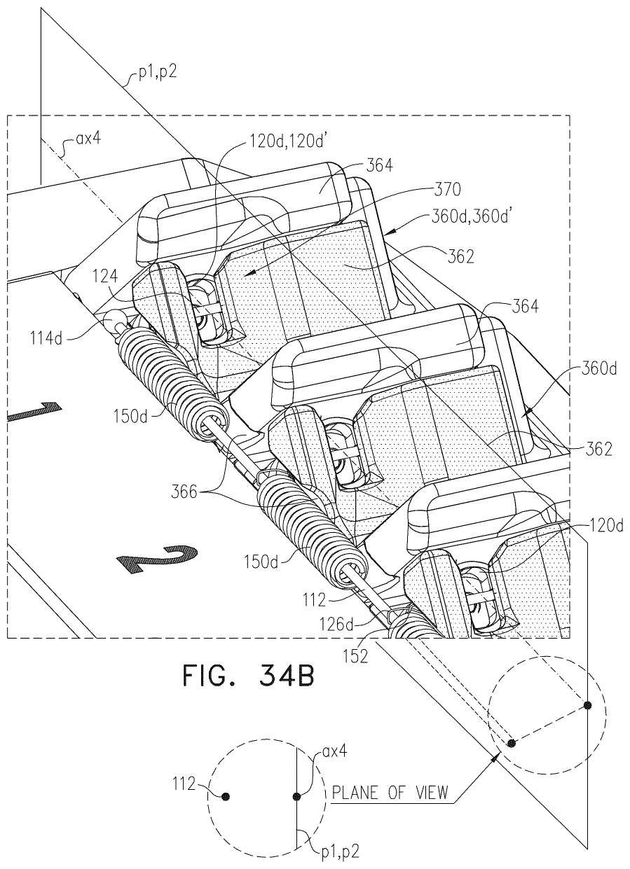

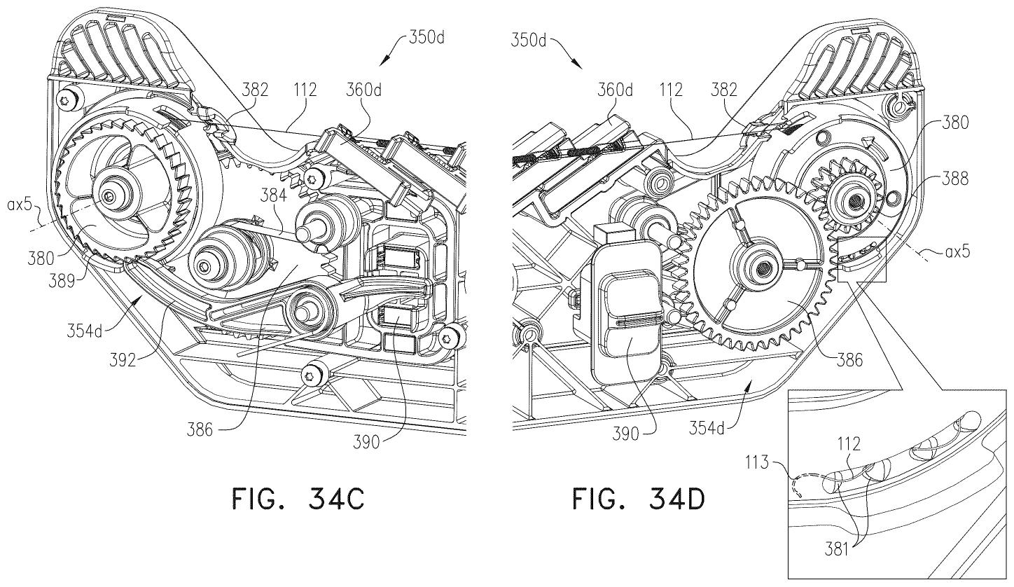

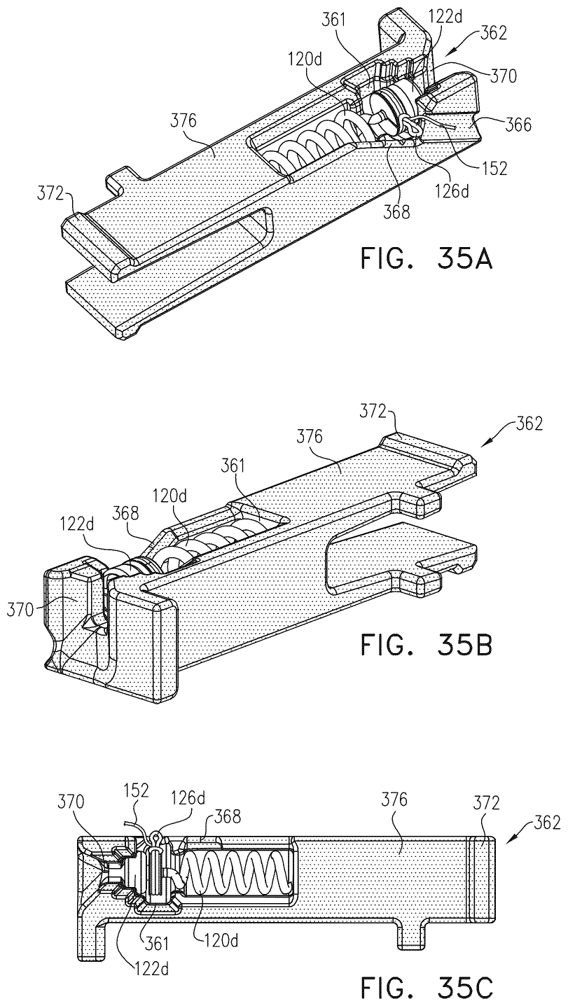

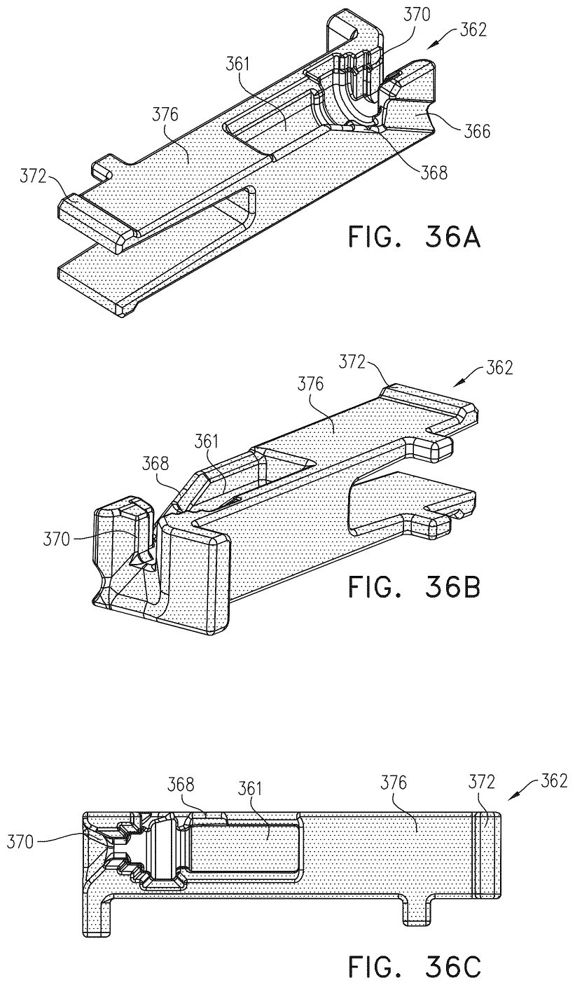

Reference is now made to FIGS. 33A-B, 34A-D, 35A-C, 36A-C, 37A-G, 38A-C, 39A-I, 40A-C, and 41A-L which are schematic illustrations of a system 1000 and techniques usable and/or for use therewith, in accordance with some implementations.

System 1000 comprises a catheter system 1002 that comprises at least one catheter. System 1000 further comprises a delivery tool and/or an adjustment tool. In the example shown, catheter system 1002 comprises an outer catheter 1020 and an inner catheter 1040, each of which has an extracorporeal unit (1022 and 1042, respectively) and a flexible tube (1024 and 1044, respectively). For each of catheters 1020 and 1040, the flexible tube can have a distal steerable region (i.e., i.e., a region that is actively deflectable) that is operatively coupled to the extracorporeal unit (e.g., via one or more pull wires) such that steering of the steerable region can be effected by operation of the extracorporeal unit (e.g., one or more user interfaces or controllers thereof, such as knobs). For some implementations in which system 1000 is used for heart valve repair, catheter 1020 can be transluminally advanced to a chamber adjacent the heart valve (e.g., an atrium upstream of the heart valve), e.g., such that the distal end of tube 1024 reaches the chamber. In some implementations, catheter 1040 can be transluminally advanced such that distal steerable region and/or the distal end of tube 1044 exits the distal end of tube 1024, and faces and/or approaches the tissue to which implant 110 is to be implanted. Catheters 1020 and 1040 can be advanced with tube 1044 already disposed through tube 1024. Alternatively, tube 1044 can be advanced through tube 1024 after the distal end of tube 1024 is already in (or at least close to) the heart chamber.

System 1000 can further comprise a support assembly 1010 configured to support catheter 1020, catheter 1040, and/or other components described hereinbelow. Support assembly 1010 can comprise a track 1012 (e.g., a rail) which itself can be supported by a pedestal 1011. Track 1012 can be linear, as shown. A height and/or slope of support assembly 1010 can be adjustable in order to optimize alignment for advancement into the subject, such as by actuating one or more control knobs 1013 on pedestal 1011. The extracorporeal units of catheters 1020 and 1040 are mounted on track 1012 such that they, and thereby the catheters as whole, are axially slidable with respect to each other and with respect to the subject. For some implementations, catheters 1020 and 1040, support assembly 1010, and techniques for use therewith, can be as described, or similar to aspects of those described, in one or more of the following publications, each of which is incorporated herein by reference:
PCT Publication WO 2013/069019 to Sheps et al.
PCT Publication WO 2014/064694 to Sheps et al.
PCT Publication WO 2020/112622 to Tyler et al.
US Patent Application 2021/0251757 to Siegel et al.
U.S. Provisional Patent Application 63/514,785 to Delgado et al.

For some implementations, system 1000 comprises a delivery tool, for implanting implant 110, that can comprise a catheter device. In the example shown, system 1000 comprises a delivery tool 200d that comprises a catheter device 300d. Delivery tool 200d is described in more detail hereinbelow but FIGS. 33A-B illustrate catheter device 300d of delivery tool 200d in the context of system 1000 as a whole, i.e., showing that a flexible tube 310d of the catheter device extends through the catheter 1020 and/or catheter 1040, and an extracorporeal unit (i.e., a catheter-device extracorporeal unit) 350d of the catheter device is mounted on track 1012 such that it is axially slidable with respect to catheters 1020 and 1040 and with respect to the subject.

For some implementations, tube 310d is transluminally advanced along with one or more of the catheters (e.g., while already extended through the one or more catheters). For some implementations, tube 310d is advanced through the one or more of the catheters after the catheters have already been transluminally advanced to the heart.

In some implementations, as in the example shown, the mounting of each of the extracorporeal units on track 1012 can be via a T-slot arrangement of a foot 1016 that engages the track in a manner that allows movement in only one direction-axial sliding along the track. Other arrangements and ways of mounting or engaging a track or mount can be used as well (e.g., a T-slot arrangement is an option, but is not required).

In some implementations, the mounting allows each component to be individually locked in a continuum of axial positions along the track. Whereas catheters 1020 and 1040 can be mounted to track 1012 by being clipped to mounts 1014 that themselves are mounted to the track, extracorporeal unit 350d can be mounted to the track directly. That is, whereas the feet 1016 for mounting catheters 1020 and 1040 are components of separate mounts 1014, extracorporeal unit 350d can have an integrated foot 1016. For each component that is slidably mounted on track 1012, its foot 1016 can be reversibly locked in place, e.g., by rotating a cam 1018 or similar that causes the component (e.g., its foot) to grip the track. This allows the axial position of the component with respect to the other components and with respect to the vasculature of the subject to be easily, controllably, and repeatedly adjusted and/or stabilized.

Therefore, in accordance with some implementations, a system for treating a subject can comprise one or more of: (a) a support assembly that comprises a track; (b) a first catheter, comprising (i) a first-catheter flexible tube, and/or (ii) a first-catheter extracorporeal unit, coupled to a proximal part of the first-catheter flexible tube, and slidably mountable on the track such that the first-catheter flexible tube extends distally away from the track and into the subject; (c) an implant catheter, comprising (i) an implant-catheter flexible tube, and/or (ii) a implant-catheter extracorporeal unit, coupled to a proximal part of the implant-catheter flexible tube, and slidably mountable on the track proximally from the first-catheter extracorporeal unit such that (1) the implant-catheter flexible tube extends distally away from the track and through the first-catheter flexible tube, and/or (2) a distance along the track between the implant-catheter extracorporeal unit and the first-catheter extracorporeal unit is adjustable; (d) an implant, mounted on the implant catheter, and transluminally implantable in the subject using the implant catheter; and/or (e) an adjustment tool, comprising (i) a flexible shaft, and/or (ii) an adjustment-tool extracorporeal unit, coupled to a proximal part of the flexible shaft, the adjustment tool being configured to be switched with the implant catheter subsequently to implantation of the implant such that (1) the adjustment-tool extracorporeal unit becomes slidably mounted on the track proximally from the first-catheter extracorporeal unit, (2) the flexible shaft becomes disposed through the first-catheter flexible tube, extending distally away from the track and toward the implant, and/or (3) a distance along the track between the adjustment-tool extracorporeal unit and the first-catheter extracorporeal unit is adjustable.

FIGS. 34A-D, 35A-C, and 36A-C show delivery tool 200d in more detail. For simplicity, some of these figures show the delivery tool and its use in the absence of some other components of system 1000, i.e., catheters 1020 and 1040 and support assembly 1010. Delivery tool 200d can be considered to be a variant of delivery tool 200, and can be used to perform similar techniques, mutatis mutandis. Furthermore, in some implementations, components and features can be added and/or substituted between these two delivery tools, mutatis mutandis. For each component of delivery tool 200d, at least in some implementations, one or more characteristics of the component can be as described for an identically-named component of delivery tool 200, unless stated otherwise.

In some implementations, delivery tool 200d comprises a catheter device 300d and an anchor driver. In the example shown, the anchor driver of delivery tool 200d is anchor driver 210, but it is to be understood that a different anchor driver can be used, mutatis mutandis. Catheter device 300d comprises flexible tube 310d (which can be any variant of tube 310), and an extracorporeal unit 350d (e.g., an extracorporeal control unit), coupled to tube 310d, and configured to remain outside the body of the subject. In some implementations, extracorporeal unit 350d defines, or is coupled to, a handle of device 300d. In some implementations, extracorporeal unit 350d shares one or more features with one or more of the extracorporeal units described in International Patent Application Publication WO 2022/064401 to Halabi et al., and/or International Patent Application Publication WO 2022/172149 to Shafigh et al., each of which is incorporated herein by reference. Furthermore, catheter device 300d can be used, mutatis mutandis, to facilitate implantation of any of the implants described in US Patent Application Publication 2021/0145584 to Kasher et al., and/or WO 2022/172149 to Shafigh et al., each of which is incorporated herein by reference.

Delivery tool 200d can be used for implanting an implant in a subject, in accordance with some implementations. Delivery tool 200 and the implant can therefore both be components of a system 100d. System 100d can therefore be considered to be a subsystem of system 1000. In the example shown, the implant of system 100d is an implant 110d (a variant of implant 110), but it is to be understood that other implants (variants of implant 110 or otherwise) can be implanted using delivery tool 200d, mutatis mutandis. Implant 110d is a variant of implant 110 in which, inter alia, the eyelet of at least some of the anchors of the implant are formed from a textile. For example, and as shown, implant 110d can comprise a series of anchors 120d (or variants thereof), threaded onto tether 112.

System 100d can be considered to be a variant of system 100, and can be used to perform similar techniques, mutatis mutandis. Furthermore, in some implementations, components and features can be added and/or substituted between these two systems, mutatis mutandis. For each component of system 100d, at least in some implementations, one or more characteristics of the component can be as described for an identically-named component of system 100, unless stated otherwise.

FIG. 34A shows implant 110d mounted on (e.g., loaded in) extracorporeal unit 350d. System 100d (and system 1000 as a whole) can be provided in this state. FIG. 34B shows part of FIG. 34A in close-up.

In system 100, described hereinabove, the series of anchors is mounted such that the leading anchor 120′ of the implant (i.e., the anchor that will be the first to be advanced to the heart) is the proximal-most anchor of the series of anchors, e.g., is housed by the proximal-most cartridge of the series of cartridges. In contrast, in some implementations of system 100d, and as shown, the series of anchors is mounted such that the leading anchor 120d′ of the implant (i.e., the anchor that will be the first to be advanced to the heart) is the distalmost anchor of the series of anchors.

In some implementations, extracorporeal unit 350d comprises a series of cartridges (or anchor holders) 360d, each of which houses a respective anchor 120d. Thus, the series of cartridges is distributed along the extracorporeal unit (e.g., along a stock or body 352 of the extracorporeal unit) in a manner that supports the arrangement of the series of anchors.

In some implementations, extracorporeal unit 350d may be considered to comprise or define a magazine that stores and dispenses a series of anchors housed in a respective series of cartridges (or anchor holders). The same may be said for extracorporeal unit 350 described hereinabove.

The cartridges/anchor holders herein can be configured in a variety of ways (e.g., from a simple receptacle or hole for holding an anchor to more involved or elaborate configurations and mechanisms).

In some implementations, leading anchor 120d′ is housed by the distalmost cartridge/anchor holder 360d′ of the series of cartridges/anchor holders. As shown, distalmost cartridge/anchor holder 360d′ can be the closest of the cartridges/anchor holders to proximal opening 320d of tube 310d.

In some implementations of system 100d, the series of anchors 120d (and/or the series of cartridges/anchor holders) is distributed along extracorporeal unit 350d in a manner that defines a proximal-distal axis ax4. In some implementations, the proximal-distal axis ax4 is a line connecting the anchors of the series by passing through the same point on each anchor 120d. Such an arrangement may advantageously help a user to more efficiently align anchors with proximal opening 320d and advance the anchors through opening 320d and along the tube.

In some implementations, the series of anchors 120d (and/or the series of cartridges/anchor holders) is distributed along the extracorporeal unit 350d in a manner that defines a proximal-distal axis with two or more parallel rows or columns of anchors parallel to the axis.

In some implementations, the series of anchors 120d (and/or the series of cartridges/anchor holders) is distributed along the extracorporeal unit 350d in a manner that does not align with a proximal-distal axis (e.g., in a curved manner, in an angled matter, in a zig zag manner, etc.)

In some implementations, one or more (e.g., one, some, or all) anchors of the series can be mounted on the extracorporeal unit such that its anchor axis ax1 (described hereinabove as being defined by the tissue-engaging element of the anchor) lies obliquely with respect to proximal-distal axis ax4. This is illustrated in the inset of FIG. 37B, in which an oblique angle alpha_1 exists between axes ax1 and ax4. In this example, axis ax4 is shown passing through the head of each anchor, but it is to be understood that axis ax1 would be oblique with respect to axis ax4 irrespective of which point on the anchor axis ax4 passes through. In some implementations, anchors 120d of the series are imbricated.

In some implementations of system 100d, the anchor axes ax1 of the series of anchors 120d collectively define a common anchor plane p1, i.e., anchors 120d are oriented such that their anchor axes all lie on plane p1. For such implementations, proximal-distal axis ax4 can lie on plane p1 or can be parallel with plane p1. In some implementations in which anchor axes ax1 collectively define common anchor plane p1, tether 112 can extend along extracorporeal unit 350d (e.g., body 352 thereof), parallel with plane p1.

In the example shown, all of the anchors have the same orientation, e.g., they are superimposable onto each other by rotation-free translation. Such orientation of the anchors can be utilized irrespective of whether the anchors are housed by cartridges/anchor holders.

In some implementations, and as shown, each anchor 120d is mounted in an orientation in which its head 122d is proximal from its tissue-engaging element 130. For the sake of clarity, although the anchor itself is already described as having its tissue-engaging element extend distally away from its head, in the context of the mounting orientation of the anchor the terms proximal and distal are with reference to catheter device 300d (e.g., extracorporeal unit 350d thereof). Thus, each anchor's head 122d (e.g., interface 124 thereof) faces proximally (albeit obliquely) and each anchor's tissue-engaging element 130 faces distally (albeit obliquely). For example, for each anchor, the tissue-engaging element can be closer than the head to opening 320d.

In implementations in which anchors 120d are housed in cartridges/anchor holders 360d, proximal-distal axis ax4 can be (or can be considered to be) defined by the arrangement in which the series of cartridges is distributed along extracorporeal unit 350d (e.g., along body 352). For example, axis ax4 can connect the cartridges of the series by passing through the same point on each cartridge.

In some implementations, and as shown, system 100d is configured such that tether 112 extends along extracorporeal unit 350d (e.g., along body 352), parallel with axis ax4 (e.g., see FIG. 34B). At least in this context, the term “parallel” is intended to be distinct from coaxial, i.e., tether 112 lies alongside axis ax4. For example, whereas in system 100 tether 112 extends through cartridges 360, in system 100d tether 112 is disposed alongside cartridges 360d.

Similarly to as described hereinabove for implant 110, implant 110d can comprise one or more spacers (or dividers) 150d between anchors 120d. Spacers 150d can be structurally and/or functionally similar to as described hereinabove for spacer 150. However, in some implementations, the arrangement of spacers 150d within the implant may differ. For example, and as shown, in implant 110d, each spacer 150d can be connected to a respective anchor 120d (i.e., in addition to their simple coupling by virtue of both being threaded onto tether 112). This connection can, for example, be provided by a connector 152 (e.g., a cord) that extends between the anchor (e.g., the head of the anchor) and the spacer. In some implementations, connector 152 is also defined by textile 140. For example, a yarn can be arranged (e.g., tied) to define eyelet 126d and connector 152 (and, optionally, collar 128d). Thus, anchor 120d and spacer 150d can be considered to collectively define an anchor-spacer assembly 108d.

The spacers can be configured in a variety of ways. In some implementations, each spacer 150d comprises and/or is defined by a helical coil. For any such helical-coil-based spacers described herein, the coil may be formed by bending a wire into the coil, by cutting the coil from a tube (e.g., such that the coil is, in effect, a laser-cut hypotube). The material from which the coil is formed can be a metal (e.g., stainless steel, nitinol, or cobalt chrome), or a polymer (e.g., ePTFE, fluorinated ethylene propylene, or a polyolefin). Polymer-based coils may advantageously be formed by injection-molding. In order to facilitate fluoroscopic guidance of the procedure, the spacer can be provided with radiopacity by mixing radiopaque (e.g., metallic) particles into the polymer, and/or by adding radiopaque components (e.g., rings) to the ends of the spacer.

In some implementations, one or more (e.g., one, some, or all) of the cartridges/anchor holders 360 can comprise one component that is movable with respect to another component of the cartridge, in order to transition the cartridge from a closed state in which the cartridge houses the anchor (e.g., securely) into an open state in which the anchor is removable from the cartridge. In the example shown, cartridge 360 comprises a chassis 364, and the movable component of the cartridge is a tray 362, which is movable with respect to the chassis.

In some implementations, system 100d is provided with anchors 120d housed within cartridges 360 in their closed state, in which tray 362 is positioned with respect to chassis 364 such that the tray and the chassis cooperatively encage the anchor. As described in more detail with reference to FIGS. 37A-F, the cartridge is transitioned into its open state by application of a pulling force that moves (e.g., slides) tray 362 out of chassis 364, exposing the anchor such that it can be lifted out of the tray and advanced into the subject. In some implementations, cartridge 360 can be configured to allow this transition only if the pulling force exceeds a certain threshold magnitude, in which case tray 362 can therefore be considered to be a retaining member of the cartridge.

In some implementations, and as shown in e.g., FIG. 37B-C, chassis 364 can be integral with body 352 while tray 362 can be a discrete component that is coupled to the body during assembly of extracorporeal unit 350.

FIGS. 35A-C are various views of tray 362 with its corresponding anchor 120d loaded therein, and FIGS. 36A-C are the same views but without the anchor.

At least part of cartridge/anchor holder 360 can be shaped to facilitate the above positioning of tether 112. For example, cartridge 360 can be shaped to define a lateral groove 366 in its surface to accommodate tether 112. This can also facilitate smooth sliding of tether 112 past the cartridges during implantation of implant 110d. The lateral groove of cartridge 360 can be colinear with those of the other cartridges, thereby defining a common groove along which tether 112 can be disposed.

For implementations in which implant 110d comprises spacers 150d, the groove 366 of cartridge 360 can be dimensioned to accommodate the spacer that is associated with (e.g., connected to) the anchor 120d that is housed by the cartridge. For implementations in which cartridge 360 comprises tray 362 and chassis 364, lateral groove 366 can be defined by the tray and/or by the chassis. In the example shown, groove 366 is defined in part by tray 362 and in part by chassis 364, e.g., see FIG. 34B.

Each cartridge of the series of cartridges can be the same as or similar to each other (e.g., with the same features), or each cartridge can be different from each other cartridge (e.g., having one or more features that are different in shape or function), or multiple cartridges can be the same while others are different.

The arrangement and orientation of the anchors in system 100 may advantageously provide simplified and reliable engagement, release, and advancement of the anchors using driver 210, e.g., as described hereinbelow.

FIGS. 37A-F show system 100d being used to implant 110d, in accordance with some implementations. FIGS. 37A-E show at least some steps in which driver 210 is used to obtain leading anchor 120d′ from its cartridge 360d′, and to deliver the anchor via tube 310d to the tissue to which the driver anchors the anchor. Driver 210 (e.g., a drive head thereof) is moved into engagement with leading anchor 120d′ (FIGS. 37A-B). For example, and as shown, drive head 214 can be inserted into the cartridge via a window 370 defined by the cartridge, where it engages the head of the anchor (e.g., interface 124 thereof).

As shown (e.g., in FIG. 34B), window 370 (e.g., its rim) can be defined partly by tray 362 and partly by chassis 364. In some implementations, the rim of window 370 can be beveled, e.g., as shown—in order to facilitate alignment smooth entry of drive head 214 therethrough and/or to translationally align the drive head with the anchor.

In some implementations, window 370 can be shaped to allow drive head 214 to enter in a limited number of rotational orientations, e.g., in order that the drive head is appropriately oriented (e.g., rotationally aligned with the anchor) when it reaches the anchor. In the example shown, window 370 is oblong, in order to allow drive head 214 to enter and reach the anchor in only two rotational orientations, 180 degrees from each other.

In some implementations, window 370 and drive head 214 are complimentarily shaped in a manner that urges the drive head into such an allowed rotational orientation as it is advanced through the window. FIG. 37A shows driver 210 being advanced toward cartridge 360d′, and FIG. 37B shows drive head 214 of the driver having passed through window 370 and become engaged with anchor 120d′ (e.g., the interface of the head of the anchor).

In some implementations, tray 362 can be shaped to define a seat (e.g., a recessed seat) 361 shaped complementarily to anchor 120d, e.g., such that the anchor is seated snugly therewithin. FIGS. 35A-C show anchor 120d seated in seat 361, and FIGS. 36A-C show the seat empty. Window 370 therefore opens to seat 361.

In some implementations, driver 210 is configured such that drive head 214 engages (e.g., locks to) interface 124 of the anchor automatically, e.g., upon being pressed onto the interface. In some implementations, driver 210 can be configured to be locked to the interface in a discrete (e.g., manual) step after having been placed into contact with the anchor.

In some implementations, once engaged with anchor 120d′, driver 210 is pulled proximally in order to pull the anchor proximally (FIG. 37C). As anchor 120d′ moves proximally, it pulls tray 362 with it, in a manner that exposes the anchor for removal from cartridge 360d′. That is, pulling anchor 120d′ proximally transitions the cartridge from its closed state into its open state.

In some implementations, cartridge 360d′, like the other cartridges 360d, can be configured to allow this transition only if the pulling force exceeds a certain threshold magnitude (which can be the same for each cartridge or the magnitude can vary between different cartridges), thereby ensuring that drive head 214 is securely engaged with the anchor. (That is, in the absence of secure engagement, the drive head will, when the driver is pulled proximally, become disengaged from the anchor and exit the cartridge.) This behavior can be provided by at least one spring-loaded detent 372 disposed in at least one corresponding notch 374, the spring-loading maintaining the detent in the notch unless the threshold magnitude of pulling force is provided.

In the example shown, detent 372 is defined by (or coupled to) tray 362, and notch 374 is defined by chassis 364. However, it is to be understood that other arrangements can be used to provide similar functionality. In the example shown, the spring-loading is provided by detent 372 being disposed on, or defined by, a cantilevered leg 376. However, other approaches to spring-loading of a detent can be used. FIG. 37C shows detent 372 having exited notch 374 (e.g., facilitated by deflection of leg 376), allowing tray 362 to be pulled proximally away from (e.g., out of) chassis 364.

In some implementations, cartridge 360d is configured to, upon reaching its open state, resist returning toward its closed state. In the example shown, this is achieved by a second notch 374′, into which the spring-loading presses detent 372 upon the detent reaching the second notch, which coincides with the cartridge reaching its open state. This may advantageously provide an indication that the cartridge is empty, e.g., in order to prevent the operator from attempting to obtain an anchor from an empty cartridge.

In some implementations, the axis along which tray 362 slides defines a cartridge vector v1 of cartridge 360. Vector v1 can be oblique with respect to proximal-distal axis ax4. In some implementations, cartridges 360 are arranged along body 352 in an imbricated manner (e.g., such that the chassis are imbricated and/or such that the trays are imbricated). This can advantageously facilitate efficient storage of multiple anchors while also providing good access to each of the anchors. In the example shown, cartridge vector v1 is colinear or parallel with anchor axis ax1.

In some implementations, cartridges 360 are aligned such that their cartridge vectors v1 collectively define a common cartridge plane p2 on which the cartridge vectors lie. In some implementations, and as shown, common cartridge plane p2 is the same as plane p1. In some implementations, common cartridge plane p2 is parallel with plane p1. In some implementations, proximal-distal axis ax4 is parallel with common cartridge plane p2. In some implementations, and as shown, proximal-distal axis ax4 lies on common cartridge plane p2. In some implementations, and as shown, tether 112 extends along body 352, parallel with common cartridge plane p2.

FIGS. 35A-36C show that, in some implementations, tray 362 defines a lateral access 368 into seat 361. For example, access 368 can be a cutaway part of a sidewall of seat 361. Access 368 is positioned and dimensioned such that, when anchor 120d is seated in seat 361, eyelet 126d can extend laterally out of the seat (and optionally out of the cartridge) to reach tether 112, which extends alongside cartridges 360 (e.g., see FIG. 34B). Similarly, for implementations in which each anchor is connected to a respective spacer 150 via a connector 152, the connector can extend laterally through access 368 to reach the spacer.

Access 368 can be aligned with groove 366. For example, for implementations in which groove 366 is defined partly by tray 362 and partly by chassis 364, for each cartridge 360, access 368 can be disposed between the part of the groove defined by the tray and the part of the groove defined by the chassis (e.g., can separate these two parts of the groove), e.g., as shown.

As shown in FIG. 37C, as anchor 120d′ and its corresponding tray 362 move proximally, tether 112 is also pulled due to its connection to eyelet 126d of the anchor. Because tether 112 is also threaded through the eyelet of the successive anchor (whose cartridge 360 remains closed), the region of the tether disposed between the eyelet of anchor 120d′ and that of the successive anchor becomes oblique to axis ax4, while the eyelet of the successive anchor (and those of the further successive anchors) maintain more proximal regions of the tether parallel with axis ax4. Spacer 150d′, which is threaded on this region of tether 112, accordingly deflects to also become oblique to axis ax4, while the successive spacers remain parallel with axis ax4.

Using driver 210, anchor 120d′ is then removed from cartridge 360d′ (e.g., is lifted out of tray 362) (FIG. 37D), and inserted through proximal opening 320d into tube 310d (FIG. 37E). The anchor can then be advanced through tube 310d to the tissue to which the anchor will be anchored, e.g., similar to as shown in FIG. 2A, mutatis mutandis.

In some implementations in which each anchor 120d is connected to a respective spacer 150d (e.g., via connector 152), the spacer can be arranged (e.g., threaded onto tether 112) such that the spacer is pulled along distally by the anchor as the anchor is advanced, the spacer trailing behind eyelet 126d, e.g., as shown. Thus, if a spacer is desired between the leading anchor and the immediately subsequent anchor, whereas for implementations in which the spacer leads the eyelet (e.g., is pushed along by the eyelet) the leading anchor can be advanced without an associated spacer, for implementations in which the spacer trails behind the eyelet, the leading anchor is advanced with an associated eyelet.

In some implementations in which each anchor 120d is connected to a respective spacer 150d, the spacer can be considered to be a component of the anchor.

Because leading anchor 120d′ is secured to tether 112 (e.g., by stopper 114d), as the anchor is advanced through tube 310d, it draws the tether along with it. In contrast, successive anchors 120d can be slid along tether 112 toward the anchor(s) that has/have already been anchored to the tissue.

Leading anchor 120d′ is shown as being identical to the other anchors of implant 110d′, and as being prevented from sliding distally off of tether 112 by stopper 114d. However, in some implementations the leading anchor can be different to the other anchors in this manner. For example, the leading anchor can be specialized and/or more directly fixed to tether 112, such as via a ball-and socket (e.g., as described for anchor 120′), or by tether 112 being looped through eyelet 126d and back on itself, with the loop being closed by knotting, crimping, or another suitable means.

In some implementations, and as shown, extracorporeal unit 350 (e.g., body 352 thereof) defines a rest 353 in which shaft 212 of driver 210 can be rested during the advancement and/or anchor of the anchor. In the example shown, rest 353 is positioned proximally from cartridges 360, i.e., such that the cartridges are disposed between the opening and the rest. Rest 353 can be positioned such that, while drive head 214 is disposed distally from opening 320d and shaft 212 rests in rest 353, at least a portion the shaft that extends along extracorporeal unit 350 does so alongside tether 112 and/or axis ax4, and/or parallel to plane p1 and/or plane p2.

Once anchor 120d′ has been anchored, driver 210 is disengaged from the anchor and withdrawn through tube 310d in readiness for performing the same process for successive anchors. FIG. 37F shows the second anchor of the series having been removed from its cartridge in readiness for being advanced into opening 320d and through tube 310d. This corresponds to the step shown in FIG. 37D but, due to the distal end of tether 112 having been secured to the tissue, the arrangement of the tether proximal from opening 320d is a little different to that shown in FIG. 37D: The tether extends in a first line away from axis ax4 to the anchor that has just been removed from its cartridge, and then in a second line away from that anchor into opening 320d. Successive anchors can be anchored in this manner.

In some implementations, for each of the anchors, removal, by the anchor driver, of the anchor from the corresponding cartridge can move the anchor away from axis ax4. Similarly, part of the tether can be pulled away from and/or out of alignment with axis ax4. As shown, in some implementations, prior to use of system 1000 tether 112 can extend straight (i.e., in a straight line) from proximal opening 320d to aperture 382, e.g., in a manner that defines a tether axis. Thus, removal of the anchor from the corresponding cartridge can move the anchor away from the tether axis and/or can deflect or reshape part of the tether (the part closest to the anchor) away from being straight. For each of the anchors other than the leading anchor (i.e., for each of the other anchors), the part of the tether can be formed into a V-shape, e.g., as shown in FIG. 37F.

In some implementations, catheter device 300d (e.g., extracorporeal unit 350d) can comprise a de-slacker 354d, which can comprise a spring-loaded winch that comprises a spool 380 on which a proximal region of tether 112 is spooled. De-slacker 354d is illustrated minimalistically and in phantom in FIG. 34A, and is shown in more detail in FIGS. 34C-D, which are cutaways from opposite sides of extracorporeal unit 350d (e.g., with respective sides of body 352 removed).

The spring-loading of de-slacker 354d is calibrated to take up (e.g., eliminate) slack on tether 112 throughout the implantation of implant 110d without hindering the implantation process. For example, the spring-loading can be sufficiently weak to let out tether 112 in response to pulling of the tether (e.g., as anchor 120d′ is advanced, as each successive anchor is moved out of its cartridge and away from axis ax4, and/or as each anchor is positioned at the tissue), while being sufficiently strong to re-spool the tether upon reduction in such pulling.

De-slacker 354d can comprise a spring 384, such as a constant-force spring (e.g., a spiral torsion spring) that provides the spring-loading. For some implementations, and as shown, spring 384 applies torque to spool 380 via intermeshed gears in order to provide an optimal transmission ratio. For example, and as shown, spring 384 can drive a first gear 386 that, in turn, drives a second gear 388 that, in turn, drives spool 380. In the example shown, gear 388 is fixed to spool 380, e.g., as a unitary structure.

In some implementations, extracorporeal unit 350d (e.g., body 352 thereof) can define an aperture 382 out of which tether 112 passes as it extends from spool 380 to anchors 120d, and through which the tether slides as it is pulled distally. Aperture 382 can be aligned with opening 320d, and can face along the series of cartridges to the opening. In the example shown, aperture 382 is positioned proximally from cartridges 360, i.e., such that the cartridges are disposed between the opening and the aperture. As shown, aperture 382 can be aligned with grooves 366. Thus, tether 112 can lie in, and slide along, a substantially straight line from aperture 382, through eyelets 126d and spacers 150, and into opening 320d.

Whereas de-slacker 354 of system 100 can be disposed toward (e.g., at) the distal end of its extracorporeal unit, de-slacker 354d can be disposed toward (e.g., at) the proximal end of extracorporeal unit 350d. For implementations in which de-slacker 354d comprises a spring-loaded winch, the axis of rotation ax5 of the winch (e.g., of its spool 380) can be transverse to the path of tether 112 along extracorporeal unit 350d and/or to proximal-distal axis ax4.

De-slacker 354d can comprise a deactivation switch 390 that is user-operable to deactivate the de-slacker in a manner that renders it unable to spool-up tether 112 (i.e., to pull the tether proximally) while allowing the tether to be unspooled (e.g., by the tether being pulled distally). For example, de-slacker 354d can have an active state in which switch 390 is in a first position (up, in the illustrated example), and an inactive state in which the switch is in a second position (down, in the illustrated example). In some implementations, switch 390 can function by engaging/disengaging a pawl 392 with/from a rack (e.g., a circular rack) 389. For example, in the active state of de-slacker 354d pawl 392 can be disengaged from rack 389, and engaging the pawl with the rack can deactivate the de-slacker. As shown, rack 389 can be fixed to spool 380, e.g., as a unitary structure. In the example shown, pawl 392 is hingedly mounted as a lever.

Switch 390 can be used as desired by the operator, but can be particularly useful to facilitate decoupling and removal of tether 112 from extracorporeal unit 350d between implantation and tensioning of implant 110d, e.g., as described hereinbelow with reference to FIGS. 38A-B. This removal can also be facilitated by the proximal end 113 of tether 112 being releasably secured to spool 380.

In the example shown, rather than tether 112 being permanently (or substantially permanently) attached to spool 380, the tether is woven through holes 381 (e.g., lateral holes) in the spool. This weaving, as well as the number, positioning, and geometry of holes 381, can be configured to (i) secure tether 112 to spool 380 sufficiently for de-slacker 354d to serve its function while the tether remains spooled on the spool (e.g., while one or more complete turns of the tether are wrapped onto the spool), but also (ii) release the tether upon the tether becoming completely unspooled (e.g., by allowing end 113 to slide out and unweave from the holes). This releasing and its advantages are discussed in further detail with reference to FIGS. 38A-B and 41A. As shown, the weaving of tether 112 through holes 381 can be such that end 113 is disposed on the inside of spool 380, e.g., reducing a chance of the end catching on another component of the extracorporeal unit as the spool rotates.

As noted hereinabove, in some implementations it can be determined to be beneficial that some regions of implant 110 (e.g., a variant thereof) contract less than others. For example, and as also noted hereinabove, it can be determined to be beneficial that an end region of the implant (and the corresponding region of tissue), such as the region between the leading anchor and the successive anchor, contract less than more proximal regions of the implant (and the corresponding regions of tissue). Such a configuration can be achieved via differential configurations of the implant's spacers. For example, the spacer threaded on tether 112 between the leading anchor and the successive anchor can be less axially compressible than one or more other spacers (e.g., can be substantially axially non-compressible).

Implant 110d is shown with an example of such a configuration in which each spacer comprises a helical coil (e.g., a helically-coiled wire), the pitch of the coil determining the compressibility of the spacer. The coil of the spacer 150d that is connected to leading anchor 120d′ has a smaller pitch than that of the spacer that is connected to the subsequent anchor (compare the inset of FIG. 36D to that of FIG. 36F). For example, the coil of the spacer 150d that is connected to leading anchor 120d′ can be a closed coil with no space between the turns of the helix (FIG. 36D), whereas the coil of the spacer that is connected to the subsequent anchor can have spaces between its turns (FIG. 36F).

FIG. 37G shows several anchors 120d having been anchored sequentially, such that tether 112 (and the implant in general) extends around valve 12. In the example shown, ten anchors have been anchored along tissue 10—in this case the annulus of valve 12. That is, in the example shown, implant 110d comprises 10 anchors. Also in the example shown, valve 12 is a mitral valve and implant 110d is implanted primarily along the posterior annulus. However, it is to be understood that implant 110d can be implanted (e.g., using system 1000) with other numbers of anchors, in other implantation arrangements, and/or at other valves (e.g., the tricuspid valves), according to the condition of the subject and/or the determination of the physician. It is to be noted that, at this phase of the procedure, although de-slacker 354d continues to eliminate slack from tether 112, the tether is not under substantial tension and the tissue at which implant 110d is not materially affected (e.g., contracted) by the implant.

Once it has been determined that no more anchors 120d are required (i.e., that implant 110d has the desirable number of anchors) tether 112, and thereby implant 110d as a whole, is decoupled and removed from catheter device 300d. As shown in FIGS. 38A-B, such decoupling can be achieved by pulling tether 112 (e.g., distally) until it becomes released from de-slacker 354d and proximal end 113 exits extracorporeal unit 350d (e.g., via aperture 382). For example, catheter device 300d (e.g., de-slacker 354d thereof) can be configured such that tether 112 automatically becomes released from spool 380 upon becoming completely unspooled from the spool, i.e., by unweaving from holes 381, as discussed with reference to FIGS. 34C-D.

In implementations in which catheter device 300d (e.g., de-slacker 354d thereof) comprises deactivation switch 390, the operator can switch the de-slacker into its inactive state by switching the deactivation switch in order to facilitate the unspooling of tether 112. Hence, in FIGS. 38A-B, switch 390 is shown in its down position. During this decoupling and removal of tether 112 from extracorporeal unit 350d, the inactive state advantageously allows the operator to pull successive lengths of tether 112 out of aperture 382 without de-slacker 354d pulling the tether back in (and without a need for restraining the already-pulled lengths of the tether). Thus, after one or more such pulls, the entirety of tether 112 becomes unspooled from spool 380.

It is to be noted that, for instances in which fewer than all of anchors 120d are implanted, those anchors that are not implanted remain within their respective cartridges 360d and tether 112 is unthreaded (in a distal direction) out of the eyelets of those anchors (and out of their corresponding spacers 150d), leaving them coupled to extracorporeal unit 350d (e.g., as shown in FIG. 38B).

Catheter device 300d can then be removed, e.g., by unlocking its foot 1016 (such as by actuating cam 1018) and then sliding (i) extracorporeal unit 350d proximally along and out of track 1012, and (ii) tube 310d proximally over and off of tether 112 and/or out of catheter system 1002. This leaves the proximal part of tether 112 extending out of at least part of catheter system 1002, with proximal end 113 exposed (FIG. 38C).

For some implementations, and as shown, catheter 1040 is similarly proximally withdrawn and removed, e.g., tube 1044 is slid proximally out of catheter 1020. For such implementations, catheter device 300d and catheter 1040 can be proximally withdrawn and removed together. Tether 112 can be manufactured such that proximal end 113 is smooth and/or rounded, e.g., the proximal end can have undergone a smoothing, sealing, and/or coating process. This, as well as tether 112 having been removed from catheter device 300d without cutting, advantageously facilitates the insertion of (intact) proximal end 113 through a lock 160d and into an adjustment tool 400d within which the lock is loaded.

At this point, an adjustment tool can be used to adjust (e.g., contract) implant 110d by applying tension to tether 112, locking the tension into the tether, and trimming excess tether. Thus, system 1000 can comprise such an adjustment tool. In the example shown, the adjustment tool of system 1000 is an adjustment tool 400d. Adjustment tool 400d can be considered to be a variant of adjustment tool 400 hereinabove. For some implementations adjustment tool 400, or another variant thereof, can be used in place of adjustment tool 400d.

FIGS. 39A-I schematically show various views of adjustment tool 400d and components thereof. FIGS. 40A-C schematically show various views of lock 160d, and components thereof, that is held and/or used by tool 400d. FIGS. 41A-L show at least some steps of a technique in which tool 400d is used to adjust implant 110d by applying tension to tether 112, locking the tension into the tether, and trimming excess tether, in accordance with some implementations.

Tool 400d comprises a tool head 420 at a distal portion of the tool, and an extracorporeal unit (i.e., an adjustment-tool extracorporeal unit) 450 at a proximal portion of the tool. A flexible shaft 402d extends between extracorporeal unit 450 and head 420, and through the shaft the extracorporeal unit is operatively coupled to the distal portion of the tool. Head 420 holds (e.g., houses) lock 160d.

As noted briefly hereinabove, proximal end 113 of tether 112 is inserted (e.g., threaded) through lock 160d and into adjustment tool 400d (FIG. 41A). For example, lock 160d can have a distal-facing aperture 164 through which proximal end 113 of tether 112 can be inserted and advanced proximally through the lock and into the adjustment tool. At this stage, lock 160d can be constrained in an open state by the presence of an obstructor 440 (e.g., an unlocker) such as an obstructor tube. For example, and as shown, lock 160d can comprise a clamp (or another locking element) 180 that is obstructed by obstructor 440, which extends into the lock.

In some implementations, clamp 180 can be spring-loaded. For implementations in which obstructor 440 is an obstructor tube, tether 112 can be inserted proximal-end-first into through obstructor 440 such that the tether passes through lock 160d (e.g., past clamp 180) within the obstructor and is thereby shielded from the clamp by the obstructor.

Lock 160d can define a path 170 extending proximally from aperture 164 past clamp 180 (e.g., between a clamp face 184 of the clamp and an opposing surface 166 that can be an interior surface of a case 162 of the lock) and obstructor 440 can extend along this path such that, in the unlocked state of the lock, tether 112 can be advanced proximally along this path while shielded from the clamp (see also to FIGS. 40A-C for reference).

Tool 400d can include an uptake assembly 600, which can include a gripper 602 and a sleeve 608, e.g., at a working end of the uptake assembly. Gripper 602 comprises a flexible rod 604 and a bulbous or otherwise wide distal portion 606 attached to the rod. Rod 604 extends proximally to extracorporeal unit 450, via which the axial position of gripper 602 with respect to sleeve 608 is controllable. For example, extracorporeal unit 450 can comprise an actuatable knob 610 that is fastened to a proximal end of rod 604 (e.g., by a fastener 611 such as a set screw).

During insertion of tether 112 into tool 400d, the uptake assembly is in a receiving state in which distal portion 606 is distal to sleeve 608. Tool 400d is configured (e.g., shaped and/or sized relative to tether 112) such that, in this state, as the tether is advanced proximally through lock 160d and into the tool, its proximal end 113 is passively guided past portion 606 and into sleeve 608 alongside rod 604, e.g., as shown in FIG. 41A. FIG. 41B shows additional views of the same state; view X showing cross-sections of the distal portion of tool 400d, and view Y showing a cross-section of a proximal part of extracorporeal unit 450.

As noted with reference to FIGS. 34C-D, tether 112 can be secured to spool 380 of de-slacker 354 by being woven through multiple holes 381 in the spool such that the tether becomes released from the spool one sufficient pulling has completely unspooled the tether from the spool. This allows tether 112 to be decoupled from extracorporeal unit 350d without cutting the tether, and results in proximal end 113 of the tether remaining intact. As also noted hereinabove, tether 112 can be manufactured such that proximal end 113 is smooth and/or rounded, i.e., the proximal end can have undergone a smoothing, sealing, and/or coating process. Thus, intact proximal end 113 can, upon simple advancement of tether 112 through distal-facing aperture 164, advantageously result in the proximal end threading its way through lock 160d and obstructor 440, past distal portion 606 and into sleeve 608 alongside rod 604.

Once tether 112 has been advanced sufficiently into sleeve 608 (which may be determined by tactile feedback), uptake assembly 600 is actuated such that it grasps the tether (FIG. 41C, which includes the same views X and Y as FIG. 41B). In the example shown, this is achieved by rotating knob 610, which is threadedly coupled to another extracorporeal component of the uptake assembly, such as a core 612. As shown, this rotation can also be relative to a body 452 of extracorporeal unit 450. Due to the threaded coupling between knob 610 and core 612, rotation of the knob causes the knob to move proximally, thereby pulling gripper 602 proximally.

A proximal end of sleeve 608 is fastened to core 612, such that the proximalward movement of gripper 602 is proximalward relative to sleeve 608 and draws distal portion 606 of the gripper into the sleeve, thereby transitioning uptake assembly 600 into its grip state. Distal portion 606 is dimensioned such that, in its grip state, tether 112 becomes gripped between an outer surface of the distal portion and an inner surface of sleeve 608—illustrated in the transverse cross-sectional inset of FIG. 41C as the tether being squashed between these surfaces. Distal portion 606 can draw tether 112 slightly further into sleeve 608 during transitioning into the grip state, e.g., as shown by the transition from FIG. 41B to FIG. 41C.

Uptake assembly 600 is configured such that the grip state is reached upon knob 610 having been rotated a predefined number of rotations. In some implementations, once the predetermined number of rotations has been reached, core 612 is released (i.e., becomes detachable) from body 452 of extracorporeal unit 450. Thus, upon the predefined number of rotations being reached, the operator can, by pulling on knob 610, proximally pull the entire of uptake assembly 600 along with tether 112 that is gripped by the uptake assembly, thereby withdrawing gripper 602 and sleeve 608 from tool 400d and drawing the tether proximally through lock 160d and the tool. FIG. 41D shows uptake assembly 600 having been completely removed, leaving tether 112 extending proximally through tool 400d and out of extracorporeal unit 450.

It is to be noted that other uptake assemblies can be used in place of uptake assembly 600, mutatis mutandis. For example, other tether- or suture-grasping devices, such as snares, can serve as the uptake assembly of tool 400d.

For implementations in which obstructor 440 is an obstructor tube, the drawing of tether 112 through tool 400d draws the tether through the obstructor tube. Obstructor 440 is operatively coupled to extracorporeal unit 450 such that the operator can trigger lock 160d to lock by operating the extracorporeal unit, e.g., as described hereinbelow. This operative coupling can be provided by obstructor 440 extending proximally from lock 160d at head 420 to the extracorporeal unit.

As shown, obstructor 440 can be tubular all the way to extracorporeal unit 450. In the example shown, obstructor 440 has a narrower distal segment (labeled 440), and a wider proximal segment (labeled 440′) that extends proximally from the distal segment to extracorporeal unit 450. The narrower and wider segments are shown connected at 421. The narrower distal segment extends into lock 160d (e.g., has a sufficiently small outer diameter to serve as an obstructor of the lock), and wider segment 440′ accommodates sleeve 608 and gripper 602, i.e., has a sufficiently large inner diameter to accommodate these components of uptake assembly 600.

It is to be noted that, in some implementations, obstructor 440 can alternatively have a substantially constant width along its length and that, in such implementations, uptake assembly 600 can be dimensioned accordingly.

For some implementations, segment 440′ can additionally and/or alternatively be considered to be a shaft that operatively couples obstructor 440 to extracorporeal unit 450. This shaft can be tubular (e.g., with uptake assembly 600 extending therethrough) or non-tubular (e.g., with the uptake assembly extending alongside).

While the system remains in the state represented by FIG. 41D, with tether 112 extending proximally out of extracorporeal unit 450, tool head 420 of tool 400d is advanced distally into and through catheter 1020 (e.g., tube 1024 thereof), sliding over and along the tether toward the final anchor 120d to have been delivered and anchored to tissue 10. Extracorporeal unit 450 can be slidably mounted to track 1012, e.g., using an integrated foot 1016, as described for extracorporeal unit 350d, mutatis mutandis. FIG. 41E shows system 1000 after this advancement of head 420 and mounting of extracorporeal unit 450d, with the head having reached the final anchor 120d to have been delivered and anchored to the tissue.

In some implementations, the mounting of extracorporeal unit 450 to track 1012 is performed after tether 112 has been drawn proximally through tool 400d and tool head 420 has been advanced at least halfway through tube 1024. In some implementations, the mounting is performed after tether 112 has been drawn proximally through tool 400d but before head 420 has reached halfway through tube 1024. In some implementations, the mounting is performed before head 420 has been inserted into catheter 1020, or even before tether 112 is inserted into the catheter. As shown, extracorporeal unit 450 can thereby assume the place on track 1012 that was previously occupied by extracorporeal unit 350 and/or the mount 1014 of catheter 1040.

The advancement of tool 400d can be accompanied and/or facilitated by pulling on tether 112 in order to reduce slack on the tether and facilitate advancement of the tool therealong. Once head 420 has reached the final anchor 120d to have been delivered and anchored to the tissue, and typically once slack in tether 112 has been eliminated, a tensioning subassembly of extracorporeal unit 450 is locked to the tether (FIG. 41F). In the example shown, this locking is achieved using a clamp 462 that can comprise a cam 464 and a press-plate 466. In addition to clamp 462, the tensioning subassembly can comprise a tensioning block 460 to which clamp 462 is attached, and/or a tensioning controller (e.g., knob) 468 that is operable (e.g., hand-operable) by the operator (e.g., physician).

To operate the clamp, cam 464 can be rotated (e.g., by pressing down on a lever) such that the cam presses press-plate 466 to clamp tether 112 between the press-plate and tensioning block 460. It is to be noted that, until the removal of uptake assembly 600, sleeve 608 (and rod 604 therewithin) can be disposed through clamp 462. Therefore, in some implementations, clamp 462 is configured both (i) to, while open, provide a sufficiently large gap (e.g., between press-plate 466 and tensioning block 460) to accommodate the presence and sliding of these components of uptake assembly 600, and (ii) to, close this gap sufficiently to clamp tether 112 after removal of these components.

In the example shown, tether 112 extends axially through tensioning block 460, which can have a long axis that is substantially coaxial or parallel with the segment of tether 112 disposed through the tensioning block (e.g., as shown).

In order to apply tension to tether 112, the operator operates tensioning controller 468, which is operatively coupled to tensioning block 460 such that operation of the tensioning controller drives proximally the tensioning block, which is clamped to the tether and therefore pulls the tether proximally. FIG. 41G shows the tensioning subassembly of extracorporeal unit 450 prior to tensioning of tether 112 (e.g., different views of the same state shown in FIG. 41F), and FIG. 41H shows the tensioning subassembly after operation of tensioning controller 468 has been operated to apply tension to tether 112. In each of these figures, view X is a perspective view and view Y is a cross-sectional view of the tensioning subassembly of extracorporeal unit 450.

In the example shown, tensioning controller 468 is a tensioning knob that is operated by rotation, which drives tensioning block 460 proximally via threading 456 that provides a linear actuator functionality. That is, the tensioning subassembly of extracorporeal unit 450 can comprise a linear actuator. However, it is to be understood that other controller and/or actuator types can be used.

Extracorporeal unit 450 (e.g., the tensioning subassembly thereof) can include a distance indicator 463 that indicates the distance by which tether 112 has been pulled by the tensioning subassembly. Distance indicator 463 can include markings (e.g., a scale) on tensioning block 460 whose position relative to body 452 are indicative of this pulled distance. For example, and as shown, these markings can become progressively exposed (e.g., from body 452) as operation of tensioning controller 468 moves the block (and therefore tether 112) progressive proximally.

Thus, the adjustment-tool extracorporeal unit includes a distance indicator by which a position of the adjustment block with respect to the body of the adjustment-tool extracorporeal unit indicates a distance by which operation of the tensioning controller has drawn the tensioning block proximally.

Extracorporeal unit 450 (e.g., the tensioning subassembly thereof) can include a tension indicator 461 that indicates the magnitude of the tension that has been applied to tether 112 by the tensioning subassembly. This indication of tension can be facilitated by the tensioning subassembly comprising a spring 459 via which force is transferred from tensioning controller 468 to tensioning block 460. For example, and as shown, the tensioning subassembly can comprise a stock 458 that is driven by tensioning controller 468 (e.g., via threaded mating that serves as a linear actuator) and that pushes spring 459 to push tensioning block 460.

Tension indicator 461 can include markings (e.g., symbols or a scale) on tensioning block 460 whose position relative to stock 458 is indicative of strain (e.g., compression) of spring 459 and therefore of tension on tether 112. For example, the markings of tension indicator 461 can become progressively concealed and/or exposed from stock 458 as operation of tensioning controller 468 increases tension on tether 112.

Thus, the adjustment-tool extracorporeal unit includes a tension indicator by which a position of the adjustment block with respect to the stock indicates a magnitude of the tension that operation of the tensioning controller has applied to the tether.

As noted elsewhere herein, application of tension to tether 112 contracts the implant and therefore the tissue (e.g., the annulus) along which it is anchored. The application of tension can be monitored using imaging techniques (e.g., ultrasound and fluoroscopy) as well as indicators 463 and 461 in order to determine how much tension to apply. For example, the operator may aim to reduce regurgitation through the heart valve (e.g., monitored via Doppler echocardiography) as much as possible by pulling tether 112 an expected distance (e.g., monitored via indicator 463) without exceeding a predetermined amount of tension on tether 112 (e.g., monitored via indicator 461), e.g., in order to avoid damage to the implant or the tissue.

Once the desired amount of tension on tether 112 has been achieved, the operator uses tool 400d to lock in the tension and trim away excess tether. This process is schematically illustrated in FIGS. 41I-K, each of which includes a view X that is a cross-sectional view of a lock-and-cut subassembly of extracorporeal unit 450, and a view Y that is a cross-sectional view of tool head 420, in accordance with some implementations. FIG. 41I shows the system prior to use of the lock-and-cut subassembly (e.g., different views of the same state shown in FIG. 41H), FIG. 41J shows the system after the tension has been locked in but before the excess tether has been trimmed, and FIG. 41K shows the system after the excess tether has been trimmed. Some of the components shown in these figures are shown in more detail in FIGS. 39D-I (which show components of tool head 420) and 40A-C (which show components of lock 160d).

While lock 160d, within tool head 420, remains disposed at the final anchor to have been delivered and anchored, the tension in tether 112 is locked in by locking lock 160d to the tether. Lock 160d is too large to fit through eyelet 126d of the final anchor, and therefore prevents the tether from unthreading from the eyelet. Lock 160d is locked by the operator operating a lock controller 448 which, as detailed hereinbelow, can be a lock-and-cut controller, i.e., a unitary controller whose operation first locks the lock and subsequently cuts the tether. Controller 448 is operatively coupled to a locking block 446 such that operation of the controller drives the locking block proximally. Locking block 446 is coupled (e.g., fixed) to obstructor 440 (e.g., to proximal segment 440′ thereof) such that operation of controller 448 withdraws the obstructor proximally over and along tether 112 and out of lock 160d, thereby allowing (e.g., triggering) clamp 180 to responsively clamp onto tether 112 (FIG. 41J).

In some implementations, and as shown, clamp 180 comprises multiple resilient beams 182 that are coupled to clamp face 184 and cooperate to push the clamp surface toward opposing surface 166. Beams 182 can be substantially parallel with each other, e.g., as shown (see also FIGS. 40A-C for reference). One or more of the beams 182 (e.g., each beam) can resemble a cantilever spring except that its otherwise free end is attached to clamp face 184. In some implementations, and as shown, when lock 160d locks, beams 182 can deflect with respect to case 162 (e.g., in unison and/or while remaining parallel with each other) such that clamp face 184 translates toward opposing surface 166. In FIG. 40C, even though obstructor 440 is absent lock 160d is shown in its unlocked state in order to illustrate path 170 between clamp 180 (e.g., clamp face 184) and opposing surface 166.

In the example shown, lock controller (e.g., lock-and-cut controller) 448 comprises a knob that is operated by rotation, which drives locking block 446 proximally via threading that provides a linear actuator functionality. That is, the lock-and-cut subassembly of extracorporeal unit 450 can comprise a linear actuator. However, it is to be understood that other controller and/or actuator types can be used.

Subsequently, extracorporeal unit 450 is operated in order to move a blade 480 within tool head 420 in order to cut excess tether 112, i.e., to trim it away (FIG. 41K). Within tool head 420 blade 480 can be disposed distally from a fixed component 490 with which the blade cooperates to shear tether 112, e.g., between a blade edge 485 of the blade and a fixed edge 494 of the fixed component. Thus, blade 480 and fixed component 490 can be considered to collectively define a guillotine. Tool 400d can be configured such that, in response to a proximally-directed force applied to blade 480, the blade moves (e.g., slides) obliquely (with respect to tether 112 and/or the overall axis of tool 400d). This can be achieved by blade 480 having an oblique surface 482 and/or fixed component 490 having a complimentary oblique surface 492, oriented such that the proximally-directed force forces the blade to slide obliquely along the fixed component. For example, oblique surface 482 can face oblique surface 492, both oblique surfaces describing similar oblique angles with respect to tether 112 and with respect to a vector of the proximally-directed force.

In the example shown, the proximally-directed force is applied to blade 480 via a cage 476 that extends around fixed component 490, the cage transferring the proximally-directed force from a cutter shaft 470 that operatively couples the cage to extracorporeal unit 450. For some implementations, cutter shaft is connected to cage 476 via a swivel connector 474, which allows the cage, as well as blade 480 and fixed component 490, to swivel with respect to cutter shaft 470 (see also FIGS. 39E-F for reference). For some implementations, the entirety of tool head 420 can swivel freely with respect to shaft 402d. Such swiveling can advantageously allow tool 400d to be used without requiring a particular rotational orientation, e.g., head 420 can responsively “find” its optimal orientation as it arrives at implant 110d.

In some implementations, cutter shaft 470 is operated (e.g., pulled) using a dedicated controller (e.g., knob) of extracorporeal unit 450. However, as noted hereinabove, in some implementations such as the example shown, controller 448 is a unitary lock-and-cut controller. For such implementations, continued operation (e.g., rotation) of controller 448 after obstructor 440 has been withdrawn to lock lock 160d pulls cutter shaft 470 to actuate blade 480. In the example shown, this is achieved by extracorporeal unit 450 comprising an adapter 472 that is connected to cutter shaft 470 and is slidably interlocked with locking block 446 such that upon a certain amount of operation of controller 448 (and thus a certain amount of proximal movement of the locking block), part of the locking block abuts part of the adapter (FIG. 41J; abutting indicated by reference numeral 473) such that any further operation of the controller (and thus any further proximal movement of the locking block) pulls the adapter, and therefore the cutter shaft, proximally, thereby applying the proximally-directed force to blade 480 (FIG. 41K).

Cage 476 can be shaped to control and/or stabilize blade 480 and its movement, e.g., by at least partly caging the blade within the cage. In the example shown, cage 476 defines a hollow chamber within which blade 480 is disposed, and blade 480 defines a flange 484 and a neck 486 that connects the flange to the rest of the blade. Cage 476 defines a transverse slit 478, transverse to the vector of the proximally-directed force. Slit 478 accommodates neck 486 extending axially through the slit, with flange 484 outside of the cage (e.g., distal to the cage) such that the slit, neck, and flange cooperate to control the above-described movement of the blade during its actuation, e.g., with the neck sliding along the transverse slit, transversely with respect to the vector of the proximally-directed force (e.g., compare FIGS. 41J and 41K; also see FIGS. 39D-F and 39I for reference).

Cage 476 can alternatively or additionally be shaped to control and/or stabilize fixed component 490, e.g., by at least partly caging the fixed component within the cage. Fixed component 490 defines one or more laterally-protruding tabs 496. Cage 476 can define one or more axial slits 479, parallel with the vector of the proximally-directed force. Each slit 479 accommodates a corresponding tab 496 extending laterally through the slit such that, upon application of the proximally-directed force via cutter shaft 470 (and optionally connector 474) cage 476 can responsively move proximally with respect to fixed component 490 (e.g., with the axial slits sliding axially along the tabs, parallel with the vector of the proximally-directed force), thereby pulling blade 480 proximally against the fixed component. Thus, during actuation of blade 480, the blade rides transversely along slit 478 while cage 476 rides axially along tabs 496.

Tool head 420 can comprise a hull 422 that houses the other components of the tool head. Fixed component 490 can be fixed in place with respect to hull 422 by tabs 496 extending laterally beyond axial slits 479 of cage 476 and protruding into corresponding recesses 426 in the hull.

As shown, blade 480 can define a channel 483 that runs axially along the blade, blade edge 485 being defined by a rim of the channel. As shown, fixed component 490 can define a channel 493 that extends axially along the fixed component, fixed edge 494 being defined by a rim of the channel. Either of these channels can be laterally open and/or groove-like (e.g., as shown for channel 483) or laterally enclosed and/or tunnel-like (e.g., as shown for channel 493). Until blade 480 is actuated, these channels remain substantially colinearly aligned such that tether 112 can extend axially through the channels and pass uninhibited through tool head 420. Actuation of blade 480 moves these channels out of alignment such that blade edge 485 (i.e., the rim of channel 483) passes fixed edge 494 (i.e., the rim of channel 493), thereby shearing tether 112.

In some implementations, extracorporeal unit 450 comprises a safety latch 498 that must be operated in order to proceed to the cutting of tether 112. For example, and as shown, safety latch 498 can be spring-loaded and can become available (e.g., may pop up) upon the certain amount of operation of controller 448 (and thus the certain amount of proximal movement of locking block 446 and obstructor 440) being reached (FIG. 41J). Further operation of controller 448 (in order to actuate blade 480) requires safety latch 498 first be depressed. In the example shown, safety latch 498 works by obstructing adapter 472 from being pulled proximally.

It is to be noted that tether 112 extends substantially linearly through lock 160d and the entirety of tool 400d. That is, lock 160d and blade 480 are configured to function without requiring that tether 112 take a tortuous or serpentine path through head 420.

Once the excess tether has been trimmed away, tool 400d is withdrawn, leaving lock 160d locked to tether 112, e.g., as a component of implant 110d (FIG. 41L). Lock 160d can comprise a latch 168 that retains the lock within tool head 420, e.g., prevents premature release of the lock. Latch 168 can be resilient. Latch 168 engages tool head 420 (e.g., hull 422 thereof), e.g., by protruding into a socket 424 of the tool head (e.g., defined by the hull).

While obstructor 440 is present within lock 160d, in addition to obstructing clamp 180, it also obstructs latch 168 from moving out of engagement with the tool head, i.e., from moving out of socket 424—see, for example, FIG. 41I. For example, obstructor 440 can maintain latch 168 protruding out of case 162, e.g., obstructing the latch from receding into the case. After obstructor 440 has been withdrawn in order to lock lock 160d, latch 168 is no longer restrained, and tool head 420 can be retracted, with the lock exiting a distal opening of the tool head.

In some implementations, latch 168 is biased to disengage from the tool head (e.g., to recede into case 162) automatically upon removal of the obstruction. In some implementations, latch 168 is biased to remain engaged with the tool head (e.g., to protrude into socket 424) but, in the absence of the obstruction, will transiently recede into the case as the tool head is pulled away from lock 160d.

In some implementations, even after obstructor 440 has been withdrawn, prior to cutting of tether 112 the tether itself, under tension, can obstruct latch 168 from disengaging from tool head 420.

In the example shown, latch 168 is cantilevered (e.g., comprises a cantilever spring), but it is to be understood that other types and configurations of latches (e.g., resilient and/or spring-loaded latches) can be used.

Reference is again made to FIG. 41J. As described hereinabove, in the state shown in FIG. 41J, tool 400 has retracted obstructor 440 out of lock 160, causing the lock to lock to tether 112, but has not yet cut the tether. As also described hereinabove, such retraction of obstructor 440 allows lock 160d to be released from head 420 (e.g., due to the obstructor no longer obstructing latch 168). Furthermore, due to the triggering of safety latch 498, the state shown in FIG. 41J can be considered to be a discrete and/or stable state. Therefore, should the operator (e.g., the physician) determine that it may be advantageous for a given implant to receive a second lock 160d, the operator is provided with the option to remove tool 400 while it remains in this state, to reintroduce it (or a second tool 400) with a second lock, and to proceed with cutting of tether 112 only after the second lock is locked to the tether.

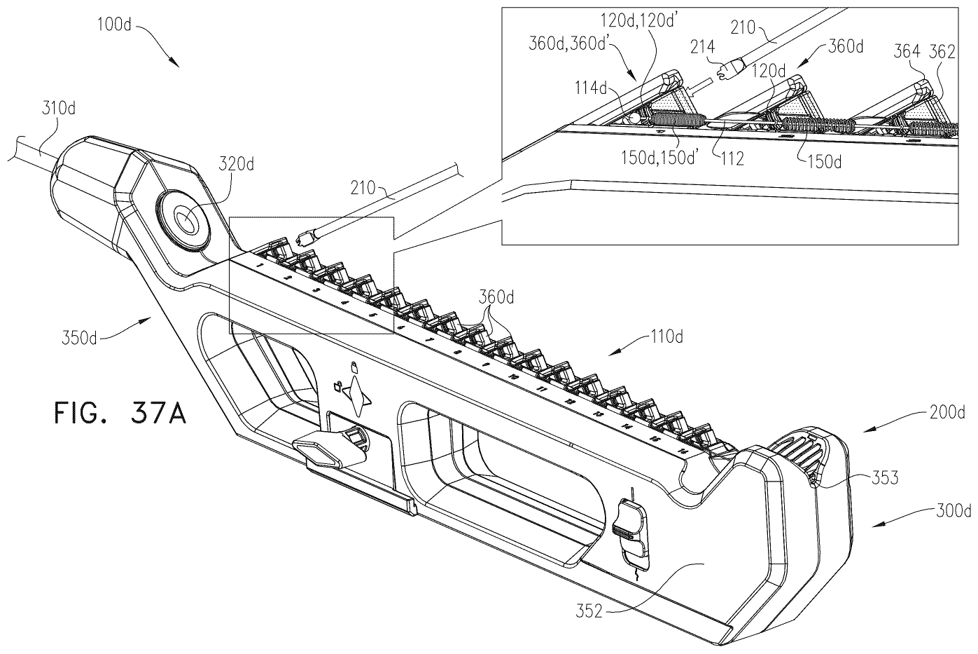

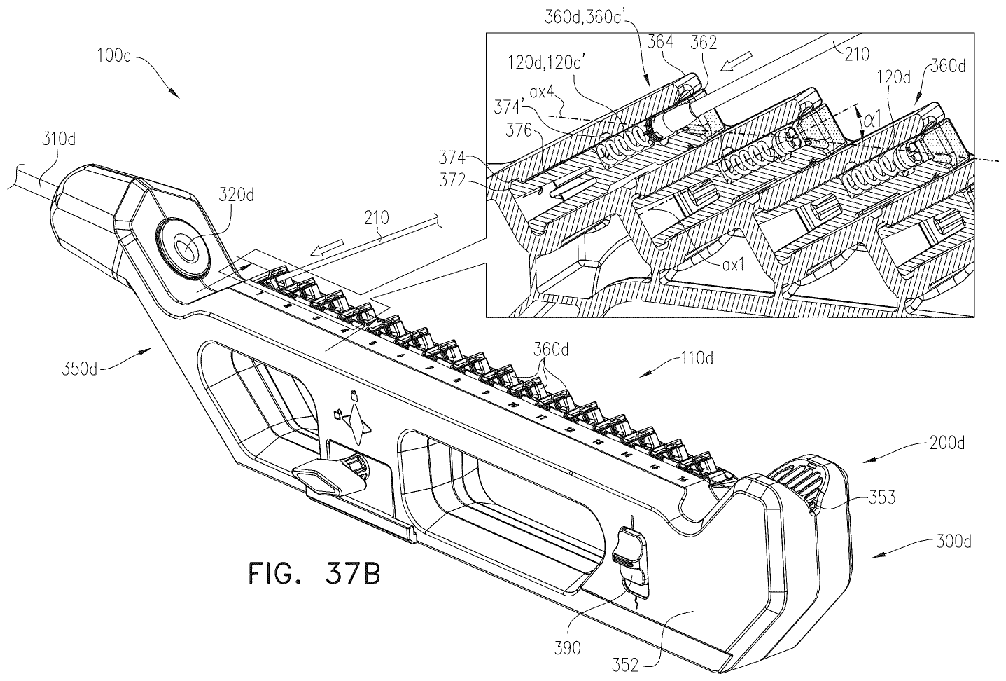

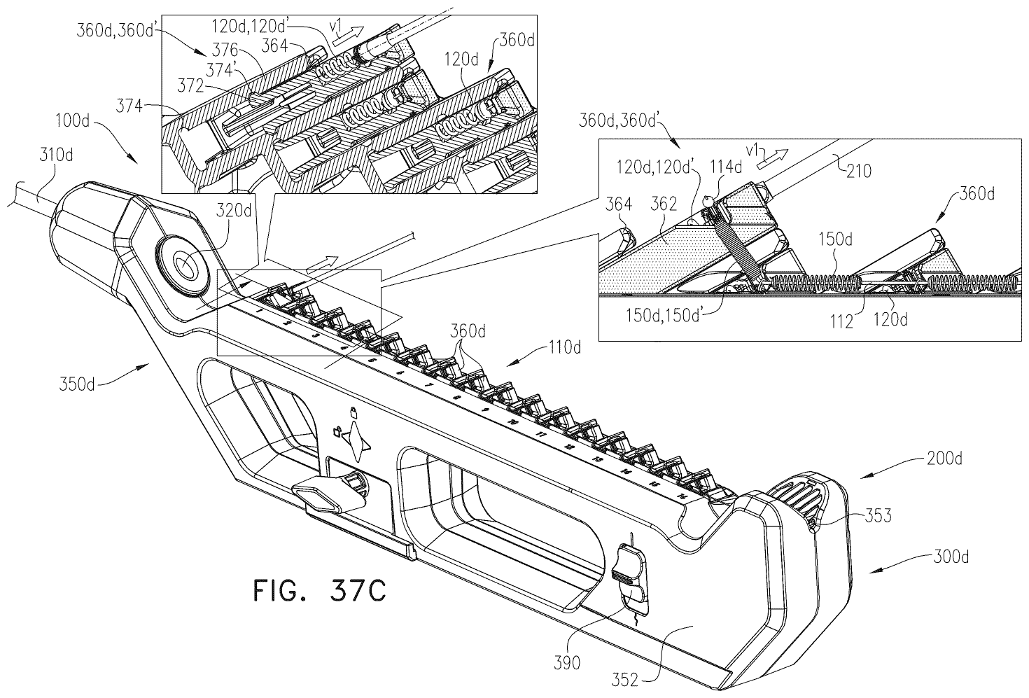

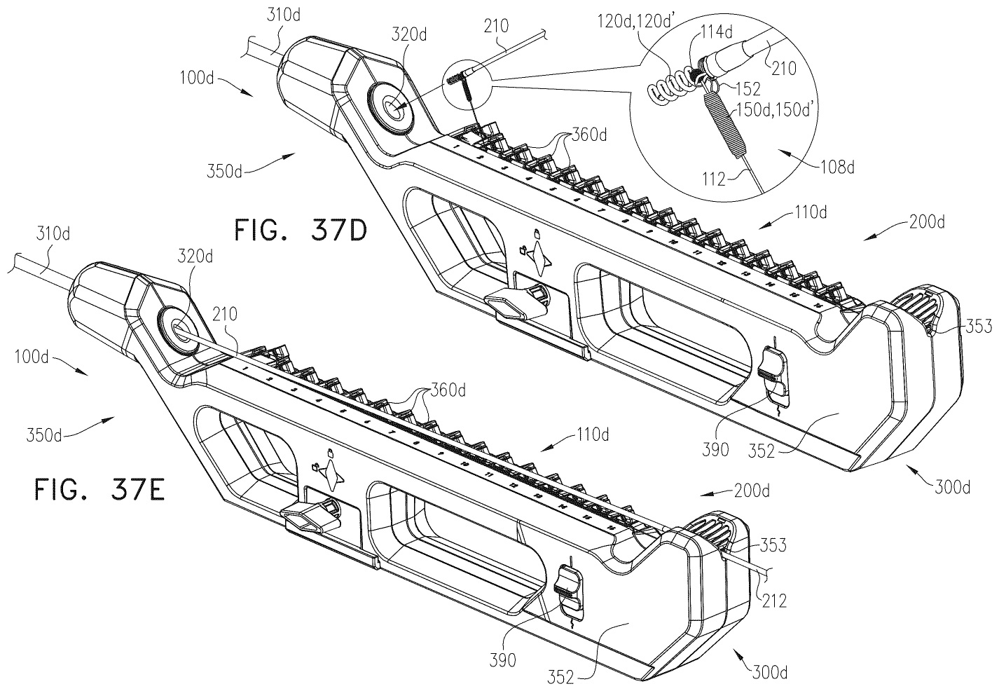

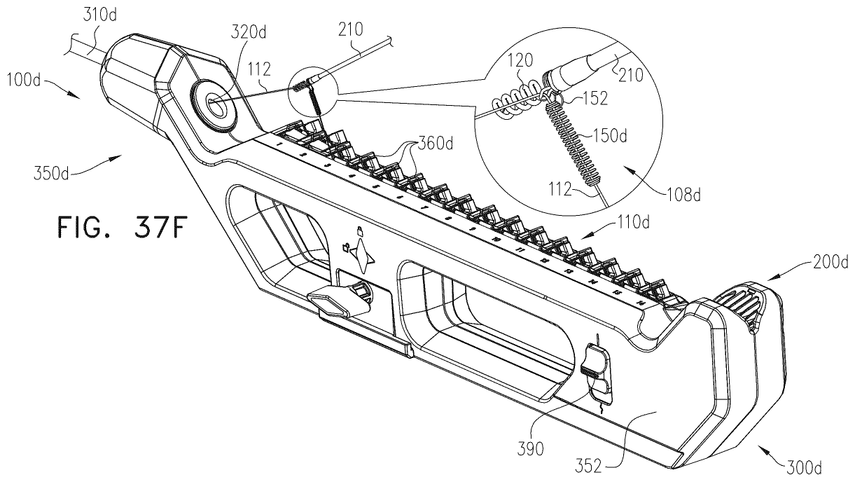

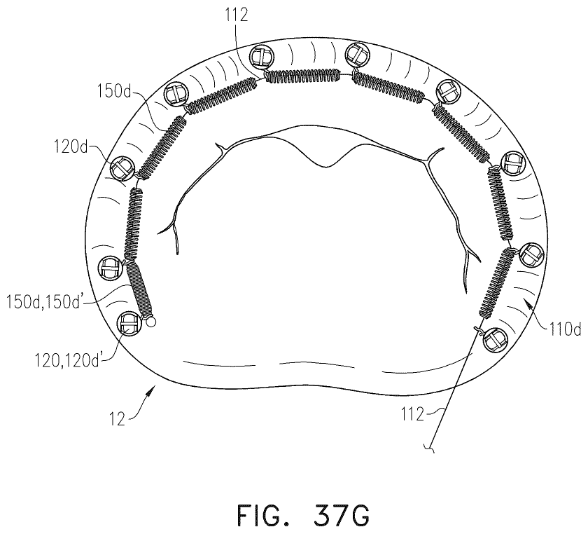

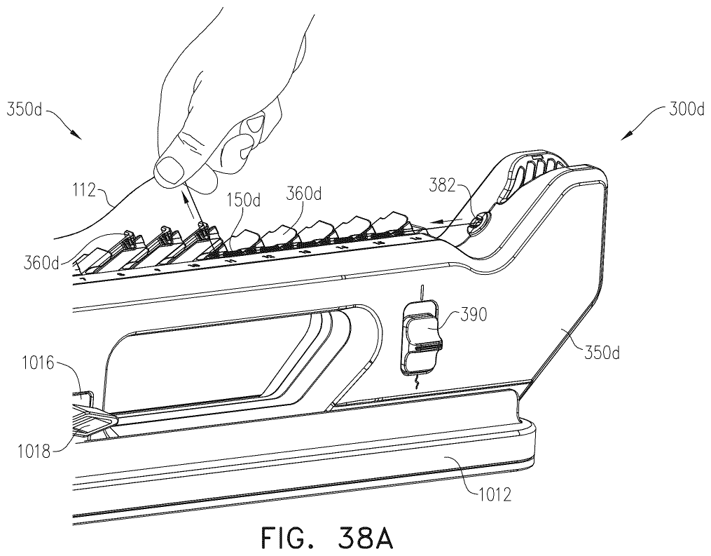

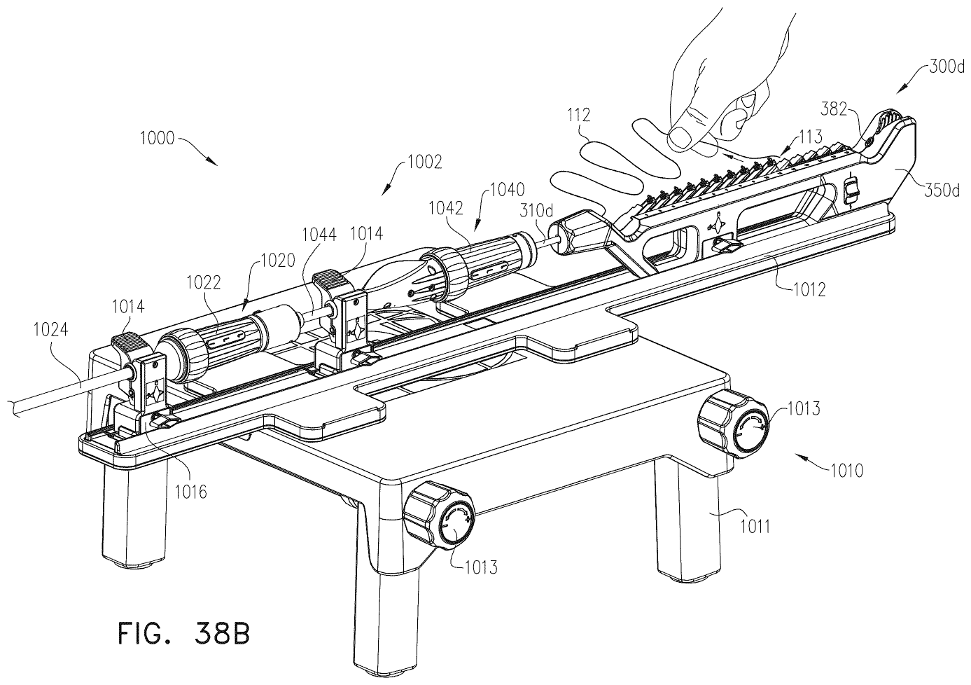

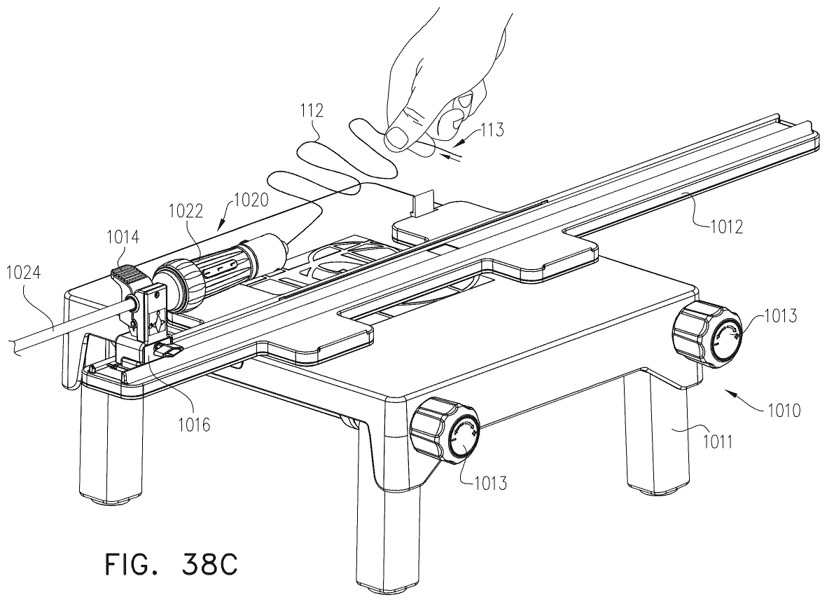

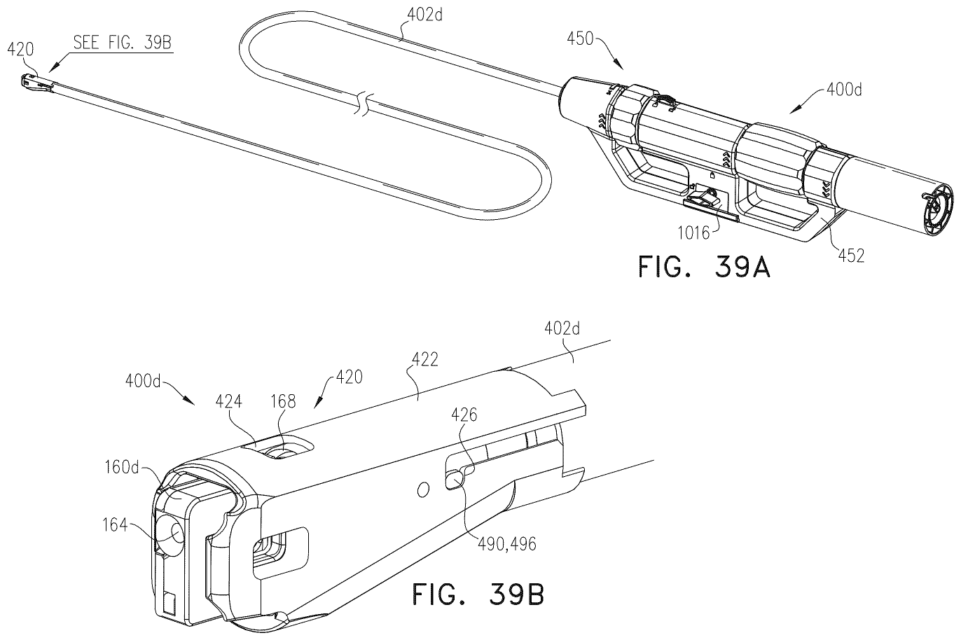

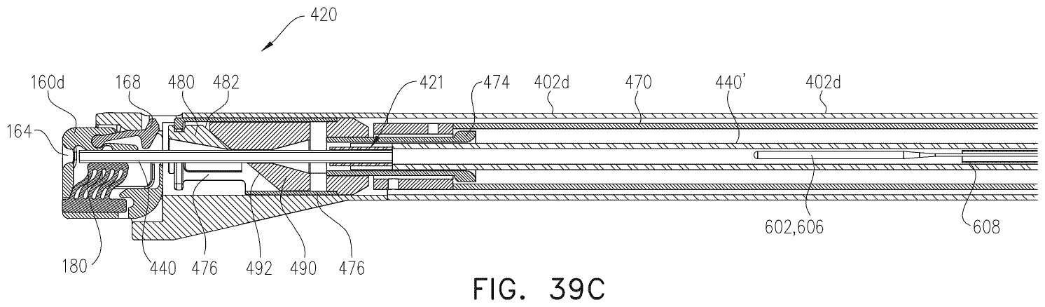

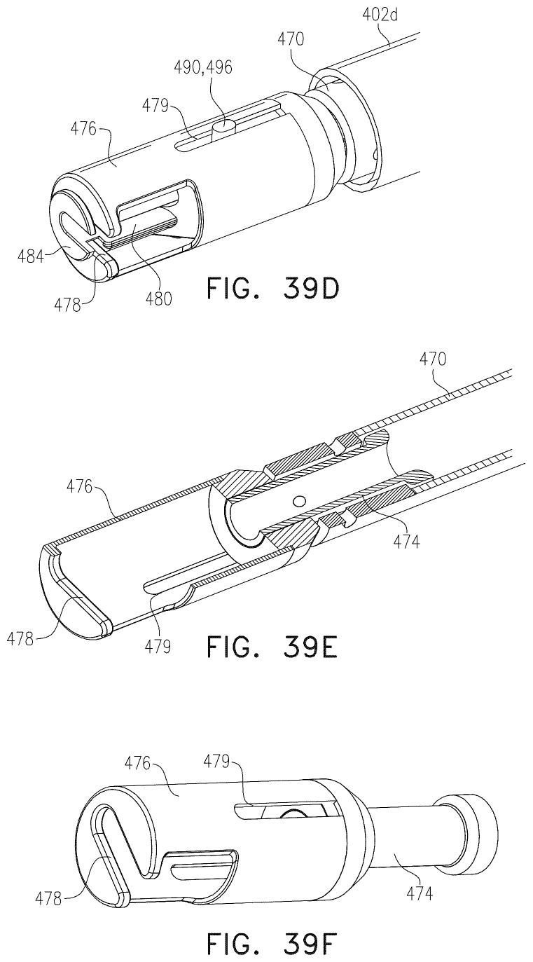

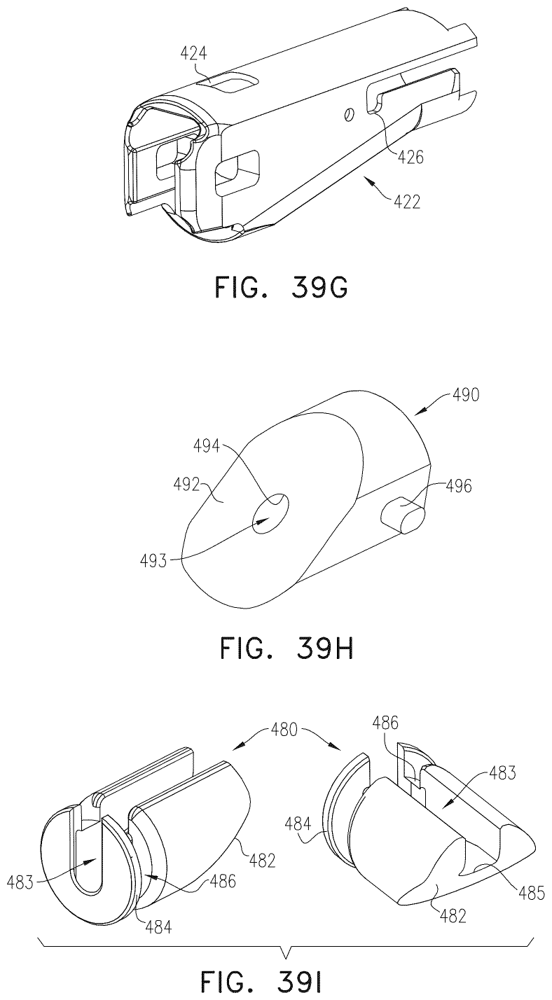

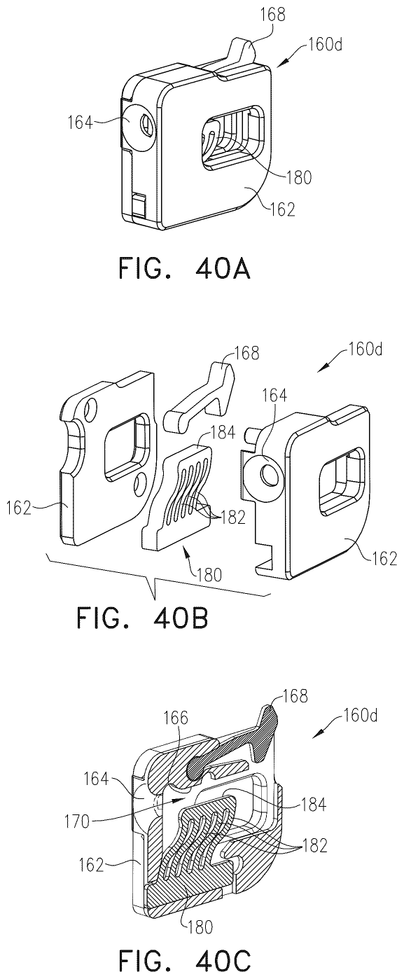

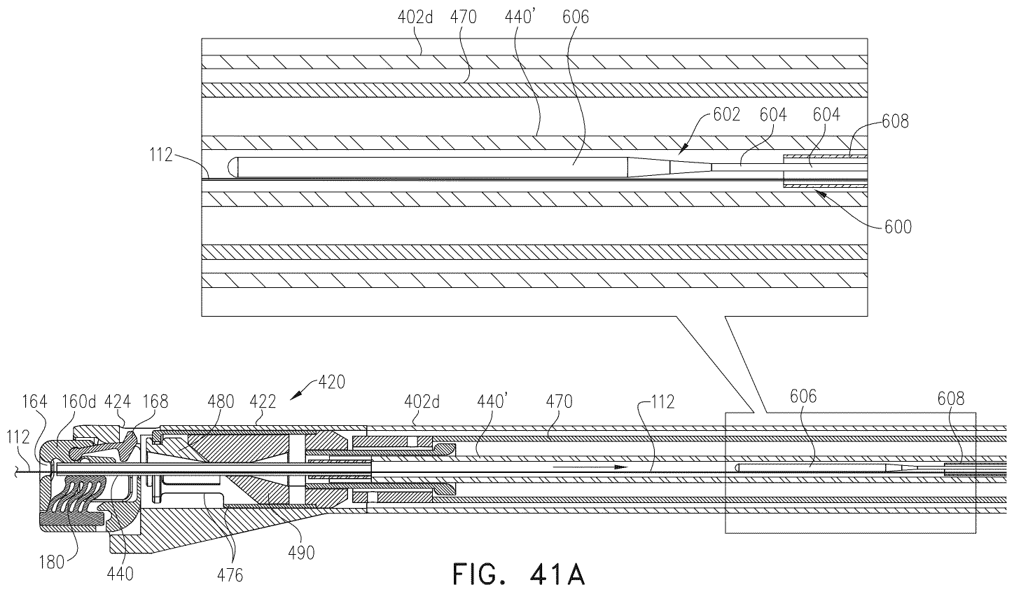

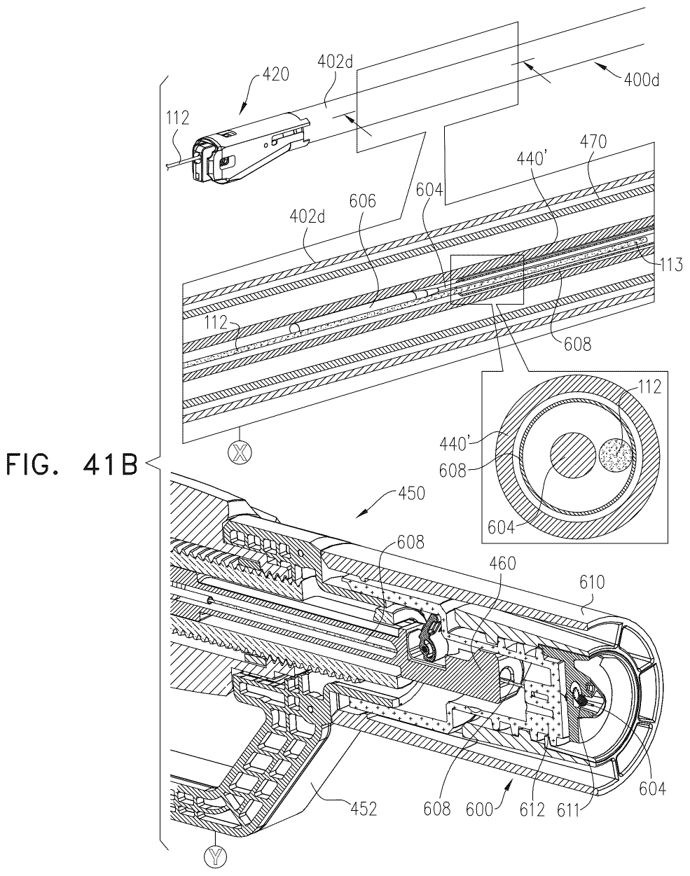

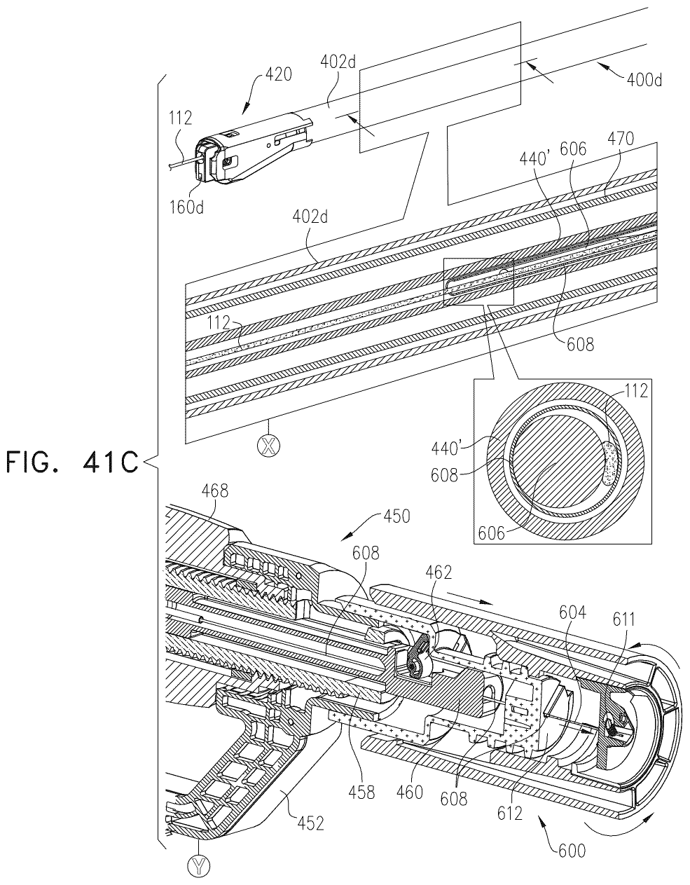

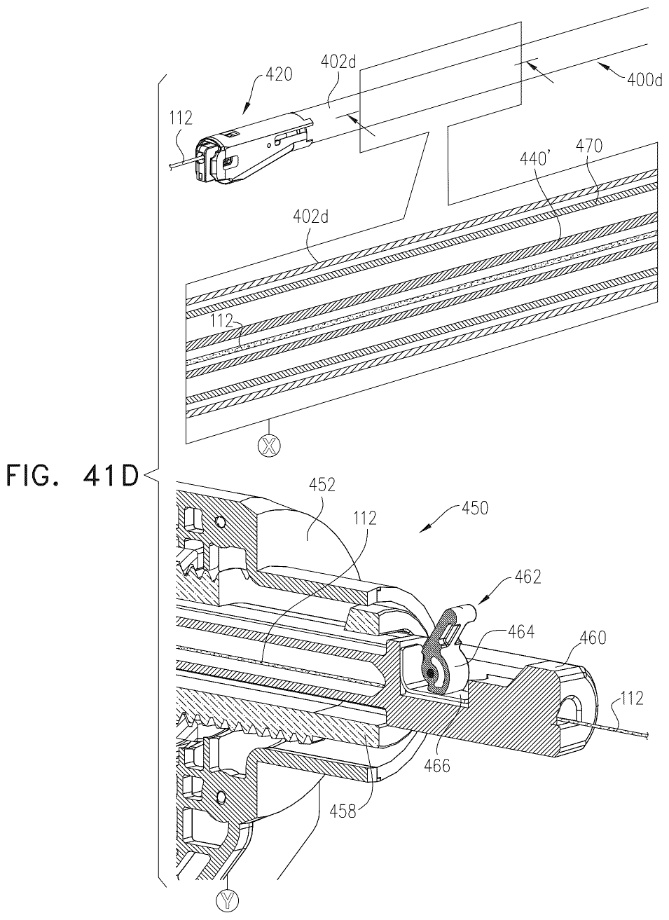

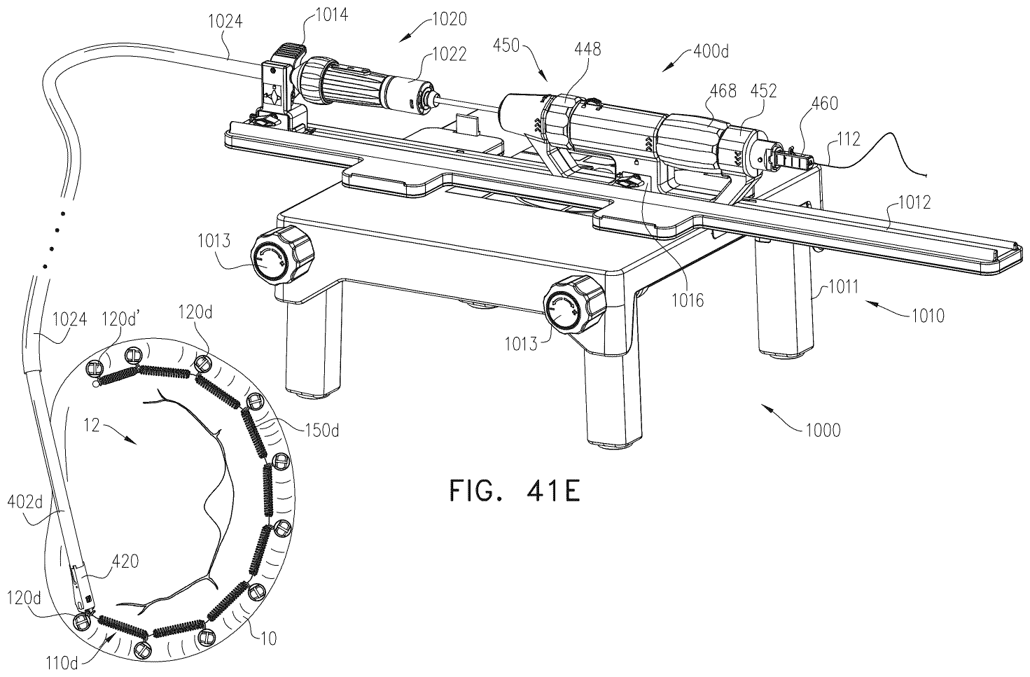

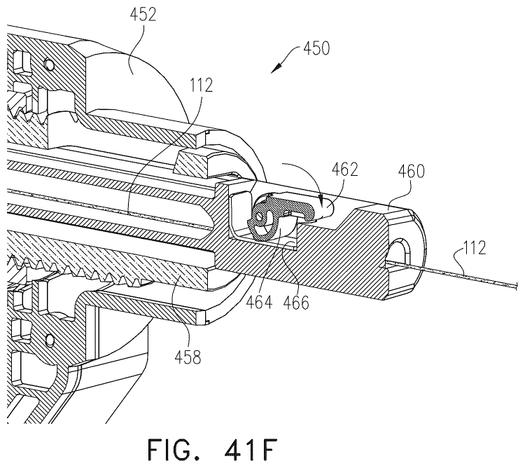

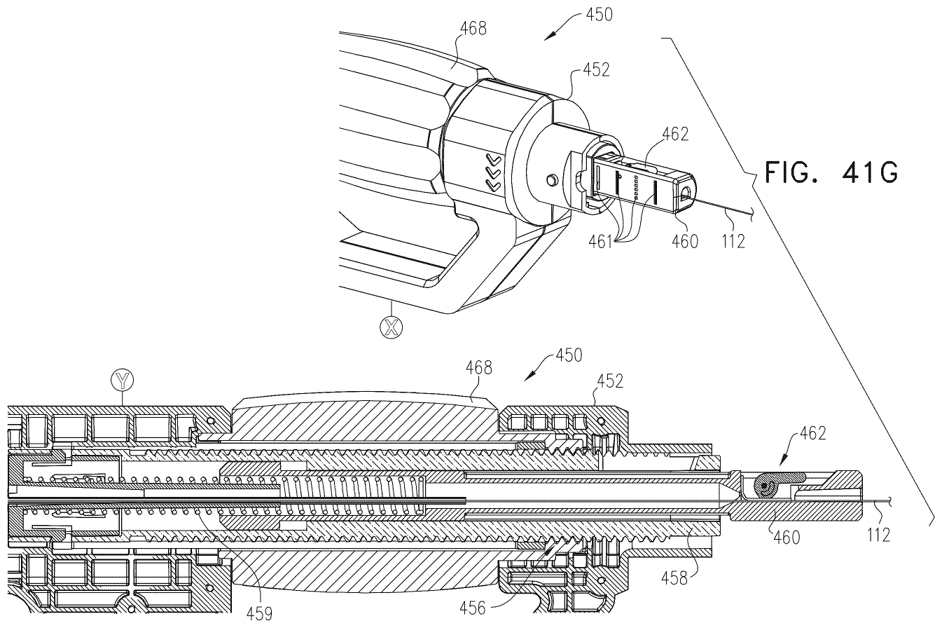

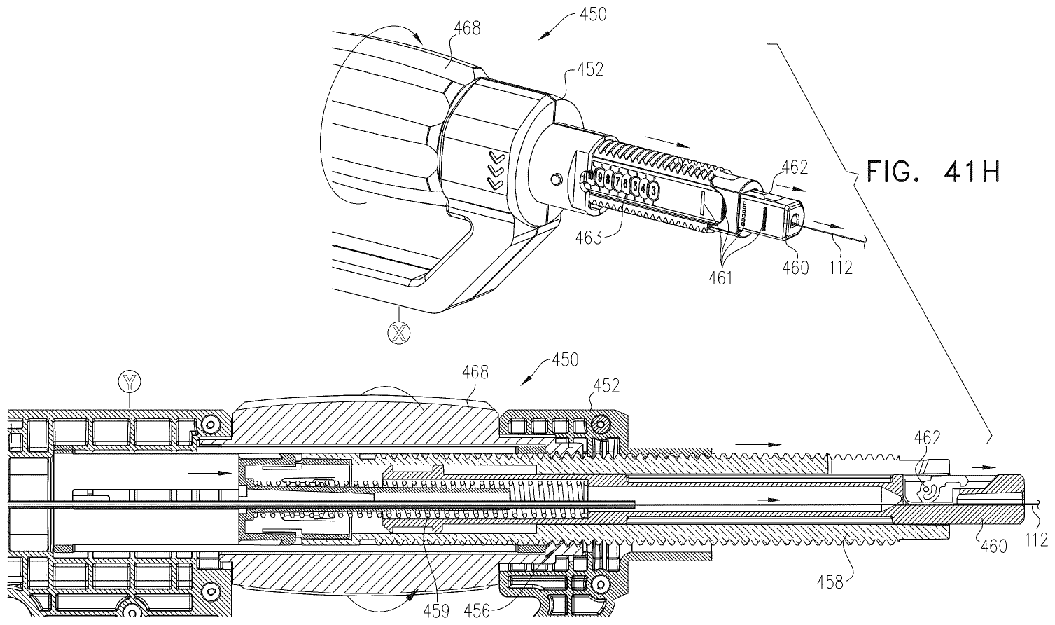

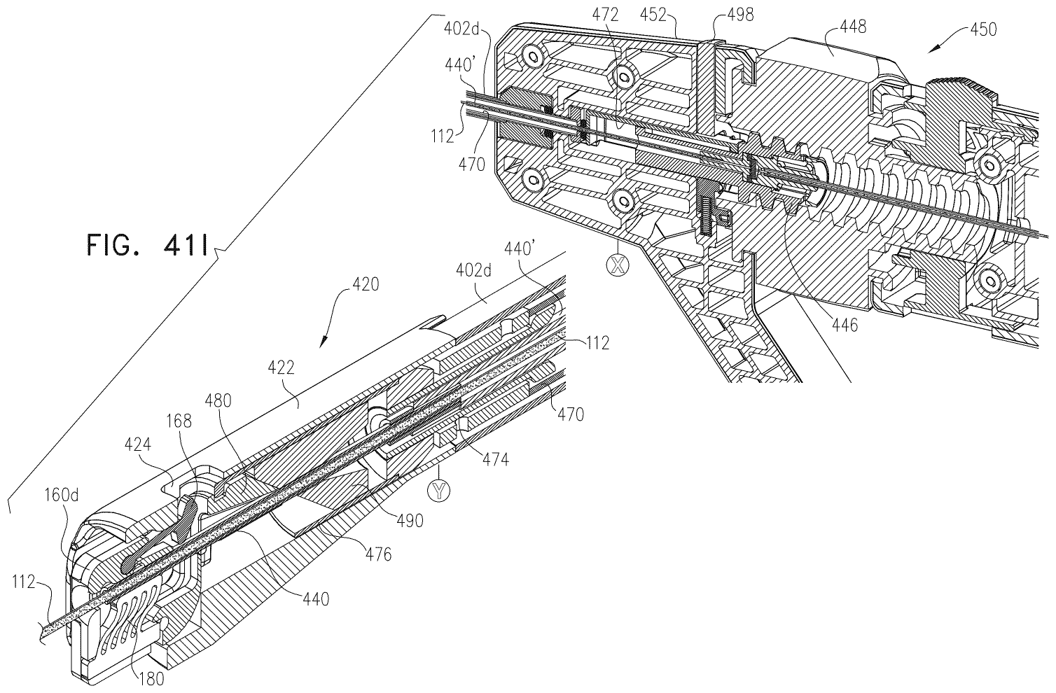

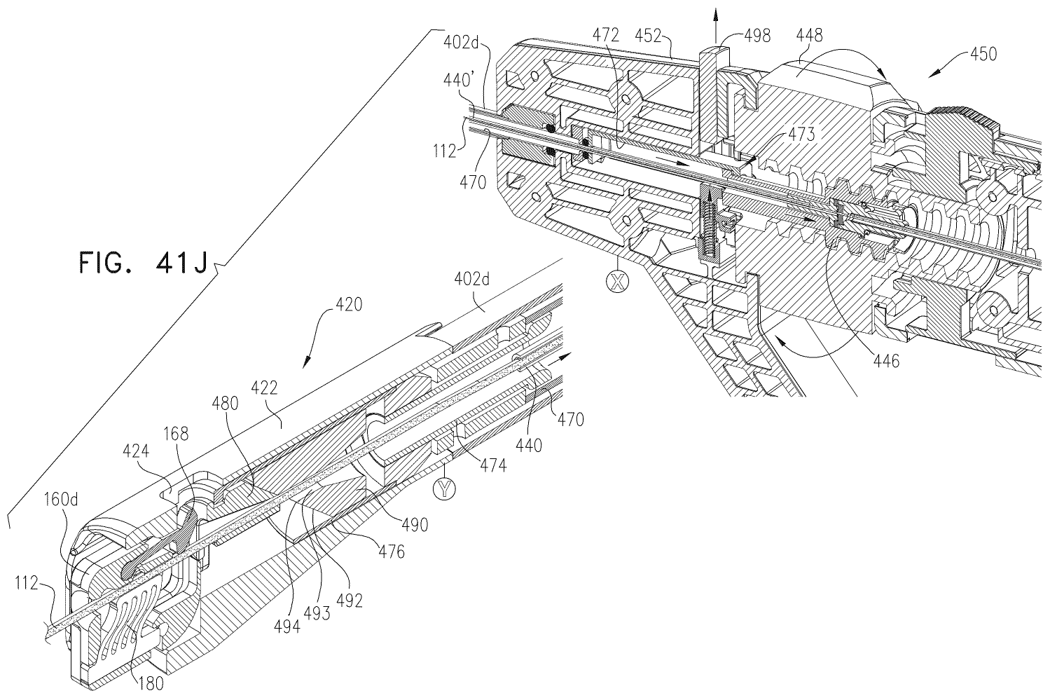

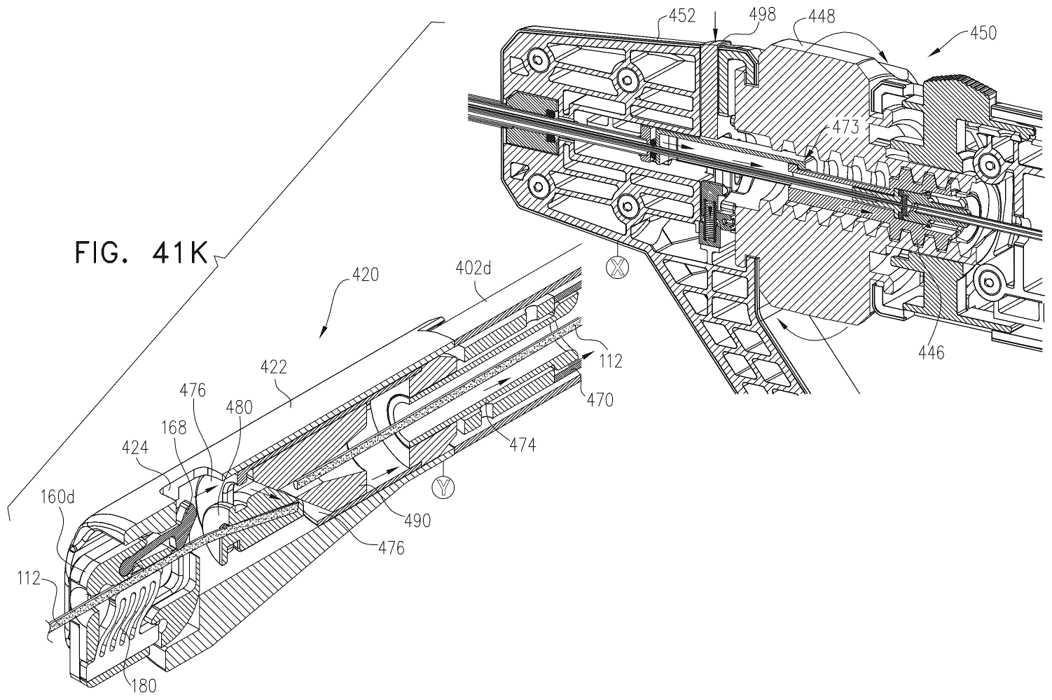

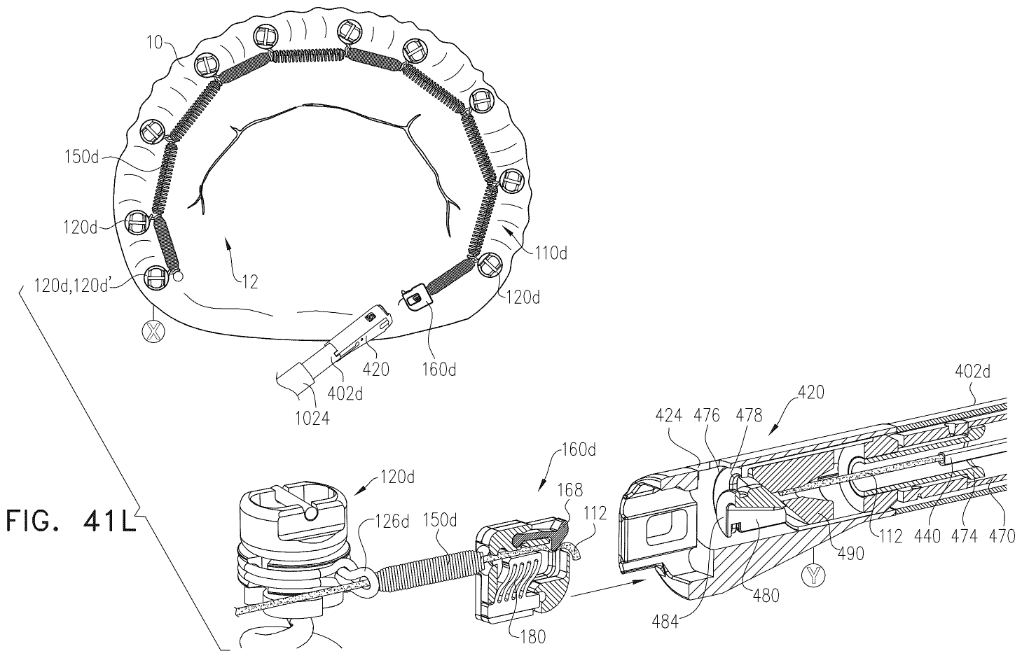

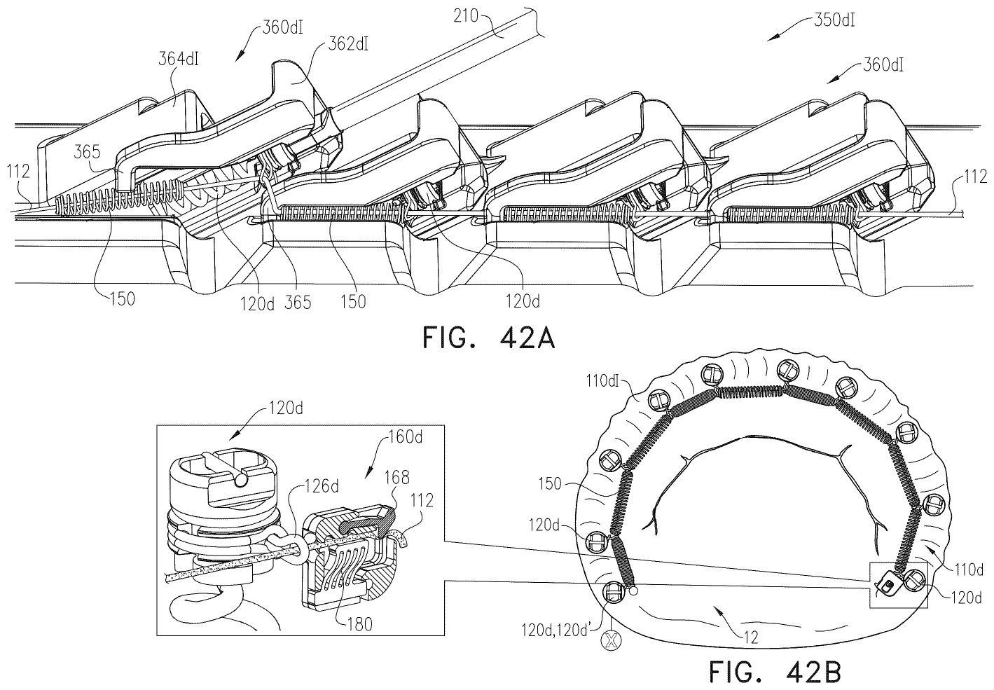

Reference is now made to FIGS. 42A-B. FIG. 42A is a schematic illustration of part of an extracorporeal unit 350dI, in accordance with some implementations. Extracorporeal unit 350dI can belong to a catheter device similar to catheter device 300d, and can be as described for extracorporeal unit 350d, mutatis mutandis, except for differences noted between cartridges 360dI of extracorporeal unit 350dI and those of extracorporeal unit 350d.

In some implementations, cartridge 360dI can, like cartridge 360d, comprise a tray 362dI and a chassis 364dI. In some implementations, cartridge 360dI can comprise a catch 365 (which can be hook-shaped) that, while the cartridge is in its closed state, lies adjacent tether 112 in a manner that obstructs spacer 150 from sliding distally past the catch—and thereby distally away from the anchor in the cartridge. This can be advantageous for implementations in which spacer 150 is not connected to an anchor of the implant, and is pushed along by an anchor, such that it lies ahead of the eyelet of the anchor. For example, catches 365 can prevent such spacers from prematurely migrating distally, e.g., as tether 112 is advanced distally with the leading anchor. FIG. 37 shows one cartridge 360dI (that on the left side of the figure) having transitioned into its open state, displacing catch 365 obliquely (e.g., proximolaterally) out of the way of spacer 150, thereby allowing the spacer to be subsequently advanced. Note that the successive spacer remains obstructed by the catch of the subsequent cartridge.

FIG. 42B shows an implant 110dI having been implanted at valve 12, in accordance with some implementations. Implant 110dI can be as described for implant 110d except that its spacers 150 are not connected to its anchors except by tether 112. Implant 110dI can be implanted using the catheter device described with reference to FIG. 42A. During implantation, each of its spacers 150 is pushed along tether 112 by an anchor 120d. That is, each of its anchors 120d (except for the leading anchor) is advanced to the heart with a corresponding spacer (e.g., a corresponding spacer disposed ahead of the anchor). Thus, the final anchor to be implanted is not followed by a spacer, and lock 160d abuts the last anchor 120d (e.g., its eyelet 126d) directly.

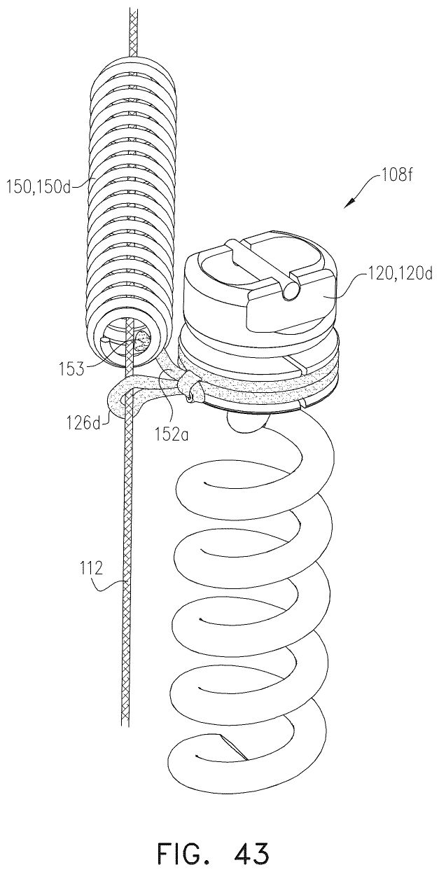

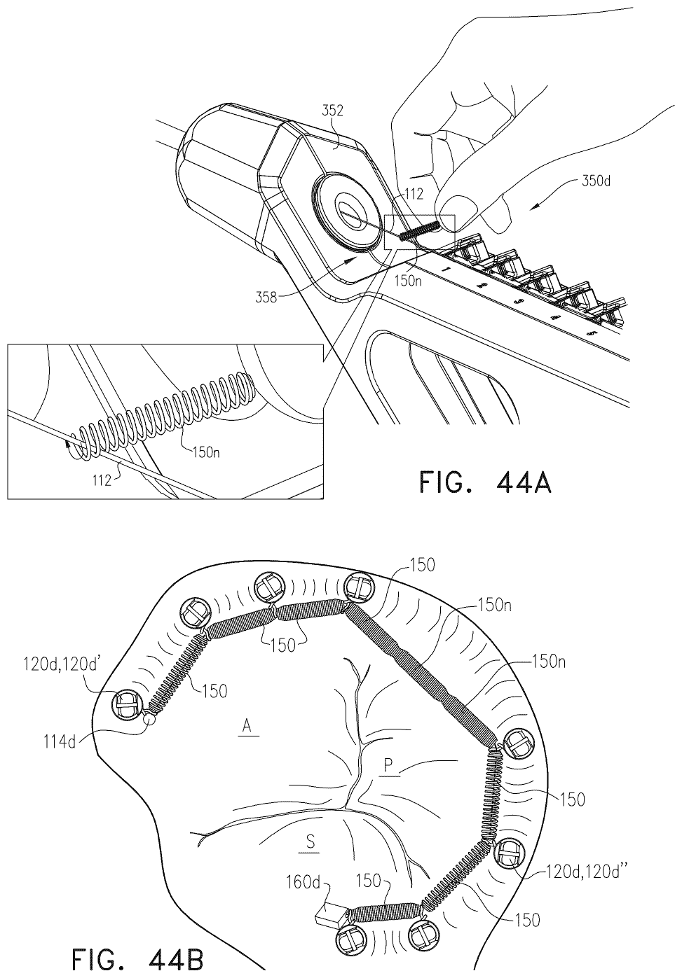

Reference is now made to FIGS. 43 and 44A-B. In some implementations, the presence of a spacer between the final anchor and the lock of the implant (e.g., as shown in FIG. 41L) may advantageously moderate forces between the lock and the final anchor, and/or reduce friction as tether 112 is pulled proximally during contraction of the implant, i.e., by dampening jerking movements, distributing forces, and/or maintaining at least a baseline radius of curvature of the tether between the final anchor and the lock, rather than allowing the tether to assume a tight curve and/or become trapped between the final anchor and the lock. However, in some implementations, the absence of a spacer between the final anchor and the lock (e.g., as shown in FIG. 42B) may be preferred.

FIGS. 43 and 44A-B are schematic illustrations of, in accordance with some implementations, techniques for changing the presence of a spacer between the final anchor and the lock of an implant, e.g., making the presence or absence of such a spacer independent of whether the system uses a spacer-trailing implant (i.e., an implant in which each spacer trails the anchor to which it is connected, e.g., as described for implant 110d) or a spacer-leading implant (e.g., as described for implants 100a and 100dI). For example, for a spacer-trailing implant, the spacer connected to the final anchor removed prior to contraction; and, for a spacer-leading implant, a spacer can be added proximal to the final anchor prior to contraction. Such removal or addition of a spacer can be by-design in some implementations. In some implementations, such removal or addition of a spacer can be according to the preference of a physician and/or according to a particular implantation procedure.

FIG. 43 is a schematic illustration of an anchor-spacer assembly 108f that comprises a connector 152a that connects a spacer 150 (e.g., spacer 150d) to a respective anchor 120 (e.g., anchor 120d), in accordance with some implementations. Connector 152a can be structurally and/or functionally as described for connector 152 except that it, or its connection to the respective spacer or to the respective anchor, is frangible. This allows the operator to disconnect the spacer that is connected to the final anchor of the implant, e.g., for a spacer-trailing implant. In the example shown, the frangible connection is between connector 152a and the spacer, and is achieved by the connector (e.g., a cord) being placed between turns of the helical coil of the spacer. Even for a spacer in which the coil is generally an open coil having space between its turns, turns at one or both ends of the spacer can have a reduced pitch.

The connector can be simply tucked between adjacent turns of the helix (e.g., such that the connector is pinched by the adjacent turns), e.g., as shown, or can be tied onto a turn. In some implementations, connector 152a can have a bulbous knot or stopper 153 on its end in order to maintain the connection. Connector 152a can be disconnected from spacer 150 by simply pulling, or by rotating the spacer such that it unwinds/unscrews from the connector. Tether 112 can then be unthreaded from the spacer.

The disconnected spacer can then be removed along with the extracorporeal unit and the remaining anchors and spacers mounted on the extracorporeal unit (e.g., as described with reference to FIGS. 38A-C, mutatis mutandis).

For some implementations, a cutting and/or unpicking implement can be used to cut the connector and/or unpick it from the spacer. For such implementations, the connector and its connection may not have a distinct or discrete frangible feature. In some implementations, a disconnector 72 can be provided with the system. Disconnector 72 can be a sharp implement, such as a blade and/or a seam-ripper. FIG. 34A shows an example in which such an implement is included with system 1000, e.g., provided within a compartment (e.g., a drawer) 356 in extracorporeal unit 350d (e.g., within body 352).

FIG. 44A is a schematic illustration of a spacer being added to an implant proximal to the final anchor 120, in accordance with some implementations. In the example shown, this is achieved by winding/screwing the spacer onto tether 112, e.g., by passing part of the tether between the first two turns of the spacer's helix and then rotating the spacer until the part of the tether exits from between the final two turns of the spacer's helix, leaving the tether extending through the lumen of the spacer.

In the example shown, the spacer being added is a spacer 150n, which is specifically configured to facilitate this procedure—e.g., by having its helical coil being open at least one end, providing a gap between the final and penultimate turns of the coil into which the tether can be introduced. Spacer 150n can therefore be referred to as a free spacer, e.g., because it is provided separate from implant 110 and can be provided separate from catheter device 300d.

Adding a spacer in such a manner may be used to provide a spacer-leading implant with a spacer between the final anchor and the lock of the implant. Similarly, adding a spacer in such a manner can be used to provide additional spacers to the implant intraprocedurally, e.g., should it be deemed advantageous. This permits an operator (e.g., physician) to customize an implant based on features of heart anatomy and/or features of the anatomy of a specific subject.

FIG. 44B is a schematic illustration of example of such addition in which, during implantation of an implant, two spacers 150n were added between the fourth and fifth anchors, such that three spacers (the spacer 150 that is connected to the fourth anchor, plus the added spacer 150n) support a large gap between the fourth and fifth anchors. This may, for example, be advantageous when the implant must span a region of tissue into which it is undesirable to drive an anchor, e.g., due to proximity of a coronary blood vessel or conductive tissue. It may also be advantageous when it has been determined that it would be preferable to limit contraction of a particular region of the tissue.

It is to be noted that such a technique can be used, mutatis mutandis, in order to add a spacer (e.g., an additional spacer) elsewhere on the implant, i.e., between one or more pairs of adjacent anchors of the implant, or between an anchor and another spacer.

It is to be further noted that such a technique can be used in reverse in order to remove from the implant a spacer other than that associated with the final anchor of the implant, optionally in combination with the disconnection techniques described with reference to FIG. 43, i.e., such that one or more pairs of adjacent anchors of the implant have no spacer therebetween.

In some implementations, a spacer-leading implant can be provided with a spacer 150 proximal to the final anchor (e.g., in order to become positioned between the final anchor and the lock of the implant) by disconnecting the spacer that is connected to the next-most-proximal anchor (i.e., the first of the anchors that is to be unused and that will remain mounted on the extracorporeal unit) so that it can be slid along the tether to the final anchor (e.g., by being pushed by the lock).

In some implementations, extracorporeal unit 350d is provided with (e.g., shaped to define) a manipulation zone 358 that facilitates manual manipulation, e.g., addition/connection and/or removal/disconnection of spacers. Body 352 of extracorporeal unit 350d can be shaped such that, at manipulation zone 358, an enlarged space exists between the body and tether 112, thereby facilitating manual manipulation of the tether, spacer, and/or anchor. For example, and as shown, manipulation zone 358 can be defined by a recess in body 352.

It is to be noted that manipulation zone 358 can similarly facilitate addition/connection of locks to tether 112—in particular, locks that are configured to be added/connected to (e.g., an intermediate region of) the tether without access to an end of the tether, such as locks 160a, 160b, and 160k, mutatis mutandis.

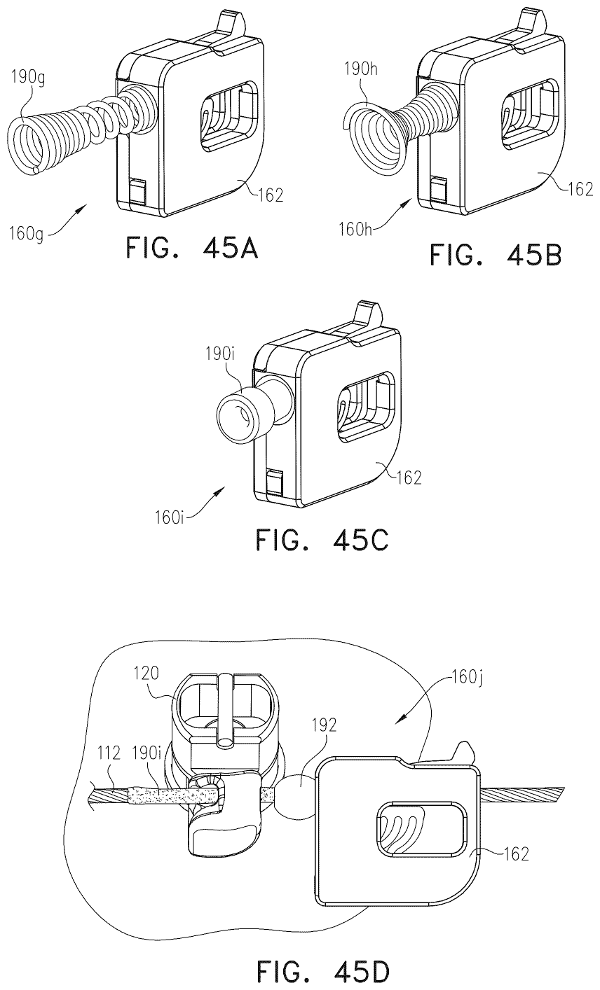

Reference is now made to FIGS. 45A-D, which are schematic illustrations of various locks (e.g., variants of lock 160d) that each comprise a leader 190, in accordance with some implementations. Leader 190 extends away from case 162 of the lock, i.e., extending distally away from distal-facing aperture 164. Leader 190 can be tubular, and/or can provide similar functionality to that provided by a spacer disposed between the final anchor and the lock of the implant (described with reference to FIGS. 43-44).

FIG. 45A shows a lock 160g that has a leader 190g that comprises an elastic helical or helicoidal coil, which can be formed from a metal such as nitinol. Like spacer 150, leader 190g can act like a compression spring and/or shock absorber, thereby moderating forces between lock 160g and the final anchor of the implant. Leader 190g can also maintain a baseline radius of curvature of tether 112 between the final anchor and lock 160g. In some implementations, leader 190g is flared at its distal end.

FIG. 45B shows a lock 160h that has a leader 190h that is similar to leader 190g except that it is flared at its distal end. In some implementations, leader 190h can maintain a greater baseline radius of curvature of tether 112 between the final anchor and the lock compared to leader 190g.

FIG. 45C shows a lock 160i that has a leader 190i that comprises a protuberant and smooth/curved rim (e.g., a flange) that maintains a baseline radius of curvature of tether 112 between the final anchor and lock 160i, and facilitates smooth sliding of the tether into the lock. Leader 190i can be formed from or coated with a low-friction material such as PTFE. Leader 190i can be rigid or can be flexible.

FIG. 45D shows a lock 160j that has a leader 190j that comprises a sleeve that is sufficiently narrow to slide through, and therefore line, the eyelet of the final anchor 120, e.g., as shown. Leader 190j can protect tether 112 at this site, and/or can provide greater engagement between the lock and the final anchor. The sleeve can be formed from and/or lined with a low-friction material such as PTFE. In addition to the sleeve, leader 190j can comprise a bulb 192, e.g., at a root of the leader. Bulb 192 can be formed from a resilient or malleable material, and can serve to cushion the eyelet against case 162 of the lock.

Optionally, rather than leader 190j being part of lock 160j, leader 190j can be a separate component that is threaded onto tether 112 prior to threading the lock onto the tether. In some implementations, bulb 192 is situated partway along the sleeve of the leader, such that a proximal part of the sleeve slides into lock 160 and a distal part of the sleeve slides into the eyelet of the final anchor. In some such implementations, when lock 160 is locked, the clamp of the lock can clamp the proximal part of the sleeve with tether 112 within.

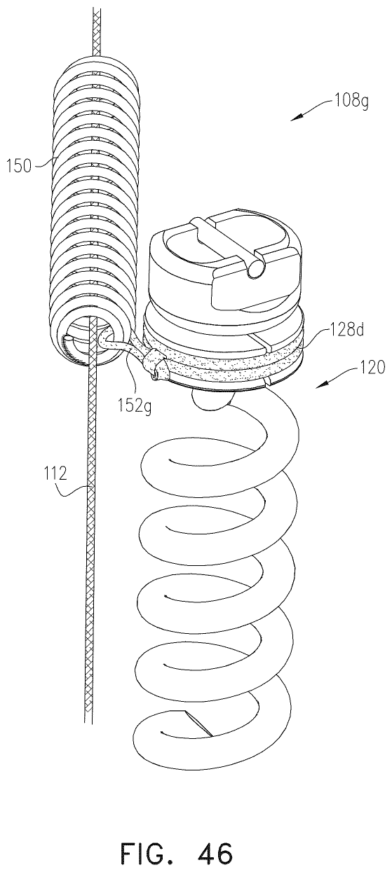

Reference is now made to FIG. 46, which is a schematic illustration of an anchor-spacer assembly 108g comprising an anchor 120 and a spacer 150, in accordance with some implementations. Assembly 108g can be similar to assembly 108d except that spacer 150 serves as the eyelet of anchor 120. That is, anchor 120 can be slidably coupled to tether 112 only via spacer 150, i.e., by the anchor being coupled to the spacer and the spacer being threaded onto the tether. In the example shown, a collar 128d of anchor 120 does not comprise a discrete eyelet, but does comprise a connector 152g that connects the anchor (e.g., the head of the anchor) to spacer 150. Connector 152g can be looped around one or more turns of the helix of spacer 150. For some implementations, the end of the helix is sealed (e.g., adjacent turns of the helix are fused, welded, soldered, brazed, bonded, etc.) in order to maintain anchor 120 connected to spacer 150 and thereby to tether 112.

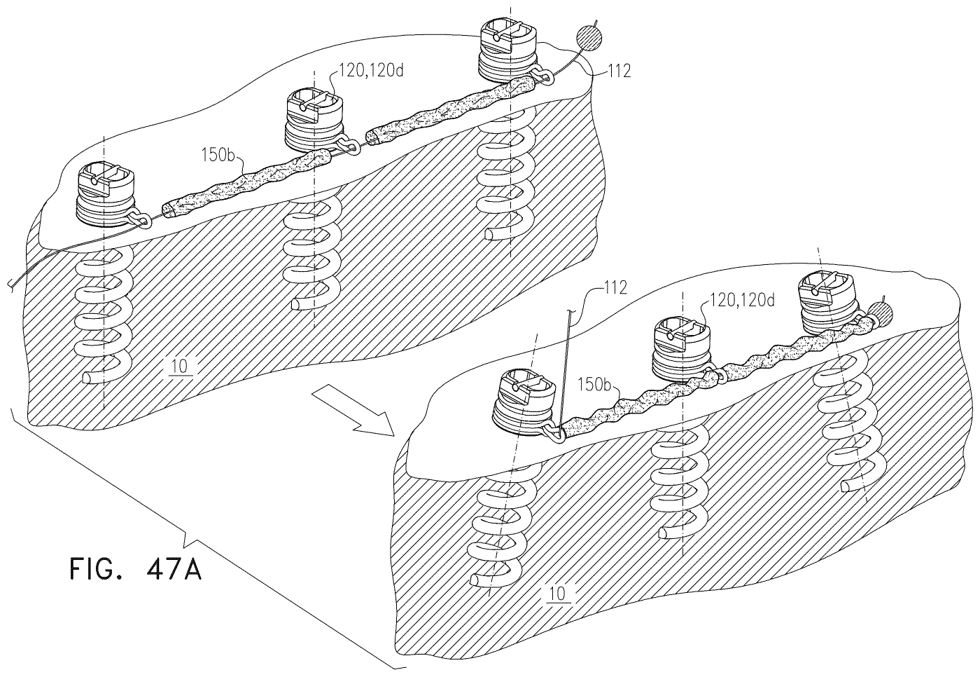

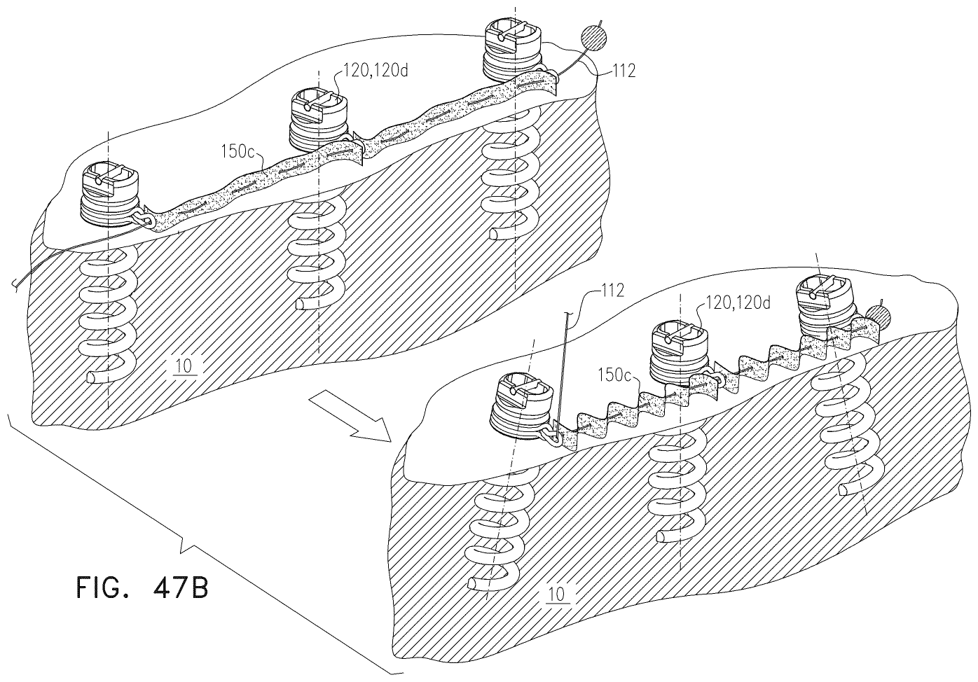

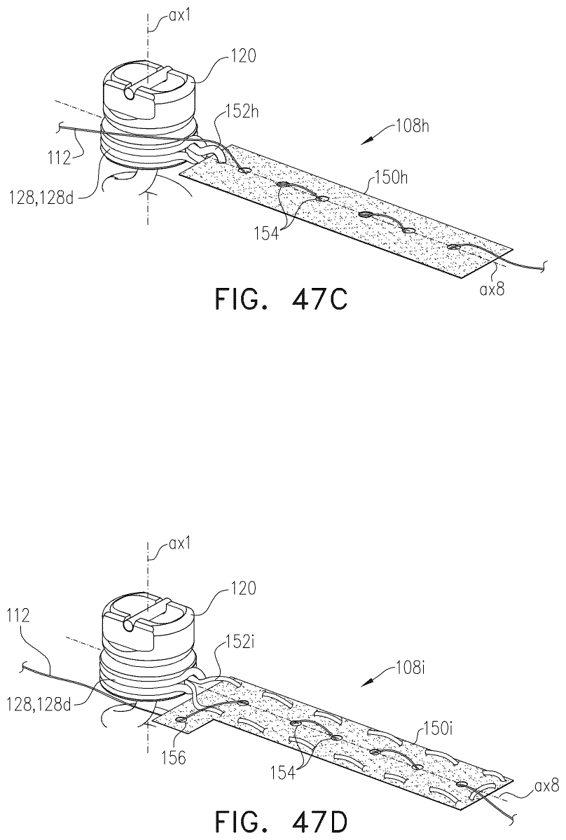

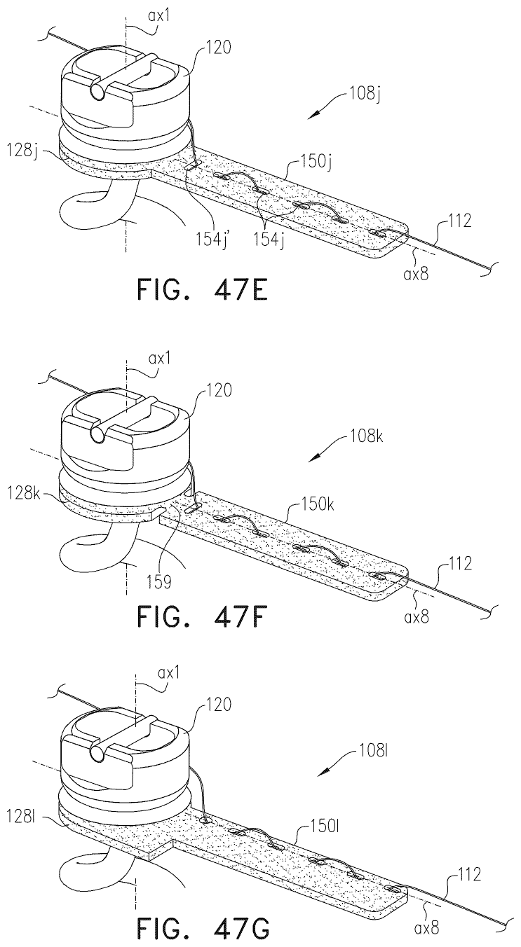

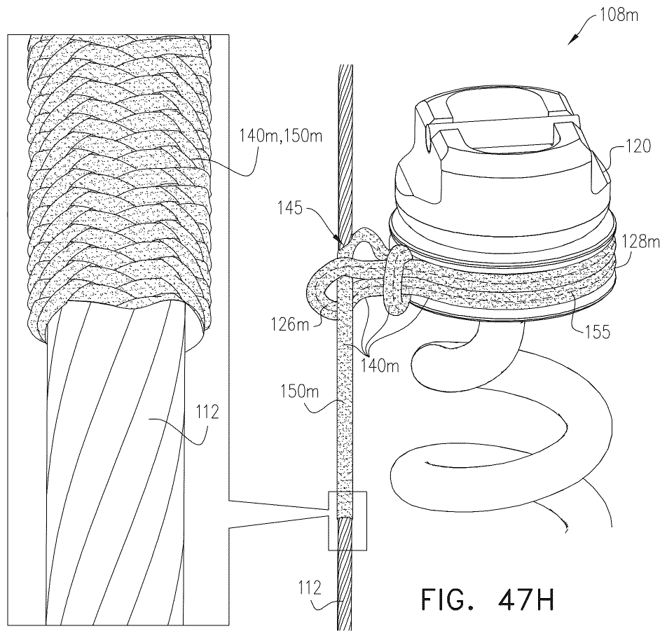

Reference is made to FIGS. 47A-H, which are schematic illustrations of various spacers and anchor-spacer assemblies, in accordance with some implementations.

FIGS. 47A-B show spacers 150b and 150c, which can be considered variants of spacer 150. Each figure shows an implant (e.g., a variant of implant 110) comprising the spacers between anchors 120d of the implant (i) before tensioning of tether 112 and (ii) after tensioning of the tether has contracted the implant and tissue 10, and the consequent compression of the spacers.

Like spacer 150, spacer 150b is tubular. However, rather than comprising (or being defined by) a helical coil, spacer 150b comprises (e.g., consists of or is defined by) a flexible sleeve that is threaded onto tether 112, i.e., the tether extends through the lumen of the sleeve. The sleeve can be formed from a textile (e.g., a fabric) or a film. For some implementations, the sleeve can comprise and/or be formed from textile 140, described hereinabove. In some implementations, the sleeve is configured to promote tissue growth thereon. In some implementations, the sleeve is more axially compressible than spacer 150, but nonetheless resists compression to some degree (e.g., beyond a certain point) in order to serve as a spacer. In some implementations, once the implant has been contracted, spacers 150b collectively define a substantially continuous lumen along the implant.

Spacer 150c can be formed from the same material(s) as spacer 150b but, rather than comprising a sleeve along whose lumen tether 112 extends, spacer 150c comprises a strip (i.e., flat and elongate) along which the tether is woven.

As described hereinabove, in some implementations, each spacer of the implant can be connected to a respective anchor of the implant (e.g., with respect to anchor 120d) and/or can serve as the eyelet of the anchor, e.g., defining an anchor-spacer assembly. These options are similarly possible for implementations in which the spacer can comprise and/or be formed from a textile such as textile 140. For example, FIGS. 47C and 47D show, respectively, anchor-spacer assemblies 108h and 108i in which spacers 150h and 150i are connected to their respective anchors 120, e.g., by respective connectors 152h and 152i. Similarly to anchor-spacer assembly 108g, the anchor is slidably coupled to tether 112 only via the respective spacer. That is, spacer 150h serves as an eyelet of the anchor.

Spacers 150h and 150i can be otherwise similar to spacer 150c. Anchor-spacer assembly 108i differs from anchor-assembly 108h in that its connector 152i extends along and/or around the perimeter of spacer 150i, providing reinforcement. Spacer 150i also differs from spacer 150h in that it defines an offset hole 156 at which tether 112 enters the spacer. In this context, “offset” means spaced laterally from an axis ax8 along which tether 112 substantially extends along the spacer. Offset hole 156 can therefore, at least for some implementations, be considered to serve as an eyelet of the anchor-spacer assembly, and thus a unitary piece of textile can be shaped to define both the eyelet and the spacer of the anchor-spacer assembly.

Axis ax8 can also be the axis along which the tether extends from the spacer to the next anchor-spacer assembly of the implant. Axis ax8 can alternatively or additionally be defined by a series of perforations 154 that are distributed along the spacer and through which tether 112 is woven. Offset hole 156 can be disposed toward (e.g., at) the end of the spacer that is closest to anchor 120. Hole 156 being offset can facilitate tether 112 passing around the head of the anchor.

FIGS. 47E, 47F, and 47G show, respectively, anchor-spacer assemblies 108j, 108k, and 1801, which are similar to anchor-spacer assembly 108h. However, the unitary piece of textile that defines the spacer and eyelet of anchor-spacer assemblies 108j, 108k, and 1801 also defines the collar of the anchor of these anchor-spacer assemblies (collars 128j, 128k, and 1281, respectively).

Spacer 150j of anchor-spacer assembly 108j has perforations 154j through which tether 112 is woven. Of these perforations, the perforation 154j′ closest to anchor 120 can be specifically configured to serve as the eyelet of the anchor. For example, the perforation can be reinforced and/or it can be oblong with its major axis transverse to axis ax8, e.g., as shown. Perforation 154j′ being oblong with its major axis transverse to axis ax8 can facilitate tether 112 passing around the head of the anchor. In some implementations, the other perforations 154j are also oblong, but with their major axes aligned (e.g., collinear) with axis ax8.

Anchor-spacer assembly 108k is identical to anchor-spacer assembly 108j except that the textile is shaped to define a narrowed neck 159 that facilitates spacer 150k pivoting about its axis ax8. Neck 159 can lie on axis ax8.

Axis ax8 can intersect axis ax1 of the anchor of the anchor-spacer assembly, e.g., as shown for anchor-spacer assemblies 108h-k. Although not obvious from the viewing angle of FIG. 47G, in anchor-spacer assembly 108l, the perforations of spacer 150l are positioned such that axis ax8 does not intersect anchor axis ax1, but instead is offset with respect to the anchor axis. This can facilitate tether 112 passing around the head of the anchor.

FIG. 47H shows an anchor-spacer assembly 108m in which a unitary piece of textile 140m defines the spacer 150m, eyelet 126m, and collar 128m of the assembly. However, whereas the textile of assemblies 108h-1 is a strip (e.g., substantially flat), the textile of assembly 108m is tubular. Similarly to as described for anchor 120d, mutatis mutandis, this tubular textile can be arranged (e.g., tied) to define a collar 128m and/or an eyelet 126m. However, a segment of the tubular textile can also serve as a spacer 150m, with tether 112 extending through the lumen of this segment of the tubular textile. For example, this region can extend from an open end of the tubular textile to a notch 145 or other opening formed in the sidewall of the tubular textile, with tether 112 passing into/out of the open end and the notch and being slidable through the lumen of this region of the tubular textile. In some implementations, in at least some segments of the tubular textile (e.g., in the segments that define collar 128m and eyelet 126m), a reinforcing line 155 (e.g., a yarn, wire, or cable) is disposed within the lumen.

In some implementations, one or more spacers (e.g., each of the spacers) described with reference to FIGS. 47A-H can incorporate radiopaque and/or echogenic markers in order to facilitate imaging-based monitoring of the location and/or contraction of the implant.

In some implementations, one or more spacers (e.g., each of the spacers) described with reference to FIGS. 47A-H can be formed from a fabric that is woven to be anisotropic. For example, the fabric can make the spacer more medially compressible than axially compressible, or vice versa. Alternatively or additionally, the fabric can make the spacer medially flexible while being resistant to axial compression, or vice versa.

In some implementations, one or more anchor-spacer complexes (e.g., each of the anchor-spacer complexes) described with reference to FIGS. 47A-H can be arranged and/or advanced in a spacer-leading manner or in a spacer-trailing manner.

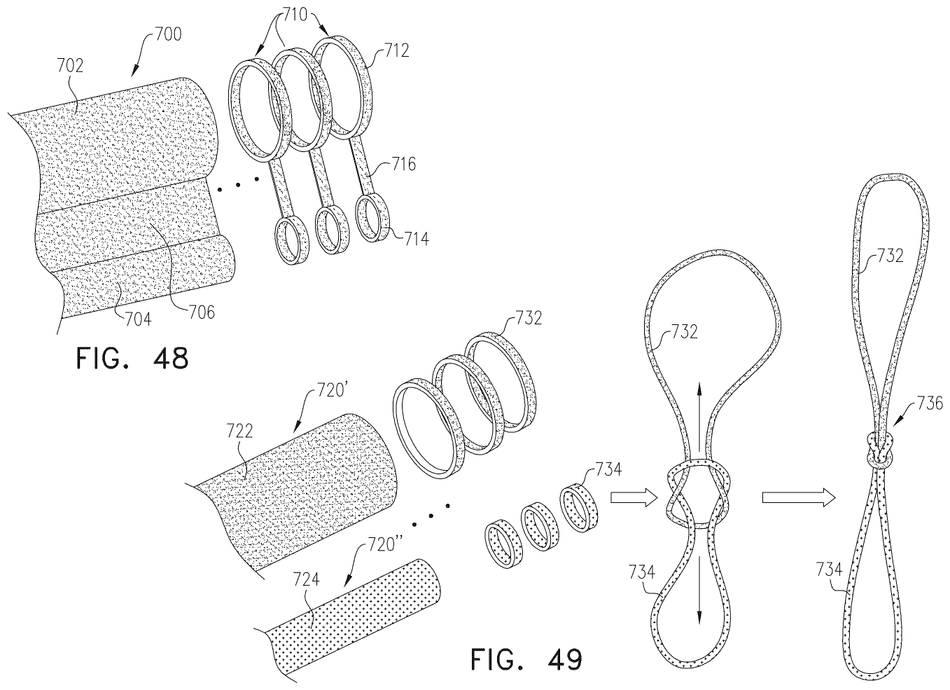

Reference is made to FIGS. 48-50, which are schematic illustrations of textile collars and eyelets, such as textile components that include both the collar and the eyelet, in accordance with some implementations. Further, these figures illustrate techniques that can advantageously increase rates and reduce costs of manufacturing textile collars and eyelets.

With reference to FIG. 48, a textile (e.g., fabric), such as textile 140, is woven into an elongate form 700 that includes (e.g., defines) two tubular structures 702 and 704 along the length of the elongate form. Form 700 can be shaped such that tubular structures 702 and 704 are parallel with each other. Because tubular structures 702 and 704 are both defined by elongate form 700, they are inherently connected to each other. Elongate form 700 can be further shaped to include (e.g., define) a belt 706 that connects tubular structure 702 to tubular structure 704. Belt 706 can be substantially flat, and can be parallel with the tubular structures.

Form 700 is then cut into transverse slices 710, each of which can be considered an individual textile component. Each slice 710 defines a first ring 712 that is a slice of (i.e., that is derived from) tubular structure 702, and a second ring 714 that is a slice of (i.e., that is derived from) tubular structure 704. In implementations in which belt 706 is included, each slice 710 also includes a tab 716 that is a slice of the belt, and that connects ring 712 to ring 714. Each slice 710 can then be mounted onto the head of an anchor such that first ring 712 serves as a collar of the anchor (e.g., circumscribing the head of the anchor) and second ring 714 serves as an eyelet of the anchor (e.g., extending laterally from the collar and the head). Thus, ring 712 defines a collar-aperture through the textile and ring 714 defines an eyelet-aperture through the textile.

Tubular structures 702 and 704 (and therefore rings 712 and 714) can have different inner diameters (e.g., as shown), or can have the same inner diameter as each other. For example, and as shown, tubular structure 702 (and therefore ring 712) can have a larger inner diameter than tubular structure 704 (and therefore ring 714).

In some implementations, tubular structures 702 and 704 (as well as belt 706, if present) are aligned with the warp axis of the textile, e.g., such that a given warp strand is included in either structure 702, structure 704, or belt 706. In such implementations, transverse slices 708 can be cut along the weft axis of the textile, e.g., such that a given weft strand is included in structure 702 and structure 704 (as well as belt 706, if present).

In some implementations, a batch manufacturing process is used, whereby the slicing is performed after the weaving of form 700 is complete. In some implementations, a continuous manufacturing process is used, whereby the slicing is performed as the weaving of form 700 continues, e.g., such that the weaving output continuously becomes the input for the slicing.

Although rings 712 and 714 and tab 716 are obtained from a single form 700, in some implementations the form can be woven with a different composition and/or structure at tubular structure 702, tubular structure 704, and/or belt 706 in order to provide different characteristics to the ring(s) and/or tab.

FIG. 49 shows a similar technique, except that the first ring 732 and the second ring 734 are formed from separate slices, and are therefore initially separate, and are subsequently connected to form a textile component 730 that includes both a collar and an eyelet and that can be mounted on the head of an anchor.

In the example shown, rings 732 and 734 are connected to each other by interlinking, i.e., passing each ring through and around the other to create a linkage that resembles a reef knot 736. In this manner, the resulting textile component can be shaped as a lemniscate, e.g., as shown. However, other means of connecting the rings (e.g., other linkages) can be used including, but not limited to, tying, stitching, or binding.

In the example shown, separate elongate forms 720′ and 720″ are (i) woven (e.g., as described for elongate form 700, mutatis mutandis), with elongate form 720′ including (e.g., defining) a first tubular structure 722 and elongate form 720″ including (e.g., defining) a second tubular structure 724, and (ii) sliced to define a first ring 732 that is a slice of first tubular structure 722 and a second ring 734 that is a slice of second tubular structure 724. Inter alia, this allows the characteristics (e.g., composition and dimensions) of rings 732 to be different from those of rings 734. However, in some implementations, modified technique can be used whereby rings 732 and rings 734 are both cut from a single elongate form that includes (e.g., defines) a single tubular structure. In such implementations, rings 732 and 734 can be identical to each other, or can differ from each other, e.g., different slice thicknesses can be used to produce rings of different thicknesses.

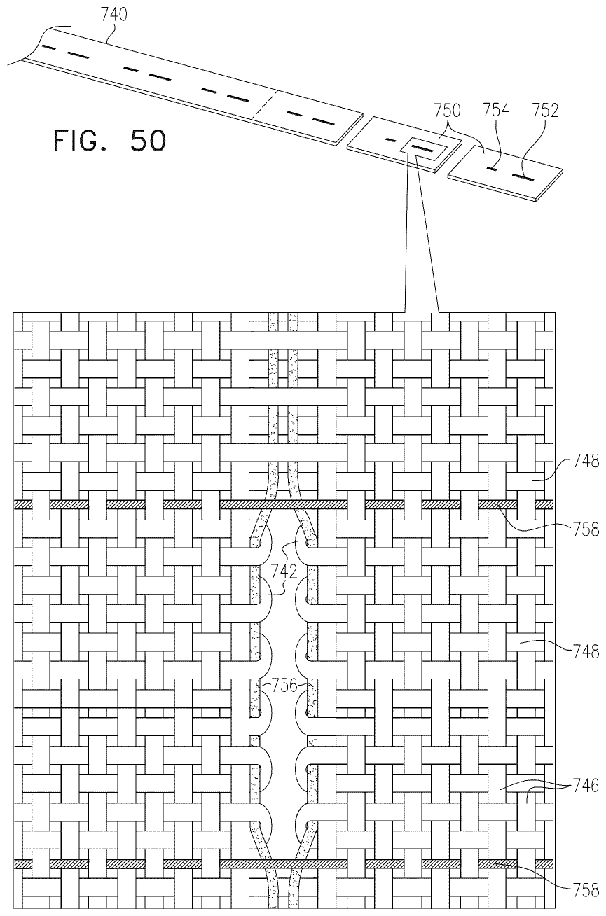

FIG. 50 shows a textile component 750 that is substantially planar (e.g., a strip of the textile) having slits (or other apertures) therein, of which one, e.g., a slit 752—will serve as the collar-aperture and the other, i.e., a slit 754—will serve as the eyelet-aperture. In some implementations, and as shown, component 750 is manufactured using a technique similar to that described with reference to FIG. 48, whereby multiple components 750 are cut from an elongate form 740 that is woven to be substantially planar, e.g., a ribbon. In some implementations, each textile component 750 is woven individually, obviating the need for a cutting step that separates an initial elongate form into individual textile components, e.g., the weaving initiates and terminates in a manner that defines the ends of each textile component.

Slits 752 and 754 can be collinear with each other. Slits 752 and 754 can lie on the long axis of textile component 750. Slits 752 and 754 can be oriented parallel with warp strands 746 of textile component 750.

Slits 752 and 754 can be formed by cutting, e.g., using the same technique that separates form 740 into components 750, such as laser cutting. However, in some implementations, and as shown, the slits can be formed by the weaving itself. For example, partway across textile component 750, weft strands can double-back rather than continuing across the textile component, e.g., as indicated by reference numeral 742.

In some implementations, in addition to components 750 comprising warp strands 746 and weft strands 748 that form the general structure of the components (i.e., general warp and weft strands), the components are woven to incorporate reinforcing warp strands 756 and/or reinforcing weft strands 758 positioned to skirt slit 752 and/or slit 754. For example, and as shown, a reinforcing warp strand 756 can run alongside one side of both slits, e.g., defining a respective side of each slit. Similarly, reinforcing weft strands 758 can run past the ends of the slits, e.g., each end of each slit can be defined at least in part by one of the reinforcing weft strands. Strands 756 can run the entire length of component 750. Strands 756 can run the entire width of component 750. Reinforcing warp strands 756 are stronger and fewer than general warp strands 746. Reinforcing weft strands 758 are stronger and fewer than general weft strands 748. The reinforcing strands can have a higher denier than the general strands. Although the reinforcing strands are shown as narrower than the general strands, they can alternatively be thicker than the general strands.

The reinforcing strands can be formed from the same substance (e.g., the same polymer) as the general strands, or can be formed from a different substance. Reinforcing strands 756 and 758 can be formed from a polymer or a metal. Reinforcing strands 756 and 758 can be monofilament strands, e.g., while general strands 746 and 748 can be polyfilament strands.

Alternatively to incorporating distinct reinforcing strands, components 750 can be woven to have a higher weave density at the edges and ends of the slits.

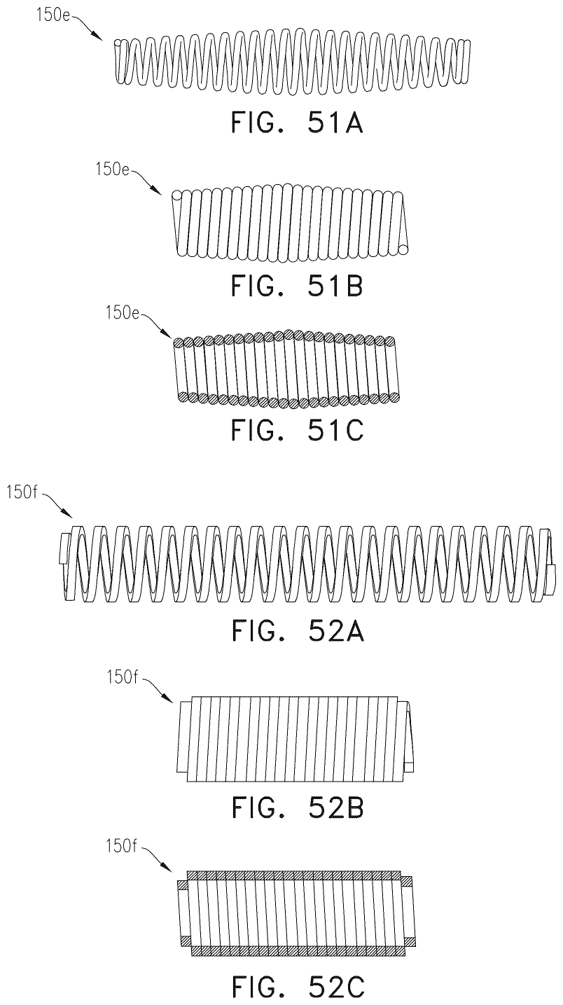

Reference is made to FIGS. 51A-C, 52A-C, 53, and 54, which are schematic illustrations of various spacers, in accordance with some implementations. Each of these spacers can be considered to be a variant of spacer 150 and can be used in place of spacer 150 or any of its variants. Any of the spacers described herein can be modified to include the features described with respect to these spacers.

Spacer 150e comprises a coil that is substantially helical but that is narrower at its ends than in its middle. FIG. 51A shows spacer 150e in its expanded state (e.g., at rest), FIG. 51B shows the spacer having been axially compressed (e.g., due to contraction of the implant), and FIG. 51C is a cross-sectional view of the state shown in FIG. 51B. In the example shown, the coil can be tapered from its middle towards its ends. The taper of the coil can be sufficiently shallow to prevent adjacent turns of the coil from axially overlapping upon axial compression of the spacer.

Other coil-based spacers described herein are shown as being formed from a wire having a substantially circular profile, e.g., such that a transverse cross-section through a turn of the coil is substantially circular. Spacer 150f comprises a coil that is non-circular in such transverse cross-section. FIG. 52A shows spacer 150f in its expanded state (e.g., at rest), FIG. 52B shows the spacer having been axially compressed (e.g., due to contraction of the implant), and FIG. 52C is a cross-sectional view of the state shown in FIG. 52B. In the example shown, a transverse cross-section through a turn of the coil of spacer 150f is quadrilateral, e.g., rectangular. In some implementations, a transverse cross-section through a turn of the coil of the spacer can be elliptical.

Spacers 150e and 150f may advantageously be more resistant buckling or collapsing during contraction of the implant/tensioning of the tether. For spacer 150e this may be at least partly due to increased rigidity toward the middle of the coil. Alternatively or additionally, for both of these spacers, the buckle-resistance may be due to stronger interactions between adjacent turns of the coil, and/or advantageous relative positioning between adjacent turns of the coil.

The coils of spacers 150e and 150f (as well as of the other coil-based spacers described herein) can be formed from a metal or from a polymer. The coil of spacer 150f can be formed from a wire that is bent into a coil, or can be formed from a tube that is laser-cut.

FIG. 53 shows a spacer 150g that is non-helical, but rather is a tube. The tube can be formed from a metal or a polymer, and can be flexible, e.g., e.g., can be made from flexible tubing. In some implementations, a sidewall of spacer 150g is inflatable (e.g., e.g., between its outer and inner surfaces) such that its flexibility and/or compressibility can be controlled by the degree to which it is inflated.

FIG. 54 shows a spacer 150h that is mounted on tether 112 such that it is disposed laterally from the tether. For example, and as shown, the spacer can have one or more laterally-positioned eyelets through which tether 112 is threaded. The body of spacer 150h can be inflatable, i.e., e.g., such that its flexibility and/or compressibility can be controlled by the degree to which it is inflated. Laterally-positioned eyelets such as those of spacer 150h can be added to any of the other spacers described herein, mutatis mutandis.

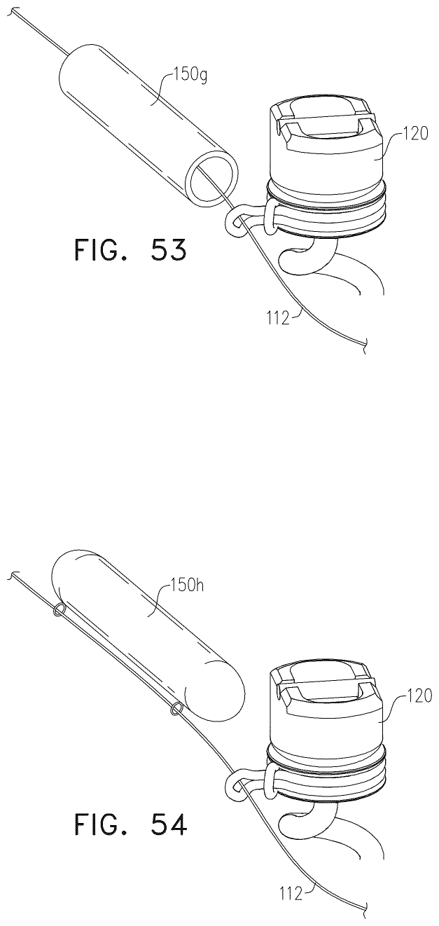

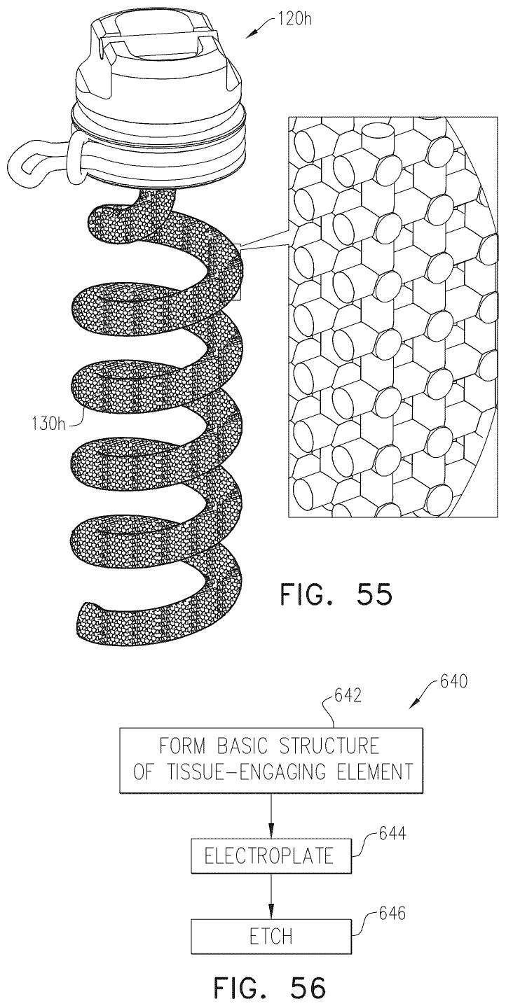

Reference is now made to FIGS. 55 and 56, which relate to tissue-engaging elements (e.g., variants of tissue-engaging element 130) having enhanced tissue-engaging properties, in accordance with some implementations. These properties can include enhanced surface interactions (e.g., gripping) and encouragement of tissue growth onto/into the tissue-engaging element. These tissue-engaging elements and their manufacturing techniques can be used for any of the anchors described herein, and also more broadly for the tissue-engaging element of other anchors, whether helical (e.g., screws) or non-helical (e.g., darts or staples).

FIG. 55 is a schematic illustration of an anchor 120h that comprises a tissue-engaging element 130h, in accordance with some implementations. Tissue-engaging element 130h is porous. It can be formed to be porous throughout its entirety. It can be formed via additive manufacturing (e.g., 3D-printing), such as powder bed fusion, electron beam melting, or powder-fed directed-energy deposition. Tissue-engaging element 130h can be formed from titanium or a titanium alloy, which may advantageously be stronger and/or more biocompatible than steel. Its porosity may advantageously encourage tissue growth throughout, and may also increase surface roughness and therefore initial gripping of the tissue.

FIG. 56 is a flow chart showing at least some steps of a technique 640 for manufacturing a tissue-engaging element, in accordance with some implementations. The basic or underlying structure of the tissue-engaging element is first formed (step 642), such as by bending (e.g., a wire), molding, or cutting (e.g., a tube). This step may simply be the way in which any of the other tissue-engaging elements described herein are manufactured. The tissue-engaging element (or part thereof) is then coated (e.g., plated, such as electroplated) with a coating material that is different to the structural material from which the basic structure is formed (step 644). The coating material can, for example, be gold.

The coating is then etched in a manner that provides the tissue-engaging element with a textured surface (step 646). Etching techniques that can be used for step 646 include, but are not limited to, laser etching or ion-beam etching. The etching can be sufficiently shallow that the coating is not completely penetrated, e.g., such that the structural material remains entirely obscured by the coating material. The resulting textured surface can advantageously encourage tissue growth, and can also increase surface roughness and therefore initial gripping of the tissue.

It is to be noted that technique 640 can be used with tissue-engaging elements other than those described herein, including helical tissue-engaging elements (e.g., screws) or non-helical tissue-engaging elements (e.g., darts or staples).

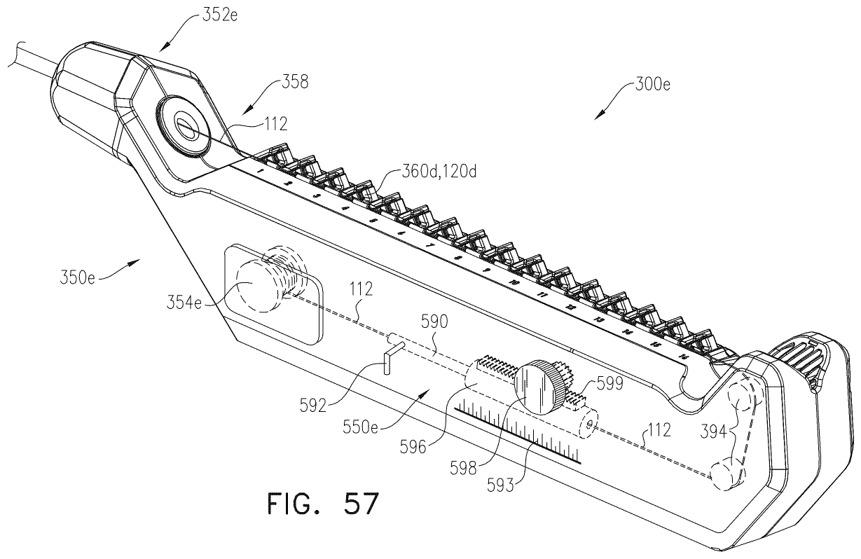

Reference is now made to FIG. 57, which is a schematic illustration of a catheter device 300e whose extracorporeal unit 350e comprises an integrated tensioner 550e, in accordance with some implementations. Like other tensioners 550 described herein (e.g., with reference to FIGS. 28-30), tensioner 550e can be operated to apply tension to tether 112, and to measure the tension. This can be utilized in order to make intraprocedural decisions, e.g., regarding the number and/or positioning of anchors and/or spacers.

In some implementations, as noted for other tensioners, this can also be utilized for the final tensioning of tether 112, during which the tension is locked into the tether using a lock and excess tether is trimmed. For example, while the proximal end of tether 112 remains engaged by extracorporeal unit 350e, a lock that is configured to receive tether 112 without access to an end of the tether (e.g., lock 160b, 160c, or 160k) can be added to the tether (e.g., at manipulation zone 358), advanced distally over and along the tether and through tube 310 to the final anchor of the implant and, while tensioner 550e applies tension to the tether, locked to the tether.

In some implementations, tensioner 550e can further facilitate differential tensioning of the implant being implanted, e.g., with tension being applied and locked into the tether after a first subset of anchors have been anchored (e.g., defining a first portion of the implant having a first tension and contracting a first portion of the tissue), and then doing the same for one or more subsequent subsets of anchors (e.g., defining one or more subsequent portions of the implant having different tensions and contracting respective other portions of the tissue). Therefore, catheter device 300e can be used in combination with, and/or to facilitate, systems and/or techniques described in Provisional U.S. Patent Application 63/370,609 to Biran et al., filed Aug. 5, 2022, and titled “Variable tissue contraction;” and/or PCT Publication WO 2023/228098 to Guerrero et al., filed May 24, 2023, and titled “Variable tissue contraction,” each of which is incorporated herein by reference.

In some implementations, tensioner 550e comprises a tensioning block 590 to which a clamp 592 is attached. In some implementations, tensioner 550e further comprises a tensioning controller (e.g., knob) 598 that is operable (e.g., hand-operable) by the operator (e.g., physician). In the example shown, tether 112 extends axially through tensioning block 590, which can have a long axis that is substantially coaxial or parallel with the segment of tether 112 disposed through the tensioning block (e.g., as shown).

In some implementations, extracorporeal unit 350e comprises a de-slacker 354e, which can be the same as or similar to other de-slackers herein, mutatis mutandis. In some implementations, de-slacker 354e can be disposed toward the distal end of extracorporeal unit 350e in order to allow the body 352e of the extracorporeal unit to accommodate tensioner 550e on tether 112.

In some implementations, extracorporeal unit 350e can comprise one or more bearings 394 (e.g., rollers) to redirect tether 112 distally within body 352. For example, and as shown, the proximal portion of tether 112 can pass proximally via aperture 382 (not visible in FIG. 57) into body 352e and the, within body 352e, around bearing(s) 394, distally through tensioner 550e, and further distally to de-slacker 354e. Thus, along tether 112, tensioner 550e is disposed between de-slacker 354e and the cartridges (e.g., cartridges 360d) housing the anchors (e.g., anchors 120d).

In some implementations, during advancement and anchoring of the anchors of the implant, clamp 592 is open, effectively making the tensioner transparent to tether 112 such that de-slacker 354e can perform its function (e.g., reduce or eliminate slack in the tether). In order to functionalize tensioner 550e, clamp 592 is operated to clamp tether 112 to tensioning block 590.

In some implementations, once tensioner 550e has been functionalized, in order apply tension to tether 112, the operator operates tensioning controller 598, which is operatively coupled to tensioning block 590 such that operation of the tensioning controller drives the tensioning block, which is clamped to the tether and therefore pulls the tether. In the example shown, tensioning controller 598 is a tensioning knob that is operated by rotation, which drives tensioning block 590 via threading 599 (e.g., a rack-and-pinion arrangement) that provides a linear actuator functionality. That is, in some implementations, the tensioner 550e can comprise a linear actuator. However, it is to be understood that other controller and/or actuator types can be used.

In some implementations, extracorporeal unit 350e (e.g., tensioner 550e thereof) can include an indicator 593. Indicator 593 is schematically illustrated as a simple scale, but can take other forms and/or can indicate more than one value. For example, indicator 593 can be or include a distance indicator that indicates a distance by which tether 112 has been pulled by the tensioner (e.g., analogous to distance indicator 463, described hereinabove), and/or can be or include a tension indicator that indicates the magnitude of the tension that has been applied to tether 112 by the tensioner (e.g., analogous to tension indicator 461, described hereinabove).

In some implementations, as described for the tensioning subassembly of extracorporeal unit 450, the magnitude of the tension on tether 112 can be determined by tensioning block 590 being spring-coupled to a stock 596 that is driven by tensioning controller 598 (e.g., via the threaded mating described above), such that operation of the tensioning controller drives the stock that, via the spring-coupling, pushes the tensioning block, whose position relative to the stock is indicative of indicative of strain on the spring-coupling and therefore of tension on tether 112.

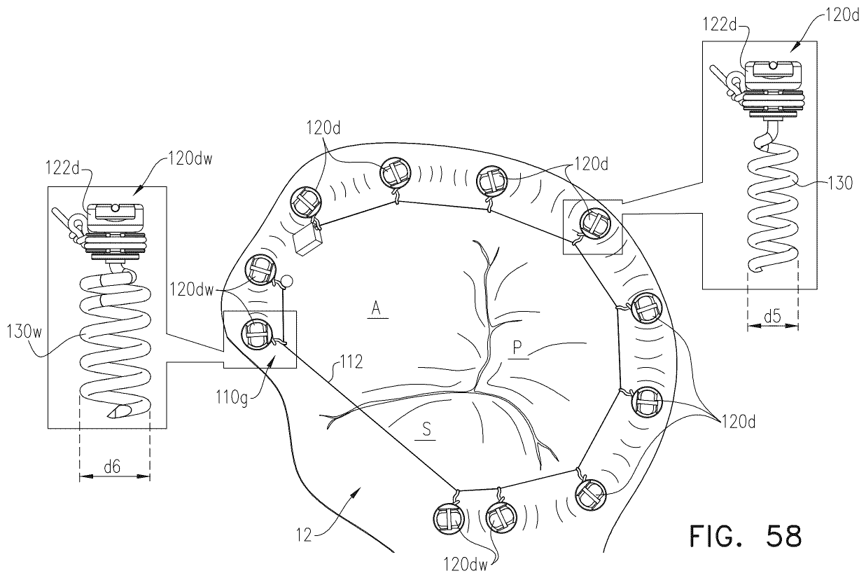

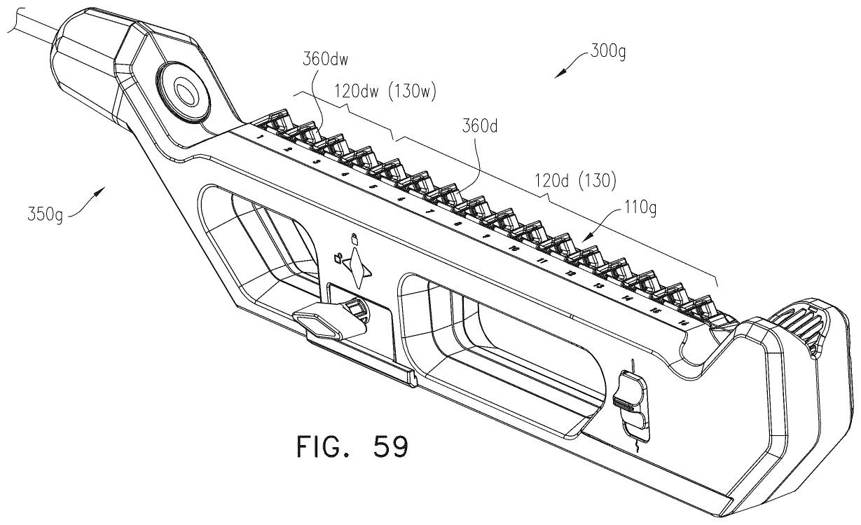

Reference is now made to FIGS. 58-59, which are schematic illustrations of an implant 110g, in accordance with some implementations. In some implementations, it may be desirable that the anchors of an implant 110 have different tissue-engaging elements optimized for different sites along the tissue at which the implant is to be implanted, e.g., around the annulus. For example, several regions of the valve annulus may be proximate to highly sensitive tissues such as coronary blood vessels or conductive tissue, and thus a tissue-engaging element having a modest width and/or length may be desirable in these regions. Conversely, a site that is free from highly sensitive tissues may be suited to receiving a larger tissue-engaging element. Furthermore, the greater anchoring strength provided by a larger tissue-engaging element may be particularly desirable if the anchor is expected to encounter increased pulling force from tether 112, e.g., force greater than that encountered by other anchors.

FIG. 58 shows an implant 110g, which comprises anchors 120d having a tissue-engaging element with first diameter as well as anchors 120dw having a tissue-engaging element with a second diameter, the second diameter wider than the first diameter. Although implant 110g is broadly shown as a variant of implant 110d, it is to be understood as a general example of how an implant can be configured and/or implanted. For the sake of clarity, implant 110g is shown in FIG. 58 without spacers, but it is to be understood that the implant may, indeed, include spacers—e.g., as described herein for other implants 110.

Implant 110g comprises multiple anchors 120d (described hereinabove), each of which comprises a tissue-engaging element 130 (also described hereinabove), which has a width d5. In contrast to implant 110d (and other anchors described herein), implant 110g further includes one or more anchors 120dw. As shown in FIG. 38, anchor 120dw has a tissue-engaging element 130w that has a width d6 that is greater than width d5.

In some implementations, one or more anchors 120dw can comprise a first subset of the anchors of implant 110g (i.e., the leading anchor and the successive anchors) that have a tissue-engaging element 130w. In some implementations, the first subset of the anchors can contain the first 2-6 anchors 120dw of the implant.

In some implementations, and as shown, other than their different widths, tissue-engaging element 130 and tissue-engaging element 130w are shaped the same as each other, e.g., they are both helical and/or have the same number of helical turns and/or the same pitch, and can be otherwise identical.

In the example shown, the first four anchors of implant 110g are anchors 120dw (i.e., the first subset contains four anchors 120dw), and the remainder of the anchors (e.g., a second subset of anchors) are anchors 120d. In some implementations, the second subset can contain more anchors than the first subset. For example, the second subset can contain 4-18 (e.g., 6-16, e.g., 8-14, e.g., 10-14) anchors 120d. In the example shown, the second subset contains 12 anchors 120d.

FIG. 59 shows implant 110g mounted on the extracorporeal unit 350g of a catheter tool 300g. In some implementations, and as shown, extracorporeal unit 350g is a variant of extracorporeal unit 350d, in which the first (e.g., distalmost) 2-6 cartridges are cartridges 360dw, specifically configured to house anchors 120dw, while the remainder of the cartridges are cartridges 360d, described hereinabove. However, in some implementations, the catheter device (e.g., the extracorporeal unit thereof) may not be tailored to the different tissue-engaging elements, e.g., each cartridge can accommodate anchors having different tissue-engaging elements.

For example, in some implementations, implant 110g can be implanted with one or more (e.g., two) anchors 120dw anchored to the annulus in a region that is the vicinity of the root of the anterior leaflet A of the tricuspid valve, and the next one or more (e.g., two) anchors 120dw anchored to the annulus in a region that is the vicinity of the root of the septal leaflet L of the tricuspid valve, such that tether 112 spans, unanchored, the annulus between these two regions, e.g., the annulus that is in the vicinity of the commissure between the anterior and septal leaflets and/or the aortic valve.

In the example shown in FIG. 58, the regions in which anchors 120dw are anchored have fewer highly sensitive tissues and/or are capable of withstanding greater pulling forces, whereas the region spanned unanchored is richer in highly sensitive tissues and/or is less capable of withstanding greater pulling forces. The increased anchoring strength provided by anchors 120dw and the tissues of the regions at which they are anchored may therefore advantageously facilitate the omission of anchors in the region therebetween.

Providing an implant that includes leading 2-6 anchors having a larger tissue-engaging element, with the remainder of the successive anchors having a more modestly-sized tissue-engaging element, may advantageously provide the operator and the procedure with flexibility even for an implant that, like those described herein, is provided with its anchors already threaded on tether 112.

For example, if it is determined that it would be preferable to avoid anchoring the implant in a particular region, the procedure can commence with the first one or more anchors 120dw being anchored on one side of that region. The procedure can continue with the next one or more anchors 120dw being anchored on the other side of that region such that the tether spans that region unanchored. The procedure can further continue with anchors 120d distributed along the remainer of the tissue along which the implant is to be secured. It is to be noted that, as for other implants described herein, the number of anchors 120d to be used can be decided according to the specific anatomy and procedure.

The systems, tools, implants, and techniques disclosed herein are described generally with respect to annuloplasty techniques in which an implant/tether is implanted along the annulus of a heart valve such that the implant/tether is arranged to circumscribe at least part of the valve. However, it is to be noted that the scope of the present disclosure includes utilization and/or modification of these systems, tools, implants, and techniques for other implantation arrangements, such as the implant/tether spanning across the orifice of the valve (e.g., to pull opposite sides of the annulus, and thereby opposing leaflets, toward each other), or even non-valvular implantation. In some implementations, the implant used can have more anchors or fewer anchors than those implants illustrated. Accordingly, the catheter device used (e.g., the extracorporeal unit thereof) can have fewer cartridges/anchor holders. For example, such implants can comprise 3-10 (e.g., 4-8) anchors and such catheter devices can comprise 3-10 (e.g., 4-8) cartridges.

It is to be noted that, although system 1000 (e.g., system 100d thereof) is shown as comprising implant 110d, it can be modified to usable and/or for use with other implants including, but not limited to, other implants 110 described herein. For example, catheter device 300 can, mutatis mutandis, be loaded with, and used to facilitate implantation of, other implants, such as other implants 110 described herein and/or those described in one or more of the following references, each of which is incorporated herein by reference: WO 2021/084407 to Kasher et al.; WO 2022/172149 to Shafigh et al; WO 2014/064694 to Sheps et al., and WO 2016/174669 to Iflah et al.

Reference is again made to FIGS. 1-56. The various systems, devices, apparatuses, etc. in this disclosure can be sterilized (e.g., with heat, radiation, ethylene oxide, hydrogen peroxide, etc.) to ensure they are safe for use with patients, and the methods herein can comprise (or consist of) such sterilization of the associated system, device, apparatus, etc. Furthermore, the scope of the present disclosure includes, in some implementations, sterilizing one or more of any of the various systems, devices, apparatuses, etc. in this disclosure.

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation, such as a cadaver, cadaver heart, simulator, imaginary person, etc. When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Various implementations or applications of systems, devices, methods, etc. are disclosed herein, and any combination of their features, components, and options can be made unless specifically excluded. For example, a given implant can comprise any of the anchors (or a combination thereof) and/or any of the locks described herein. Furthermore, the catheter devices and adjustment tools that are described for use with a given implant may alternatively be used, mutatis mutandis, with a different implant described herein. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

### Example Implementations (Some Non-Limiting Examples of the Concepts Herein are Recited Below):

Example 1. A system useable and/or for use with a real or simulated tissue of a real or simulated subject, the system comprising: (A) a catheter device, comprising: (i) a flexible tube that has: (a) a distal opening that is configured to be transluminally advanced toward the tissue, and/or (b) a proximal end that defines a proximal opening; and/or (ii) an extracorporeal unit, coupled to the proximal end of the tube, and/or comprising: (a) a body, and/or (b) a series of cartridges, distributed along or parallel to a proximal-distal axis of the body, with a distalmost cartridge of the series of cartridges being closest to the proximal opening; (B) a tether; and/or (C) a series of anchors, including a leading anchor and other anchors, each anchor of the series of anchors: (i) housed by a corresponding cartridge of the series of cartridges, with the leading anchor housed by the distalmost cartridge, and/or (ii) coupled to the tether such that the tether extends along the body, parallel with the proximal-distal axis.

Example 2. The system according to example 1, wherein the cartridges of the series of cartridges are imbricated.

Example 3. The system according to any one of examples 1-2, wherein the anchors of the series of anchors are imbricated.

Example 4. The system according to any one of examples 1-3, wherein the system is sterilized.

Example 5. The system according to any one of examples 1-4, wherein the catheter device is sterilized.

Example 6. The system according to any one of examples 1-5, wherein the flexible tube is flared toward the distal opening.

Example 7. The system according to any one of examples 1-6, wherein at least some of the anchors of the series each comprise: (i) an anchor head; (ii) a tissue-engaging element: (a) extending distally away from the anchor head to define an anchor axis of the anchor, and/or (b) configured to be driven along the anchor axis into the tissue; and/or (iii) a textile and/or polymer, shaped to define an eyelet threaded onto the tether in a manner that slidably couples the anchor to the tether.

Example 8. The system according to any one of examples 1-7, wherein each of the cartridges: (i) has a closed state in which the cartridge securely houses the corresponding anchor, (ii) defines a respective cartridge vector that is oblique with respect to the proximal-distal axis, and/or (iii) is, by at least part of the cartridge being slid along the cartridge vector, transitionable into an open state in which the corresponding anchor is removable from the cartridge.

Example 9. The system according to example 8, wherein the cartridge defines a threshold force, and/or is configured to transition into the open state upon the anchor being pulled with a pulling force that exceeds the threshold force.

Example 10. The system according to example 8, wherein the cartridge is configured to resist returning from the open state into the closed state.

Example 11. The system according to example 8, wherein the cartridge vector is oblique with respect to the proximal-distal axis.

Example 12. The system according to example 8, wherein the cartridge vectors of the series of cartridges collectively define a common cartridge plane on which the cartridge vectors lie.

Example 13. The system according to example 12, wherein the proximal-distal axis is parallel with the common cartridge plane.

Example 14. The system according to example 12, wherein the proximal-distal axis lies on the common cartridge plane.

Example 15. The system according to example 12, wherein the tether extends along the body, parallel with the common cartridge plane.

Example 16. The system according to any one of examples 1-15, wherein each anchor of the series: (i) comprises: (a) a head, coupled to the tether, and/or (b) a tissue-engaging element, extending away from the head to define an anchor axis of the anchor, and/or (ii) is housed by a corresponding cartridge such that the anchor axis lies obliquely with respect to the proximal-distal axis.

Example 17. The system according to example 16, wherein, for each anchor of the series of anchors, the anchor is oriented with the head proximal from the tissue-engaging element.

Example 18. The system according to example 16, wherein, for each anchor of the series of anchors, the anchor is oriented with the tissue-engaging element closer than the head to the proximal opening.

Example 19. The system according to example 16, wherein the anchor axes of the series of anchors collectively define a common anchor plane on which the anchor axes lie.

Example 20. The system according to example 19, wherein the proximal-distal axis is parallel with the common anchor plane.

Example 21. The system according to example 19, wherein the proximal-distal axis lies on the common anchor plane.

Example 22. The system according to example 19, wherein the tether extends, along the extracorporeal unit, parallel with the common anchor plane.

Example 23. The system according to any one of examples 1-22, wherein the tether has (i) a distal end at the leading anchor, and/or (ii) a proximal end releasably secured within the extracorporeal unit.

Example 24. The system according to example 23, wherein the extracorporeal unit comprises a de-slacker that comprises a winch that is spring-loaded in a manner that takes up slack in the tether.

Example 25. The system according to example 24, wherein the de-slacker comprises a deactivation switch that is user-operable to deactivate the de-slacker in a manner that allows slack to be introduced to the tether and not taken up by the winch.

Example 26. The system according to any one of examples 1-25, further comprising multiple spacers threaded on the tether, alternatingly with the anchors of the series.

Example 27. The system according to example 26, further comprising at least one free spacer, separate from the tether, and/or manually threadable onto the tether between anchors without access to an end of the tether.

Example 28. The system according to example 26, wherein each of the spacers is tubular, and/or is threaded on the tether by the tether extending through a lumen defined by the spacer.

Example 29. The system according to example 28, wherein the spacer is substantially axially incompressible.

Example 30. The system according to example 28, wherein the spacer comprises a flexible sleeve that is substantially axially compressible.

Example 31. The system according to example 28, wherein the spacer comprises a fabric tube.

Example 32. The system according to example 28, wherein the spacer has a sidewall that is inflatable in a manner that adjusts a compressibility of the spacer.

Example 33. The system according to example 26, wherein each of the spacers is a ribbon, and/or is threaded on the tether by the tether weaving along the ribbon.

Example 34. The system according to example 26, wherein each of the spacers is inflatable in a manner that adjusts a compressibility of the spacer.

Example 35. The system according to example 26, wherein each of the spacers has a body and one or more laterally-positioned eyelets through which the tether is threaded such that the body is mounted laterally from the tether.

Example 36. The system according to example 26, further comprising multiple connectors, each connecting a corresponding one of the spacers to a corresponding anchor of the series.

Example 37. The system according to example 36, further comprising a disconnector, housed within and removable from a compartment in the extracorporeal unit, and/or configured to cut one or more of the connectors.

Example 38. The system according to example 26, wherein each of the connectors provides a frangible connection between the corresponding spacer and the corresponding anchor.

Example 39. The system according to example 38, wherein the frangible connection is configured to be broken by pulling the connector away from the corresponding spacer.

Example 40. The system according to example 38, wherein each of the spacers comprises a helical coil, and/or wherein the frangible connection is provided by the connector being tucked between adjacent turns of the helical coil of the corresponding spacer.

Example 41. The system according to example 38, wherein each of the spacers comprises a helical coil, and/or wherein the frangible connection is configured to be broken by rotating the spacer to unwind the helical coil from the corresponding connector.

Example 42. The system according to example 36, wherein each of the spacers is arranged on the tether such that, upon advancement of the corresponding anchor distally along the tether toward the proximal opening, the spacer trails the anchor.

Example 43. The system according to example 42, wherein a first of the spacers is connected to the leading anchor and is less axially compressible than at least another of the spacers.

Example 44. The system according to any one of examples 1-43, wherein: (i) each anchor of the series comprises: (a) an anchor head; and/or (b) a helical tissue-engaging element, extending away from the anchor head to define an anchor axis of the anchor, and/or configured to be screwed along the anchor axis into the tissue; (ii) the flexible tube has a distal portion that includes the distal opening, the flexible tube defining: (a) along a tube axis of the flexible tube, a channel through which the anchor is slidable toward the distal opening, and/or (b) at the distal portion, a grip zone at which the flexible tube has a grip surface that inhibits sliding of the anchor through the grip zone by gripping a lateral surface of the helical tissue-engaging element; and/or (iii) the system further comprises an anchor driver configured to: (A) slide the anchor distally through the channel to the grip zone, and/or (B) drive the anchor through the grip zone by screwing the helical tissue-engaging element over the grip surface.

Example 45. The system according to example 44, wherein the grip surface is configured such that, as the driver screws the helical tissue-engaging element over the grip surface, the helical tissue-engaging element temporarily compresses parts of the grip surface with which the helical tissue-engaging element is in contact.

Example 46. The system according to example 44, wherein the grip surface comprises and/or is formed from a polymer.

Example 47. The system according to example 46, wherein the flexible tube is lined with the polymer.

Example 48. The system according to example 46, wherein the flexible tube comprises and/or is formed from the polymer.

Example 49. The system according to example 46, wherein the polymer is a thermoplastic elastomer.

Example 50. The system according to example 46, wherein the polymer is a block copolymer.

Example 51. The system according to example 50, wherein the block copolymer is polyether block amide.

Example 52. The system according to example 44, wherein the grip surface is provided by at least one resilient nub that protrudes medially into the channel.

Example 53. The system according to example 44, wherein the grip surface is provided by at least one resilient rib that protrudes medially into the channel.

Example 54. The system according to example 53, wherein: (i) the rib extends medially into the channel in a manner that defines, adjacent the rib, a niche in the grip zone, and/or (ii) the system is configured such that, while the anchor driver screws the helical tissue-engaging element over the grip surface: (a) the rib excludes the helical tissue-engaging element from the niche, and/or (b) the tether extends through the grip zone sheltered within the niche, laterally from the helical tissue-engaging element.

Example 55. The system according to example 54, wherein: (i) the anchor further comprises an eyelet, mounted on the anchor head so as to be revolvable about the anchor axis, and/or (ii) proximal from the rib, the flexible tube further defines an abutment that protrudes medially into the channel in a manner that, as the anchor driver screws the tissue-engaging element over the grip surface, inhibits revolution of the eyelet about the anchor axis.

Example 56. The system according to example 55, wherein a unitary structure defines both the rib and the abutment.

Example 57. The system according to example 55, wherein the rib protrudes further medially into the channel than the abutment.

Example 58. The system according to example 55, wherein the abutment is longer, along the channel, than the rib.

Example 59. The system according to example 53, wherein the rib has a proximal face that is shaped to define a shoulder.

Example 60. The system according to example 53, wherein the rib has a distal face that is tapered.

Example 61. The system according to example 53, wherein the rib is a first of multiple ribs defined by the distal portion in the grip zone.

Example 62. The system according to example 61, wherein the multiple ribs are exactly two ribs.

Example 63. The system according to example 61, wherein the multiple ribs are exactly three ribs.

Example 64. The system according to example 61, wherein the multiple ribs are exactly four ribs.

Example 65. The system according to example 61, wherein the multiple ribs are exactly five ribs.

Example 66. The system according to example 61, wherein the multiple ribs are exactly six ribs.

Example 67. The system according to example 61, wherein the multiple ribs are distributed circumferentially around the tube axis.

Example 68. The system according to example 61, wherein the multiple ribs are distributed along the tube axis.

Example 69. The system according to example 53, wherein the rib extends around at least part of the tube axis.

Example 70. The system according to example 69, wherein the rib is toroidal, extending circumferentially around the entire tube axis.

Example 71. The system according to example 53, wherein the rib extends alongside the tube axis.

Example 72. The system according to example 71, wherein the rib is parallel with the tube axis.

Example 73. The system according to any one of examples 1-72, wherein, each of the anchors comprises: (a) an anchor head; (b) a tissue-engaging element: (i) extending distally away from the anchor head to define an anchor axis of the anchor, and/or (ii) configured to be driven along the anchor axis into the tissue; and/or (c) a textile and/or polymer, shaped to define an eyelet, the anchor being coupled to the tether by the eyelet being coupled to the tether.

Example 74. The system according to example 73, wherein, for at least some of the anchors, the textile and/or polymer is a unitary piece of textile/polymer that is further shaped to define a spacer that extends away from the anchor head along the tether.

Example 75. The system according to example 74, wherein the unitary piece of textile/polymer is a strip along which the tether is woven.

Example 76. The system according to example 75, wherein the unitary piece of textile/polymer further defines a collar that rotatably couples the unitary piece of textile/polymer to the anchor such that the eyelet and the spacer are revolvable around the anchor axis.

Example 77. The system according to example 76, wherein the unitary piece of textile/polymer further defines a narrowed neck between the collar and the spacer that facilitates pivoting of the spacer.

Example 78. The system according to example 74, wherein the spacer extends away from the anchor head along the tether toward a subsequent anchor of the series.

Example 79. The system according to example 73, wherein the tissue-engaging element is porous.

Example 80. The system according to example 79, wherein the tissue-engaging element is formed to be helical and porous via additive manufacturing.

Example 81. The system according to example 80, wherein the tissue-engaging element comprises and/or is formed from titanium.

Example 82. The system according to example 73, wherein the tissue-engaging element comprises and/or is formed from a structural material and has a coating with which the structural material is coated, the coating being (i) of a material other than the structural material, and/or (ii) etched in a manner that provides the tissue-engaging element with a textured surface.

Example 83. The system according to example 82, wherein the coating is a gold coating.

Example 84. The system according to example 82, wherein the coating is laser etched.

Example 85. The system according to example 82, wherein the coating is ion-beam etched.

Example 86. The system according to example 73, further comprising multiple spacers threaded on the tether, each of the spacers connected via a corresponding connector to a corresponding anchor of the series.

Example 87. The system according to example 86, wherein, for at least some of the spacers, the spacer is a textile spacer.

Example 88. The system according to example 87, wherein the spacer is formed from the same textile as the eyelet.

Example 89. The system according to example 86, wherein, for at least some of the spacers, the spacer comprises a coil that is substantially helical.

Example 90. The system according to example 89, wherein, for at least some of the spacers, the coil is formed from a wire that is bent to define the coil such that a transverse cross-section through a turn of the coil is substantially elliptical.

Example 91. The system according to example 89, wherein, for at least some of the spacers, the coil comprises and/or is formed from a tube that is cut to define the coil such that a transverse cross-section through a turn of the coil is substantially quadrilateral.

Example 92. The system according to example 89, wherein, for at least some of the spacers, the spacer has a first end, a second end, and/or a middle therebetween, the first end and the second end being narrower than the middle.

Example 93. The system according to example 89, wherein, for at least some of the spacers, the spacer is tapered from its middle towards its ends.

Example 94. The system according to example 93, wherein the taper is sufficiently shallow to prevent adjacent turns of the coil from axially overlapping upon axial compression of the spacer.

Example 95. The system according to example 89, wherein the coil comprises and/or is formed from a metal.

Example 96. The system according to example 89, wherein the coil comprises and/or is formed from a polymer.

Example 97. The system according to example 86, wherein, for each of the anchors, the textile also defines at least part of the corresponding connector.

Example 98. The system according to example 97, wherein, for each of the anchors, the textile also defines at least part of the corresponding spacer.

Example 99. The system according to example 86, wherein each of the spacers is arranged on the tether such that, upon advancement of the corresponding anchor distally along the tether toward the proximal opening, the spacer trails the anchor.

Example 100. The system according to example 73, wherein the anchor head comprises an interface that is coupled to the tissue-engaging element, the tissue-engaging element being configured to be driven along the anchor axis into the tissue by an anchoring force applied to the interface.

Example 101. The system according to example 100, wherein the anchor further comprises a snood disposed around the anchor head in a manner that preserves accessibility to the interface.

Example 102. The system according to example 101, wherein the snood is absorbent.

Example 103. The system according to example 101, wherein the snood comprises a sponge.

Example 104. The system according to example 101, wherein the snood comprises a multilaminar material.

Example 105. The system according to example 101, wherein the snood comprises cellulose sheets.

Example 106. The system according to example 101, wherein the snood is impregnated with a substance and is configured to progressively release the substance within the subject.

Example 107. The system according to example 106, wherein the substance comprises a medicament.

Example 108. The system according to example 106, wherein the substance comprises a radiopaque dye.

Example 109. The system according to example 73, wherein the textile and/or polymer is shaped such that the eyelet is pivotable over the anchor head.

Example 110. The system according to example 73, wherein the textile is a fabric.

Example 111. The system according to example 73, wherein the textile comprises filaments of a synthetic polymer.

Example 112. The system according to example 73, wherein the textile comprises filaments of a natural fiber.

Example 113. The system according to example 73, wherein the textile is a yarn.

Example 114. The system according to example 113, wherein the eyelet is formed by securing the yarn into a loop.

Example 115. The system according to example 73, wherein, for each of the anchors, the textile and/or polymer is further shaped to define a collar that couples the eyelet to the anchor head.

Example 116. The system according to example 115, wherein the textile and/or polymer forms two rings, one of the rings serving as the collar and the other of the rings serving as the eyelet, the eyelet and the collar being interlinked by passing the rings through and around each other.

Example 117. The system according to example 115, wherein the textile and/or polymer is a fabric.

Example 118. The system according to example 117, wherein the fabric is a substantially flat fabric sheet.

Example 119. The system according to example 118, wherein the collar and the eyelet are formed by cutting the fabric sheet.

Example 120. The system according to example 117, wherein the fabric is woven in a manner that integrally defines the collar and the eyelet.

Example 121. The system according to example 120, wherein: (i) the eyelet has an eyelet-aperture through the fabric, (ii) the collar has a collar-aperture through the fabric, and/or (iii) the fabric is woven in a manner that provides the eyelet-aperture and the collar-aperture.

Example 122. The system according to example 121, wherein the fabric has general warp strands and reinforcement warp strands, the reinforcement warp strands being stronger and fewer than the general warp strands, and/or the fabric being woven such that the reinforcement warp strands skirt the eyelet-aperture and the collar-aperture.

Example 123. The system according to example 122, wherein the fabric has general weft strands and reinforcement weft strands, the reinforcement weft strands being stronger and fewer than the general weft strands, and/or the fabric being woven such that the reinforcement weft strands skirt the eyelet-aperture and the collar-aperture.

Example 124. The system according to example 115, wherein the textile is further shaped to define a spacer through which the tether is threaded, the spacer inhibiting approximation between the anchor and an adjacent anchor of the series.

Example 125. The system according to example 115, wherein the textile comprises a textile tube that is (i) wrapped around the anchor head in a manner that defines the collar, (ii) formed into a loop in a manner that defines the eyelet, and/or (iii) threaded coaxially onto the tether in a manner that defines the spacer.

Example 126. The system according to example 115, wherein the textile is a yarn.

Example 127. The system according to example 126, wherein the collar and the eyelet are defined by respective loops of the yarn.

Example 128. The system according to example 126, wherein the collar and the eyelet are formed integrally during formation of the yarn.

Example 129. The system according to example 126, wherein the collar and the eyelet are formed by knotting the yarn.

Example 130. The system according to example 126, wherein the collar is formed by securing the yarn into a loop.

Example 131. The system according to example 126, wherein the eyelet is formed by securing the yarn into a loop.

Example 132. The system according to example 115, wherein the collar and the eyelet are formed integrally during formation of the textile.

Example 133. The system according to example 115, wherein the eyelet is revolvable about the anchor axis by the collar rotating about the anchor axis.

Example 134. The system according to example 115, wherein the eyelet is connected to two places on the collar in a manner that defines a hinge axis on which the two places lie, and/or wherein the eyelet is pivotable about the hinge axis.

Example 135. The system according to any one of examples 1-134, wherein the catheter device further comprises a de-slacker, coupled to the tether, and/or configured to eliminate slack in the tether.

Example 136. The system according to example 135, wherein the de-slacker is disposed at a proximal part of the extracorporeal unit.

Example 137. The system according to example 135, wherein the extracorporeal unit defines an aperture, the tether extending from the de-slacker, through the aperture and along the body.

Example 138. The system according to example 137, wherein the aperture faces along the series of cartridges to the proximal opening.

Example 139. The system according to example 137, wherein the aperture is aligned with the proximal opening.

Example 140. The system according to any one of examples 1-139, further comprising an anchor driver: (a) comprising a flexible shaft, and/or a drive head at a distal end of the shaft, and/or (b) configured to, for each of the anchors sequentially, beginning with the leading anchor: (i) engage the drive head with the anchor, (ii) remove the anchor from the corresponding cartridge, and/or (iii) while the anchor remains coupled to the tether, advance the anchor into the proximal opening and through the flexible tube toward the tissue, and/or anchor the anchor to the tissue.

Example 141. The system according to example 140, wherein the extracorporeal unit is configured such that, for each of the anchors, removal, by the anchor driver, of the anchor from the corresponding cartridge moves the anchor away from the proximal-distal axis.

Example 142. The system according to example 140, wherein the extracorporeal unit is configured such that, for each of the anchors, removal, by the anchor driver, of the anchor from the corresponding cartridge pulls part of the tether away from the proximal-distal axis.

Example 143. The system according to example 140, wherein the tether extends along the body in a manner that defines a tether axis that is parallel with the proximal-distal axis, and/or wherein the extracorporeal unit is configured such that, for each of the anchors, removal, by the anchor driver, of the anchor from the corresponding cartridge pulls part of the tether away from the tether axis.

Example 144. The system according to example 140, wherein the tether extends along the body such that, along the body, the tether is straight, and/or wherein the extracorporeal unit is configured such that, for each of the anchors, removal, by the anchor driver, of the anchor from the corresponding cartridge reshapes part of the tether away from being straight.

Example 145. The system according to example 140, wherein the tether extends along the body of the extracorporeal unit such that, along the body, the tether is straight, and/or wherein the extracorporeal unit is configured such that, for each of the other anchors, removal, by the anchor driver, of the other anchor from the corresponding cartridge forms part of the tether into a V-shape.

Example 146. The system according to example 140, wherein, for each of the cartridges: (a) the cartridge comprises a chassis and a tray, (b) the cartridge has a closed state in which the cartridge securely houses the corresponding anchor, with the corresponding anchor seated in the tray, and/or (c) the anchor driver is configured to remove the corresponding anchor from the cartridge by applying a pulling force to the anchor such that the cartridge transitions into an open state by the tray sliding with respect to the chassis in a manner that exposes the corresponding anchor from the cartridge.

Example 147. The system according to example 146, wherein: (a) the system comprises multiple spacers threaded on the tether alternatingly with the anchors of the series such that each of the spacers is disposed adjacent to a corresponding cartridge of the series, and/or (b) for each of the cartridges: (i) the tray is shaped to define a catch that, in the closed state of the cartridge, obstructs the corresponding spacer from sliding distally away from the cartridge, and/or (ii) transitioning of the cartridge into the open state displaces the catch so that the catch ceases to obstruct the corresponding spacer from sliding distally away from the corresponding cartridge.

Example 148. The system according to example 146, wherein the cartridge defines a threshold force, and/or is configured to transition into the open state only upon the pulling force exceeding the threshold force.

Example 149. The system according to example 146, wherein the cartridge is configured to resist returning from the open state into the closed state.

Example 150. The system according to example 140, further comprising an elongate adjustment tool and a lock, the adjustment tool configured to: (a) advance the lock distally along the tether into a real or simulated heart of the subject and toward the tissue, (b) apply tension to the tether, (c) lock the tension in the tether by locking the lock to the tether, (d) cut the tether proximally from the lock, and/or (d) leave the lock in the heart locked to the tether.

Example 151. The system according to example 150, wherein the lock is configured to be placed onto and advanced along the tether by the adjustment tool without access to an end of the tether.

Example 152. The system according to example 151, wherein the lock comprises: (a) a frame; (b) a first set of hooked fingers extending from a first side of the frame toward a second side of the frame, the second side being opposite the first side; (c) a second set of hooked fingers extending from the second side toward the first side, the fingers of the second set arranged along the frame alternatingly with the fingers of the first set, wherein the lock: (i) has an unlocked state in which the frame is constrained to be narrowed and in which the tether is placeable and slidable between the fingers of the first and second sets, and/or (ii) is lockable to the tether by unconstraining the frame to widen such that the first and second sides of the frame responsively move away from each other, pulling with them the first and second sets of fingers, respectively.

Example 153. The system according to example 152, wherein the first and second sides of the frame, moving away from each other, pull with them the first and second sets of fingers such that the tether becomes clamped between the fingers of the first set and the fingers of the second set.

Example 154. The system according to example 152, wherein the first and second sides of the frame, moving away from each other, pull with them the first and second sets of fingers such that the tether becomes forced into a tortuous path.

Example 155. The system according to example 152, wherein the adjustment tool is configured to advance the lock distally along the tether into the heart of the subject and toward the tissue while maintaining the lock in the unlocked state by constraining the frame to be narrowed.

Example 156. The system according to example 150, wherein: (i) the extracorporeal unit comprises a catheter-device extracorporeal unit, (ii) the adjustment tool comprises an adjustment-tool extracorporeal unit, a shaft extending distally from the adjustment-tool extracorporeal unit, and/or a tool head at a distal end of the shaft, and/or (iii) the adjustment tool is configured to advance the lock distally along the tether into the heart and toward the tissue while the lock is housed within the tool head.

Example 157. The system according to example 156, wherein the tether has (i) a distal end at the leading anchor, and/or (ii) a proximal end secured within the extracorporeal unit, and/or releasable from within the extracorporeal unit so as to be threadable proximally into an aperture of the lock, through the lock and the tool head, and/or into the shaft of the adjustment tool.

Example 158. The system according to example 157, wherein: (i) the adjustment tool comprises an uptake assembly that comprises: (a) at a working end of the uptake assembly, a gripper disposed proximally from the lock such that, in a receiving state of the uptake assembly, threading of the proximal end of the tether proximally into the aperture of the lock, through the lock and the tool head, and/or into the shaft of the adjustment tool, causes the working end of the uptake assembly to receive the proximal end of the tether, and/or (b) a knob: (A) mounted on a body of the adjustment-tool extracorporeal unit, and/or (B) operably coupled to a proximal part of the gripper such that operation of the knob transitions the uptake assembly into a grip state in which the gripper grips the tether, (ii) the mounting of the knob on the adjustment-tool extracorporeal unit is such that transitioning of the uptake assembly into the grip state releases the knob from the adjustment-tool extracorporeal unit, and/or (iii) once released from the adjustment-tool extracorporeal unit, the knob is removable from the adjustment-tool extracorporeal unit in a manner that withdraws the working end of the uptake assembly, along with the proximal end of the tether, proximally through and out from the shaft and the adjustment-tool extracorporeal unit such that the tether becomes positioned through the lock, the tool head, the shaft, and/or the adjustment-tool extracorporeal unit.

Example 159. The system according to example 158, wherein: (i) the lock is biased to a locked position, (ii) the adjustment tool comprises an obstructor tube extending distally through the shaft and into the tool head such that a distal part of the obstructor tube is disposed within the lock in a manner that constrains the lock unlocked, and/or (iii) while the knob of the uptake assembly remains mounted on the adjustment-tool extracorporeal unit, the working end of the uptake assembly is disposed within the obstructor tube, such that removal of the knob from the adjustment-tool extracorporeal unit withdraws the working end of the uptake assembly, along with the proximal end of the tether, proximally through and out from the obstructor tube such that the tether becomes positioned through the lock, the tool head, the obstructor tube within the shaft, and/or the adjustment-tool extracorporeal unit.

Example 160. The system according to example 158, wherein: (i) the lock is biased to lock, (ii) the adjustment tool comprises: (a) a guillotine within the tool head and proximal from the lock, and/or (b) an obstructor extending distally through the shaft and the guillotine such that a distal part of the obstructor is disposed within the lock in a manner that constrains the lock unlocked, (iii) the adjustment-tool extracorporeal unit comprises a lock-and-cut subassembly that comprises: (a) a locking block, coupled to the obstructor, and/or (b) a lock-and-cut controller, (iv) withdrawal of the working end of the uptake assembly along with the proximal end of the tether, proximally through and out from the shaft and the adjustment-tool extracorporeal unit, leaves the tether positioned through the lock and the guillotine such that (a) subsequent locking of the lock locks the lock to the tether, and/or (b) subsequent actuation of the guillotine cuts the tether proximally from the lock, and/or (v) the lock-and-cut controller is operatively coupled to the locking block such that operation of the lock-and-cut controller draws the locking block proximally such that the obstructor becomes withdrawn from the lock and the lock responsively locks to the tether.

Example 161. The system according to example 160, wherein: (i) the lock comprises a latch that, via engagement with the tool head, retains the lock within the tool head, and/or (ii) the obstructor and the lock are configured such that: (a) while the distal part of the obstructor is disposed within the lock in a manner that constrains the lock unlocked, the distal part of the obstructor also obstructs the latch from disengaging from the tool head, and/or (b) upon withdrawal of the obstructor from the lock, the lock becomes deployable from the tool head.

Example 162. The system according to example 160, wherein: (i) the shaft of the adjustment tool is a primary shaft, (ii) the adjustment tool further comprises a cutter shaft that extends from the adjustment-tool extracorporeal unit through the primary shaft to the guillotine, and/or (iii) the lock-and-cut subassembly further comprises an adapter, coupled to the cutter shaft, and/or shaped and positioned with respect to the locking block such that: (a) operation of the lock-and-cut controller to by a first amount draws the locking block proximally such that the obstructor becomes withdrawn from the lock and the lock responsively locks to the tether while the guillotine remains unactuated, and/or (b) further operation of the lock-and-cut controller, beyond the first amount, engages the locking block with the adapter such that, via the locking block, the adapter, and/or the cutter shaft, the further operation of the lock-and-cutter controller actuates the guillotine.

Example 163. The system according to example 162, wherein the cutter shaft is coupled to the guillotine via a swivel connector.

Example 164. The system according to example 158, wherein the adjustment-tool extracorporeal unit comprises a tensioning subassembly that comprises: (i) a tensioning block, (ii) a clamp, attached to the tensioning block, and/or (iii) a tensioning controller, wherein: (a) while the knob of the uptake assembly remains mounted on the adjustment-tool extracorporeal unit, the gripper extends from the knob, distally through the clamp and the shaft to the working end, (b) withdrawal of the working end of the uptake assembly, along with the proximal end of the tether, proximally through and out from the shaft and the adjustment-tool extracorporeal unit withdraws the gripper from the clamp, leaving the tether positioned through the clamp such that subsequent operation of the clamp locks the tether to the tensioning block, and/or (c) the tensioning controller is operatively coupled to the tensioning block such that, while the tether remains locked to the tensioning block, operation of the tensioning controller applies tension to the tether by drawing the tensioning block and the tether proximally.

Example 165. The system according to example 164, wherein the adjustment-tool extracorporeal unit includes a distance indicator by which a position of the tensioning block with respect to the body of the adjustment-tool extracorporeal unit indicates a distance by which operation of the tensioning controller has drawn the tensioning block proximally.

Example 166. The system according to example 164, wherein the tensioning subassembly further comprises: (a) a spring, (b) a stock, driven by the tensioning controller such that operation of the tensioning controller causes the stock to push, via the spring, the tensioning block proximally, and/or (c) a tension indicator by which a position of the tensioning block with respect to the stock indicates a magnitude of the tension that operation of the tensioning controller has applied to the tether.

Example 167. The system according to example 157, wherein: (i) the adjustment tool comprises an uptake assembly that comprises: (a) a sleeve extending distally through the shaft and terminating proximally from the lock, (b) a gripper extending distally through the sleeve and having a widened distal portion disposed distally outside of the sleeve, the sleeve and the gripper being shaped and positioned such that threading of the proximal end of the tether proximally into the distal-facing aperture of the lock, through the lock and the tool head, and/or into the shaft of the adjustment tool advances the proximal end of the tether proximally around the widened distal portion of the gripper and into the sleeve, and/or (c) a knob: (A) mounted on the adjustment-tool extracorporeal unit, and/or (B) operably coupled to a proximal part of the sleeve and to a proximal part of the gripper such that operation of the knob grips the tether within the sleeve by transitioning the uptake assembly into a grip state by drawing the widened distal portion of the gripper proximally into the sleeve, (ii) the mounting of the knob on the adjustment-tool extracorporeal unit is such that transitioning of the uptake assembly into the grip state releases the knob from the adjustment-tool extracorporeal unit, and/or (iii) once released from the adjustment-tool extracorporeal unit, the knob is removable from the adjustment-tool extracorporeal unit in a manner that pulls the sleeve and the gripper, along with the proximal end of the tether, proximally through the shaft and the adjustment-tool extracorporeal unit, and/or out of the adjustment tool such that the tether extends through the lock, the tool head, the shaft, and/or the adjustment-tool extracorporeal unit.

Example 168. The system according to example 150, wherein: (i) the adjustment tool comprises an obstructor tube disposed within the lock, (ii) the lock comprises: (a) a housing, shaped to define a distal-facing aperture via which the tether is insertable through the lock and into the obstructor tube, and/or (b) a spring-loaded clamp, disposed within the housing, and/or biased to clamp onto the tether within the lock, the presence of the obstructor tube within the lock obstructing the clamp from clamping onto the tether within the lock.

Example 169. The system according to example 168, wherein the lock further comprises a tubular leader that extends, from the distal-facing aperture, away from the body, the tether being insertable through the aperture via the tubular leader.

Example 170. The system according to example 169, wherein the tubular leader comprises a helical coil.

Example 171. The system according to example 169, wherein the tubular leader comprises a protuberant and smooth rim.

Example 172. The system according to example 169, wherein the tubular leader has a flared distal end.

Example 173. The system according to example 169, wherein the tubular leader comprises a sleeve.

Example 174. The system according to example 169, wherein the tubular leader is rigid.

Example 175. The system according to example 169, wherein the tubular leader is flexible.

Example 176. The system according to example 169, wherein the tubular leader comprises and/or is formed from a metal.

Example 177. The system according to example 169, wherein the tubular leader comprises and/or is formed from a polymer.

Example 178. The system according to example 140, wherein the extracorporeal unit is shaped to define a rest in which the shaft is restable while the anchor driver anchors the anchor to the tissue.

Example 179. The system according to example 178, wherein the rest is positioned proximally from the series of cartridges.

Example 180. The system according to example 178, wherein the rest is shaped and positioned such that, while the anchor driver anchors the anchor to the tissue and the shaft is resting in the rest, at least a portion of the shaft extends along the extracorporeal unit alongside the tether.

Example 181. The system according to example 178, wherein the rest is shaped and positioned such that, while the anchor driver anchors the anchor to the tissue and the shaft is resting in the rest, at least a portion of the shaft extends along the extracorporeal unit alongside the proximal-distal axis.

Example 182. The system according to example 140, wherein each of the cartridges is shaped to define a window, the drive head being advanceable through the window to engage, inside the cartridge, the anchor housed by the cartridge.

Example 183. The system according to example 182, wherein the window has a beveled rim that facilitates translational alignment of the drive head with the anchor.

Example 184. The system according to example 182, wherein the window is shaped to allow the drive head to reach the anchor housed by the cartridge only when the drive head is rotationally aligned with the anchor.

Example 185. The system according to example 182, wherein, for each of the cartridges: (a) the cartridge comprises a chassis and a tray, the window being defined at least partly by the chassis and at least partly by the tray, (b) the cartridge has a closed state in which the cartridge securely houses the corresponding anchor, with the corresponding anchor seated in the tray, and/or (c) the anchor driver is configured to remove the corresponding anchor from the cartridge by applying a pulling force to the anchor such that the cartridge transitions into an open state by the tray sliding with respect to the chassis in a manner that exposes the corresponding anchor from the cartridge.

Example 186. A system comprising an implant that comprises: (A) a tether; and/or (B) an anchor, comprising: (i) an anchor head comprising a stock, (ii) a tissue-engaging element: (a) coupled to the stock, (b) extending distally away from the anchor head to define an anchor axis of the anchor, and/or (c) configured to be driven along the anchor axis into tissue of a real or simulated subject, and/or (iii) a textile and/or polymer, shaped to define: (a) a collar, circumscribing the stock, and/or (b) an eyelet, through which the tether is threaded.

Example 187. The system according to example 186, wherein the textile and/or polymer is shaped such that the eyelet is pivotable over the anchor head.

Example 188. The system according to example 186, wherein the eyelet is connected to two places on the collar in a manner that defines a hinge axis on which the two places lie, and/or wherein the eyelet is pivotable about the hinge axis.

Example 189. The system according to any one of examples 186-188, wherein the eyelet is revolvable about the anchor axis by the collar rotating about the anchor axis.

Example 190. The system according to any one of examples 186-189, wherein the textile and/or polymer is a fabric.

Example 191. The system according to any one of examples 186-190, wherein the implant is sterilized.

Example 192. The system according to any one of examples 186-191, wherein the textile comprises filaments of a synthetic polymer.

Example 193. The system according to any one of examples 186-192, wherein the textile comprises filaments of a natural fiber.

Example 194. The system according to any one of examples 186-193, wherein the anchor head comprises an interface that is coupled to the tissue-engaging element via the stock, the tissue-engaging element being configured to be driven along the anchor axis into the tissue by an anchoring force applied to the interface.

Example 195. The system according to any one of examples 186-194, wherein the collar and the eyelet are formed integrally during formation of the textile and/or polymer.

Example 196. The system according to any one of examples 186-195, wherein the eyelet is slidable along the tether.

Example 197. The system according to example 196, wherein the anchor is a second anchor, the implant further comprising a leading anchor coupled to the tether.

Example 198. The system according to any one of examples 186-197, wherein the textile and/or polymer is a yarn.

Example 199. The system according to example 198, wherein the collar and the eyelet are defined by respective loops of the yarn.

Example 200. The system according to example 198, wherein the collar and the eyelet are formed integrally during formation of the yarn.

Example 201. The system according to example 198, wherein the collar and the eyelet are formed by knotting the yarn.

Example 202. The system according to example 198, wherein the collar is formed by securing the yarn into a loop.

Example 203. The system according to example 198, wherein the eyelet is formed by securing the yarn into a loop.

Example 204. The system according to any one of examples 186-203, wherein the anchor further comprises a bushing that is disposed concentrically between the eyelet and the stock.

Example 205. The system according to example 204, wherein the bushing is rotatable about the stock.

Example 206. The system according to example 204, wherein the bushing is annular.

Example 207. The system according to example 204, wherein the textile and/or polymer defines a knot, and/or the bushing defines a recess that is shaped to receive the knot.

Example 208. The system according to example 207, wherein the recess is defined by a cropped part of the bushing, the cropped part having a reduced radius from the anchor axis.

Example 209. The system according to example 207, wherein the recess faces laterally away from the anchor axis.

Example 210. The system according to example 207, wherein the recess is a cubby defined by a bulge of the bushing, the bulge bulging laterally.

Example 211. The system according to example 207, wherein the recess faces medially toward the anchor axis.

Example 212. The system according to example 204, wherein the bushing defines a radially-facing groove, the eyelet residing in the groove.

Example 213. The system according to example 212, wherein the bushing is shaped such that part of the groove is covered in a manner that secures the eyelet in the groove.

Example 214. A system comprising an implant that comprises: (A) a tether; (B) a first anchor, coupled to the tether and configured to anchor the tether to tissue of a real or simulated subject; and/or (C) a second anchor, coupled to the tether, and/or comprising: (i) an anchor head, comprising an interface, (ii) a tissue-engaging element that extends distally away from the anchor head to define an anchor axis of the anchor, the tissue-engaging element being configured to be driven along the anchor axis into tissue of a real or simulated subject by an anchoring force applied to the interface, and/or (iii) a spacer, extending away from the anchor head along the tether toward the first anchor in a manner that inhibits approximation of the second anchor and the first anchor.

Example 215. The system according to example 214, wherein the spacer is connected to the anchor head by a connector formed from a textile.

Example 216. The system according to example 215, wherein the textile defines at least part of the spacer.

Example 217. The system according to example 214, wherein the spacer comprises and/or is formed from a textile.

Example 218. The system according to example 214, wherein the implant is sterilized.

Example 219. The system according to any one of examples 214-218, wherein the spacer is axially compressible.

Example 220. The system according to any one of examples 214-219, wherein the spacer is longer than the tissue-engaging element.

Example 221. The system according to any one of examples 214-220, wherein the second anchor is fixedly coupled to the tether.

Example 222. The system according to any one of examples 214-221, wherein the second anchor is slidably coupled to the tether.

Example 223. The system according to any one of examples 214-222, wherein the implant is configured such that, once the first anchor has anchored the tether to the tissue, the second anchor is advanceable along the tether toward the first anchor while the spacer faces toward the first anchor.

Example 224. The system according to any one of examples 214-223, wherein the implant is configured such that, once the tissue-engaging element has been driven into the tissue, the first anchor is advanceable along the tether toward the second anchor while the spacer faces toward the first anchor.

Example 225. The system according to any one of examples 214-224, wherein the tether has a distal end, and/or the spacer extends away from the anchor head distally along the tether.

Example 226. The system according to any one of examples 214-225, wherein the tether has a distal end, and/or the spacer extends away from the anchor head proximally along the tether.

Example 227. The system according to any one of examples 214-226, wherein the interface is disposed on the anchor axis.

Example 228. The system according to any one of examples 214-227, wherein the tissue-engaging element is helical, defines the anchor axis by extending in a helix around and along the anchor axis, and/or is configured to be screwed into the tissue of the subject.

Example 229. The system according to any one of examples 214-228, wherein the spacer resists axial compression.

Example 230. The system according to any one of examples 214-229, wherein the spacer is mounted to be revolvable around the anchor axis.

Example 231. The system according to example 230, wherein: (i) the anchor comprises a collar that circumscribes the anchor axis, (ii) the spacer is coupled to the collar, and/or (iii) the spacer is mounted to be revolvable around the anchor axis by rotation of the collar about the anchor axis.

Example 232. The system according to example 231, wherein: (i) the anchor head comprises a stock that fixedly couples the interface to the tissue-engaging element, and/or (ii) the collar circumscribes, and/or is rotatable about, the stock.

Example 233. The system according to any one of examples 214-232, wherein the second anchor is slidable along the tether while in a delivery state in which the spacer extends away from the anchor head alongside the tissue-engaging element.

Example 234. The system according to example 233, wherein: (i) at a distal end of the tissue-engaging element, the tissue-engaging element has a sharp point, and/or (ii) in the delivery state, the spacer extends beyond the sharp point.

Example 235. The system according to example 233, wherein the spacer is pivotable, from the delivery state, to become substantially orthogonal to the tissue-engaging element.

Example 236. The system according to any one of examples 214-235, wherein the spacer is flexible in deflection.

Example 237. The system according to example 236, wherein the spacer is resilient.

Example 238. The system according to any one of examples 214-237, wherein the spacer is tubular.

Example 239. The system according to example 238, wherein the spacer is defined by a helical wire shaped as a coil.

Example 240. The system according to any one of examples 214-239, further comprising a delivery tool that comprises an anchor driver and a percutaneously-advanceable tube, anchor driver configured to engage the interface, to advance the second anchor through the tube, and/or to drive the anchor into the tissue by applying the anchoring force to the interface.

Example 241. The system according to example 240, wherein the anchoring force includes torque, and/or wherein the anchor driver is configured to drive the anchor into the tissue by applying the torque to the interface.

Example 242. The system according to example 240, wherein: (i) the tube defines an internal channel that has a keyhole-shaped orthogonal cross-section that defines a major channel-region and a minor channel-region, (ii) the major channel-region has a larger cross-sectional area than does the minor channel-region, and/or (iii) the anchor driver is configured to advance the second anchor through the internal channel with the anchor head sliding snugly through the major channel-region, and/or the spacer sliding snugly through the minor channel-region.

Example 243. The system according to example 242, wherein the spacer is configured to restrain the tether within the minor channel-region as the second anchor is advanced by the anchor driver through the internal channel.

Example 244. A system, useable and/or for use with tissue of a real or simulated subject, the system comprising: (A) an implant, comprising: (i) a tether, being radiopaque, and/or biased toward assuming a regular wavy shape, and/or (ii) multiple anchors, each comprising an anchor head and a tissue-engaging element extending distally from the anchor head; (B) an anchor driver, configured to anchor the multiple anchors to the tissue: (a) by, for each of the multiple anchors, via engagement with the anchor head, driving the tissue-engaging element into the tissue, and/or (b) such that the anchor head of each of the multiple anchors is threaded onto the tether, with the tether assuming its regular wavy shape; and (C) an adjustment tool, configured to: (i) apply tension to the tether in a manner that straightens the tether and draws the multiple anchors toward each other, and/or (ii) lock the tension in the tether.

Example 245. The system according to example 244, wherein the implant is sterilized.

Example 246. The system according to any one of examples 244-245, wherein the anchor driver is sterilized.

Example 247. The system according to any one of examples 244-246, wherein the adjustment tool is sterilized.

Example 248. The system according to any one of examples 244-247, wherein the tether comprises a drawn filled tube that has a radiopaque core.

Example 249. The system according to any one of examples 244-248, wherein the tether comprises a cable that includes a radiopaque strand and a shape memory strand.

Example 250. The system according to any one of examples 244-249, wherein the tether comprises a shape memory alloy, and/or is shape-set to the regular wavy shape.

Example 251. The system according to any one of examples 244-250, wherein the regular wavy shape is sinusoidal, and/or wherein the tether is biased toward assuming the sinusoidal shape.

Example 252. The system according to any one of examples 244-251, wherein the regular wavy shape is a zigzag, and/or wherein the tether is biased toward assuming the zigzag shape.

Example 253. An apparatus comprising an implant, the implant comprising: (A) an anchor, comprising: (i) an anchor head, comprising a socket, and/or (ii) a tissue-engaging element extending distally from the anchor head; (B) a tether; and/or (C) a stopper, attached to an end of the tether, and/or secured within the socket in a manner that couples the anchor to the end of the tether.

Example 254. The apparatus according to example 253, wherein the implant is sterilized.

Example 255. The apparatus according to any one of examples 253-254, wherein the stopper is bulbous.

Example 256. The apparatus according to any one of examples 253-255, wherein the stopper is substantially spherical.

Example 257. The apparatus according to any one of examples 253-256, wherein the anchor is a leading anchor, and/or wherein the implant further comprises one or more successive anchors.

Example 258. The apparatus according to any one of examples 253-257, wherein the stopper is rotatable within the socket.

Example 259. The apparatus according to any one of examples 253-258, wherein the stopper is snap-fitted into the socket.

Example 260. The apparatus according to any one of examples 253-259, wherein the stopper is a bead.

Example 261. The apparatus according to any one of examples 253-260, wherein the stopper is attached to the end of the tether by crimping.

Example 262. The apparatus according to any one of examples 253-261, wherein the stopper is attached to the end of the tether by welding.

Example 263. The apparatus according to any one of examples 253-262, wherein the stopper is attached to the end of the tether by brazing.

Example 264. The apparatus according to any one of examples 253-263, wherein the anchor is a fixed anchor, and/or wherein the implant further comprises one or more sliding anchors, each of the sliding anchors being slidably coupled to the tether.

Example 265. The apparatus according to example 264, wherein each of the one or more sliding anchors comprises an eyelet and is slidably coupled to the tether by the eyelet being threaded onto the tether.

Example 266. The apparatus according to any one of examples 253-265, wherein: (i) the anchor head comprises a casing that defines the socket, (ii) the casing further defines a window into the socket, and/or (iii) the tether extends, from the stopper, through the window to exit the socket.

Example 267. The apparatus according to example 266, wherein, across from the window, the socket has an open side.

Example 268. The apparatus according to example 267, wherein the casing comprises a cantilever that obstructs the stopper from exiting the socket via the open side.

Example 269. The apparatus according to example 266, wherein the window extends at least a fifth of the way around the stopper.

Example 270. The apparatus according to example 266, wherein the window curves in an arc around the socket.

Example 271. The apparatus according to example 266, wherein the window is sized, and/or the socket is configured, to allow the tether to pivot with respect to the anchor head via rotation of the stopper within the socket.

Example 272. The apparatus according to example 266, wherein the window is sized, and/or the socket is configured, to allow pivoting of the tether with respect to the anchor head to rotate the stopper within the socket.

Example 273. The apparatus according to example 266, wherein: (a) the tissue-engaging element extends distally from the anchor head to define an anchor axis along which the tissue-engaging element is advanceable into tissue of a real or simulated subject, and/or (b) the window is shaped to allow the tether to pivot between: (i) an axial state in which the tether extends through the window in a trajectory that is parallel with the anchor axis, and/or (ii) a lateral state in which the tether extends through the window in a trajectory that is orthogonal with the anchor axis.

Example 274. The apparatus according to any one of examples 253-273, wherein the end of the tether does not protrude from the stopper.

Example 275. The apparatus according to example 274, wherein the end of the tether is flush with an external surface of the stopper.

Example 276. The apparatus according to example 274, wherein the end of the tether is disposed within the stopper.

Example 277. A system useable and/or for use with a real or simulated heart of a real or simulated subject, the system comprising: (A) an implant, comprising an anchor, the anchor comprising: (i) a head, comprising an interface, and/or (ii) and a helical tissue-engaging element, extending distally away from the head to define an anchor axis of the anchor; and/or (B) a delivery tool, comprising: (a) a catheter device, comprising: (i) an extracorporeal portion at a proximal part of the catheter device, and/or (ii) a flexible tube, extending distally from the extracorporeal portion, and/or having a distal portion that: (I) is configured to be transluminally advanced to the heart, (II) has a distal opening, (III) defines, along a tube axis of the tube, a channel through which the anchor is slidable toward the distal opening, and/or (IV) defines, proximal from the distal opening, a grip zone at which the distal portion has a resilient rib that protrudes medially into the channel in a manner that inhibits sliding of the anchor through the grip zone by gripping the helical tissue-engaging element; and/or (b) an anchor driver configured, via engagement with the interface, to: (i) slide the anchor distally through the channel toward the grip zone, and/or (ii) drive the anchor through the grip zone by screwing the tissue-engaging element over the rib.

Example 278. The system according to example 277, wherein the implant is sterilized.

Example 279. The system according to any one of examples 277-278, wherein the catheter device is sterilized.

Example 280. The system according to any one of examples 277-279, wherein the anchor driver is sterilized.

Example 281. The system according to any one of examples 277-280, wherein the distal opening has a rim, and/or the tube is shaped such that the rim is undulating.

Example 282. The system according to any one of examples 277-281, wherein the distal portion is flared toward the distal opening.

Example 283. The system according to any one of examples 277-282, wherein the rib is configured such that, as the driver screws the tissue-engaging element over the rib, the tissue-engaging element compresses parts of the rib with which the tissue-engaging element is in contact.

Example 284. The system according to any one of examples 277-283, wherein the rib has a proximal face that is shaped to define a shoulder.

Example 285. The system according to any one of examples 277-284, wherein the rib has a distal face that is tapered.

Example 286. The system according to example 277, wherein: (i) the anchor further comprises an eyelet, mounted on the head so as to be revolvable about the anchor axis, and/or (ii) the implant further comprises a tether, threaded through the eyelet such that the eyelet is slidable along the tether.

Example 287. The system according to example 286, wherein: (i) the rib extends medially into the channel in a manner that defines, adjacent the rib, a niche in the grip zone, and/or (ii) the system is configured such that, while the anchor driver screws the tissue-engaging element over the rib: (a) the tissue-engaging element is excluded from the niche, and/or (b) the tether extends through the grip zone within the niche, laterally from the tissue-engaging element.

Example 288. The system according to example 286, wherein, proximal from the rib, the tube further defines an abutment that protrudes medially into the channel in a manner that, as the anchor driver screws the tissue-engaging element over the rib, inhibits revolution of the eyelet about the anchor axis.

Example 289. The system according to example 288, wherein a unitary structure defines both the rib and the abutment.

Example 290. The system according to example 288, wherein the rib protrudes further medially into the channel than the abutment.

Example 291. The system according to example 288, wherein the abutment is longer, along the channel, than the rib.

Example 292. The system according to any one of examples 277-291, wherein the rib comprises and/or is formed from a polymer.

Example 293. The system according to example 292, wherein the tube is lined with the polymer.

Example 294. The system according to example 292, wherein the tube comprises and/or is formed from the polymer.

Example 295. The system according to example 292, wherein the polymer is a thermoplastic elastomer.

Example 296. The system according to example 292, wherein the polymer is a block copolymer.

Example 297. The system according to example 296, wherein the block copolymer is polyether block amide.

Example 298. The system according to any one of examples 277-297, wherein the rib is a first of multiple ribs defined by the distal portion in the grip zone.

Example 299. The system according to example 298, wherein the multiple ribs are exactly two ribs.

Example 300. The system according to example 298, wherein the multiple ribs are exactly three ribs.

Example 301. The system according to example 298, wherein the multiple ribs are exactly four ribs.

Example 302. The system according to example 298, wherein the multiple ribs are exactly five ribs.

Example 303. The system according to example 298, wherein the multiple ribs are exactly six ribs.

Example 304. The system according to example 298, wherein the multiple ribs are distributed circumferentially around the tube axis.

Example 305. The system according to example 298, wherein the multiple ribs are distributed along the tube axis.

Example 306. The system according to any one of examples 277-305, wherein the rib extends around at least part of the tube axis.

Example 307. The system according to example 306, wherein the rib is toroidal, extending circumferentially around the entire tube axis.

Example 308. The system according to any one of examples 277-307, wherein the rib extends alongside the tube axis.

Example 309. The system according to example 308, wherein the rib is parallel with the tube axis.

Example 310. A system useable and/or for use with a real or simulated heart of a real or simulated subject, the system comprising: (A) an implant, comprising an anchor, the anchor comprising: (i) a head, comprising an interface, and/or (ii) and a tissue-engaging element, extending distally away from the head to define an anchor axis of the anchor; and/or (B) a delivery tool, comprising: (i) a catheter device, comprising: (a) an extracorporeal portion at a proximal part of the catheter device, (b) a flexible tube, extending distally from the extracorporeal portion, and/or having a distal portion that: (I) is configured to be transluminally advanced to the heart, (II) has a distal opening, and/or (III) defines, along a tube axis of the tube, a channel through which the anchor is slidable toward the distal opening, and/or (c) a membrane, disposed over the distal opening, and/or having one or more slits that divide the membrane into multiple flaps; and/or (ii) an anchor driver configured, via engagement with the interface, to slide the anchor distally through the channel, and/or distally through the membrane via the one or more slits, the membrane configured such that the flaps transiently separate responsively to passage of the anchor through the membrane.

Example 311. The system according to example 310, wherein the implant is sterilized.

Example 312. The system according to any one of examples 310-311, wherein the catheter device is sterilized.

Example 313. The system according to any one of examples 310-312, wherein the anchor driver is sterilized.

Example 314. The system according to any one of examples 310-313, wherein the membrane has multiple slits.

Example 315. The system according to example 314, wherein the multiple slits divide the membrane into four flaps.

Example 316. The system according to example 314, wherein the multiple slits converge to define a convergence point.

Example 317. The system according to example 316, wherein the membrane has a hole at the convergence point.

Example 318. The system according to example 317, wherein the anchor driver is configured to slide the anchor distally through the channel such that the tissue-engaging element aligns with the hole.

Example 319. The system according to example 316, wherein the membrane defines a notch, disposed eccentrically.

Example 320. The system according to example 319, wherein the notch extends laterally from the convergence point.

Example 321. The system according to example 319, wherein the notch is defined in a single one of the flaps.

Example 322. The system according to example 319, wherein the notch is defined partly in one of the flaps and partly in another of the flaps.

Example 323. The system according to example 319, wherein: (i) the head lies on the anchor axis, (ii) the anchor comprises an eyelet, mounted laterally from the anchor axis, and/or (iii) the anchor driver is configured to slide the anchor distally through the channel such that the eyelet aligns with the notch.

Example 324. A system useable and/or for use with a tether secured to a real or simulated tissue of a real or simulated subject, the system comprising: (A) a tool; and/or (B) a lock: (i) comprising a passage through the lock, the passage configured to receive the tether therethrough, (ii) having an unlocked state in which the lock is transluminally slidable along the tether to the tissue by the tether sliding through the passage, (iii) comprising: (a) a clamp face, (b) a blade, and/or (c) an interface, engageable by the tool in a manner that configures the tool to actuate the lock by applying an actuating force to the interface, and/or (iv) configured such that, while the tether is disposed through the passage, actuation of the lock (a) locks the tether to the lock by clamping the clamp face to the tether, and/or (b) cuts the tether with the blade.

Example 325. The system according to example 324, wherein the tool is sterilized.

Example 326. The system according to any one of examples 324-325, wherein the lock is sterilized.

Example 327. The system according to any one of examples 324-326, wherein the actuating force is torque, and/or the tool is configured to actuate the lock by applying torque to the interface.

Example 328. The system according to any one of examples 324-327, wherein: (i) the lock comprises an opposing face, actuation of the lock locking the tether to the lock by advancing the clamp face toward the opposing face, and/or (ii) the lock is configured such that, following the clamping of the tether between the clamp face and the opposing face, further actuation of the lock causes the clamp face to push the opposing face to move along with the clamp face.

Example 329. The system according to any one of examples 324-328, wherein actuation of the lock: (i) clamps the clamp face to the tether via axial movement of the clamp face, and/or (ii) cuts the tether via axial movement of the blade.

Example 330. The system according to any one of examples 324-329, wherein actuation of the lock: (i) clamps the clamp face to the tether via planar movement of the clamp face, and/or (ii) cuts the tether via planar movement of the blade.

Example 331. The system according to any one of examples 324-330, wherein the lock comprises a mechanical linkage that includes a first bar and a second bar, the first bar providing the clamp face and the second bar providing the blade.

Example 332. The system according to example 331, wherein the mechanical linkage is a planar linkage.

Example 333. The system according to example 331, wherein the first bar is hingedly connected to the second bar.

Example 334. The system according to example 333, wherein the mechanical linkage is configured such that actuation of the lock clamps the tether between the clamp face and the second bar.

Example 335. The system according to example 331, wherein the lock comprises a casing, and/or wherein the mechanical linkage is configured such that actuation of the lock clamps the tether between the clamp face and the casing.

Example 336. The system according to example 331, wherein the blade faces away from the first bar.

Example 337. The system according to example 331, wherein the interface is coupled to a threaded rod that cooperates with the mechanical linkage as a linear actuator, such that rotation of the interface rotates the threaded rod and pivots the first bar with respect to the second bar.

Example 338. The system according to any one of examples 324-337, wherein the actuation of the lock clamps the clamp face to the tether prior to cutting the tether with the blade by the lock being configured such that: (i) a first amount of the actuation clamps the clamp face to the tether, and/or (ii) further actuation of the lock, beyond the first amount of the actuation, is required for the blade to cut the tether.

Example 339. The system according to example 338, wherein the lock is configured such that the actuation of the lock clamps the clamp face to the tether prior to cutting the tether with the blade, wherein a distance-of-movement of the blade to cut the tether is greater than a distance-of-movement of the clamp face to clamp the clamp face to the tether.

Example 340. The system according to example 338, wherein the lock is configured such that the actuation of the lock clamps the clamp face to the tether prior to cutting the tether with the blade, wherein the lock comprises a mechanism that, responsive to actuation of the lock, moves the clamp face at a first rate, and/or moves the blade at a second rate that is different to the first rate.

Example 341. The system according to example 338, wherein the lock comprises an opposing face, and/or wherein actuating the lock comprises by clamping the tether between the clamp face and the opposing face, thereby locking the tether to the stopper.

Example 342. The system according to example 341, wherein: (i) the lock comprises a spring, and/or (ii) the further actuation of the lock strains the spring, the straining of the spring functionalizing the blade.

Example 343. The system according to example 341, wherein: (i) the opposing face is supported by a compressible member, and/or (ii) the further actuation of the lock drives the clamp face to compress the compressible member in a manner that: (a) maintains the tether clamped between the clamp face and the opposing face, and/or (b) enables movement of the blade to cut the tether.

Example 344. The system according to any one of examples 324-343, wherein the lock defines, extending from a first end of the passage to a second end of the passage, lateral access via which the tether is introducible into the passage.

Example 345. The system according to example 344, wherein the lock comprises a casing that defines: (i) at the first end of the passage, an entrance into the passage, (ii) at the second end of the passage, an exit from the passage, and/or (iii) connecting the entrance to the exit, a lateral slit that provides the lateral access.

Example 346. The system according to any one of examples 324-345, wherein actuation of the lock cuts the tether by revolving the blade around an axis.

Example 347. The system according to example 346, wherein actuation of the lock clamps the clamp face to the tether by moving the clamp face along the axis.

Example 348. A system useable and/or for use with a tether secured to a real or simulated tissue of a real or simulated subject, the system comprising: (A) a malleable lock, shaped to define a passage therethrough; and/or (B) a tool, comprising: (i) a shaft, (ii) a collet, housed within the shaft, the lock held within the collet, and/or (iii) a grasper, extendable distally through the passage and out of the shaft, and/or configured to grasp a bight of the tether and draw the bight proximally through the lock and into the shaft, thereby retaining the tether as a loop within the shaft, the tool being configured to, while the tether remains retained as the loop within the shaft: (a) be intracorporeally advanced distally along the tether such that progressive regions of the tether are fed around the grasper, (b) subsequently lock the lock to the tether by actuating the collet to crimp the lock, (c) subsequently release the lock from the collet and the tether from the grasper, and/or (d) be subsequently withdrawn from the subject.

Example 349. The system according to example 348, wherein the tool is sterilized.

Example 350. The system according to any one of examples 348-349, wherein the lock is sterilized.

Example 351. The system according to any one of examples 348-350, wherein the tool is configured to actuate the collet via rotation of the shaft.

Example 352. The system according to any one of examples 348-351, wherein the grasper is a hook.

Example 353. The system according to any one of examples 348-352, wherein the grasper is a snare.

Example 354. A system useable and/or for use with a real or simulated subject, the system comprising: (A) a catheter device, comprising: (i) a tube that has: (a) a distal opening that is configured to be transluminally advanced into the subject, and/or (b) a proximal end that defines a proximal opening, and/or (ii) an extracorporeal unit that is coupled to the proximal end of the tube; (B) a tether, having a leading end and a second end, the second end being coupled to the extracorporeal unit; (C) a series of anchors including: (i) a leading anchor, coupled to the leading end of the tether, and/or (ii) multiple successive anchors, slidably coupled to the tether; (D) an anchor driver, configured to: (i) advance the leading anchor with the leading end of the tether through the tube, and/or anchor the leading anchor to tissue of the subject, and/or (ii) subsequently, for each of the successive anchors sequentially, advance the successive anchor along the tether and through the tube; and/or (E) a tensioner, configured to: (a) engage an intermediate region of the tether, the intermediate region of the tether being at the extracorporeal unit and between the leading end and the second end, and/or (b) apply tension to the tether by pulling on the intermediate region of the tether.

Example 355. The system according to example 354, wherein the tensioner is housed by the extracorporeal unit.

Example 356. The system according to any one of examples 354-355, wherein the catheter device is sterilized.

Example 357. The system according to any one of examples 354-356, wherein the tether is sterilized.

Example 358. The system according to any one of examples 354-357, wherein the anchors of the series are sterilized.

Example 359. The system according to any one of examples 354-358, wherein the anchor driver is sterilized.

Example 360. The system according to any one of examples 354-359, wherein the tensioner is sterilized.

Example 361. The system according to any one of examples 354-360, wherein the tensioner comprises a gripper, configured to grip the tether in a manner that defines an isolated region of the tether between the grip and the second end, and/or that isolates the isolated region from the tension applied by the tensioner.

Example 362. The system according to any one of examples 354-361, wherein the tensioner comprises a sheave, and/or is configured to engage the intermediate region of the tether by engaging the sheave with the tether.

Example 363. The system according to any one of examples 354-362, wherein the tensioner is a component of the extracorporeal unit.

Example 364. The system according to any one of examples 354-363, wherein the tensioner comprises a linear actuator.

Example 365. The system according to any one of examples 354-364, wherein the tensioner comprises a knob and complementary screw threads and is actuatable via rotation of the knob.

Example 366. The system according to any one of examples 354-365, wherein the tensioner is configured to apply the tension to the tether by pulling the intermediate region of the tether laterally.

Example 367. The system according to any one of examples 354-366, wherein the tensioner comprises a force gauge that indicates a magnitude of the tension.

Example 368. The system according to any one of examples 354-367, wherein the extracorporeal unit comprises a winch, the second end of the tether operatively coupled to the winch.

Example 369. The system according to example 368, wherein the winch is spring-loaded in a manner that reduces slack in the tether.

Example 370. The system according to example 369, wherein the winch comprises a lock, actuation of the lock locking the winch.

Example 371. The system according to any one of examples 354-370, wherein the anchors are mounted on the extracorporeal unit.

Example 372. The system according to example 371, wherein each of the anchors is stored in a respective cartridge that is mounted on the extracorporeal unit.

Example 373. The system according to any one of examples 354-372, wherein the tensioner is configured: (i) to be actuated to apply the tension, and/or (ii) to subsequently maintain the tension.

Example 374. The system according to example 373, wherein the tensioner comprises a latch, and/or is configured to maintain the tension by the latch being latched after the tensioner is actuated.

Example 375. The system according to example 373, wherein the tensioner comprises a ratchet, the ratchet maintaining the tension.

Example 376. The system according to any one of examples 354-375, wherein the tensioner is reversibly mountable on the extracorporeal unit.

Example 377. The system according to example 376, wherein: (i) the extracorporeal unit defines an access site at which, once the anchor driver advances the leading anchor with the leading end of the tether through the tube and anchors the leading anchor, the intermediate region of the tether extends through the access site, and/or (ii) the tensioner is reversibly mountable at the access site of the extracorporeal unit.

Example 378. An apparatus comprising an anchor that comprises: (i) an anchor head comprising a stock, (ii) a tissue-engaging element: (a) coupled to the stock, (b) extending distally away from the anchor head to define an anchor axis of the anchor, and/or (c) configured to be driven along the anchor axis into tissue of a real or simulated subject, and/or (iii) an eyelet, mounted eccentrically from the stock, and/or being saddle-shaped.

Example 379. The apparatus according to example 378, wherein the anchor is sterilized.

Example 380. The apparatus according to any one of examples 378-379, further comprising a tether threaded through the eyelet.

Example 381. The apparatus according to any one of examples 378-380, wherein the eyelet is revolvable about the anchor axis.

Example 382. The apparatus according to example 381, wherein the anchor further comprises a collar that circumscribes the stock, the eyelet being revolvable about the anchor axis by the collar rotating about the anchor axis.

Example 383. An apparatus comprising an anchor useable and/or for use with tissue of a real or simulated heart of a real or simulated subject, the anchor comprising: (A) an anchor head: (i) defining an interface, and/or (ii) formed substantially from a polymer; and/or (B) a tissue-engaging element: (i) extending distally away from the anchor head to define an anchor axis of the anchor, (ii) configured to be driven along the anchor axis into tissue of a real or simulated subject by an anchoring force applied to the interface, and/or (iii) formed substantially from the polymer.

Example 384. The apparatus according to example 383, wherein the anchor is sterilized.

Example 385. The apparatus according to any one of examples 383-384, wherein the anchor head comprises a metal pin that serves as part of the interface.

Example 386. The apparatus according to any one of examples 383-385, wherein the polymer is a polyaryletherketone.

Example 387. The apparatus according to example 386, wherein the polymer is polyether ether ketone.

Example 388. The apparatus according to any one of examples 383-387, wherein, in at least part of the anchor, a radiopaque substance is mixed with the polymer.

Example 389. The apparatus according to example 388, wherein the radiopaque substance is barium sulfate.

Example 390. The apparatus according to any one of examples 383-389, wherein the anchor head includes a stock that fixedly couples the interface to the tissue-engaging element, and/or wherein the anchor further comprises: (i) a collar: (a) rotatably coupled to the anchor head by circumscribing the stock, and/or (b) formed substantially from the polymer; and/or (ii) an eyelet: (a) mounted eccentrically by being coupled to the collar, (b) revolvable about the anchor axis by rotation of the collar around the stock, and/or (c) formed substantially from the polymer.

Example 391. The apparatus according to example 390, wherein the collar and the eyelet are formed as a monolithic piece of the polymer.

Example 392. The apparatus according to any one of examples 383-391, wherein the tissue-engaging element comprises a central shaft and an external self-tapping screw thread extending helically around and along the central shaft.

Example 393. The apparatus according to example 392, wherein the central shaft defines a distal point and has a tapered region that tapers distally toward the distal point.

Example 394. The apparatus according to example 393, wherein the distal point lies on the anchor axis.

Example 395. The apparatus according to example 393, wherein the central shaft tapers more steeply at the distal point, compared with at the tapered region.

Example 396. The apparatus according to example 392, wherein the screw thread protrudes laterally from the central shaft by a distance, and/or wherein the central shaft has a diameter that is 2-4 times greater than the distance.

Example 397. The apparatus according to example 396, wherein the diameter of the central shaft is approximately 3 times greater than the distance.

Example 398. A system comprising an implant that comprises: (A) a tether; and/or (B) an anchor, comprising: (i) an anchor head comprising: (a) a stock, and/or (b) an interface, (ii) a tissue-engaging element: (a) coupled to the interface via the stock, (b) extending distally away from the anchor head to define an anchor axis of the anchor, and/or (c) configured to be driven along the anchor axis into tissue of a real or simulated subject by an anchoring force applied to the interface, and/or (iii) a textile and/or polymer, shaped to define an eyelet, through which the tether is threaded, the eyelet being pivotable over the interface.

Example 399. The system according to example 398, wherein the textile and/or polymer is a fabric.

Example 400. The system according to example 398, wherein the textile and/or polymer is a yarn.

Example 401. The system according to example 398, wherein the textile and/or polymer is elongate, has two ends and a bight therebetween, and/or is shaped to define a loop at each end, the loops being threaded onto the stock such that the bight defines the eyelet.

Example 402. The system according to example 398, wherein the anchor further comprises a collar that circumscribes the stock, the eyelet being connected to the collar such that the eyelet is revolvable about the anchor axis by the collar rotating about the anchor axis.

Example 403. The system according to example 402, wherein the textile and/or polymer is elongate, having two ends and a bight therebetween, the ends being connected to the collar such that the bight defines the eyelet.

Example 404. The system according to example 402, wherein the eyelet: (i) extends from two places on the collar, and/or (ii) is pivotable over the interface by pivoting about a hinge axis on which the two places lie.

Example 405. The system according to example 404, wherein the collar is defined by the textile and/or polymer.

Example 406. The system according to example 404, wherein the collar is rigid.

Example 407. The system according to example 404, wherein the collar is shaped to define at least one bore through which the textile and/or polymer passes.

Example 408. The system according to example 404, wherein the collar is shaped to define at least one tab to which the textile and/or polymer is tied.

Example 409. The system according to example 404, wherein the collar is flexible.

Example 410. The system according to example 409, wherein the collar is defined by a flexible tube, and/or wherein: (i) the tube has: (a) a tube-lumen along the tube, the tube-lumen having two ends, (b) an end-opening at each end of the tube, and/or (c) a transverse channel, the stock extending transversely through the tube via the transverse channel; and/or (ii) the textile defines a closed loop onto which the tube is threaded by the textile extending through the lumen and out of both end-openings.

Example 411. A system, useable and/or for use with tissue of a real or simulated heart of a real or simulated subject, the system comprising an implant that comprises: (A) a tether, having a series of beads distributed along the tether and fixed to the tether, (B) multiple anchors, each comprising: (i) a tissue-engaging element that defines an anchor axis of the anchor, and/or (ii) a head, coupled to the tissue-engaging element, and/or having a geometry that: (a) facilitates sliding of the head over and along the tether while the anchor axis is parallel with the tether by allowing the beads to pass through the head, and/or (b) inhibits sliding of the head over and along the tether while the anchor axis is transverse to the tether by obstructing the beads from passing through the head.

Example 412. The system according to example 411, further comprising an anchor driver, configured to implant the implant at the tissue such that the tether is nonparallel with the tissue-engaging element of each of the anchors by, for each of the anchors sequentially, via engagement with the head: (i) transluminally sliding the head over and along the tether to the heart while the anchor axis is parallel with the tether, and/or (ii) driving the tissue-engaging element into the tissue.

Example 413. The system according to example 411, further comprising an adjustment tool, configured to apply tension to the tether after the implant has been implanted at the tissue, the implant being configured such that tensioning of the tether after the implant has been implanted at the tissue causes at least one of the beads to become obstructed by the head of at least one of the anchors.

Example 414. The system according to example 411, wherein each of the beads is oval.

Example 415. The system according to example 411, wherein each of the beads is a prolate spheroid.

Example 416. The system according to example 411, wherein each of the beads is radiopaque.

Example 417. The system according to example 411, wherein each of the beads is echogenic.

Example 418. The system according to example 411, wherein the implant is sterilized.

Example 419. The system according to example 411, wherein the anchor driver is sterilized.

Example 420. The system according to example 411, wherein the adjustment tool is sterilized.

Example 421. A system useable and/or for use with a real or simulated tissue of a real or simulated subject, the system comprising: (A) a catheter device, comprising: (i) a tube that has: (a) a distal opening that is configured to be transluminally advanced into the subject, and/or (b) a proximal end that defines a proximal opening; and/or (ii) an extracorporeal unit, coupled to the proximal end of the tube, and/or comprising: (I) a body; and/or (II) a series of cartridges, distributed along or parallel to a proximal-distal axis of the body, each of the cartridges: (a) defining a respective cartridge vector that is oblique with respect to the proximal-distal axis, (b) having a closed state, and/or (c) being transitionable into an open state by at least part of the cartridge being slid along its cartridge vector, (B) a tether, extending along the body; and/or (C) a series of anchors, each anchor of the series of anchors being: (i) coupled to the tether, (ii) housed by a corresponding cartridge of the series of cartridges, and/or (iii) removable from the corresponding cartridge upon transitioning of the corresponding cartridge into its open state.

Example 422. The system according to example 421, wherein the cartridge vectors of the series of cartridges collectively define a common cartridge plane on which the cartridge vectors lie.

Example 423. The system according to example 422, wherein the proximal-distal axis is parallel with the common cartridge plane.

Example 424. The system according to example 422, wherein the proximal-distal axis lies on the common cartridge plane.

Example 425. The system according to example 422, wherein the tether extends along the body, parallel with the common cartridge plane.

Example 426. A system useable and/or for use with a real or simulated tissue of a real or simulated subject, the system comprising: (A) a catheter device, comprising: (i) a tube that has: (a) a distal opening that is configured to be transluminally advanced into the subject, and/or (b) a proximal end that defines a proximal opening; and/or (ii) an extracorporeal unit, coupled to the proximal end of the tube, and/or comprising: (I) a body; and/or (II) a series of cartridges, distributed along or parallel to a proximal-distal axis of the body; (B) a tether, extending along the body; and/or (C) a series of anchors, each anchor of the series of anchors being: (i) coupled to the tether, (ii) housed by a corresponding cartridge of the series of cartridges, and/or (iii) releasable from the corresponding cartridge by pulling of the anchor such that at least part of the corresponding cartridge slides along a respective cartridge vector that is oblique with respect to the proximal-distal axis.

Example 427. A system useable and/or for use with a real or simulated tissue of a real or simulated subject, the system comprising: (A) a catheter device, comprising: (i) a tube that has: (a) a distal opening that is configured to be transluminally advanced into the subject, and/or (b) a proximal end that defines a proximal opening; and/or (ii) an extracorporeal unit, coupled to the proximal end of the tube; (B) a tether, extending along the extracorporeal unit; and/or (C) a series of anchors, distributed along the extracorporeal unit along or parallel to a proximal-distal axis of the extracorporeal unit, each anchor of the series of anchors: (i) comprising: (a) a head, coupled to the tether, and/or (b) a tissue-engaging element, extending away from the head to define an anchor axis of the anchor, and/or (ii) mounted on the extracorporeal unit such that the anchor axis lies obliquely with respect to the proximal-distal axis.

Example 428. The system according to example 427, wherein the tether extends along the extracorporeal unit alongside the proximal-distal axis.

Example 429. The system according to any one of examples 427-428, wherein: (i) the extracorporeal unit comprises a series of cartridges, distributed along the proximal-distal axis, and/or (ii) each of the anchors is mounted on the extracorporeal unit by being housed by a corresponding cartridge of the series of cartridges.

Example 430. The system according to any one of examples 427-429, wherein, for each anchor of the series of anchors, the anchor is oriented with the head proximal from the tissue-engaging element.

Example 431. The system according to any one of examples 427-430, wherein, for each anchor of the series of anchors, the anchor is oriented with the tissue-engaging element closer than the head to the proximal opening.

Example 432. The system according to any one of examples 427-431, wherein the anchor axes of the series of anchors collectively define a common anchor plane.

Example 433. The system according to example 432, wherein the proximal-distal axis is parallel with the common anchor plane.

Example 434. The system according to example 432, wherein the proximal-distal axis lies on the common anchor plane.

Example 435. The system according to example 432, wherein the tether extends, along the extracorporeal body, parallel with the common anchor plane.

Example 436. An anchor usable and/or for use at a real or simulated tissue of a real or simulated heart of a real or simulated subject, the anchor comprising: (A) a head; and (B) a tissue-engaging element: (i) extending away from the head to define an anchor axis of the anchor along which the tissue-engaging element is configured to be driven into the tissue, and/or (ii) formed, by additive manufacturing, to be porous.

Example 437. The apparatus according to example 436, wherein the tissue-engaging element is helical and configured to be screwed along the anchor axis into the tissue.

Example 438. The apparatus according to example 436, wherein the tissue-engaging element is a dart.

Example 439. The apparatus according to example 436, wherein the tissue-engaging element is a staple.

Example 440. The apparatus according to any one of examples 436-439, wherein the tissue-engaging element comprises and/or is formed from titanium.

Example 441. The apparatus according to any one of examples 436-440, wherein the tissue-engaging element is formed by powder bed fusion.

Example 442. The apparatus according to any one of examples 436-440, wherein the tissue-engaging element is formed by electron beam melting.

Example 443. The apparatus according to any one of examples 436-440, wherein the tissue-engaging element is formed by powder-fed directed-energy deposition.

Example 444. An anchor usable and/or for use at a real or simulated tissue of a real or simulated heart of a real or simulated subject, the anchor comprising: (A) a head; and/or (B) a tissue-engaging element formed from a structural material, and/or having a coating with which the structural material is coated, the coating being: (i) of a material other than the structural material, and/or (ii) etched in a manner that provides the tissue-engaging element with a textured surface.

Example 445. The apparatus according to example 444, wherein the tissue-engaging element is helical and configured to be screwed along the anchor axis into the tissue.

Example 446. The apparatus according to example 444, wherein the tissue-engaging element is a dart.

Example 447. The apparatus according to example 444, wherein the tissue-engaging element is a staple.

Example 448. The apparatus according to any one of examples 444-447, wherein the structural material is coated with the coating by electroplating.

Example 449. The apparatus according to any one of examples 444-448, wherein the structural material is steel.

Example 450. The apparatus according to any one of examples 444-448, wherein the coating is gold.

Example 451. The apparatus according to any one of examples 444-450, wherein the tissue-engaging element is etched by laser etching.

Example 452. The apparatus according to any one of examples 444-450, wherein the tissue-engaging element is etched by ion-beam etching.

Example 453. The apparatus according to any one of examples 444-452, wherein the tissue-engaging element is etched sufficiently shallowly that the coating is not completely penetrated by the etching.

Example 454. A method, comprising: (A) absorbing a substance into a snood disposed around a head of an anchor, the head including an interface, the anchor including a tissue-engaging element that is coupled to the interface, and/or the snood being disposed around the anchor head in a manner that preserves accessibility to the interface; and/or (B) performing a procedure on a real or simulated subject, the procedure comprising: (i) to a real or simulated heart of the subject, transluminally advancing the anchor with the snood carrying the absorbed substance; and/or (ii) driving the tissue-engaging element into tissue of the heart by applying an anchoring force to the interface.

Example 455. The system according to example 454, wherein the substance includes a medicament, and/or wherein absorbing the substance into the snood comprises absorbing the medicament into the snood.

Example 456. The system according to any one of examples 454-455, wherein the substance includes a radiopaque dye, and/or wherein absorbing the substance into the snood comprises absorbing the radiopaque dye into the snood.

Example 457. The method according to any one of examples 454-456, wherein the procedure is performed in an operating theater, and/or the step of absorbing is performed in the operating theater.

Example 458. The method according to any one of examples 454-457, wherein the step of absorbing is performed no more than two hours prior to transluminally advancing the anchor.

Example 459. The method according to any one of examples 454-458, wherein: (i) advancing the anchor comprises advancing the anchor using a driver that is engaged with the interface, (ii) driving the tissue-engaging element comprises driving the tissue-engaging element by applying the anchoring force to the interface using the driver, and/or (iii) absorbing the substance comprises absorbing the substance while the driver is engaged with the interface.

Example 460. The method according to example 459, wherein absorbing the substance comprises dipping the anchor into the substance using the driver while the driver is engaged with the interface.

Example 461. A method, comprising manufacturing textile components for implantable anchors by: (A) weaving a textile into an elongate form that includes a first tubular structure and a second tubular structure that are connected to, and/or parallel with, each other; and/or (B) slicing the elongate form into transverse slices, each slice defining a respective textile component that includes: (i) a first ring that is derived from the first tubular structure and that is configured to serve as a collar of the anchor; and/or (ii) a second ring that is derived from the second tubular structure, that is connected to the first ring, and/or that is configured to serve as an eyelet of the anchor.

Example 462. The method according to example 461, wherein weaving the textile into the elongate form comprises weaving the textile into the elongate form such that the first tubular structure has a larger inner diameter than the second tubular structure.

Example 463. The method according to any one of examples 461-462, wherein weaving the textile into the elongate form comprises weaving the textile into the elongate form such that the first tubular structure and the second tubular structure extend in parallel along a warp axis of the textile.

Example 464. The method according to any one of examples 461-463, further comprising, for each of the textile components, rotatably mounting the first ring on a head of the anchor such that the first ring couples the second ring to the head in a manner in which the second ring is revolvable around the head.

Example 465. A method, comprising manufacturing textile components for implantable anchors by: (i) weaving a first elongate form that includes a first tubular structure; (ii) weaving a second elongate form that includes a second tubular structure; (iii) slicing the first elongate form into first transverse slices, each defining a first ring that is derived from the first tubular structure; (iv) slicing the second elongate form into second transverse slices, each defining a second ring that is derived from the second tubular structure; and (v) for each of the textile components, forming the textile component by interlinking one of the first rings with one of the second rings such that the first ring is configured to serve as a collar of the anchor, and/or the second ring is configured to serve as an eyelet of the anchor.

Example 466. The method according to example 465, wherein weaving the second elongate form comprises weaving the second elongate form such that the second tubular structure has a smaller inner diameter than the first tubular structure.

Example 467. The method according to any one of examples 465-466, wherein weaving the first elongate form comprises weaving the textile into the first elongate form such that the first tubular structure extends along a warp axis of the textile.

Example 468. The method according to any one of examples 465-467, wherein weaving the second elongate form comprises weaving the textile into the second elongate form such that the second tubular structure extends along a warp axis of the textile.

Example 469. The method according to any one of examples 465-468, further comprising, for each of the textile components, rotatably mounting the first ring on a head of the anchor such that the first ring couples the second ring to the head in a manner in which the second ring is revolvable around the head.

Example 470. A method, comprising: (i) weaving a textile into a strip having a first slit and a second slit defined therethrough; and/or (ii) rotatably mounting the strip on a head of an implantable anchor by placing the head through the first slit such that the first slit serves as a collar-aperture, and/or the second slit serves as an eyelet-aperture that is revolvable around the head.

Example 471. The method according to example 470, wherein weaving the textile comprises weaving the textile such that the first slit is longer than the second slit.

Example 472. The method according to any one of examples 470-471, wherein weaving the textile comprises weaving the textile such that the first slit and the second slit are collinear with each other.

Example 473. The method according to any one of examples 470-472, wherein weaving the textile comprises weaving the textile such that the first slit and the second slit are parallel with a warp axis of the textile.

Example 474. A system useable and/or for use with a real or simulated tissue of a real or simulated subject, the system comprising: (A) a catheter device, comprising: (i) a flexible tube that has: (a) a distal opening that is configured to be transluminally advanced toward the tissue, and/or (b) a proximal end that defines a proximal opening; and/or (ii) an extracorporeal unit, coupled to the proximal end of the tube, and/or comprising: (a) a body; and/or (b) a series of cartridges, mounted on the body in an imbricated manner; and/or (B) a series of anchors, each housed by a corresponding cartridge of the series of cartridges.

Example 475. The system according to example 474, further comprising a tether threaded through each of the anchors of the series.

Example 476. A system useable and/or for use with a real or simulated tissue of a real or simulated subject, the system comprising: (A) a catheter device, comprising: (i) a flexible tube that has a distal opening that is configured to be transluminally advanced toward the tissue, and/or (ii) an extracorporeal unit, coupled to a proximal end of the tube; and/or (B) a series of anchors, mounted on the body in an imbricated manner.

Example 477. The system according to example 474, further comprising a tether threaded through each of the anchors of the series.

Example 478. Apparatus, comprising an implant that comprises: (A) a tether; and/or (B) a series of anchors, each comprising: (i) an anchor head; (ii) a tissue-engaging element: (a) extending distally away from the anchor head to define an anchor axis of the anchor, and/or (b) configured to be driven along the anchor axis into tissue of a real or simulated subject; and/or (iii) a textile and/or polymer through which the tether is threaded in a manner that slidably couples the anchor to the tether.

Example 479. A system for treating a real or simulated subject, the system comprising: (A) a support assembly that comprises a track; (B) a first catheter, comprising: (i) a first-catheter flexible tube, and/or (ii) a first-catheter extracorporeal unit, coupled to a proximal part of the first-catheter flexible tube, and/or slidably mountable on the track such that the first-catheter flexible tube extends distally away from the track and into the subject; (C) an implant catheter, comprising: (i) an implant-catheter flexible tube, and/or (ii) an implant-catheter extracorporeal unit, coupled to a proximal part of the implant-catheter flexible tube, and/or slidably mountable on the track proximally from the first-catheter extracorporeal unit such that: (a) the implant-catheter flexible tube extends distally away from the track and through the first-catheter flexible tube, and/or (b) a distance along the track between the implant-catheter extracorporeal unit and the first-catheter extracorporeal unit is adjustable; (D) an implant, mounted on the implant catheter, and/or transluminally implantable in the subject using the implant catheter; and/or (E) an adjustment tool, comprising: (i) a flexible shaft, and/or (ii) an adjustment-tool extracorporeal unit, coupled to a proximal part of the flexible shaft, the adjustment tool being configured to be switched with the implant catheter subsequent to implantation of the implant such that: (a) the adjustment-tool extracorporeal unit becomes slidably mounted on the track proximally from the first-catheter extracorporeal unit, (b) the flexible shaft becomes disposed through the first-catheter flexible tube, extending distally away from the track and toward the implant, and/or (c) a distance along the track between the adjustment-tool extracorporeal unit and the first-catheter extracorporeal unit is adjustable.

Example 480. The system according to example 479, wherein: (i) the system further comprises a second catheter, comprising: (a) a second-catheter flexible tube, and/or (b) a second-catheter extracorporeal unit, coupled to a proximal part of the second-catheter flexible tube, and/or slidably mountable on the track proximally from the first-catheter extracorporeal unit such that: (I) the second-catheter flexible tube extends distally away from the track and through the first-catheter flexible tube, and/or (II) a distance along the track between the second-catheter extracorporeal unit and the first-catheter extracorporeal unit is adjustable, (ii) the implant-catheter extracorporeal unit is slidably mountable on the track proximally from the first-catheter extracorporeal unit and the second-catheter extracorporeal unit such that: (a) the implant-catheter flexible tube extends distally away from the track and, within the second-catheter flexible tube, through the first-catheter flexible tube, and/or (b) a distance along the track between the implant-catheter extracorporeal unit and the second-catheter extracorporeal unit is adjustable, and/or (iii) the adjustment-tool is configured to be switched with the implant catheter and the second catheter subsequently to implantation of the implant such that: (a) the adjustment-tool extracorporeal unit becomes slidably mounted on the track proximally from the first-catheter extracorporeal unit, and/or (b) the flexible shaft becomes disposed through the first-catheter flexible tube, absent the second-catheter flexible tube, extending distally away from the track and toward the implant.

Example 481. A system comprising: (A) a catheter device, comprising: (i) a flexible tube, the flexible tube comprising a distal opening positioned at a distal end of the flexible tube and a proximal opening positioned at a proximal end of the flexible tube; and/or (ii) an extracorporeal unit, coupled to the proximal end of the flexible tube, the extracorporeal unit comprising: (a) a body, and/or (b) a series of cartridges, distributed along a proximal-distal axis of the body in a manner that defines a proximal-distal axis, such that a distalmost cartridge of the series of cartridges is closest to the proximal opening; (B) a tether; and/or (C) a series of anchors, wherein an anchor of the series of anchors: (i) is housed in a cartridge of the series of cartridges, and/or (ii) is coupled to the tether such that the tether extends along the body, parallel with the proximal-distal axis.

Example 482. The system according to example 481, wherein the anchor comprises: (i) an anchor head; (ii) a tissue-engaging element: (a) extending distally away from the anchor head to define an anchor axis of the anchor, and/or (b) configured to be driven along the anchor axis into the tissue; and/or (iii) a textile and/or polymer, shaped to define an eyelet threaded onto the tether in a manner that slidably couples the anchor to the tether.

Example 483. The system according to any one of examples 481-482, wherein the series of anchors comprises a leading anchor and one or more subsequent anchors, such that the leading anchor is housed in the distalmost cartridge and is fixed to the tether.

Example 484. The system according to example 483, wherein the one or more subsequent anchors are configured to be slidably coupled to tether.

Example 485. The system according to any one of examples 481-484, wherein the anchor comprises: (i) a head, slidably coupled to the tether, and/or (ii) a tissue-engaging element, extending away from the head to define an anchor axis of the anchor, and wherein the anchor is housed in the cartridge such that the anchor axis lies obliquely with respect to the proximal-distal axis.

Example 486. The system according to any one of examples 481-485, wherein the cartridge: (i) has a closed state in which the cartridge securely houses the anchor, (ii) defines a respective cartridge vector that is oblique with respect to the proximal-distal axis, and/or (iii) is transitionable into an open state in which the corresponding anchor is removable from the cartridge, the transition in response to at least part of the cartridge sliding along the cartridge vector.

Example 487. The system according to example 486, wherein the cartridge is associated with a threshold force, and/or is further configured to transition into the open state responsive to the anchor being pulled with a force that exceeds the threshold force.

Example 488. The system according to any one of examples 481-487, wherein the tether comprises (i) a distal end coupled to a leading anchor, and/or (ii) a proximal end releasably secured within the extracorporeal unit.

Example 489. The system according to example 488, wherein the extracorporeal unit comprises a de-slacker that comprises a winch that is spring-loaded in a manner that takes up slack in the tether.

Example 490. The system according to example 489, wherein the de-slacker comprises a deactivation switch configured to deactivate the de-slacker in a manner that allows slack to be introduced to the tether and not taken up by the winch.

Example 491. The system according to any one of examples 481-490, further comprising multiple spacers threaded on the tether, alternatingly with anchors of the series of anchors.

Example 492. The system according to example 491, wherein a spacer of the multiple spacers is tubular, and/or is threaded on the tether by the tether extending through a lumen defined by the spacer.

Example 493. The system according to example 492, wherein the spacer is arranged on the tether such that, upon advancement of the anchor distally along the tether toward the proximal opening, the spacer trails the anchor.

Example 494. The system according to example 491, wherein a first spacer of the multiple spacers is connected to a leading anchor of the series of anchors, and/or the first spacer of the multiple spacers is less axially compressible than at least another of the multiple spacers.

Example 495. The system according to any one of examples 481-494, wherein: (i) the anchor of the series of anchors comprises: (a) an anchor head; and/or (b) a helical tissue-engaging element, extending away from the anchor head to define an anchor axis of the anchor, and/or configured to be screwed along the anchor axis into the tissue; (ii) the flexible tube comprises: (a) along a tube axis of the flexible tube, a channel, through which the anchor is slidable toward the distal opening, and/or (b) at the distal end, a grip zone at which the flexible tube comprises a grip surface, the grip surface configured to inhibit sliding of the anchor through the grip zone by gripping a lateral surface of the helical tissue-engaging element; and/or (iii) the system further comprises an anchor driver configured to: (a) slide the anchor distally through the channel to the grip zone, and/or (b) drive the anchor through the grip zone by screwing the helical tissue-engaging element over the grip surface.

Example 496. The system according to example 495, wherein the grip surface is configured such that, as the driver screws the helical tissue-engaging element over the grip surface, the helical tissue-engaging element temporarily compresses parts of the grip surface with which the helical tissue-engaging element is in contact.

Example 497. The system according to any one of examples 495-496, wherein the grip surface comprises at least one resilient nub that protrudes medially into the channel.

Example 498. The system according to any one of examples 495-496, wherein the grip surface comprises at least one resilient rib that protrudes medially into the channel.

Example 499. The system according to any one of examples 495-498, wherein: (i) the anchor further comprises an eyelet, mounted on the anchor head so as to be revolvable about the anchor axis, and/or (ii) proximal from the rib, the flexible tube further defines an abutment that protrudes medially into the channel in a manner that, as the anchor driver screws the tissue-engaging element over the grip surface, inhibits revolution of the eyelet about the anchor axis.

Example 500. The system according to any one of examples 481-499, wherein the anchor comprises: (i) an anchor head; (ii) a tissue-engaging element: (a) extending distally away from the anchor head to define an anchor axis of the anchor, and/or (b) configured to be driven along the anchor axis into the tissue; and/or (iii) a textile and/or polymer, shaped to define an eyelet, the anchor being coupled to the tether via the eyelet.

Example 501. The system according to example 500, wherein the anchor head comprises an interface that is coupled to the tissue-engaging element, the tissue-engaging element configured to be driven along the anchor axis into the tissue by an anchoring force applied to the interface.

Example 502. The system according to any one of examples 481-501, wherein the catheter device further comprises a de-slacker, coupled to the tether, and/or configured to eliminate slack in the tether.

Example 503. The system according to any one of examples 481-502, further comprising an anchor driver: (i) comprising a flexible shaft, and/or a drive head at a distal end of the shaft, and/or (ii) configured to: (a) engage the drive head with the anchor, (b) remove the anchor from the corresponding cartridge, and/or (c) while the anchor remains coupled to the tether, advance the anchor into the proximal opening and through the flexible tube toward the tissue, and/or anchor the anchor to the tissue.

Example 504. The system according to example 503, wherein the anchor driver is configured to remove the anchor from the cartridge by applying a pulling force to the anchor such that the cartridge transitions into an open state.

Example 505. The system according to example 505, further comprising an elongate adjustment tool and a lock, the adjustment tool configured to: (i) advance the lock distally along the tether into the subject and toward the tissue, (ii) apply tension to the tether, (iii) lock the tension in the tether by locking the lock to the tether, (iv) cut the tether proximally from the lock, and/or (v) leave the lock in the subject locked to the tether.

Example 506. The system according to example 505, wherein: (i) the extracorporeal unit comprises a catheter-device extracorporeal unit, (ii) the adjustment tool comprises an adjustment-tool extracorporeal unit, a shaft extending distally from the adjustment-tool extracorporeal unit, and/or a tool head at a distal end of the shaft, and/or (iii) the adjustment tool is configured to advance the lock distally along the tether into the subject and toward the tissue while the lock is housed within the tool head.

Example 507. The system according to example 506, wherein the tether has (i) a distal end at a leading anchor of the series of anchors, and/or (ii) a proximal end secured within the extracorporeal unit, and/or releasable from within the extracorporeal unit so as to be threadable proximally into an aperture of the lock, through the lock and into the shaft of the adjustment tool.

Example 508. The system according to example 507, wherein: (i) the adjustment tool comprises an uptake assembly that comprises: (a) at a working end of the uptake assembly, a gripper disposed proximally from the lock such that, in a receiving state of the uptake assembly, threading of the proximal end of the tether proximally into the aperture of the lock, through the lock and the tool head, and/or into the shaft of the adjustment tool, causes the working end of the uptake assembly to receive the proximal end of the tether, and/or (b) a knob: (I) mounted on a body of the adjustment-tool extracorporeal unit, and/or (II) operably coupled to a proximal part of the gripper such that operation of the knob transitions the uptake assembly into a grip state in which the gripper grips the tether, (ii) the mounting of the knob on the adjustment-tool extracorporeal unit is such that transitioning of the uptake assembly into the grip state releases the knob from the adjustment-tool extracorporeal unit, and/or (iii) once released from the adjustment-tool extracorporeal unit, the knob is removable from the adjustment-tool extracorporeal unit in a manner that withdraws the working end of the uptake assembly, along with the proximal end of the tether, proximally through and out from the shaft and the adjustment-tool extracorporeal unit such that the tether becomes positioned through the lock, the tool head, the shaft, and/or the adjustment-tool extracorporeal unit.

Example 509. The system according to example 508, wherein: (i) the lock is biased to a locked position, (ii) the adjustment tool comprises an obstructor tube extending distally through the shaft and into the tool head such that a distal part of the obstructor tube is disposed within the lock in a manner that constrains the lock unlocked, and/or (iii) while the knob of the uptake assembly remains mounted on the adjustment-tool extracorporeal unit, the working end of the uptake assembly is disposed within the obstructor tube, such that removal of the knob from the adjustment-tool extracorporeal unit withdraws the working end of the uptake assembly, along with the proximal end of the tether, proximally through and out from the obstructor tube such that the tether becomes positioned through one or more of the lock, the tool head, the obstructor tube within the shaft, and/or the adjustment-tool extracorporeal unit.

Example 510. The system according to example 508, wherein: (i) the lock is biased to lock, (ii) the adjustment tool comprises: (a) a guillotine within the tool head and proximal from the lock, and/or (b) an obstructor extending distally through the shaft and the guillotine such that a distal part of the obstructor is disposed within the lock in a manner that constrains the lock unlocked, (iii) the adjustment-tool extracorporeal unit comprises a lock-and-cut subassembly that comprises: (a) a locking block, coupled to the obstructor, and/or (b) a lock-and-cut controller, (iv) withdrawal of the working end of the uptake assembly along with the proximal end of the tether, proximally through and out from the shaft and the adjustment-tool extracorporeal unit, leaves the tether positioned through the lock and the guillotine such that (I) subsequent locking of the lock locks the lock to the tether, and/or (II) subsequent actuation of the guillotine cuts the tether proximally from the lock, and/or (v) the lock-and-cut controller is operatively coupled to the locking block such that operation of the lock-and-cut controller draws the locking block proximally such that the obstructor becomes withdrawn from the lock and the lock responsively locks to the tether.

Example 511. The system according to example 508, wherein the adjustment-tool extracorporeal unit comprises a tensioning subassembly that comprises: (A) a tensioning block, (B) a clamp, attached to the tensioning block, and/or (C) a tensioning controller, wherein: (i) while the knob of the uptake assembly remains mounted on the adjustment-tool extracorporeal unit, the gripper extends from the knob, distally through the clamp and the shaft to the working end, (ii) withdrawal of the working end of the uptake assembly, along with the proximal end of the tether, proximally through and out from the shaft and the adjustment-tool extracorporeal unit withdraws the gripper from the clamp, leaving the tether positioned through the clamp such that subsequent operation of the clamp locks the tether to the tensioning block, and/or (iii) the tensioning controller is operatively coupled to the tensioning block such that, while the tether remains locked to the tensioning block, operation of the tensioning controller applies tension to the tether by drawing the tensioning block and the tether proximally.

Example 512. The system according to example 507, wherein: (i) the adjustment tool comprises an uptake assembly that comprises: (a) a sleeve extending distally through the shaft and terminating proximally from the lock, (b) a gripper extending distally through the sleeve and having a widened distal portion disposed distally outside of the sleeve, the sleeve and the gripper being shaped and positioned such that threading of the proximal end of the tether proximally into the shaft of the adjustment tool advances the proximal end of the tether proximally around the widened distal portion of the gripper and into the sleeve, and/or (c) a knob: (I) mounted on the adjustment-tool extracorporeal unit, and/or (II) operably coupled to a proximal part of the sleeve and to a proximal part of the gripper such that operation of the knob grips the tether within the sleeve by transitioning the uptake assembly into a grip state by drawing the widened distal portion of the gripper proximally into the sleeve, (ii) the mounting of the knob on the adjustment-tool extracorporeal unit is such that transitioning of the uptake assembly into the grip state releases the knob from the adjustment-tool extracorporeal unit, and/or (iii) once released from the adjustment-tool extracorporeal unit, the knob is removable from the adjustment-tool extracorporeal unit in a manner that pulls the sleeve and the gripper, along with the proximal end of the tether, proximally through the shaft and the adjustment-tool extracorporeal unit, and/or out of the adjustment tool such that the tether extends through the lock, the tool head, the shaft, and/or the adjustment-tool extracorporeal unit.

Example 513. A system useable and/or for use with a real or simulated tissue of a real or simulated subject, the system comprising: (A) a catheter device, comprising: (i) a flexible tube that has: (a) a distal opening that is configured to be transluminally advanced toward the tissue, and/or (b) a proximal end that defines a proximal opening; and/or (ii) an extracorporeal unit, coupled to the proximal end of the tube, and/or comprising: (a) a body, and/or (b) a series of cartridges; (B) a tether; and/or (C) a series of anchors coupled to the tether, each anchor of the series of anchors: (i) comprising an anchor head, and/or a tissue-engaging element that extends distally away from the anchor head to define an anchor axis of the anchor, and/or (ii) housed by a corresponding cartridge of the series of cartridges, wherein: (a) the series of anchors comprises a first subset of the anchors and a second subset of the anchors, (b) the first subset contains a first 2-6 of the anchors of the series, (c) the second subset contains more anchors than does the first subset, (d) for each of the anchors of the first subset, the tissue-engaging element has a first width, and/or (e) for each of the anchors of the second subset, the tissue-engaging element has a second width that is smaller than the first width.

Example 514. The system according to example 513, wherein, for each of the anchors of the series, the tissue-engaging element is a helical tissue-engaging element that extends helically away from the anchor head and that is configured to be screwed along the anchor axis into the tissue.

Example 515. The system according to any one of examples 513-514, wherein the first 2-6 of the anchors of the series is a first 4 of the anchors of the series, and/or wherein the second subset contains the first 4 anchors of the series.

Example 516. The system according to any one of examples 513-515, wherein the second subset contains 4-18 of the anchors of the series.

Example 517. The system according to any one of examples 513-516, wherein the second subset contains a remainder of the anchors of the series.

Example 518. The system according to any one of examples 513-517, further comprising an anchor driver: (i) comprising a flexible shaft, and/or a drive head at a distal end of the shaft, and/or (ii) configured to, for each of the anchors of the series sequentially, beginning with the anchors of the first subset: (a) engage the drive head with the anchor head, (b) remove the anchor from the corresponding cartridge, and/or (c) while the anchor remains coupled to the tether, advance the anchor into the proximal opening and through the flexible tube toward the tissue, and/or drive the tissue-engaging element into the tissue.

Example 519. Apparatus, useable and/or for use with a real or simulated tissue of a real or simulated subject, the apparatus comprising: (A) an implant, comprising: (i) a tether; and/or (ii) a series of anchors coupled to the tether, each anchor of the series of anchors comprising: (a) an anchor head, and/or (b) a tissue-engaging element that extends distally away from the anchor head to define an anchor axis of the anchor, the series of anchors comprising (I) a first subset of the anchors of the series, which contains a first 2-6 of the anchors of the series, the tissue-engaging element of each anchor of the first subset having a first width, and/or (II) a second subset of the anchors of the series, which contains more anchors than does the first subset, the tissue-engaging element of each anchor of the second subset having a second width that is smaller than the first width; and/or (B) an anchor driver: (i) comprising a flexible shaft, and/or a drive head at a distal end of the shaft, and/or (ii) configured to, for each of the anchors of the series sequentially, beginning with the anchors of the first subset: (a) engage the drive head with the anchor head, and/or (b) while the anchor remains coupled to the tether, advance the anchor transluminally toward the tissue, and/or drive the tissue-engaging element into the tissue.

Example 520. The system, apparatus, or method according to any one of the above examples, wherein the catheter device, implant, anchors, tether, anchor driver, adjustment tool, and/or support assembly is sterilized.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A system for use with a tissue of a subject, the system comprising:
a catheter device, comprising:
a flexible tube that has:
a distal opening that is configured to be transluminally advanced toward the tissue, and
a proximal end that defines a proximal opening; and
an extracorporeal unit, coupled to the proximal end of the tube, and comprising:
a body, and
a series of cartridges, distributed along a proximal-distal axis of the body, with a distalmost cartridge of the series of cartridges being closest to the proximal opening;
a tether; and
a series of anchors, including a leading anchor and other anchors, each anchor of the series of anchors:
housed by a corresponding cartridge of the series of cartridges, with the leading anchor housed by the distalmost cartridge, and
coupled to the tether such that the tether extends along the body, parallel with the proximal-distal axis.

2. The system according to claim 1, wherein the cartridges of the series of cartridges are imbricated.

3-5. (canceled)

6. The system according to claim 1, further comprising a tensioner, housed by the extracorporeal unit, and configured to:
engage an intermediate region of the tether, the intermediate region of the tether being at the extracorporeal unit, and
apply tension to the tether by pulling on the intermediate region of the tether.

7-8. (canceled)

9. The system according to claim 1, wherein each of the cartridges:
has a closed state in which the cartridge securely houses the corresponding anchor,
defines a respective cartridge vector that is oblique with respect to the proximal-distal axis, and
is, by at least part of the cartridge being slid along the cartridge vector, transitionable into an open state in which the corresponding anchor is removable from the cartridge.

10-11. (canceled)

12. The system according to claim 9, wherein the cartridge vector is oblique with respect to the proximal-distal axis.

13. The system according to claim 9, wherein the cartridge vectors of the series of cartridges collectively define a common cartridge plane on which the cartridge vectors lie, and the tether extends along the body, parallel with the common cartridge plane.

14-16. (canceled)

17. The system according to claim 1, wherein each anchor of the series:
comprises:
a head, coupled to the tether, and
a tissue-engaging element, extending away from the head to define an anchor axis of the anchor, and
is housed by a corresponding cartridge such that the anchor axis lies obliquely with respect to the proximal-distal axis.

18. The system according to claim 1, wherein;
the tether has (i) a distal end at the leading anchor, and (ii) a proximal end releasably secured within the extracorporeal unit, and
the extracorporeal unit comprises a de-slacker that comprises:
a winch that is spring-loaded in a manner that takes up slack in the tether, and
a deactivation switch that is user-operable to deactivate the de-slacker in a manner that allows slack to be introduced to the tether and not taken up by the winch.

19-20. (canceled)

21. The system according to claim 1, further comprising:
multiple spacers threaded on the tether, alternatingly with the anchors of the series, and
multiple connectors, each connecting a corresponding one of the spacers to a corresponding anchor of the series.

22. The system according to claim 21, further comprising at least one free spacer, separate from the tether, and manually threadable onto the tether between anchors without access to an end of the tether.

23. (canceled)

24. The system according to claim 2321, wherein each of the spacers is formed from a fabric.

25-28. (canceled)

29. The system according to claim 21, wherein each of the connectors provides a frangible connection between the corresponding spacer and the corresponding anchor.

30. (canceled)

31. The system according to claim 21, wherein each of the spacers is arranged on the tether such that, upon advancement of the corresponding anchor distally along the tether toward the proximal opening, the corresponding anchor tows the corresponding spacer via the corresponding connector, the corresponding spacer trailing the corresponding anchor distally along the tether.

32. (canceled)

33. The system according to claim 1, wherein:
each anchor of the series comprises:
an anchor head; and
a helical tissue-engaging element, extending away from the anchor head to define an anchor axis of the anchor, and configured to be screwed along the anchor axis into the tissue;
the flexible tube has a distal portion that includes the distal opening, the flexible tube defining:
along a tube axis of the flexible tube, a channel through which the anchor is slidable toward the distal opening, and
at the distal portion, a grip zone at which the flexible tube has a grip surface that inhibits sliding of the anchor through the grip zone by gripping a lateral surface of the helical tissue-engaging element; and
the system further comprises an anchor driver configured to:
slide the anchor distally through the channel to the grip zone, and
drive the anchor through the grip zone by screwing the helical tissue-engaging element over the grip surface.

34-42. (canceled)

43. The system according to claim 1, wherein, each of the anchors comprises:
an anchor head;
a tissue-engaging element:
extending distally away from the anchor head to define an anchor axis of the anchor, and
configured to be driven along the anchor axis into the tissue; and
a textile, shaped to define an eyelet, the anchor being coupled to the tether by the eyelet being coupled to the tether.

44-56. (canceled)

57. The system according to claim 43, wherein, for each of the anchors, the textile is further shaped to define a collar that couples the eyelet to the anchor head such that the eyelet is revolvable about the anchor axis by the collar rotating about the anchor axis.

58-63. (canceled)

64. The system according to claim 57, wherein the textile is a yarn, the collar and the eyelet being formed by knotting the yarn.

65-70. (canceled)

71. The system according to claim 1, further comprising an anchor driver:
comprising a flexible shaft, and a drive head at a distal end of the shaft, and
configured to, for each of the anchors sequentially, beginning with the leading anchor:
engage the drive head with the anchor,
remove the anchor from the corresponding cartridge, and
while the anchor remains coupled to the tether, advance the anchor into the proximal opening and through the flexible tube toward the tissue, and anchor the anchor to the tissue.

72. The system according to claim 71, further comprising an elongate adjustment tool and a lock, the adjustment tool configured to:
advance the lock distally along the tether into a heart of the subject and toward the tissue,
apply tension to the tether,
lock the tension in the tether by locking the lock to the tether,
cut the tether proximally from the lock, and
leave the lock in the heart locked to the tether.

73-78. (canceled)

79. The system according to claim 71, wherein the extracorporeal unit is shaped to define, proximally from the series of cartridges, a rest in which the shaft is restable while the anchor driver anchors the anchor to the tissue.

80-87. (canceled)
